(19) United States
(12) Patent Application Publication
(10) Pub. No.: US 2017/0184604 A1
(43) Pub. Date: Jun. 29, 2017

(54) DD1ALPHA RECEPTOR AND USES THEREOF IN IMMUNE DISORDERS

(71) Applicant: THE GENERAL HOSPITAL CORPORATION, Boston, MA (US)

(72) Inventors: Sam W LEE, Newton, MA (US); Anna MANDINOVA, Newton, MA (US)

(73) Assignee: THE GENERAL HOSPITAL CORPORATION, Boston, MA (US)

(21) Appl. No.: 15/312,940

(22) PCT Filed: May 22, 2015
(86) PCT No.: PCT/US15/32243

§ 371 Date: Nov. 21, 2016

Related U.S. Application Data
(60) Provisional application No. 62/001,943, filed on May 22, 2014.

(51) Int. Cl. G01N 33/574; C12N 15/113

(52) U.S. Cl. CPC G01N 33/57496; C12N 15/113; C12N 2310/11; C12N 2310/531; G01N 2800/52; G01N 2333/4748

(57) ABSTRACT

Described herein are methods and compositions for treatment of immune-related diseases or disorders by modulating DD1α activity, alone or in combination with modulation of PD-1 activity. In some embodiments, the methods and compositions described herein are directed to treatment of cancer and/or infections (e.g., bacterial infection, and/or fungal infection). In some embodiments, the methods and compositions described herein are directed to treatment of autoimmune diseases and/or inflammation. In some embodiments, the methods and compositions described herein are directed to treatment of asthma, and allergy. Methods for identifying patients who are more likely to be responsive to and benefit from an immunotherapy that targets DD1α and/or PD-1 activity or expression are also described herein.

### CROSS-REFERENCE TO RELATED APPLICATIONS

This Application claims benefit under 35 U.S.C. §119(e) of the U.S. Provisional Application No. 62/001,943 filed May 22, 2014, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jul. 10, 2015, is named 030258-080941-PCT_SL.txt and is 24,240 bytes in size.

### FIELD OF THE INVENTION

Various aspects described herein provide for methods and compositions for treatment of immune-related diseases or disorders and/or therapy monitoring. In some embodiments, the methods and compositions described herein are directed to treatment and/or therapy monitoring of cancer. In some embodiments, the methods and compositions described herein are directed to treatment and/or therapy monitoring of inflammatory diseases such as infections, allergy, asthma, autoimmune diseases and/or inflammation. Methods for identifying patients who are more likely to be responsive to and benefit from a treatment that modulates DD1α activity and/or PD-1 activity are also described herein.

### BACKGROUND

During development, tissue restoration, and response to injury, numerous cells are damaged and destined to undergo cell death/apoptosis, demanding rapid and efficient clearance of cell corpses as a vital prerequisite for homeostatic maintenance of tissue health (1, 2). Over the past decades, enormous efforts have been made toward understanding various mechanisms of cell death at the level of the dying cells (3). It has also been recognized that subsequent failure to properly clear dying cells can lead to the accumulation of autoantigens in tissues that foster diseases such as chronic inflammatory diseases, autoimmune diseases and developmental abnormalities (1, 4, 5). Engulfment of apoptotic cells is an active process coordinated by receptors on phagocytes and ligands on apoptotic cells (6). The exposure of phosphatidylserine (PtdSer) on the surface of dying cells is known to be a key “eat-me” signal facilitating recognition and clearance by neighboring phagocytes (7). PtdSer on apoptotic cells can be recognized directly via phagocytic receptors, such as BAIL TIM1, TIM3, TIM4 and Stabilin-2, or indirectly via soluble bridging proteins (MFG-E8 and C1q) to engage in engulfment of apoptotic cells (8-14). In addition to recognizing the “eat-me” signals on apoptotic cells, phagocytes can discriminate between live and dead cell targets by the occurrence of “don't-eat-me” signals on the surface of living cells (15). CD47 is a broadly expressed membrane protein that interacts with the myeloid inhibitory immunoreceptor SIRPα and engagement of SIRPα by CD47 provides a downregulatory signal that inhibits host cell phagocytosis (16, 17). Therefore, CD47 functions as a “don't-eat-me” signal and is down-regulated to permit apoptosis. Recently a cancer therapeutic strategy to block the “don't eat me” signal with monoclonal antibody or SIRPα monomers against CD47 has been successful by preventing cancer cells from escaping engulfment by macrophages (18-20).

The p53 tumor suppressor is indispensable for maintenance of genomic integrity (21, 22). p53 functions as a transcription factor that is activated by various cellular stresses and governs multiple core programs in cells, including cell cycle arrest and apoptosis (23-25). In recent years, p53 has been implicated in immune responses and inflammatory diseases, with various roles in the immune system becoming apparent (26-32). Several IFN-inducible genes such as IRF5, IRF9, ISG15, and TLR3 are known to be p53 targets (26, 33-35). Among others, it has been reported that the expression of Foxp3, a master regulator of regulatory T cells (Tregs), is upregulated by p53 and the number of Tregs was significantly reduced in p53-deficient mice, suggesting that p53 induces Foxp3+ Tregs and suppresses autoimmunity (36, 37). Further, a connection between p53 tumor suppression and enhanced immune response has been proposed recently, showing that p53 stimulates the innate immunity by modulating macrophage function to non-cell-autonomously suppress tumorigenesis (38, 39). However, little is known about key factors of the immune systems that are associated with the level and/or activity of p53. There remains a need for novel compositions and approaches to treating immune-related disorders including, e.g., cancer.

### SUMMARY

Provided herein are compositions and methods based, in part, on the discovery that there is a T cell and macrophage signaling axis involving p53, DD1α and PD-1/PD-L1. In one aspect, the inventors have identified DD1α receptor as a post-apoptotic target gene of p53, which is induced in apoptotic cells and highly expressed in immune cells, e.g., but not limited to macrophages, dendritic cells, monocytes, myeloid cells and T cells. Further, the inventors have shown that DD1α can function as an “eat-me” signal-engulfment ligand of apoptotic cells. In another aspect, the inventors have discovered that DD1α, which shares homology to B7 family member PD-L1, functions as a negative immune checkpoint regulator that is involved in modulating immune response and/or T cell function associated with PD-1, a co-inhibitory immunoreceptor of T cell tolerance. The inventors have also shown that p53 induces expression and/or activity of DD1α, as well as PD-1 and its ligand PD-L1. Thus, p53 can serve as a guardian for immune integrity via p53, DD1α, PD-1 and/or PD-L1 signaling axis. Accordingly, not only can agents that modulate the activity and/or expression of p53, DD1α and PD-1/PD-L1 be used for treatment of immune related diseases or disorders such as autoimmune disease, infection, chronic inflammation, cancer, asthma, and allergy, but p53 can also be used as a predictive marker to identify subjects who are more likely to benefit from an immunotherapy targeting DD1α, PD-1, and/or PD-L1. Accordingly, various aspects described herein provide for methods of identifying subjects with an immune-related disease or disorder who are more likely to be responsive to an immunotherapy or a therapy that targets DD1α, PD-1, and/or PD-L1, as well as monitoring the treatment efficacy. Methods and compositions for treating subjects with an immune-related disease or disorder are also provided herein.

In some immune-related diseases or disorders, e.g., but not limited to cancer, asthma, allergy, and/or infection (e.g., bacterial and fungal infection), it can be desirable to enhance or promote one or more specific types of an immune response to produce a therapeutic effect. Examples of enhancing or promoting one or more specific types of an immune response include, but are not limited to increasing T cell proliferation, activating T cells, reversing T cell exhaustion, promoting a Th1 response, promoting a Th2 response, shifting Th1/Th2 balance in either direction, and a combination of two or more thereof. For example, in some embodiments, for cancer and chronic infections (e.g., bacterial and fungal infection), it can be desirable to enhance or promote an immune response, e.g., by modulating the Th1/Th2 balance toward a Th1 response. In some embodiments, for asthma and/or allergy, it can be desirable to enhance or promote an immune response, e.g., by dampening a Th2 response and/or shifting Th1/Th2 balance. In one aspect, the inventors have discovered that DD1α expressed on the surface of a tumor cell can reduce T-cell proliferation and prevent anti-tumor effects mediated by the immune system. This action can be attributed to the formation of a homodimer of DD1α/DD1α or a heterodimer of DD1α/PD-1. In contrast, in some other immune-related diseases or disorders, e.g., but not limited to autoimmune diseases, it can be desirable to suppress an immune response. Accordingly, some aspects provided herein relate to methods for determining whether a subject is amenable to treatment with an immunotherapy targeting PD-1 and/or DD1α. In some aspects, provided herein are methods and compositions for treating cancer and infection that target the homophilic binding interaction of DD1α with DD1α and/or the heterophilic interactions of PD-1 (e.g., binding interaction between DD1α and PD-1, PD-1 and PD-L1, and/or PD-1 and PD-L2).

One aspect provided herein relates to a method of identifying a cancer patient who is more likely to respond to an anti-DD1α and/or anti-PD-1 therapy. The method comprises: measuring the level of p53 activity or expression in a sample from a cancer patient; and comparing the level of p53 or expression in the sample with a p53 reference, and: (i) when the level of p53 activity or expression is greater than the p53 reference, the cancer patient is identified to be more likely to respond to an anti-DD1α and/or anti-PD-1 therapy; or (ii) when the level of p53 activity or expression is the same as or less than the p53 reference, the cancer patient is identified to be more likely to respond to an alternative, proinflammatory immunotherapy without an anti-DD1α and/or anti-PD-1 therapy. In some embodiments, the method further comprises identifying the cancer patient who is more likely to respond to an anti-DD1α and/or anti-PD-1 therapy, or who is more likely to benefit from an alternative proinflammatory immunotherapy without an anti-DD1α and/or anti-PD-1 therapy, based on the level of p53 activity and/or expression measured in the patient's sample.

Also provided herein, in another aspect, is a method of identifying a patient diagnosed to have asthma or allergy who is more likely to respond to an anti-DD1α and/or anti-PD-1 therapy, the method comprising: measuring the level of p53 activity or expression in a sample from a patient diagnosed to have asthma or allergy; and comparing the level of p53 or expression in the sample with a p53 reference, and: (i) when the level of p53 activity or expression is greater than the p53 reference, the patient is identified to be more likely to respond to an anti-DD1α and/or anti-PD-1 therapy; or (ii) when the level of p53 activity or expression is the same as or less than the p53 reference, the patient is identified to be more likely to respond to an alternative, proinflammatory immunotherapy without an anti-DD1α or anti-PD-1 therapy. In some embodiments, the method further comprises identifying the patient who is more likely to respond to an anti-DD1α and/or anti-PD-1 therapy, or who is more likely to benefit from an alternative proinflammatory immunotherapy without an anti-DD1α and/or anti-PD-1 therapy, based on the level of p53 activity and/or expression measured in the patient's sample.

In one embodiment of this aspect and all other aspects described herein, the method further comprises administering an anti-DD1α and/or anti-PD-1 therapy to the patient when the level of p53 activity or expression is greater than the p53 reference. In another embodiment of this aspect and all other aspects described herein, the method can further comprise increasing the dose of the anti-DD1α and/or anti-PD-1 therapy over a period of time. An anti-PD-1 therapy can comprise an agent that antagonizes the binding of PD-1 with PD-L1 and/or PD-L2. In some embodiments, the anti-PD-1 therapy can comprise a PD-1 inhibitor, a PD-L1 inhibitor, a PD-L2 inhibitor, or a combination of two or more thereof. In some embodiments, an anti-DD1α therapy can comprise an agent that inhibits homophilic interactions between DD1α molecules and/or an agent that inhibits heterophilic interactions between DD1α molecules and PD-1 molecules. In another embodiment of this aspect and all other aspects described herein, the inhibitor or agent used in the anti-DD1α and/or anti-PD-1 therapy can comprise a protein, a peptide, a nucleic acid, an antibody, a small molecule, a vaccine, and combinations thereof.

In one embodiment of this aspect and all other aspects described herein, the method further comprises administering an alternative, proinflammatory immunotherapy without an anti-DD1α and/or anti-PD-1 therapy when the level of p53 activity or expression is the same as or less than the p53 reference. An exemplary alternative, proinflammatory immunotherapy can comprise an activator of a proinflammatory T cell response pathway and/or a suppressor of an anti-inflammatory T cell response pathway. Non-limiting examples of the activator of the proinflammatory T cell response pathway and/or suppressor of the anti-inflammatory T cell response pathway include a TIGIT inhibitor, a Fgl2 inhibitor, a TIM-3 inhibitor, an anti-galectin-9 molecule, a CTLA-4 antagonist, a Lag-3 antagonist, an agonist of an immune checkpoint activating molecule, an antagonist of an immune checkpoint inhibitory molecule, or any combination thereof.

In another embodiment of this aspect and all other aspects described herein, the patient amenable to the methods described herein can be a patient that has been receiving a therapy to treat the target immune-related disease or disorder, e.g., anti-cancer therapy, anti-asthma therapy, or anti-allergy therapy. In some embodiments, the treatment can be an immunotherapy.

In this aspect and all other aspects described herein, a p53 reference used for comparison to a measured level of p53 activity or expression in a patient's sample generally involves a positive control, a negative control, and/or a threshold value. In some embodiments, the p53 reference can correspond to the level of p53 activity or expression in a normal healthy subject. In some embodiments, the p53 reference can correspond to the level of p53 activity or expression in a normal tissue of the same type or lineage as the sample (e.g., same type or lineage as a tissue biopsy obtained from a target site (e.g., a tumor or an inflammatory tissue). In some embodiments, the normal tissue of the same type or lineage as the sample can be obtained from a patient subjected to at least one aspect of the methods described herein. In some embodiments, the p53 reference can correspond to the level of p53 expression or activity in a tissue biopsy with a known level of p53 expression or activity. In some embodiments, the p53 reference can correspond to the level of p53 expression or activity measured in a patient's sample taken at a prior time point. In some embodiments, the p53 reference can correspond to a threshold level of p53 activity or expression or a standard numeric level.

In this aspect and all other aspects described herein, the sample analyzed in the methods described herein can be a bodily fluid sample (e.g., blood or urine) or a sample of a tissue at a target site from a patient. For example, for treatment of cancer, the sample can be a blood sample or a tumor biopsy from a patient. For treatment of other immune-related diseases or disorders, including, e.g., asthma, allergy, and infections, the sample can be a blood sample or a tissue biopsy from a target site to be treated in a patient.

In some immune-related diseases or disorders where suppression of immune response is desirable, e.g., autoimmune diseases, a DD1α agonist and/or PD-1 agonist therapy can be administered. Accordingly, another aspect provided herein relates to a method of identifying a patient diagnosed to have an inflammatory disease or disorder who is more likely to respond to a DD1α agonist and/or PD-1 agonist therapy. Examples of an inflammatory disease or disorder include, but are not limited to autoimmune diseases, acute inflammation, chronic inflammation, and combinations thereof. The method comprises: measuring the level of p53 activity or expression in a sample from a patient diagnosed to have an inflammatory disease or disorder; and comparing the level of p53 or expression in the sample with a p53 reference, and: (i) when the level of p53 activity or expression is lower than the p53 reference, the patient is identified to be more likely to respond to a DD1α agonist and/or PD-1 agonist therapy; or (ii) when the level of p53 activity or expression is the same as or greater than the p53 reference, the patient is identified to be more likely to respond to an alternative, anti-inflammatory immunotherapy without a DD1α agonist or PD-1 agonist therapy. In some embodiments, the method further comprises identifying the patient who is more likely to respond to a DD1α agonist and/or PD-1 agonist therapy, or who is more likely to benefit from an alternative, anti-inflammatory immunotherapy without a DD1α agonist and/or PD-1 agonist therapy, based on the level of p53 activity and/or expression measured in the patient's sample.

In some embodiments, the method can further comprise administering a DD1α agonist and/or PD-1 agonist therapy to the patient when the level of p53 activity or expression is lower than the p53 reference. A PD-1 agonist therapy can comprise an agent that enhances or induces binding of PD-1 with PD-L1 and/or PD-L2. In some embodiments, the PD-1 agonist therapy can comprise a PD-1 agonist, a PD-L1 agonist, and/or a PD-L2 agonist. In some embodiments, the DD1α agonist therapy can comprise an agent that increases homophilic interactions between DD1α molecules and/or an agent that increases heterophilic interactions between DD1α molecules and PD-1 molecules. In some embodiments, the agonist or agent used in the DD1α agonist and/or PD-1 agonist therapy can comprise a protein, a peptide, a nucleic acid, an antibody, a small molecule, a vaccine, and combinations thereof.

In some embodiments, the method can further comprise administering an alternative, anti-inflammatory immunotherapy without a DD1α agonist and/or PD-1 agonist therapy when the level of p53 activity or expression is the same as or greater than the p53 reference. An exemplary alternative, anti-inflammatory immunotherapy can comprise a suppressor of a proinflammatory T cell response pathway and/or an activator of an anti-inflammatory T cell response pathway. Non-limiting examples of the suppressor of the proinflammatory T cell response pathway and/or activator of the anti-inflammatory T cell response pathway include a TIGIT agonist, a Fgl2 agonist, a TIM-3 agonist, a galectin-9 molecule, a CTLA-4 agonist, a Lag-3 agonist, an antagonist of an immune checkpoint activating molecule, an agonist of an immune checkpoint inhibitory molecule, or any combination thereof.

In some embodiments, the patient amenable to the methods described herein can be a patient who has been receiving an anti-inflammatory treatment, e.g., an anti-inflammatory immunotherapy.

A p53 reference used for comparison to a measured level of p53 activity or expression in a patient's sample generally involves a positive control, a negative control, and/or a threshold value. In some embodiments, the p53 reference can correspond to the level of p53 activity or expression in a normal healthy subject. In some embodiments, the p53 reference can correspond to the level of p53 activity or expression in a normal tissue of the same type or lineage as the sample (e.g., same type or lineage as a tissue biopsy obtained from a target site (e.g., an inflammatory tissue). In some embodiments, the normal tissue of the same type or lineage as the sample can be obtained from a patient subjected to at least one aspect of the methods described herein. In some embodiments, the p53 reference can correspond to the level of p53 expression or activity in a tissue biopsy with a known level of p53 expression or activity. In some embodiments, the p53 reference can correspond to the level of p53 expression or activity measured in a patient's sample taken at a prior time point. In some embodiments, the p53 reference can correspond to a threshold level of p53 activity or expression or a standard numeric level.

The sample analyzed in this aspect of the methods described herein can be a bodily fluid sample (e.g., blood) or a sample of a tissue at a target site from a patient. For example, for treatment of autoimmune diseases, the sample can be a blood sample or a tissue biopsy from a target site to be treated in a patient.

In another aspect, the inventors have discovered that DD1α functions as an inhibitory signal for T cells via a homophilic, intercellular DD1α-DD1α interaction and/or a heterophilic, intercellular DD1α-PD-1 interaction. These findings provide insights into therapeutic treatments for immune-related diseases or disorders. For example, p53-overexpressing cancer cells express DD1α and related immune checkpoint inhibitors such as PD-1 and PD-L1 molecules, which enables the cancer cells to interact with T cells through intercellular homophilic DD1α binding and/or heterophilic PD-1 binding (e.g., PD-1/DD1α; PD-1/PD-L1; and/or PD-1/PD-L2), and thus suppresses the immune response, allowing the cancer cells to escape from immune surveillance. While existing anti-PD-1 therapy, alone or in combination with an anti-cancer agent, can be used to treat cancer, that approach does not target homophilic DD1α interaction and/or heterophilic DD1α/PD-1 interaction. Thus, the cancer cells that overexpress DD1α, PD-1, and/or PD-L1 (e.g., cancer cells that overexpress p53) can still escape from immune surveillance via the DD1αsignaling pathway if only PD1 and/or PD-L1 are targeted without targeting interaction of DD1α with DD1α and/or PD.

Accordingly, one aspect provided herein is a method of treating cancer involving inhibition of DD1α interaction on cancer cells with other immune cells. The method comprises administering to a cancer patient in need thereof a treatment comprising an agent that antagonizes the homophilic interaction of DD1α with DD1α. In some embodiments, the cancer patient administered the treatment can be determined to have a level of p53 activity or expression greater than a p53 reference. In some embodiments, the cancer patient administered the treatment can be determined to have a level of DD1α activity or expression greater than a DD1α reference. In some embodiments, the cancer patient administered the treatment can be determined have a level of PD-1 activity or expression greater than a PD-1 reference.

In some embodiments, the agent that antagonizes the homophilic interaction of DD1α with DD1α can further antagonize the functional interaction of DD1α with PD-1. In these embodiments, the agent can comprise a moiety that binds DD1α and a moiety that binds PD-1. In some embodiments, the agent can be a peptide or an antibody. In some embodiments, the moiety that binds DD1α can be attached to the moiety that binds PD-1 via a linker moiety. In some embodiments, the moieties that bind DD1α and PD-1 can comprise antigen-binding domains of antibodies that specifically bind DD1α and PD-1, respectively.

In some embodiments, the treatment can further be adapted to disrupt binding of PD-1 with PD-L1 and/or PD-L2. In some embodiments, the agent that antagonizes the homophilic interaction of DD1α with DD1αcan be adapted to further disrupt binding of PD-1 with PD-L1 and/or PD-L2.

Without wishing to be bound by theory, it is also contemplated that cells infected with a bacterial or fungal pathogen, similarly to cancer cells, overexpress DD1α and related immune checkpoint inhibitors such as PD-1 and PD-L1 molecules, which permits the infected cells to interact with T cells through intercellular homophilic DD1αbinding and/or heterophilic PD-1 binding (e.g., PD-1/DD1α; PD-1/PD-L1; and/or PD-1/PD-L2), and thus suppresses the immune response, allowing the infected cells to escape from immune surveillance. Accordingly, another aspect provided herein relates to a method of treating infection with a bacterial and/or fungal pathogen. The method comprises administering a treatment comprising an agent that antagonizes DD1α activity to a subject infected with a bacterial and/or fungal pathogen.

In some embodiments, the treatment can inhibit macrophage activity against host cell constituents while permitting pathogen phagocytosis by macrophages.

In some embodiments, the agent that antagonizes DD1α activity can antagonize the homophilic interaction of DD1α with DD1α.

In some embodiments, the agent that antagonizes DD1α activity can antagonize the functional interaction of DD1α with PD-1.

In some embodiments, the agent that antagonizes DD1α activity can antagonize the homophilic interaction of DD1α with DD1α and antagonizes the functional interaction of DD1α with PD-1. In these embodiments, the agent can comprise a moiety that binds DD1α and a moiety that binds PD-1. In some embodiments, the agent can be a peptide or an antibody. In some embodiments, the moiety that binds DD1αcan be attached to the moiety that binds PD-1 via a linker moiety. In some embodiments, the moieties that bind DD1α and PD-1 can comprise antigen-binding domains of antibodies that specifically bind DD1α and PD-1, respectively.

In some embodiments, the treatment can be adapted to also antagonize PD-1 activity. For example, in some embodiments, the treatment can be also adapted to disrupt binding of PD-1 with PD-L1 and/or PD-L2. In some embodiments, the agent that antagonizes DD1α activity can be adapted to further disrupt binding of PD-1 with PD-L1 and/or PD-L2.

### BRIEF DESCRIPTIONS OF THE DRAWINGS

FIGS. 1A-1D show identification of DD1α as a p53 target gene. (FIG. 1A) p53-dependent expression of DD1α. DD1α mRNA and protein were assessed after tetracycline (tet) removal in EJ-p53tet cells (tet-off) (left). MCF7 (Wt-p53) cells were transfected with either siRNA targeting p53 or luciferase control for 24 hours, then treated with camptothecin (CPT, 500 nM). MCF7 cells were treated with Nutlin-3 (10 μM) for the indicated times. Western blot analyses were performed with specific antibodies against p53, DD1α, p21, and β-actin. (FIG. 1B) p53 binds to and transactivates the DD1α promoter (SEQ ID NOS 32 and 33, respectively, in order of appearance). Luciferase reporter constructs containing the putative p53 recognition sites in the DD1α promoter were cotransfected with either WT-p53, mutant p53 (V143A), or control pcDNA3.1 empty vector into U2OS cells. Results represent mean±SD from three independent experiments. ChIP was performed on MCF7 cells exposed to ionizing radiation (IR, 13 Gy). Immunoprecipitation carried out with anti-p53 antibody (DO-1) or mouse IgG (negative control). The % input of coprecipitating DNAs were measured by qPCR and presented as mean±SD (n=3). (FIG. 1C) A schematic representation of DD1α indicating the signal peptide (1-32), the immunoglobulin V domain, and the transmembrane region is shown. The expression of human DD1α mRNA was analyzed by northern blotting from various human tissues including blood leukocyte (Lk), lung (Lu), placenta (Pl), small intestine (SI), liver (Li), kidney (Ki), spleen (Sp), thymus (Tm), colon (Co), skeletal muscle (Sm), heart (He) and brain (Br). (FIG. 1D) Multiple sequence alignment of the IgV domains of DD1α and its homologous proteins (SEQ ID NOS 34-39, respectively, in order of appearance). The predicted secondary structures show the alignment as “e” for β strand and “h” for α-helix. The identical amino acids are in black box, the conserved amino acids are outlined in a box, and the consensus amino acids are in grey box.

FIGS. 2A-2F show that DD1α plays essential roles in apoptotic cell engulfment. (FIG. 2A) DD1α on apoptotic cells contributes to apoptotic cell engulfment. MCF7 cells was transfected with shRNAs including control (luciferase), DD1α (two different target sequences: #1, #2) or p53 (two different target sequences: #1, #2) and were induced apoptosis by the treatment of CPT (10-20 μM) for 48 hours. Then, apoptotic MCF7 cells were labeled with pHrodo, incubated with human macrophages for 2 hours, and examined by immunofluorescence microscopy to detect phagocytosis (arrows: engulfed MCF7 cells). Where indicated, MCF7 cells expressing DD1α shRNA (#1) were transfected for 24 hours with a vector encoding DD1αbefore the phagocytosis assay. Phagocytic Index indicates the number of MCF7 cells phagocytosed per 100 macrophages. More than 400 macrophages were counted. Data are mean±SD from three different experiments. The representative images of phagocytosis with control, DD1α, p53 knockdown and DD1α-reintroduced MCF7 cells plus primary human macrophages are shown. Scale bar, 100 μm. (FIG. 2B) DD1α-null cancer cells is resistant to phagocytosis. Phagocytic indices of DD1α Wt cancer cells (MCF7, ZR75-1, A375), DD1α-null cancer cells (BxPC-3, Hs888.T), and DD1α-reintroduced DD1α-null cancer cells were determined using human macrophages, as shown in FIG. 2A. The levels of DD1α protein were examined by western blot analysis. (FIG. 2C) Engulfment of Wt, DD1α−/−, and p53−/− apoptotic thymocytes by BMDMs (Bone Marrow Derived Macrophages isolated from Wt-mice) was assessed by flow cytometry analysis. Thymocytes isolated from Wt, DD1α−/−, or p53−/− mice were exposed to ionizing irradiation (2-100 Gy) to induce similar amounts of apoptotic populations. The pHrodo-labeled thymocytes (live or apoptotic: Wt, dead Wt, dead DD1α−/−, or dead p53−/−) were incubated with Wt-BMDMs for 30 min. The phagocytosis was determined by the percentage of macrophages containing positive pHrodo signal. Data are shown as mean±SD and representative of three independent experiments. (FIG. 2D) Engulfment of Wt, DD1α−/−, and p53−/− apoptotic thymocytes by BMDMs was assessed by time lapse imaging analysis. CFSE-labeled apoptotic Wt, DD1α−/−, or p53−/− thymocytes were incubated with PKH26 fluorescently-labeled Wt BMDMs. The images of phagocytosis were taken every 1 min after incubation. The representative images of engulfment were shown and arrowheads indicate the engulfed thymocytes. Data represents mean±SD from three different experiments. (FIG. 2E) Impaired clearance of apoptotic cells in the DD1α−/− mice. Photographs of representative thymus from Wt and DD1α−/− mice at indicated time points after exposure or unexposure of ionizing irradiation (6.6 Gy). Total cell numbers per thymus from Wt and DD1α and DD1α were determined at 8 h after ionizing irradiation. Mean±SD, n=5 per group. Bottom panels represent whole sections of thymus from Wt and DD1α−/− mice exposed to IR were stained with TUNEL and 4′,6-diamidino-2-phenylindole (DAPI). Scale bar, 1 mm. The percentage of TUNEL positive cells were determined by percent of the TUNEL positive cells per DAPI-positive cells using imaging analysis program (CELLSENSE DIMENSION™, OLYMPUS™). Data represents mean±SD, n=4. (FIG. 2F) Apoptotic cells in lymph nodes and colon of Wt and DD1α−/− mice. The cryosections were stained with TUNEL and DAPI. The apoptotic cells were determined by counting TUNEL-positive cells per mm² under fluorescence microscope. Mean±SD, n=3. Scale bar, 50 μm.

FIGS. 3A-3E show that intercellular homophilic DD1α interaction between apoptotic cells and phagocytes mediates apoptotic cell engulfment. (FIG. 3A) Defective apoptotic cell engulfment of DD1α−/− m-BMDMs. Engulfments of Wt and DD1α−/− apoptotic thymocytes by Wt and DD1α−/− BMDMs were assessed by flow cytometry as described previously. The pHrodo-labeled apoptotic thymocytes were incubated with m-BMDMs for 30 min and the phagocytosis was determined by measuring the positive pHrodo-containing macrophages. Graph represents mean±SD from three independent experiments. (FIG. 3B) Homophilic DD1α interaction. Binding of color latex beads coated with DD1α-Ig fusion proteins (the extracellular region of DD1α fused with the immunoglobulin G Fc segment) to 293T cells transfected with empty vector (EV), the complete DD1α (2 or 10 μg), or a mutant lacking the IgV domain (DD1α-ΔIgV). Ig protein-coated beads were included as control. After 30 min, unbound beads were washed. The binding was examined under an inverted microscope (left panels) and also determined from the optical density (O.D.) at 492 nm (shown at the right). Data are shown as mean±SD and are representative of three experiments. Scale bar, 50 μm. (FIG. 3C) Intercellular DD1α interaction. Binding of CFSE-labeled DD1α-overexpressing apoptotic MCF7 cells to U2OS cells expressing empty vector (EV), full length DD1α, DD1α-ΔIgV. Scale bar, 100 μm. Binding was determined by counting the bound cells per 100 μm² and normalized by cell number bound to untreated plate. Mean±SD of three experiments is shown. (FIG. 3D) Mapping of binding site for homophilic DD1α interaction (left panel). His-DD1α (33-194) protein was reacted with GST-DD1α variants (the extracellular region; 33-94, the immunoglobulin domain; 37-146, IgV-deleted mutant, the cytoplasmic region; 215-311) immobilized on glutathione-agarose beads. The bead bound His-DD1α (33-194) proteins were eluted and detected by immunoblotting using anti-His antibody. A portion (5%) of the proteins input to the binding reaction was also subjected to immunoblotting. Dimerization of DD1α is examined in intact cells. DD1α-transfected cells were treated or untreated with 1 mM bis(maleinido)hexane (BMH) for 1 hour, and DD1α protein was analyzed by western blotting under non-reducing condition. (FIG. 3E) Extracellular IgV domain is required for engulfment of homophilic DD1α interaction. DD1α, DD1α-ΔIgV (IgV-defective DD1α mutant), and control empty vector were reintroduced into DD1α depleted MCF7 cells by DD1α-shRNA, and the cells were treated with CPT (10 μM) for 48 hrs. The phagocytosis of MCF7/DD1α KD cells expressing empty vector (EV), DD1α or DD1α-ΔIgV was determined as in FIG. 2A. Graph represents mean±SD of three experiments. Scale bar, 50 μm.

FIGS. 4A-4E show that DD1α-deficient mice develop autoimmune and severe inflammatory disorder. (FIG. 4A) Inflammatory phenotype of DD1α−/− mice. Photographs of 7 or 10 month-old female Wt and DD1α−/− mice are shown. (FIG. 4B) Survival of DD1α−/− mice (43 females, 51 males) and control Wt mice (33 females, 38 males) monitored over a 19 month-period. *P<0.001 (FIG. 4C) Auto-nuclear autoantibodies (ANA) in sera of affected female Wt and DD1α−/− mice were examined by immunofluorescence experiment using HEp-2 cells. Scale bar, 50 μm. The levels of ANA and anti-double-stranded DNA (anti-dsDNA) autoantibodies in sera of affected 8 to 10-month old female Wt and DD1α−/− mice were also measured by ELISA. (n=10 mice for ANA, n=11 mice for anti-dsDNA). Each dot represents the value for a single mouse. Serum IgG levels of 10-month old female Wt and DD1α−/− mice (n=7) assessed by ELISA. The level of albumin in urine collected for 24 hours from affected 10-12 month-old female mice (n=8) were analyzed by SDS-PAGE and imaging analysis. Data represents mean±SD. (FIG. 4D) Dermatitis incidence in DD1α−/− mice (40 females and 48 males) and control Wt mice (31 females and 37 males). *P<0.001 (FIG. 4E) Splenomegaly and lymphadenopathy observed in DD1α−/− mice. Spleens and lymph nodes of 4 or 10 month-old female Wt and DD1α−/− mice from one littermate were shown. The weight of spleen and lymph nodes were measured as indicated. Data represents mean±SD. n=6 to ˜11 mice per group.

FIGS. 5A-5D show that DD1α regulates T cell activation and development of induced regulatory T cells. (FIG. 5A) DD1α inhibits the activation of human CD4+ T cells and CD8+ T cells. Enriched human CD4+ T cells and CD8+ T cells were stained with CFSE (1 μM) and stimulated with plate-bound anti-CD3 antibody (3 μg/ml) together with 2, 5, or 10 μg/ml of DD1α-Ig proteins or control Ig proteins. The proliferations of CD4+ T or CD8+ T cells were determined by the percentages of CFSE-labeled CD4+ T or CD8+ T cells at the 72-hour culture. The levels of cytokines IFN-γ and TNF-α in culture supernatant were analyzed by ELISA at the 48-hour culture. (FIG. 5B) DD1α-mediated inhibition of T cell activation is modulated by α-PD1 antibodies. Human CD4+ T cells were pre-incubated with 150 μg/ml of anti-PD1 antibody for 30 min and then stimulated by plate-bound anti-CD3 antibody (3 μg/ml) alone or together with DD1α-Ig, PD-L1-Ig or control Ig protein with the indicated combinations. The proliferation and cytokine production of CD4+ T cells were determined as FIG. 5A. (FIG. 5C) Loss of DD1α receptor in CD4+ T cells partially blocks DD1α mediated inhibition of T-cell activation but did not affect PD-L1 mediated CD4+ T cell inhibition. Purified CD4+ T cells from Wt and DD1α−/− mice were stimulated with 2.5 μg/ml anti-CD3 alone or together with DD1α-Ig, PD-L1-Ig or control Ig as indicated. The proliferations of CD4+ T cells were determined by percentage of CFSE-diluted CD4+ cells on day 3. The levels of cytokines IFN-γ and IL-2 in culture supernatant were analyzed by ELISA on day 2. (FIG. 5D) DD1α mediates Foxp3+ iTreg cell development. Development of Foxp3+ iTreg cells was assessed by flow cytometric analysis of Foxp3 expression after stimulation with anti-CD3 (5 μg/ml) and anti-CD28 (2 μg/ml) plus the indicated concentrations of TGF-β together with 10 μg/ml of control Ig, DD1α-Ig or PD-L1-Ig for 3 days. Data are shown as mean±SD and representative of three experiments.

FIGS. 6A-6E show physical association of DD1α with PD1 but not with PD-L1. (FIG. 6A) Interaction of DD1α with PD1 or DD1α. Left, HEK293T cells transfected with plasmids expressing DD1α, PD1, or TIM-3 or control empty vector pcDNA3.1 were stained with Ig fusion proteins (control Ig, DD1α-Ig, PD-L1-Ig, or PD1-Ig). The bound Ig fusion proteins were detected with anti-Ig-APC. Empty vector (pcDNA3.1)-transfected cells stained with Ig proteins are shown as a negative control (gray filled) and other staining is shown as black open. (FIG. 6B) DD1α binds to both DD1α and PD1 on CD4+ T cells. Purified CD4+ T cells from Wt and DD1α−/− mice were assayed for binding to Ig fused DD1α or PD1 proteins. Cells were incubated with DD1α-Ig (open), PD1-Ig (open), or control Ig proteins (gray filled) and the bound Ig fusion proteins were detected by anti-Ig-APC. Also, purified human CD4+ T cells were pre-incubated with 150 μg/ml anti-PD1 antibody or control mouse IgG1 antibody for 30 min and stained with DD1α-Ig (open), PD-L1-Ig (open), or control Ig proteins (gray filled). The bound Ig fusion proteins were detected by anti-Ig-APC. Binding amounts were determined by percentage of APC positive cells compared to control Ig protein-bound cells. (FIG. 6C) Ig-pulldown experiments show DD1α interaction with both DD1α and PD1. Ig fusion proteins (control Ig, DD1α-Ig, PD1-Ig, or PD-L1-Ig) were incubated with lysates from HEK293T cells transfected with PD1-HA, DD1α-HA, or TIM-3-HA as indicated and pulled down with protein A/G agarose. The bead bound proteins were eluted and determined by western blot analysis using anti-HA antibody (asterisks). (FIG. 6D) Ig fusion proteins (control Ig, DD1α-Ig, or PD1-Ig) were incubated with lysates from Nutlin-3-treated MCF7 cells and pulled down using protein A/G agarose. The bead bound proteins were eluted and detected by western blot analysis with anti-DD1α or anti-PD1 antibody (asterisk). (FIG. 6E) p53-mediated induction of PD1 and PD-L1. MCF7 cells were treated by Nutlin-3 (10 μM) for 1 to 3 days. MCF7 cells were transfected with control siRNA or p53 siRNA for 24 hours and treated with CPT (500 nM) or DMSO (control) for 1 and 2 days. The levels of indicated proteins were analyzed by western blot analysis. Total RNAs from control siRNA or p53 siRNA transfected MCF7 cells were assessed for mRNA levels of PD1 or PD-L1 by real-time quantitative PCR. PD1 or PD-L1 mRNA levels were normalized to 36B4 expression and shown as mean±SD (n=3). Protein levels of PD1 and PD-L1 were examined in EJ-p53tet cells in the absence or presence of tetracycline (tet), ZR75-1 cells treated with CPT (500 nM), or A375 cells exposed to 13 Gy ionizing irradiation for the indicated times.

FIG. 7 is a schematic depicting that redundant signaling of PD-1 results in incomplete checkpoint release with specific antibodies.

FIGS. 8A-8B show the regulation of PD1 axis and data indicating that PD1, PDL1 and DD1α are p53 target genes. These data indicate that rational targeting of p53 wildtype tumors and p53-inducing chemotherapeutic/radiotherapy combinations can segment a responder population.

FIGS. 9A-9B shows intercellular homophilic interaction of DD1α. (FIG. 9A) DD1α-Myc and DD1α-HA were transfected into 293T cells separately. DD1α-Myc-transfected 293T cells and DD1α-HA-transfected 293T cells were co-cultured for 24 hours and lysed. DD1α-HA proteins were immunoprecipitated with cross-linked HA agarose. The resulting immune complexes and inputs were resolved by SDS-PAGE and blotted against indicated antibodies. The reciprocal Co-IP was performed using cross-linked Myc agarose. (FIG. 9B) Imaging study of intercellular homophilic DD1α binding was performed by proximity ligation assay (DUOLINK™). To express HA-tag or Flag-tag in the extracellular region of DD1α, HA or Flag tag was fused between DD1α signal peptide (1-32) and DD1α N-terminus peptide (33-311), resulting in the construction of SP(1-32)-HA-DD1α (33-311) and SP(1-32)-Flag-DD1α(33-311). SP-HA-DD1α was transfected into J774.1 macrophage cells, and SP-Flag-DD1α was transfected into ZR75-1 breast carcinoma cells. After 24 hours, the transfected J774.1 and ZR75-1 cells were co-cultured for 24 hours and subjected to proximity ligation assay using anti-HA and anti-Flag antibodies. The detected interaction between HA-DD1α of J774.1 and Flag-DD1α of ZR75-1 is shown. The expression levels of HA-DD1α in J774.1 and Flag-DD1α in ZR75-1 were confirmed by western blot analysis.

FIGS. 10A-10B shows flow cytometric analysis of DD1αsurface expression. (FIG. 10A) DD1α and PD-1 surface expression on human CD14+ monocytes, CD4+ T cells and CD8+ T cells. Human peripheral blood mononuclear cells (PBMC) were stained with anti-DD1α antibody or anti-PD-1 antibody together with anti-CD14, anti-CD4, or anti-CD8 antibody. Control mouse IgG staining was included as a negative control (gray filled). DD1 expression is shown as open lines and open lines represent PD-1 expression at the surface. (FIG. 10B) DD1α surface expression on F4/80+ macrophage, B220+ B cells, CD4+ T cells and CD8+ T cells from Wt and DD1α−/− mouse. Splenocytes isolated from Wt or DD1α−/− mouse were stained with anti-DD1α antibody together with anti-F4/80, anti-B220, anti-CD4, or anti-CD8 antibody. Control mouse IgG staining is shown as gray filled. DD1α expression was indicated as an open line.

FIGS. 11A-11B show inhibitory effect of DD1α on the activation of mouse CD8+ T cells. (FIG. 11A) Purified mouse CD8⁺ T cells were stained with 1 μM CFSE and stimulated with plate-bound 2.5 μg/ml anti-CD3 antibody together with 2, 5, or 10 μg/ml of DD1α-Ig protein or control Ig protein. The proliferations of CD8⁺ T cells were determined by percentage of CD8⁺ T cells containing diluted CFSE signal on day 3. (FIG. 11B) The indicated cytokine levels in culture supernatant were analyzed by ELISA on day 2. Data represents mean±SD (n=3).

FIGS. 12A-12B show p53-dependent induction of DD1α. (FIG. 12A) Induction of DD1αmRNA in EJ-p53 cell after tetracycline withdrawal. Total RNA was prepared from EJ-CAT or EJ-p53 cells grown in the presence or absence of tetracycline (tet, 1 μg/ml) for 12, 24 and 48 hours. Real time quantitative PCR was performed using specific primer set for human DD1α. Data represent mean±SD (n=3). (FIG. 12B) p53-dependent induction of DD1α in response to DNA damage. Saos2 (p53-null) and MCF7 (Wt-p53) cells were treated with CPT (500 nM) or ETO (25 μM) or exposed to IR (13 Gy) for the indicated times. Cell lysates were analyzed by western blot analysis for the levels of p53, DD1α, p21, and

FIGS. 13A-13D show DD1α is induced in response to DNA damage in a wt-p53-dependent manner. (FIG. 13A) Human cancer cells (ZR75-1, A549, or LOX-IMVI) containing Wt-p53 were treated with ETO (25 μM), CPT (500 nM) or exposed to IR (13 Gy) for 24, 48 and 72 hours. The levels of indicated proteins were determined by western blot analysis. (FIG. 13B) LOX-IMVI (Wt-p53) cells were transfected with luciferase reporter constructs (P1, P2) containing the putative p53 recognition sites in the DD1αpromoter for 24 hours, followed by 24-hours treatment of 25 μM ETO. The p21 promoter was included as a control. The results represent mean±SD from three independent experiments. (FIG. 13C) Nuclear extracts (N.E.) from MCF7 cells treated with ETO were incubated with radiolabeled Wt-DD1α BS1 oligonucleotides in the presence of cold specific (Wt-BS) or nonspecific mutant (Mt-BS) competitor. (FIG. 13D) GST-fused p53 (93-312; DNA-binding domain) or control GST recombinant proteins were incubated with radiolabeled DD1αBS1, mutated-DD1αBS1, DD1αBS2, and control p21-BS oligonucleotides as indicated. The p53-oligonucleotide complex (arrow) was detected by SDS-PAGE and autoradiograph.

FIG. 14 shows glycosylation of DD1αprotein. U2OS cells were transfected with DD1α-HA or control empty vector for 24 hours and treated with tunicamycin at 5 or 10 μg/ml for 24 hours. DD1α-HA was visualized by western blot analysis with anti-HA antibody.

FIGS. 15A-15B show apoptotic responses in MCF7 cells with sh-DD1α or sh-p53. (FIG. 15A) Western blot analysis after depletion of DD1α or p53. MCF7 cells were stably transfected with shRNAs including control (luciferase), DD1α (two different target sequences: #1, #2) or p53 (two different target sequences: #1, #2). Vector encoding DD1α was transfected into MCF7 cells with DD1α shRNA (#1). Cells were treated with CPT (10 μM for MCF7/sh-lucif, MCF7/sh-DD1α, DD1α− reintroduced MCF7/sh-DD1α, 20 μM for MCF7/sh-p53) for 48 hours. Cell lysates were analyzed for the levels of DD1α, p53, p21 and β-actin by western blot analysis. (FIG. 15B) The effect of DD1α knockdown on CPT-induced apoptosis. After 48 hour-treatment of CPT with the indicated concentrations, MCF7 cells were stained with TUNEL, and TUNEL-positive cells were analyzed by flow cytometry. Data represent mean±SD (n=3).

FIGS. 16A-16C show contribution of DD1α on apoptotic ZR75-1 cells to macrophage-mediated engulfment. (FIG. 16A) ZR75-1 cells were transfected with shRNAs including control luciferase (sh-lucif), sh-DD1α#1 or sh-p53#1 and treated with CPT (5, 10 μM) for 48 hours. Then, pHrodo-labeled apoptotic ZR75-1 were subjected to phagocytosis assay using human macrophages, and the phagocytic indices were determined, as described in FIG. 2A. Data are mean±SD (n=3). Engulfed ZR75-1 cells were indicated by arrows. Scale bar, 50 μM. (FIG. 16B) Expression analysis after knockdown by shRNAs (control, DD1α, p53). Knockdowned ZR75-1 cells were treated with CPT (5 μM for control and DD1αshRNA cells, 10 μM for p53 shRNA cells) for 48 hours. The protein levels of DD1α, p53, p21 or β-actin were tested by western blot analysis. (FIG. 16C) Apoptosis assay by TUNEL staining in CPT-treated ZR75-1 cells. ZR75-1 cells were treated with CPT for 48 hours and TUNEL staining was performed and analyzed by flow cytometry. Data are shown mean±SD (n=3).

FIG. 17 shows validation of apoptosis of human cancer cells (Wt DD1α and DD1α-null) for phagocytosis. Cells from Wt DD1α cancer cell lines (MCF7, ZR75-1, A375), DD1α-null cancer cell lines (BxPC-3, Hs888.T), and DD1α-reintroduced DD1α-null cancer cell clones were treated with CPT as indicated. After 48 hours, apoptosis was analyzed by TUNEL staining and flow cytometry. Graph represents mean±SD (n=3).

FIGS. 18A-18B show the effect of DD1α- or p53-knockout on IR-induced apoptosis in thymocytes. (FIG. 18A) Freshly isolated thymocytes from Wt, DD1α−/−, or p53−/− mice were exposed to IR at indicated dose and then incubated for 6 hours. The expression levels of DD1α were analyzed by real time quantitative RCR using specific primer set for mouse DD1α. Data represent mean±SD (n=3). (FIG. 18B) Apoptotic responses of IR-exposed thymocytes (Wt, DD1α−/− and p53−/−) were determined by Annexin V/PI staining. Annexin V-positive and PI-negative cells were counted as apoptotic cells. Data are mean±SD (n=3).

FIGS. 19A-19C show generation of DD1α-deficient mouse. (FIG. 19A) Genomic structure of DD1α (exons 1-6) with Wt allele, targeted allele, floxed allele and DD1α KO allele. A PGK-neomycin cassette (pink box) flanked by loxP and FRT sites was inserted downstream of exon 3 and a third loxP site was inserted upstream of exon 2. Chimeric mice derived from homologous recombinant ES cells were crossed with FLP recombinase mice to remove the PGK-neomycin cassette inserted between exons 3 and 4. Mice carrying the floxed DD1α allele (DD1αflox) were then crossed to Cre recombinase mice to obtain the DD1α KO allele (DD1α−). Genotyping primers (P1, P2, P3) were indicated as arrows. (FIG. 19B) The agarose gel shows the Wt and KO bands obtained from genotyping of mice from DD1α+/−×DD1α+/− matings. (FIG. 19C) Real time quantitative PCR analysis of DD1α expression in DD1α+/+, DD1α+/−, or DD1α−/− mouse embryonic fibroblasts (MEFs). Data represents mean±SD (n=3).

FIG. 20 shows spleen of Wt and DD1α−/− mice exposed to IR. Four to five weeks old Wt and DD1α−/− mice were treated with 6.6 Gy IR. After 6 hours, spleens were isolated from mice and the splenic weight was measured. The photograph of representative spleens of Wt and DD1α−/− mice exposed to IR is shown. Each dot represents the value for a single mouse and mean±SD (n=5) is shown.

FIGS. 21A-21B show that DD1α does not bind to phosphatidylserine. (FIG. 21A) The identity of lipid species on membrane lipid strips is shown. The binding of DD1α for phospholipids was assessed by protein-lipid overlay assay. (FIG. 21B) Recombinant DD1αproteins (DD1α-Ig, DD1α-His, His-DD1α) were purified from three different sources (293T cells, yeast, and E. coli). mTIM1-Ig proteins were included as a positive control. The purified proteins were incubated with membrane lipid strips. The bound proteins were detected using the indicated antibodies. The input proteins were confirmed by western blot analysis.

FIGS. 22A-22B show intercellular homophilic interaction of DD1α receptor. (FIG. 22A) DD1α-Myc and DD1α-HA were transfected into 293T cells separately. DD1α− Myc-transfected 293T cells and DD1α-HA-transfected 293T cells were co-cultured for 24 hours and lysed. DD1α-HA proteins were immunoprecipitated with cross-linked HA agarose. The resulting immune complexes and inputs were resolved by SDS-PAGE and blotted against indicated antibodies. The reciprocal co-IP was performed using cross-linked Myc agarose. (FIG. 22B) Imaging study of intercellular homophilic DD1α binding was performed by proximity ligation assay (Duolink). To express HA-tag or Flag-tag in the extracellular region of DD1α, HA or Flag tag was fused between DD1α signal peptide (1-32) and DD1α N-terminus peptide (33-311), resulting in the construction of SP(1-32)-HA-DD1α(33-311) and SP(1-32)-Flag-DD1α(33-311). SP-HA-DD1α was transfected into J774.1 macrophage cells, and SP-Flag-DD1α was transfected into ZR75-1 breast carcinoma cells. After 24 hours, the transfected J774.1 and ZR75-1 cells were co-cultured for 24 hours and subjected to proximity ligation assay using anti-HA and anti-Flag antibodies. The detected interaction between HA-DD1α of J774.1 and Flag-DD1α of ZR75-1 is circumscribed by a thick dotted line. The expression levels of HA-DD1α in J774.1 and Flag-DD1α in ZR75-1 were confirmed by western blot analysis.

FIGS. 23A-23B show validation and apoptotic response of DD1α− or DD1α-ΔIgV-reintroduced MCF7/sh-DD1α cells. (FIG. 23A) DD1α knockdown in MCF7 cells stably expressing DD1α shRNA #1. DD1α knockdowned MCF7 cells were transfected with DD1α-HA, DD1α-ΔIgV-HA, or empty vector and treated with 10 μM CPT or DMSO for 48 hours. The expression levels of reintroduced DD1α or DD1α-ΔIgV was checked by western blot analysis. (FIG. 23B) Apoptosis of the CPT-treated MCF7/sh-DD1αcells were analyzed by TUNEL staining and flow cytometry. Data are mean±SD (n=3).

FIGS. 24A-24B show DD1α-deficiency does not influence on engulfment of synthetic beads or E. coli. (FIG. 24A) Bone marrow-derived macrophages (m-BMDM) from Wt and DD1α−/− mice were incubated with carboxylate-modified fluorescent beads (synthetic beads) for indicated times. The phagocytosis was determined by the percentage of macrophages containing positive fluorescence signal. Data are shown as mean±SD and representative of two independent experiments. (FIG. 24B) The phagocytic potential of Wt and DD1α−/− BMDMs for E. coli was assessed using pHrodo-labeled E. coli. The phagocytosis was determined by the percentage of macrophages containing positive pHrodo signal. Data represents mean±SD (n=3).

FIGS. 25A-25D show spontaneous glomerulonephritis in DD1α−/− mice. (FIG. 25A) Immune complex deposits in glomeruli of DD1α−/− mice. Representative images of kidney sections from 10-month-old Wt and DD1α−/− mice stained with anti-mouse IgG antibody are shown. Scale bar, 20 μm. (FIG. 25B) Kidney sections from Wt and DD1α−/− mouse stained with PAS. Glomeruli from WT mice have a regular architecture with delicate mesangium. Glomeruli from DD1−/− mice show diffuse mesangial expansion by PAS-positive material and cellular debris (arrowhead) as well as occasional neutrophils within capillary lumens (arrow). Scale bar, 50 μm. (FIG. 25C) Low magnification electron micrograph of Wt and DD1α−/− glomeruli. A normal architecture with delicate mesangium and intact filtration barrier of Wt glomeruli and an expanded mesangium with electron-dense deposits, and neutrophils within capillary lumens (arrows) of DD1α−/− glomeruli are shown. Scale bar, 2 μm. (FIG. 25D) High magnification electron micrograph of the glomerular mesangium of Wt and DD1α−/− glomeruli. Large electron-dense deposits (arrows), often with tubular substructure in the glomerular mesangium of DD1α−/− glomeruli are shown. Scale bars, 2 μm (left) and 500 nm (right).

FIG. 26 shows multi-organ inflammation in DD1α−/− mice. H&E stained sections of skin from Wt and DD1α−/− mice are shown (left panels). Thin epidermis and no significant inflammation involving dermis or subcutaneous tissue are shown in Wt skin (top). The mild thickening epidermis, hyperkeratosis and significant mixed acute inflammation with predominance of neutrophils in the dermis are shown in DD1α−/− skin (lower panels). Cutaneous ulceration with bacterial infection, adjacent spongiotic dermatitis with hyperkeratosis, and an accompanying mixed inflammatory infiltrate extending deep into the subcutaneous tissue in DD1α−/− skin is shown (lower bottom panel). Scale bar, 100 μm. H&E stained sections of lung from Wt and DD1α−/− mice are shown (middle panels). Normal alveolar, bronchiolar and vascular architecture and no inflammation were observed in Wt mice. Focal non-specific bronchiolitis with a mixed inflammatory infiltrate in DD1α−/− mouse was detected and is shown as arrow. Scale bars, 200 μm. H&E stained sections of spleen from Wt and DD1α−/− mouse. Normal size and architecture of pulp is shown in Wt spleen. Greatly increased extramedullary hematopoesis including both erythroid and myeloid (inset) hyperplasia is shown in DD1α−/− spleen. Scale bar, 200 μm.

FIGS. 27A-27B show flow cytometric analysis of DD1α surface expression. (FIG. 27A) DD1α and PD-1 surface expression on human CD14+ monocytes, CD4+ T cells and CD8+ T cells. Human peripheral blood mononuclear cells (PBMC) were stained with anti-DD1α antibody or anti-PD-1 antibody together with anti-CD14, anti-CD4, or anti-CD8 antibody. Control mouse IgG staining was included as a negative control (gray filled). DD1αexpression is shown as an open line on the top panels and PD-1 expression at the surface is shown as an open line in the bottom panels. (FIG. 27B) DD1αsurface expression on F4/80+ macrophage, B220+ B cells, CD4+ T cells and CD8+ T cells from Wt and DD1α−/− mouse. Splenocytes isolated from Wt or DD1α−/− mouse were stained with anti-DD1α antibody together with anti-F4/80, anti-B220, anti-CD4, or anti-CD8 antibody. Control mouse IgG staining is shown as gray filled DD1αexpression was indicated as open line.

FIG. 28 shows DD1α-mediated inhibition of CD4+ and CD8+ T cell activation. Enriched CD4+ and CD8+ T cells from Wt and DD1α−/− mice were stimulated, with 2.5 μg/ml anti-CD3 alone or together with DD1α-Ig or control Ig. The proliferations of CD4+ T cells (top graphs) or CD8+ T cells (bottom graphs) were determined by the percentage of CFSE-diluted CD8+ cells on day 3. The levels of cytokines (IFN-γ and TNF-α) in culture supernatant on day 2 were analyzed by ELISA. The results represent mean±SD from three experiments.

FIGS. 29A-29B show inhibitory effect of DD1α on the activation of mouse CD8+ T cells. (FIG. 29A) Purified mouse CD8+ T cells were stained with 1 μM CFSE and stimulated with plate-bound 2.5 μg/ml anti-CD3 antibody together with 2, 5, or 10 μg/ml of DD1α-Ig protein or control Ig protein. The proliferations of CD8+ T cells were determined by percentage of CD8+ T cells containing diluted CFSE signal on day 3. (FIG. 29B) The indicated cytokine levels in culture supernatant were analyzed by ELISA on day 2. Data represents mean±SD (n=3).

FIG. 30 shows association between PD-1 and DD1α in MCF7 cells. Ig fusion proteins (control Ig, DD1α-Ig, or PD1-Ig) were incubated with lysates from Nutlin-3-treated MCF7 cells and pulled down using proten A/G agarose. The bead bound proteins were eluted and detected by western blot analysis with anti-DD1α or anti-PD1 antibody.

FIG. 31 shows co-immunoprecipitation of DD1α and PD-1, indicating binding of DD1α with PD-1.

FIG. 32 shows an exemplary nucleotide sequence encoding human DD1α protein of SEQ ID NO: 1.

FIG. 33 shows that DD1αblocking monoclonal antibodies developed can rescue DD1α-DD1α and DD1α-PD-1 binding (assayed by adhesion assay). Adhesion assay shows specific binding of DD1α-DD1α and DD1α-PD-1. 293 cells expressing DD1α, PD-1 or control empty vector were labeled with BCECF and some cells were pre-incubated with 40 μg/ml of anti-DD1α mAb (2B11, 5C2, 6F10), anti-PD-1 mAb, or isotype control IgG, as indicated. Plate wells were coated with DD1α-Ig, PD-1-Ig, PD-L1-Ig, or control Ig and blocked. Fluorescent 293 cells were introduced into the wells. Fluorescence was measured before and after washing. Mean±SD of three experiments is shown.

FIGS. 34A-34B show that DD1α protein binding on CD4+ T cells is diminished by the treatment of DD1α-blocking monoclonal antibody or in DD1α−/− T cells. (FIG. 34A) DD1α binds on CD4+ T cells via DD1α receptor. Left, purified human CD4⁺ T cells were stimulated by plate-bound 5 μg/ml anti-CD3 antibody for 3 days, collected and then pre-incubated with 100 μg/ml anti-DD1α or control mouse IgG1 antibody for 30 min. Cells were washed and assayed for Ig proteins binding by staining with DD1α-Ig (open), PD-L1-Ig (open) or control Ig proteins (gray filled). The bound Ig fusion proteins were detected by anti-Ig-PE (cross absorbed against mouse Ig). (FIG. 34B) Purified CD4⁺ T cells from Wt and DD1α−/− mice were assayed for binding to Ig fused DD1α or PD-L1 proteins. Cells were incubated with DD1α-Ig (open), PD-L1-Ig (open), or control Ig proteins (gray filled) and the bound Ig fusion proteins were detected by anti-Ig-APC. Binding amounts were determined by percentage of fluorescence-positive cells compared to control Ig protein-bound cells.

FIG. 35 shows that DD1α-mediated inhibition of T cell activation is modulated by PD-1 blocking antibody (α-PD-1). Human CD4⁺ T cells were pre-incubated with 0, 2, 5, and 10 μg/ml of anti-PD1 antibody for 30 min and then stimulated by plate-bound anti-CD3 antibody (3 μg/ml) alone or together with DD1α-Ig, PD-L1-Ig or control Ig protein with the indicated combinations. The proliferations of CD4⁺ T cells were determined by percentage of CFSE-diluted CD4⁺ cells on day 3.

FIGS. 36A-36B show that DD1α-mediated inhibition of cytokines such as IFN-γ (FIG. 36A) and TNF-α (FIG. 36B) can be rescued by PD-1 blocking antibodies (α-PD-1). Human CD4⁺ T cells were pre-incubated with 0, 2, 5, and 10 μg/ml of anti-PD1 antibody for 30 min and then stimulated by plate-bound anti-CD3 antibody (3 μg/ml) alone or together with DD1α-Ig, PD-L1-Ig or control Ig protein with the indicated combinations. The cytokine production of CD4⁺ T cells were then determined IFN-γ and TNF-alpha levels in culture supernatants on day 2 were analyzed by ELISA.

FIG. 37 shows that both DD1α-Ig and PD-1-Ig proteins bind to Jurkat cells expressing DD1α receptor. Jurkat cells overexpressing DD1alpha (transfected with DD1alpha cDNA) or control Jurkat cells were assayed for binding to Ig-fused DD1α or PD-1 proteins. Cells were incubated with several different concentrations of DD1α-Ig (left panels) or PD1-Ig (right panels), and the bound Ig fusion proteins were detected by anti-Ig-APC. Binding amounts were determined by percentage of APC positive cells.

FIG. 38 shows experimental data on identification of DD1α blocking antibodies, tand that peripheral blood mononuclear cell (PBMC) activation by anti-CD3 antibodies (α-CD-3) can be increased by DD1α blocking monoclonal antibodies as similar to PD-1 blocking antibodies. PMBCs were isolated from human blood samples and treated with the indicated antibodies. The cells were then activated with or without α-CD-3. IFN-γ production was measured by ELISA.

### DETAILED DESCRIPTION OF THE INVENTION

The compositions and methods described herein are based, in part, on the discovery that there is a T cell and macrophage signaling axis involving p53, DD1α and PD-1/PD-L1. In one aspect, the inventors have identified DD1α receptor as a post-apoptotic target gene of p53, which is induced in apoptotic cells and highly expressed in immune cells, including, but not limited to macrophages, dendritic cells, monocytes, myeloid cells and T cells. Further, the inventors have shown that DD1α can function as an “eat-me” signal-engulfment ligand of apoptotic cells. In another aspect, the inventors have discovered that DD1α, which shares homology to B7 family member PD-L1, functions as a negative immune checkpoint regulator that is involved in modulating immune response and/or T cell function associated with PD-1, a co-inhibitory immunoreceptor of T cell tolerance. The inventors have also shown that p53 induces expression and/or activity of DD1α, as well as PD-1 and its ligand PD-L1. Thus, p53 can serve as a guardian for immune integrity via p53, DD1α, PD-1 and/or PD-L1 signaling axis. Accordingly, not only can agents that modulate the activity and/or expression of p53, DD1α and PD-1/PD-L1 be used for treatment of immune related diseases or disorders such as autoimmune disease, infection, chronic inflammation, cancer, asthma, and allergy, but p53 can also be used as a predictive marker to identify subjects who are more likely to benefit from an immunotherapy targeting DD1α, PD-1, and/or PD-L1. Accordingly, various aspects described herein provide for methods of identifying subjects with an immune-related disease or disorder who are more likely to be responsive to an immunotherapy or a therapy that targets DD1α, PD-1, and/or PD-L1, as well as monitoring the treatment efficacy. Methods and compositions for treating subjects with an immune-related disease or disorder are also provided herein.

In some immune-related diseases or disorders, including, but not limited to cancer, asthma, allergy, and/or infection (e.g., bacterial and fungal infection), it can be desirable to induce immune response, e.g., by increasing T-cell proliferation, for a therapeutic effect. For example, in one aspect, the inventors have discovered that DD1α expressed on the surface of a tumor cell can reduce T-cell proliferation and prevent anti-tumor effects mediated by the immune system. Such effects can be attributed to the homophilic intercellular interaction of DD1α with DD1α and/or heterophilic intercellular interaction of DD1α with PD-1. In contrast, in some other immune-related diseases or disorders, including, but not limited to autoimmune diseases, it can be desirable to suppress an immune response. Accordingly, some aspects provided herein relate to methods for determining whether a subject is amenable to treatment with an immunotherapy targeting PD-1 and/or DD1α, e.g., a bispecific or multispecific agent targeting DD1α/DD1α intercellular interaction and/or DD1α/PD-1 intercellular interaction. In some aspects, provided herein are methods and compositions for treating cancer and infection that target the homophilic binding interaction of DD1α with DD1α and/or the heterophilic interactions of PD-1 (e.g., binding interaction between DD1α and PD-1, PD-1 and PD-L1, and/or PD-1 and PD-L2). For example, bispecific or multispecific agents that target DD1α and PD-1, including agents that specifically or selectively target DD1α homophilic interactions and target heterophilic DD1α: PD-1 interactions can be used in treatment of cancer and infection.

### Some Selected Definitions

As used herein, the term “immunotherapy” generally refers to a treatment of a condition, e.g., a disease or disorder, that comprises an agent for inducing or suppressing an immune response. The agent can be an antibody, an antibody fragment, a peptide, a small molecule, a nucleic acid molecule, an aptamer, a vaccine, a peptidomimetic, or any combinations thereof. Immunotherapy takes advantages of aspects of the immune system and one or more of its cells for its effectiveness.

As used herein, an “immune response” being induced or suppressed refers to a response by a cell of the immune system, such as a B cell, T cell (CD4 or CD8), regulatory T cell, antigen-presenting cell, dendritic cell, monocyte, macrophage, NK T cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some embodiments, the immunotherapy can be a proinflammatory immunotherapy. In other embodiments, the immunotherapy can be an anti-inflammatory immunotherapy.

In accordance with various aspects described herein, the term “immunotherapy” refers to a treatment of a condition, e.g., a disease or disorder, comprising activation or suppression of one or more immune responses through the DD1α/PD-1 axis, i.e., activating or suppressing DD1α activity, alone or in combination with PD-1 activities. In some embodiments, modulating the DD1α/PD-1 axis can encompass activating or inhibiting DD1α: DD1α homophilic interaction and DD1α: PD 1 heterophilic interaction. In some embodiments, the term “immunotherapy” can further comprise activating or suppressing the functional interaction of PD-1 with its ligands, e.g., PD-L1 and/or PD-L2.

As used herein, the term “proinflammatory immunotherapy” generally refers to an immunotherapy treatment comprising either an agent that activates an inflammatory response, or an agent that suppresses an anti-inflammatory response. In some embodiments, the proinflammatory immunotherapy does not inhibit DD1α or PD-1 activity or interaction, but activates a proinflammatory T cell response pathway or inhibits an anti-inflammatory T cell response pathway via other immune checkpoints. In some embodiments, a proinflammatory immunotherapy can be used to treat a subject with cancer. In some embodiments, a proinflammatory immunotherapy can be used to treat a subject with asthma or allergy.

In some embodiments, a proinflammatory immunotherapy can induce T cells to produce proinflammatory factors such as Th1 and/or Th17 cytokines, e.g., but not limited to IFNγ, TNFα, GM-CSF, IL-2, IL-9, IL-17, IL-21, and IL-22. In some embodiments, a proinflammatory immunotherapy can activate at least one or more of the “stimulatory immune checkpoints,” including, but not limited to CD28, ICOS, 4-1BB, OX40, and/or CD27.

As used herein, the term “anti-inflammatory immunotherapy” generally refers to an immunotherapy treatment comprising either an agent that suppresses an inflammatory response, or an agent that activates an anti-inflammatory response. In some embodiments, the anti-inflammatory immunotherapy does not induce DD1α or PD-1 activity or interaction, but suppresses a proinflammatory T cell response pathway or activates an anti-inflammatory T cell response pathway via other immune checkpoints. In some embodiments, an anti-inflammatory immunotherapy can be used to treat a subject with an autoimmune disease.

In some embodiments, an anti-inflammatory immunotherapy can induce T cells to produce anti-inflammatory factors such as Th2 cytokines or immunosuppressive cytokines, e.g., but not limited to IL-4, IL-5, IL-6, IL-10, IL-13, TGFβ, IL-35, and/or IL-27. In some embodiments, an anti-inflammatory immunotherapy can activate at least one or more of the “inhibitory immune checkpoints,” including, but not limited to PD-1, CTLA-4, BTLA, LAG-3, and/or TIM-3.

As used herein, the term “bispecific polypeptide agent” refers to a polypeptide that comprises a first polypeptide domain which has a binding site that has binding specificity for a first target, and a second polypeptide domain which has a binding site that has binding specificity for a second target, i.e., the agent has specific binding sites for two targets. In some embodiments, the first target and the second target are expressed on different cells. In some embodiments, the first and the second targets bind each other. Thus, a bispecific polypeptide agent can disrupt or block the binding interaction between the first target and the second target. For example, in some embodiments, a bispecific polypeptide agent can bind the first target and/or the second target, prior to the interaction between the first target and the second target. In some embodiments, a bispecific polypeptide agent can displace the first target or the second target from the binding complex formed between the first target and the second target, when the first polypeptide domain and/or the second polypeptide domain of the bispecific polypeptide agent has a higher binding affinity than the first target and/or the second target binding to each other.

In one embodiment, the first target and the second target are not the same (i.e., are different targets (e.g., different proteins, or the same proteins with different post-translational processing such as glycosylation)), but are expressed on a cell or cells, such that the two different targets are in proximity to permit a heterophilic interaction (e.g., PD-1:DD1α binding). A bispecific polypeptide agent that binds both DD1α and PD-1 can disrupt or block the binding between DD1α and PD-1. Such an agent can, in some embodiments, disrupt or block the interaction of DD1α and PD-1 with other ligands. A non-limiting example of a bispecific polypeptide agent is a bispecific antibody construct or bispecific antigen-binding fragment thereof.

While homophilic interaction between, e.g., DD1α monomers, can, in some embodiments, be disrupted by an agent with a single DD1α-specific binding site, it is also contemplated that a bispecific agent with two binding sites for the same target polypeptide can be used, i.e., two binding sites for DD1α. In some embodiments, the target DD1α protein can have different glycosylation patterns on different cell types, e.g., cancer cells vs. immune cells. In these embodiments, disrupting a homophilic interaction of DD1α on a first cell type (e.g., cancer cells) with DD1α on a second cell type (e.g., immune cells) can employ a bispecific polypeptide agent with two binding sites specifically designed for differentially glycosylated DD1α on respective cells.

As used herein, the term “multispecific polypeptide agent” refers to a polypeptide that comprises at least a first polypeptide domain having a binding site that has binding specificity for a first target, and a second polypeptide domain having a binding site that has binding specificity for a second target. In some embodiments, the first and the second targets are the same protein, but permit binding to at least two monomers. In other embodiments, the first target and the second target are not the same (i.e., are different targets (e.g., different proteins, or the same proteins with different post-translational processing such as glycosylation)). A multispecific polypeptide agent as described herein can in addition specifically bind one or more additional targets, i.e., a multispecific polypeptide can bind at least two, at least three, at least four, at least five, at least six, or more targets, wherein the multispecific polypeptide agent has at least two, at least, at least three, at least four, at least five, at least six, or more target binding sites respectively. In one embodiment, a multispecific polypeptide agent can comprise (i) one or more target binding sites that disrupt the binding of first DD1α with second DD1α, and/or the binding of DD1α with PD-1, and (ii) further comprises one or more target binding sites that disrupt the binding of PD1 to its ligands, e.g., PD-L1 and/or PD-L2. A non-limiting example of a multispecific polypeptide agent is a multispecific antibody construct or antigen-binding fragment thereof. For the avoidance of doubt, a bispecific polypeptide agent is a type of multispecific polypeptide agent.

It should be noted that peptide or non-polypeptide agents, e.g., siRNA's, aptamers, small molecules, etc., can also be prepared as bispecific or multispecific agents if so desired.

The term “specificity” refers to the number of different types of antigens or antigenic determinants to which a particular antibody or antigen-binding fragment thereof can bind. The specificity of an antibody or antigen-binding fragment or portion thereof, alone or in the context of a bispecific or multispecific polypeptide agent, can be determined based on affinity and/or avidity. The affinity, represented by the equilibrium constant for the dissociation (K_{D}) of an antigen with an antigen-binding protein (such as a bispecific or multispecific polypeptide agent), is a measure for the binding strength between an antigenic determinant and an antigen-binding site on the antigen-binding protein: the lesser the value of the K_{D}, the stronger the binding strength between an antigenic determinant and the antigen-binding molecule. Alternatively, the affinity can also be expressed as the affinity constant (K_{A}), which is 1/K_{D}). As will be clear to the skilled person, affinity can be determined in a manner known per se, depending on the specific antigen of interest. Accordingly, a bispecific or multispecific polypeptide agent as defined herein is said to be “specific for” a first target or antigen compared to a second target or antigen when it binds to the first antigen with an affinity (as described above, and suitably expressed, for example as a K_{D} value) that is at least 10 times, such as at least 100 times, and preferably at least 1000 times, and up to 10,000 times or more better than the affinity with which said amino acid sequence or polypeptide binds to another target or polypeptide. Preferably, when a bispecific or multispecific polypeptide agent is “specific for” a target or antigen compared to another target or antigen, it is directed against said target or antigen, but not directed against such other target or antigen.

Avidity is the measure of the strength of binding between an antigen-binding molecule (such as a bispecific polypeptide agent described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as a bispecific polypeptide agent described herein) will bind to their cognate or specific antigen with a dissociation constant (K_{D} of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g. specific) are typically in the range of 10⁻¹⁰ M (0.1 nM) to 10⁻⁵ M (10000 nM). The stronger an interaction is, the lower is its K_{D}. Preferably, a binding site on a bispecific or multispecific polypeptide agent described herein will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known in the art, including, for example, Scatchard analysis and/or competitive binding assays, such as radioimmunoassays (RIA), enzyme immunoassays (EIA) and sandwich competition assays, and the different variants thereof known in the art; as well as other techniques as mentioned herein.

Accordingly, as used herein, “selectively binds” or “specifically binds” refers to the ability of a polypeptide domain described herein to bind to a target, such as a molecule present on the cell-surface, with a K_{D} 10⁻⁵ M (10000 nM) or less, e.g., 10⁻⁶ M or less, 10⁻⁷ M or less, 10⁻⁸ M or less, 10⁻⁹ M or less, 10⁻¹ M or less, 10⁻¹¹ M or less, or 10¹² M or less. For example, if a polypeptide domain or agent described herein binds to DD1α with a K_{D} of 10⁻⁵ M or lower, but not to other proteins such as PD-1, or binds to other protein(s) with a K_{D} greater than 10⁻⁵ M (e.g., 10⁻⁴M or higher), then the agent is said to specifically bind DD1α. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

As used herein, “modulating” or “to modulate” generally means either reducing or inhibiting the activity of, or alternatively increasing the activity of, a target or antigen, as measured using a suitable in vitro, cellular or in vivo assay. In particular, “modulating” or “to modulate” can mean either reducing or inhibiting the activity of, or alternatively increasing a (relevant or intended) biological activity of, a target or antigen, as measured using a suitable in vitro, cellular or in vivo assay (which will usually depend on the target or antigen involved), by at least 5%, at least 10%, at least 25%, at least 50%, at least 60%, at least 70%, at least 80%, or 90% or more, compared to activity of the target or antigen in the same assay under the same conditions but without the presence of a bispecific or multispecific polypeptide agent described herein. In some embodiments where the modulation is to increase the activity of a target or antigen, the increase as measured by a suitable in vitro, cellular, or in vivo assay can be at least about 1.1-fold or higher, including, e.g., at least about 1.5-fold, at least about 2-fold, at least about 3-fold, at least about 4-fold, at least about 5-fold, at least about 10-fold, at least about 50-fold, at least about 100-fold, as compared to activity of the target or antigen in the same assay under the same conditions but without the presence of an agent that causes the increased activity of the target or antigen.

As will be clear to the skilled person, “modulating” can also involve effecting a change (which can either be an increase or a decrease) in affinity, avidity, specificity and/or selectivity of a target or antigen for one or more of its ligands, binding partners, partners for association into a homomultimeric or heteromultimeric form, or substrates; and/or effecting a change (which can either be an increase or a decrease) in the sensitivity of the target or antigen for one or more conditions in the medium or surroundings in which the target or antigen is present (such as pH, ion strength, the presence of co-factors, etc.), compared to the same conditions but without the presence of a bispecific or multispecific polypeptide agent. Again, this can be determined in any suitable manner and/or using any suitable assay known per se, depending on the target or antigen involved.

“Modulating” can also mean effecting a change (i.e. an activity as an agonist, as an antagonist or as a reverse agonist, respectively, depending on the target or antigen and the desired biological or physiological effect) with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable (in vitro and usually cellular or in vivo assay) assay known in the art, depending on the target or antigen involved. In particular, an action as an agonist or antagonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the agonist or antagonist agent. In some embodiments where the modulation by an agonist is to increase an intended biological or physiological activity, the increase as measured by a suitable in vitro, cellular, or in vivo assay can be at least about 1.1-fold or higher, including, e.g., at least about 1.5-fold, at least about 2-fold, at least about 3-fold, at least about 4-fold, at least about 5-fold, at least about 10-fold, at least about 50-fold, at least about 100-fold, as compared to the biological or physiological activity in the same assay under the same conditions but without the presence of the agonist agent.

Modulating can for example also involve allosteric modulation of the target or antigen; and/or reducing or inhibiting the binding of the target or antigen to one of its substrates or ligands and/or competing with a natural ligand, substrate for binding to the target or antigen. Modulating can also involve activating the target or antigen or the mechanism or pathway in which it is involved. Modulating can for example also involve effecting a change in respect of the folding or conformation of the target or antigen, or in respect of the ability of the target or antigen to fold, to change its conformation (for example, upon binding of a ligand), to associate with other (sub)units, or to disassociate. Modulating can for example also involve effecting a change in the ability of the target or antigen to transport other compounds or to serve as a channel for other compounds (such as ions).

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, the term “greater than” in the context of an increase in the activity level and/or expression of a target molecule (e.g., p53, DD1α and/or PD-1) relative to its corresponding reference (e.g., a p53 reference, a DD1α reference and/or a PD-1 reference), the increase can be at least about 30% or more, including, e.g., at least about 40%, at least about 50%, at least about 60%, at least about 70%, at least about 80%, at least about 90%, at least about 95%, at least about 100% or more. In some embodiments, the increase can be at least about 1.1-fold or more, including, e.g., at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, or more.

As used herein, the term “homophilic interaction” refers to interaction (e.g., binding) between a first DD1α molecule and a second DD1α molecule. In some embodiments, the first DD1α molecule and the second DD1α molecule can be present on different cells. For example, the first DD1α molecule can be present on the surface of a cancer cell while the second DD1α molecule can be present on the surface of an immune cell. In some embodiments, the first DD1α molecule and the second DD1α molecule can be identical. In some embodiments, the first DD1α molecule and the second DD1α molecule can refer to the same or homologous protein sequence, but with different post-translation processing such as glycosylation. In some embodiments, at least one or both of the first DD1α molecule and the second DD1α molecule can refer to a functional portion of DD1α protein sequence that binds to PD-1.

As used herein, the term “heterophilic interaction” refers to interaction (e.g., binding) between a first molecule and a second molecule, wherein the first molecule and the second molecule are different. In some embodiments, the first molecule can be DD1α while the second molecule can be PD-1. In some embodiments, the first molecule can be PD-1 and the second molecule can be PD-L1. In some embodiments, the first molecule can be PD-1 and the second molecule can be PD-L2. In some embodiments, the first and the second molecules can be present on different cells. For example, the first molecule, e.g., DD1α, can be present on the surface of a cancer cell while the second molecule, e.g., PD-1, can be present on the surface of an immune cell.

As used herein, the term “lower than” or “less than” in the context of a decrease in the activity level and/or expression of a target molecule (e.g., p53, DD1α and/or PD-1) relative to its corresponding reference (e.g., a p53 reference, a DD1α reference and/or a PD-1 reference), the decrease can be at least about 30% or more, including, e.g., at least about 40%, at least about 50%, at least about 60%, at least about 70%, at least about 80%, at least about 90%, at least about 95%, or higher.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment of the invention.

All references cited herein are incorporated by reference in their entirety as though fully set forth. Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The term “or” is inclusive unless modified, for example, by “either.” Other than in the operating examples, or where otherwise indicated, all numbers expressing quantities of ingredients or reaction conditions used herein should be understood as modified in all instances by the term “about.” The term “about” with respect to numerical values means within 5%.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

Methods and Compositions for Treating Immune-Related Diseases where a Proinflammatory Response is Desirable

In some immune-related diseases or disorders, including, but not limited to cancer, asthma, allergy, and/or infection (e.g., bacterial and fungal infection), it can be desirable to induce immune response, e.g., by increasing T-cell proliferation, for a therapeutic effect. Accordingly, these immune-related diseases or disorders, e.g., but not limited to cancer, asthma, allergy, and/or infections (e.g., bacterial and/or fungal infections), where upregulation of immune responses is desirable, can be treated by inhibiting or reducing the expression or activity of DD1α and/or PD-1. However, not every patient, e.g., not every patient with cancer, asthma, allergy, and/or infection, would necessarily benefit from a treatment that inhibits the level of DD1α and/or PD-1 expression and/or activity. The inventors discovered that p53 induces expression and/or activity of DD1α as well as PD-1 and its ligands such as PD-L1 and/or PD-L2. For example, p53-overexpressing tumors which include high levels of DD1α and/or PD-1 expression and/or activity would be expected to respond better to immunotherapies designed to inhibit or reduce the expression or activity of DD1α and/or PD-1, because overexpression of DD1α and/or PD-1 on cancer cells enables the cancer cells to interact with T-cells through intercellular homophilic interaction of DD1α with DD1α and/or heterophilic interactions of PD-1 with DD1α and/or PD-1 ligands such as PD-L1 and/or PD-L2, and thus suppresses the immune response and escape of tumor cells from immune surveillance. In these instances, only after the DD1α interaction (homophilic and/or heterophilic interactions) is inhibited would one expect a strong anti-tumor immune response.

Accordingly, one aspect provided herein relates to a method of identifying a cancer patient who is more likely to respond to an anti-DD1α and/or anti-PD-1 therapy. The method comprises: measuring the level of p53 activity or expression in a sample from a cancer patient; and comparing the level of p53 or expression in the sample with a p53 reference, and: (i) when the level of p53 activity or expression is greater than the p53 reference, the cancer patient is identified to be more likely to respond to an anti-DD1α and/or anti-PD-1 therapy; or (ii) when the level of p53 activity or expression is the same as or less than the p53 reference, the cancer patient is identified to be more likely to respond to an alternative, proinflammatory immunotherapy without an anti-DD1α and/or anti-PD-1 therapy. In some embodiments, the method further comprises identifying the cancer patient who is more likely to respond to an anti-DD1α and/or anti-PD-1 therapy, or who is more likely to benefit from an alternative proinflammatory immunotherapy without an anti-DD1α and/or anti-PD-1 therapy, based on the level of p53 activity and/or expression measured in the patient's sample.

In some embodiments of this aspect and other aspects described herein, p53 can be a wild-type p53. In some embodiments of this aspect and other aspects described herein, p53 can be an isoform of wild-type 53, including, but not limited to p73 and p63.

In some embodiments of this aspect and other aspects described herein, the cancer patient is diagnosed with a cancer type expressing wild-type p53.

Another aspect provided herein relates to a method of identifying a patient who is diagnosed with an infection, e.g., caused by a bacterial and/or fungal pathogen, and is more likely to respond to an anti-DD1α and/or anti-PD-1 therapy. The method comprises: measuring the level of p53 activity or expression in a sample from a patient diagnosed with an infection, e.g., caused by a bacterial and/or fungal pathogen; and comparing the level of p53 or expression in the sample with a p53 reference, and: (i) when the level of p53 activity or expression is greater than the p53 reference, the patient is identified to be more likely to respond to an anti-DD1α and/or anti-PD-1 therapy; or (ii) when the level of p53 activity or expression is the same as or less than the p53 reference, the patient is identified to be more likely to respond to an alternative, proinflammatory immunotherapy without an anti-DD1α and/or anti-PD-1 therapy. In some embodiments, the method further comprises identifying the patient who is more likely to respond to an anti-DD1α and/or anti-PD-1 therapy, or who is more likely to benefit from an alternative proinflammatory immunotherapy without an anti-DD1α and/or anti-PD-1 therapy, based on the level of p53 activity and/or expression measured in the patient's sample.

Also provided herein, in another aspect, is a method of identifying a patient diagnosed to have asthma or allergy who is more likely to respond to an anti-DD1α and/or anti-PD-1 therapy, the method comprising: measuring the level of p53 activity or expression in a sample from a patient diagnosed to have asthma or allergy; and comparing the level of p53 or expression in the sample with a p53 reference, and: (i) when the level of p53 activity or expression is greater than the p53 reference, the patient is identified to be more likely to respond to an anti-DD1α and/or anti-PD-1 therapy; or (ii) when the level of p53 activity or expression is the same as or less than the p53 reference, the patient is identified to be more likely to respond to an alternative, proinflammatory immunotherapy without an anti-DD1 or anti-PD-1 therapy. In some embodiments, the method further comprises identifying the patient who is more likely to respond to an anti-DD1α and/or anti-PD-1 therapy, or who is more likely to benefit from an alternative proinflammatory immunotherapy without an anti-DD1α and/or anti-PD-1 therapy, based on the level of p53 activity and/or expression measured in the patient's sample.

As used herein, the phrase “more likely to be responsive” generally refers to likelihood of a subject to respond to a treatment. In accordance with one aspect of the discovery that DD1α is a target of p53, and that DD1αfunctions as a negative T cell checkpoint regulator associated with an inhibitor of immune checkpoint, PD-1, by determining the level of p53 expression or activity, one can predict the immune response of a subject administered an agent that modulates DD1α and/or PD-1 expression or activity, which can in turn produce an effect on a disease or condition.

As used herein, the term “expression” refers to the protein or mRNA amount of a target molecule (e.g., p53) in a sample.

As used herein, the term “activity” refers to the ability of a target molecule (e.g., p53) to directly or indirectly modulate an immune response in a subject. Non-limiting examples of such modulation include activities or a trans-acting factor, receptor, or ligand.

In one embodiment of this aspect and all other aspects described herein, the method further comprises administering an anti-DD1α and/or anti-PD-1 therapy to the patient when the level of p53 activity or expression is greater than the p53 reference. In another embodiment of this aspect and all other aspects described herein, the method can further comprise increasing the dose of the anti-DD1α and/or anti-PD-1 therapy over a period of time. An anti-PD-1 therapy can comprise an agent that antagonizes the binding of PD-1 with PD-L1 and/or PD-L2. In some embodiments, the anti-PD-1 therapy can comprise a PD-1 inhibitor, a PD-L1 inhibitor, a PD-L2 inhibitor, or a combination of two or more thereof. In some embodiments, an anti-DD1α therapy can comprise an agent that inhibits homophilic interactions between DD1α molecules and/or an agent that inhibits heterophilic interactions between DD1α molecules and PD-1 molecules. In another embodiment of this aspect and all other aspects described herein, the inhibitor or agent used in the anti-DD1α and/or anti-PD-1 therapy can comprise a protein, a peptide, a nucleic acid, an antibody, a small molecule, a vaccine, and combinations thereof.

In one embodiment of this aspect and all other aspects described herein, the method further comprises administering an alternative, proinflammatory immunotherapy without an anti-DD1α and/or anti-PD-1 therapy when the level of p53 activity or expression is the same as or less than the p53 reference. An exemplary alternative, proinflammatory immunotherapy can comprise an activator of a proinflammatory T cell response pathway and/or a suppressor of an anti-inflammatory T cell response pathway. Non-limiting examples of the activator of the proinflammatory T cell response pathway and/or suppressor of the anti-inflammatory T cell response pathway include a TIGIT inhibitor, a Fgl2 inhibitor, a TIM-3 inhibitor, an anti-galectin-9 molecule, a CTLA-4 antagonist, a Lag-3 antagonist, an agonist of an immune checkpoint activating molecule, an antagonist of an immune checkpoint inhibitory molecule, or any combination thereof. Soluble versions of membrane-bound targets are specifically contemplated herein as inhibitors.

In another embodiment of this aspect and all other aspects described herein, the patient amenable to the methods described herein can be a patient that has been receiving a therapy to treat the target immune-related disease or disorder, e.g., anti-cancer therapy, anti-asthma therapy, or anti-allergy therapy. In some embodiments, the treatment can be an immunotherapy.

In this aspect and all other aspects described herein, a p53 reference used for comparison to a measured level of p53 activity or expression in a patient's sample generally involves a positive control, a negative control, and/or a threshold value. In some embodiments, the p53 reference can correspond to the level of p53 activity or expression in a normal healthy subject. The term “normal healthy subject” generally refers to a subject who has no symptoms of any diseases or disorders, or who is not identified with any diseases or disorders, or who is not on any medication treatment, or a subject who is identified as healthy by a physician based on medical examinations. In some embodiments, the p53 reference can correspond to the level of p53 activity or expression in a normal tissue of the same type or lineage as the sample (e.g., same type or lineage as a tissue biopsy obtained from a target site (e.g., a tumor or an inflammatory tissue). In some embodiments, the normal tissue of the same type or lineage as the sample can be obtained from a patient subjected to at least one aspect of the methods described herein. In some embodiments, the p53 reference can correspond to the level of p53 expression or activity in a tissue biopsy with a known level of p53 expression or activity. In some embodiments, the p53 reference can correspond to the level of p53 expression or activity measured in a patient's sample taken at a prior time point. In some embodiments, the p53 reference can correspond to a threshold level of p53 activity or expression or a standard numeric level.

When the level of p53 activity or expression is greater than the p53 reference, e.g., by at least about 10% or more, including, e.g., at least about 20%, at least about 30%, at least about 40%, at least about 50%, at least about 60%, at least about 70%, at least about 80%, at least about 90%, at least about 100% or more, the patient diagnosed with a disease or disorder where an upregulation of immune response is desirable (e.g., cancer, allergy, asthma, and/or infection) is identified to be more likely to be responsive to an anti-DD1α and/or anti-PD-1 therapy. In some embodiments, when the level of p53 activity or expression is greater than the p53 reference, e.g., by at least about 1.1-fold or more, including, e.g., at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, or more, the patient diagnosed with a disease or disorder where an upregulation of immune response is desirable (e.g., cancer, allergy, asthma, and/or infection) can be identified to be more likely to be responsive to an anti-DD1α and/or anti-PD-ltherapy. On the other hand, when the level of p53 activity or expression is substantially the same as or less than the p53 reference, e.g., by at least about 10% or more, including, e.g., at least about 20%, at least about 30%, at least about 40%, at least about 50%, at least about 60%, at least about 70%, at least about 80%, at least about 90%, or more, the patient diagnosed with a disease or disorder where an upregulation of immune response is desirable (e.g., cancer, allergy, asthma, and/or infection) is identified as likely to respond to an alternative, proinflammatory immunotherapy comprising an activator of a proinflammatory T cell response pathway and/or a suppressor of an anti-inflammatory T cell response pathway, e.g., without the need to suppress DD1α and/or PD-1 activity.

In this aspect and all other aspects described herein, the sample analyzed in the methods described herein can be a bodily fluid sample (e.g., blood) or a sample of a tissue at a target site from a patient. For example, for treatment of cancer, the sample can be a blood sample or a tumor biopsy from a patient. For treatment of other immune-related diseases or disorders, including, e.g., asthma, allergy, and infections, the sample can be a blood sample or a tissue biopsy from a target site to be treated in a patient.

While existing anti-PD-1 therapy, alone or in combination with an anti-cancer agent, can be used to treat cancer, the treatment does not target homophilic DD1α interaction and/or heterophilic DD1α/PD-1 interaction. Thus, the cancer cells that overexpress DD1α, PD-1, and/or PD-L1 (e.g., cancer cells that overexpress p53) can still escape from immune surveillance via the action of the DD1α signaling pathway when administered an anti-PD-1 therapy without targeting interaction of DD1α with DD1α and/or PD. Accordingly, another aspect provided herein is a method of treating cancer involving inhibition interaction of DD1α on a cancer cell with DD1α and/or PD-1 on an immune cell. The method comprises administering to a cancer patient in need thereof a treatment comprising an agent that antagonizes the homophilic interaction of DD1α with DD1α. In some embodiments, the agent can antagonize the homophilic interaction of DD1α with DD1α, e.g., by at least about 30% or more, including, e g., at least about 40%, at least about 50%, at least about 60%, at least about 70%, at least about 80%, at least about 90%, at least about 95% or more, as compared to a treatment without such agent.

In some embodiments, the cancer patient administered the treatment can be determined to have a level of p53 activity or expression greater than a p53 reference. In some embodiments, the cancer patient administered the treatment can be determined to have a level of DD1α activity or expression greater than a DD1α reference. In some embodiments, the cancer patient administered the treatment can be determined have a level of PD-1 activity or expression greater than a PD-1 reference.

In some embodiments, the agent that antagonizes the homophilic interaction of DD1α with DD1αcan further antagonize the functional interaction of DD1α with PD-1, e.g., by at least about 30% or more, including, e g., at least about 40%, at least about 50%, at least about 60%, at least about 70%, at least about 80%, at least about 90%, at least about 95% or more, as compared to a treatment without such agent. In these embodiments, the agent can comprise a moiety that binds DD1α and a moiety that binds PD-1. In some embodiments, the agent can be a peptide or an antibody. In some embodiments, the moiety that binds DD1αcan be attached to the moiety that binds PD-1 via a linker moiety. In some embodiments, the moieties that bind DD1α and PD-1 can comprise antigen-binding domains of antibodies that specifically bind DD1α and PD-1, respectively.

In some embodiments of this aspect and other aspects described where both DD1α/DD1α interaction and DD1α/PD-1 interaction are targeted, the therapeutic effects (e.g., reducing at least one symptom, associated with an immune-related disease or disorder such as reduced tumor growth in cancer) can be additive. As used herein, the term “additive” as used herein generally refers to the combined effect of targeting at least two or more target molecules and/or target interactions/signaling (e.g., both DD1α/DD1α interaction and DD1α/PD-1 interaction) being substantially equal to the sum of their individual effects.

In some embodiments of this aspect and other aspects described where both DD1α/DD1α interaction and DD1α/PD-1 interaction are targeted, the therapeutic effects (e.g., reducing at least one of the symptoms associated with an immune-related disease or disorder, such as reduced tumor growth in cancer) can be synergistic. The term “synergy” or “synergistic” as used herein generally refers to the combined effect of targeting at least two or more target molecules and/or target interactions/signaling (e.g., both DD1α/DD1α interaction and DD1α/PD-1 interaction) being greater than the sum of their individual effects. In particular, the term “synergy” or “synergistic” as used herein refers to the combined therapeutic effect associated with an immune-related disease or disorder when a patient is treated with a therapy that targets both DD1α/DD1α interaction and DD1α/PD-1 interaction, in which the effect is greater than the sum of the therapeutic effect associated with the individual DD1α/DD1α interaction and DD1α/PD-1 interaction (additive effect). In some embodiments, the synergistic effect can be greater than the additive effect by at least about 10%, at least about 20%, at least about 30%, at least about 40%, at least about 50%, at least about 60%, at least about 70%, at least about 80%, at least about 90%, at least about 95% or more. In some embodiments, the synergistic effect can be greater than the additive effect by at least about 1.5-fold, at least about 2-fold, at least about 3-fold, at least about 4-fold, at least about 5-fold, at least about 10-fold or higher.

As used herein, the term “linker moiety” refers to an entity that can directly or indirectly connect two parts of a composition, e.g., a moiety that binds DD1α and a moiety and binds PD-1. The linker moiety can be configured to have an appropriate length such that the two parts of the composition can function properly. For example, the linker moiety can be configured to have an appropriate length such that the moiety that binds DD1α does not interfere with the moiety that binds PD-1. Examples of a linker moiety can include, but are not limited to a peptide, a peptidomimetic, an aptamer, a protein, a nucleic acid, a small molecule, or any combination thereof.

In some embodiments, the treatment can be further adapted to disrupt binding of PD-1 with PD-L1 and/or PD-L2. In some embodiments, the agent that antagonizes the homophilic interaction of DD1α with DD1αcan be adapted to further disrupt binding of PD-1 with PD-L1 and/or PD-L2.

In some embodiments, the treatment for cancer can be co-administered with an anti-cancer agent. As used herein, the term “co-administer” or “in combination with” in the context of therapy administration generally refers to administering a first agent and at least a second agent. The first agent and the second agent can be administered concurrently or simultaneously (e.g., in the same or separate unit dosage forms), or separately at different times. The first agent and the second agent can be administered by the same or different route.

As used herein, an “anti-cancer agent” or “anti-cancer therapy” is generally an agent or a therapy for treatment of cancer, e.g., an agent that kills cancer cells, and/or reduces or prohibits tumor growth and/or progression. Examples of anti-cancer agents include, but are not limited to cancer vaccines, chemotherapy, targeted therapy (e.g., kinase inhibitors), radiation therapy, surgery, immunotherapy, and any combinations thereof. One of skill in the art can readily identify a chemotherapeutic agent for use in treatment of cancer (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloffs Clinical Oncology, 2013 Elsevier; and Fischer D S (ed): The Cancer Chemotherapy Handbook, 4th ed. St. Louis, Mosby-Year Book, 2003).

Without wishing to be bound by theory, it is also contemplated that cells infected with a bacterial or fungal pathogen, similarly to cancer cells, overexpress DD1α and related immune checkpoint inhibitors such as PD-1 and PD-L1 molecules, which permits the infected cells to interact with T cells through intercellular homophilic DD1αbinding and/or heterophilic PD-1 binding (e.g., PD-1/DD1α; PD-1/PD-L1; and/or PD-1/PD-L2), and thus suppresses the immune response, allowing the infected cells to escape from immune surveillance. Accordingly, another aspect provided herein relates to a method of treating infection with a bacterial and/or fungal pathogen. The method comprises administering a treatment comprising an agent that antagonizes DD1α activity to a subject infected with a bacterial and/or fungal pathogen.

In some embodiments, the treatment can inhibit macrophage activity against host cell constituents while permitting pathogen phagocytosis by macrophages.

In some embodiments, the agent that antagonizes DD1α activity can antagonize the homophilic interaction of DD1α with DD1α, e.g., by at least about 30% or more, including, e.g., at least about 40%, at least about 50%, at least about 60%, at least about 70%, at least about 80%, at least about 90%, at least about 95% or more, as compared to a treatment without such agent.

In some embodiments, the agent that antagonizes DD1α activity can antagonize the functional interaction of DD1α with PD-1, e.g., by at least about 30% or more, including, e.g., at least about 40%, at least about 50%, at least about 60%, at least about 70%, at least about 80%, at least about 90%, at least about 95% or more, as compared to a treatment without such agent.

In some embodiments, the agent that antagonizes DD1α activity can antagonize the homophilic interaction of DD1α with DD1α and antagonizes the functional interaction of DD1α with PD-1. In these embodiments, the agent can comprise a moiety that binds DD1α and a moiety that binds PD-1. In some embodiments, the agent can be a peptide or an antibody. In some embodiments, the moiety that binds DD1αcan be attached to the moiety that binds PD-1 via a linker moiety. In some embodiments, the moieties that bind DD1α and PD-1 can comprise antigen-binding domains of antibodies that specifically bind DD1α and PD-1, respectively.

In some embodiments, the treatment can be adapted to also antagonize PD-1 activity. For example, in some embodiments, the treatment can be also adapted to disrupt binding of PD-1 with PD-L1 and/or PD-L2, e.g., by at least about 30% or more, including, e.g., at least about 40%, at least about 50%, at least about 60%, at least about 70%, at least about 80%, at least about 90%, at least about 95% or more, as compared to a treatment without such disruption. In some embodiments, the agent that antagonizes DD1α activity can be adapted to further disrupt binding of PD-1 with PD-L1 and/or PD-L2.

In some embodiments of various aspects described herein, at least one or more DD1α inhibitor(s) and/or PD-1 inhibitor(s) can be administered to a patient with a bacterial infection. The bacterial infection can be caused by intracellular bacteria and/or extracellular bacteria. Examples of infectious bacteria include: Helicobacterpyloris, Borelia burgdorferi, Chlamydia trachomatis, Legionella pneumophilia, Mycobacteria sps (such as M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, corynebacterium diphtheriae, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, and Actinomyces israelli.

In some embodiments of various aspects described herein, at least one or more DD1α inhibitor(s) and/or PD-1 inhibitor(s) can be administered to a patient with a fungal infection. Examples of fungal infections include but are not limited to: aspergillosis; thrush (caused by Candida albicans); cryptococcosis (caused by Cryptococcus); and histoplasmosis. Thus, examples of infectious fungi include, but are not limited to Cryptococcus neoformans, Histoplasma capsulatum, Coccidioides immitis, Blastomyces dermatitidis, Candida albicans.

In some embodiments, the treatment for bacterial and/or fungal infection can be co-administered with an anti-infection agent. As used herein, an “anti-infection agent” or “anti-infection therapy” is generally an agent or a therapy that kills a pathogen or inhibits a pathogen's cellular process, development and/or replication. Examples of an anti-infection agent or therapy include, but are not limited to anti-bacterial agent or therapy, anti-fungal agent or therapy, and a combination of two.

As used herein, the term “anti-bacterial agent” or “anti-bacterial therapy” refers to an agent that has bactericidal and/or bacteriostatic activity. The anti-bacterial agent can be naturally occurring or synthetic. In some embodiments, an anti-bacterial agent or therapy can comprise an antibiotic, e.g., to suppress the growth of other microorganisms. Non-limiting examples of anti-bacterial agents include β-lactam antibacterial agents including, e.g., ampicillin, cloxacillin, oxacillin, and piperacillin, cephalosporins and other cephems including, e.g., cefaclor, cefamandole, cefazolin, cefoperazone, cefotaxime, cefoxitin, ceftazidime, ceftriaxone, and cephalothin; carbapenems including, e.g., imipenem and meropenem; and glycopeptides, macrolides, quinolones, tetracyclines, and aminoglycosides. In general, if an antibacterial agent is bacteriostatic, it means that the agent essentially stops bacterial cell growth (but does not necessarily kill the bacteria); if the agent is bacteriocidal, it means that the agent kills the bacterial cells (and may stop growth before killing the bacteria).

As used herein, the term “anti-fungal agent” or “anti-fungal therapy” refers to an agent that is able to exert an inhibitory effect on the growth and/or development of a fungus. Such an effect can be classified as fungicidal, fungistatic, sporocidal, sporostatic, or a combination thereof. Examples of anti-fungal agent or therapy include, but are not limited to polyene-based, imidazole-based, triazole-based, thiazole-based, allyalmine-based, echinocandin-based, and a combination of two or more thereof.

It is also contemplated that the methods described herein to treat cancer and/or bacterial and/or fungal infections can be used to treat allergy and/or asthma, where modulation of a specific type of an immune response, e.g., shifting Th1/Th2 balance and/or dampening a Th2 response, is desirable to produce a therapeutic effect. The method comprises administering a treatment comprising an agent that antagonizes DD1α activity to a subject infected with asthma and/or allergy.

As used herein, the term “asthma” is intended to cover all types of asthma. Asthma is a chronic lung disease or disorder that inflames and narrows the airways.

As used herein, the term “allergy” refers to a disorder (or improper reaction) of the immune system often also referred to as “atopy.” Allergic reactions can occur when a subject's immune system reacts to environmental substances that are normally harmless to those without allergy. The substances that cause such allergic reactions are known as allergens. In some embodiments, allergy refers to type I (or immediate) hypersensitivity. Allergic reactions occur when there is excessive activation of certain white blood cells (e.g., mast cells and basophils) by immunoglobulin E (IgE). Common allergic reactions include eczema, hives, hay fever, asthma, food allergies, and reactions to the venom of stinging insects such as wasps and bees. Mild allergies like hay fever are highly prevalent in the human population and cause symptoms such as allergic conjunctivitis, itchiness, and runny nose. Allergies can play a role in conditions such as asthma.

Methods and Compositions for Treating Autoimmune Diseases and Other Immune-Related Diseases where a Suppression of Immune Response is Desirable

In some immune-related diseases or disorders where suppression of immune response is desirable, e.g., autoimmune diseases, a DD1α agonist and/or PD-1 agonist therapy can be administered. Accordingly, another aspect provided herein relates to a method of identifying a patient diagnosed to have an inflammatory disease or disorder who is more likely to respond to a DD1α agonist and/or PD-1 agonist therapy. Examples of an inflammatory disease or disorder include, but are not limited to infection, autoimmune diseases, acute inflammation, chronic inflammation, and combinations thereof. The method comprises: measuring the level of p53 activity or expression in a sample from a patient diagnosed to have an inflammatory disease or disorder; and comparing the level of p53 or expression in the sample with a p53 reference, and: (i) when the level of p53 activity or expression is lower than the p53 reference, the patient is identified to be more likely to respond to a DD1 agonist and/or PD-1 agonist therapy; or (ii) when the level of p53 activity or expression is the same as or greater than the p53 reference, the patient is identified to be more likely to respond to an alternative, anti-inflammatory immunotherapy without a DD1α agonist or PD-1 agonist therapy. In some embodiments, the method further comprises identifying the patient who is more likely to respond to a DD1α agonist and/or PD-1 agonist therapy, or who is more likely to benefit from an alternative, anti-inflammatory immunotherapy without a DD1α agonist and/or PD-1 agonist therapy, based on the level of p53 activity and/or expression measured in the patient's sample.

In some embodiments, the method can further comprise administering a DD1α agonist and/or PD-1 agonist therapy to the patient when the level of p53 activity or expression is lower than the p53 reference. A PD-1 agonist therapy can comprise an agent that enhances or induces binding of PD-1 with PD-L1 and/or PD-L2. In some embodiments, the PD-1 agonist therapy can comprise a PD-1 agonist, a PD-L1 agonist, and/or a PD-L2 agonist. In some embodiments, the DD1α agonist therapy can comprise an agent that increases homophilic interactions between DD1α molecules and/or an agent that increases heterophilic interactions between DD1αmolecules and PD-1 molecules. In some embodiments, the agonist or agent used in the DD1α agonist and/or PD-1 agonist therapy can comprise a protein, a peptide, a nucleic acid, an antibody, a small molecule, a vaccine, and combinations thereof.

In some embodiments, the method can further comprise administering an alternative, anti-inflammatory immunotherapy without a DD1α agonist and/or PD-1 agonist therapy when the level of p53 activity or expression is the same as or greater than the p53 reference. An exemplary alternative, anti-inflammatory immunotherapy can comprise a suppressor of a proinflammatory T cell response pathway and/or an activator of an anti-inflammatory T cell response pathway. Non-limiting examples of the suppressor of the proinflammatory T cell response pathway and/or activator of the anti-inflammatory T cell response pathway include a TIGIT agonist, a Fgl2 agonist, a TIM-3 agonist, a galectin-9 molecule, a CTLA-4 agonist, a Lag-3 agonist, an antagonist of an immune checkpoint activating molecule, an agonist of an immune checkpoint inhibitory molecule, or any combination thereof.

In some embodiments, the patient amenable to the methods described herein can be a patient who has been receiving an anti-inflammatory treatment, e.g., an anti-inflammatory immunotherapy.

A p53 reference used for comparison to a measured level of p53 activity or expression in a patient's sample generally involves a positive control, a negative control, and/or a threshold value. In some embodiments, the p53 reference can correspond to the level of p53 activity or expression in a normal healthy subject. In some embodiments, the p53 reference can correspond to the level of p53 activity or expression in a normal tissue of the same type or lineage as the sample (e.g., same type or lineage as a tissue biopsy obtained from a target site (e.g., an inflammatory tissue). In some embodiments, the normal tissue of the same type or lineage as the sample can be obtained from a patient subjected to at least one aspect of the methods described herein. In some embodiments, the p53 reference can correspond to the level of p53 expression or activity in a tissue biopsy with a known level of p53 expression or activity. In some embodiments, the p53 reference can correspond to the level of p53 expression or activity measured in a patient's sample taken at a prior time point. In some embodiments, the p53 reference can correspond to a threshold level of p53 activity or expression or a standard numeric level.

The sample analyzed in this aspect of the methods described herein can be a bodily fluid sample (e.g., blood) or a sample of a tissue at a target site from a patient. For example, for treatment of autoimmune diseases, the sample can be a blood sample or a tissue biopsy from a target site to be treated in a patient.

In some embodiments, the autoimmune diseases to be treated or prevented using the methods described herein, include, but are not limited to: rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus).

Autoimmune disease has been recognized also to encompass atherosclerosis and Alzheimer's disease. In one embodiment of the aspects described herein, the autoimmune disease is selected from the group consisting of multiple sclerosis, type-I diabetes, Hashinoto's thyroiditis, Crohn's disease, rheumatoid arthritis, systemic lupus erythematosus, gastritis, autoimmune hepatitis, hemolytic anemia, autoimmune hemophilia, autoimmune lymphoproliferative syndrome (ALPS), autoimmune uveoretinitis, glomerulonephritis, Guillain-Barre syndrome, psoriasis and myasthenia gravis.

In some embodiments of this aspect and other aspects described where both DD1α/DD1α interaction and DD1α/PD-1 interaction are targeted, the therapeutic effects (e.g., reducing at least one of the symptoms associated with an immune-related disease or disorder such as reduced tumor growth in cancer) can be additive.

In some embodiments of this aspect and other aspects described where both DD1α/DD1αinteraction and DD1α/PD-1 interaction are targeted, the therapeutic effects (e.g., reducing at least one of the symptoms associated with an immune-related disease or disorder, such as reduced tumor growth in cancer) can be synergistic.

### DD1α

DD1α, also known as V-region Immunoglobulin-containing Suppressor of T cell Activation (VISTA), PD-L3, or PD1H (programmed death-1 homolog), was originally discovered as a hematopoietically-restricted Ig superfamily inhibitory ligand. The extracellular domain has been shown to bear homology to the B7 family ligand PD-L1, and like PD-L1, DD1α has a profound impact on immunity. However, unlike PD-L1, expression of DD1α was thought to be expressed exclusively within the hematopoietic compartment. Expression is most prominent on myeloid antigen-presenting cells (APCs), although expression on CD4+ T cells and on a subset of Foxp3+ regulatory T cells (Treg) has also been identified to be of significant interest. A soluble DD1α-Ig fusion protein, or VISTA expression on APCs, has been shown to inhibit in vitro T cell proliferation, cytokine production and induce Foxp3 expression in T cells. Conversely, a newly developed anti-DD1α monoclonal antibody interfered with DD1α-induced immune suppression of T cell responses by DD1α +APCs in vitro. Furthermore, in vivo anti-DD1α intensified the development of the T cell mediated autoimmune disease experimental allergic encephalomyelitis (EAE), and facilitated the development of a protective, tumor-specific immune response with subsequent tumor remission. Initial studies of DD1α−/− mice are revealing early indications of spontaneous inflammatory disease.

Analysis of the peptide sequence of DD1α indicated that DD1α contains a signal peptide and a transmembrane region located in the middle (from 195 aa to 215 aa). The extracellular region of DD1αincludes the immunoglobulin variable (IgV) set (from 45 aa to 168 aa), which contains several potential N-linked glycosylation sites. As shown herein in the Examples section, the DD1αprotein migrated at approximately 50 kDa by western blot analysis due to glycosylation since after treatment with the glycosylation inhibitor tunicamycin, it migrated at the predicted size of ˜30 kDa. Northern blotting of various human tissues demonstrated high expression levels of DD1α in blood leukocytes, placenta, spleen and heart, and low levels in lung, kidney, small intestine and brain (see e.g., the working Examples).

In addition, as discussed herein in the Examples section, the inventors have found that DD1α on the surface of e.g., a tumor cell or macrophage can form a homodimer with another DD1α molecule (e.g., DD1α/DD1α) on the surface of a T-cell, thereby reducing proliferation of the T-cells. Thus, the presence of DD1α on the tumor cell can act to decrease immune cell mediated clearance of the tumor cell. In addition, DD1α interaction with DD1α on macrophages induces an “eat me” signal to initiate phagocytosis. Alternatively, DD1α on the surface of e.g., a tumor can form a heterodimer with PD-1 (e.g., PD-1/DD1α or DD1α/PD-1) present on a T-cell. Similar to the effects of DD1α homodimerization, heterodimerization of PD-1 and DD1α also reduces T-cell proliferation and induces an “eat me” signal in macrophages.

Accordingly, as used herein, the term “DD1α” generally refers to a DD1αpolypeptide or a DD1α polynucleotide that is similar or identical in sequence to a wild-type DD1α.

In some embodiments, the term “DD1α” refers to a DD1α polypeptide having an amino acid sequence that is at least 70% or more (including at least 75%, at least 80%, at least 85%, at least 90%, at least 95%, at least 97%, at least 99%, or 100%) identical to that of a wild-type DD1α, and is capable of forming DD1α/DD1α homodimers (e.g., to reduce proliferation of T-cells) and/or DD1α/PD-1 heterodimers. Accordingly, in some embodiments, a DD1α polypeptide can be a full-length DD1α. In some embodiments, a DD1α polypeptide refers to a functional domain or domains of DD1α that is capable of forming DD1α/DD1α homodimers (e.g., to reduce proliferation of T-cells) and/or DD1α/PD-1 heterodimers.

In one embodiment, the term “DD1α” refers to the 311 amino acid polypeptide having the amino acid sequence of (corresponding to Genbank Accession No. AFQ73336.1 and/or AAH20568):

(SEQ ID NO: 1)
mgvptaleag swrwgsllfa lflaaslgpv aafkvatpys
lyvcpegqnv tltcrllgpv dkghdvtfyk twyrssrgev
qtcserrpir nitfqdlhlh hgghqaants hdlaqrhgle
sasdhhgnfs itmrnltlld sglycclvve irhhhsehrv
hgamelqvqt gkdapsncvv ypsssqdsen itaaalatga
civgilclpl illlvykqrq aasnrraqel vrmdsniqgi
enpgfeaspp aggipeakvr hplsyvaqrq psesgrhlls
epstplsppg pgdvffpsld pvpdspnfev i

In some embodiments, the term “DD1α” refers to a polypeptide having an amino acid sequence that is at least 70% or more (including at least 75%, at least 80%, at least 85%, at least 90%, at least 95%, at least 97%, at least 99%, or 100%) identical to that of SEQ ID NO. 1, and is capable of forming DD1α/DD1α homodimers (e.g., to reduce proliferation of T-cells) and/or DD1α/PD-1 heterodimers.

### PD-1 and Ligands Thereof

PD-1 (or CD279) is a 288 amino acid type I transmembrane protein comprising an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif.

Splice variants of PD-1 have been cloned from activated human T cells. These transcripts lack exon 2, exon 3, exons 2 and 3, or exons 2 through 4. All these variants, except for the splice variant lacking exon 3 only (PD-1Δex3), are expressed at similar levels as full-length PD-1 in resting peripheral blood mononuclear cells (PBMCs). All variants are significantly induced upon activation of human T cells with anti-CD3 and anti-CD28 (Keir M E et al., 2008. Annu Rev Immunol. 26:677-704).

Accordingly, as used herein, the term “PD-1” generally refers to a PD-1 polypeptide or a PD-1 polynucleotide that is similar or identical in sequence to a wild-type PD-1.

In some embodiments, the term “PD-1” refers to a PD-1 polypeptide having an amino acid sequence that is at least 70% or more (including at least 75%, at least 80%, at least 85%, at least 90%, at least 95%, at least 97%, at least 99%, or 100%) identical to that of a wild-type PD-1, and is capable of binding DD1α, PD-L1 and/or PD-L2. Accordingly, in some embodiments, a PD-1 polypeptide can be a full-length PD-1. In some embodiments, a PD-1 polypeptide refers to a functional domain or domains of PD-1 that is capable of binding DD1α, PD-L1, and/or PD-L2.

In some embodiments, the term “PD-1” as used herein, refers to the 288 amino acid polypeptide having the amino acid sequence of: MQIPQAPWPVVWAVLQLGWRPGWFLDSPDRPWNPPTFSPALLVVTEGDNATFTCSFSNTSES FVLNWYRMSPSNQTDKLAAFPEDRSQPGQDCRFRVTQLPNGRDFHMSVVRARRNDSGTYLC GAISLAPKAQIKESLRAELRVTERRAEVPTAHPSPSPRPAGQFQTLVVGVVGGLLGSLVLLVW VLAVICSRAARGTIGARRTGQPLKEDPSAVPVFSVDYGELDFQWREKTPEPPVPCVPEQTEYA TIVFPSGMGTSSPARRGSADGPRSAQPLRPEDGHCSWPL (SEQ ID NO:2), as described by, e.g., NP_005009, together with any naturally occurring allelic, splice variants, and processed forms thereof. Typically, PD-1 refers to human PD-1. The term “PD-1” is also used to refer to truncated forms or fragments of the PD-1 polypeptide.

In some embodiments, the term “PD-1” refers to a polypeptide having an amino acid sequence that is at least 70% or more (including at least 75%, at least 80%, at least 85%, at least 90%, at least 95%, at least 97%, at least 99%, or 100%) identical to that of SEQ ID NO. 2, and is capable of binding DD1α, PD-L1 and/or PD-L2.

PD-1 has been shown to be expressed on T cells, B cells, natural killer T cells, activated monocytes, and dendritic cells (DCs). PD-1 is not expressed on resting T cells but is inducibly expressed after activation. Ligation of the T cell receptor or B cell receptor can upregulate PD-1 on T and B lymphocytes. In normal human reactive lymphoid tissue, PD-1 is expressed on germinal center-associated T cells. PD-1 compartmentalization in intracellular stores has been described in a regulatory T cell population. PD-1 is inducibly expressed on APCs on myeloid CD11c+ DCs and monocytes in humans (Keir M E et al., 2008. Annu Rev Immunol. 26:677-704).

PD-1 has two known ligands, PD-L1 and PD-L2, which are also members of the B7 family. The binding interface of PD-1 to PD-L1 is via its IgV-like domain (i.e., PD-1(42-136)). Residues important for binding of PD-1 to these ligands include residues 64, 66, 68, 73, 74, 75, 76, 78, 90, 122, 124, 126, 128, 130, 131, 132, 134, and 136. PD-L1/CD274 has been shown to be constitutively expressed on mouse T and B cells, DCs, macrophages, mesenchymal stem cells, and bone marrow-derived mast cells. CD274/PD-L1 expression is also found on a wide range of nonhematopoietic cells and is upregulated on a number of cell types after activation. PD-L1 is expressed on almost all murine tumor cell lines, including PA1 myeloma, P815 mastocytoma, and B16 melanoma upon treatment with IFN-γ. Loss or inhibition of phosphatase and tensin homolog (PTEN), a cellular phosphatase that modifies phosphatidylinositol 3-kinase (PI3K) and Akt signaling, increases post-transcriptional PD-L1 expression in cancers (Keir M E et al., 2008. Annu Rev Immunol. 26:677-704). Residues of PD-L1 important for binding to PD-1 include PD-L1(67), PD-L1(121), PD-L1(122), PD-L1(123), PD-L1(123), PD-L1(124), and PD-L1(126).

PD-L2 expression is more restricted than PD-L1 expression. PD-L2 is inducibly expressed on DCs, macrophages, and bone marrow-derived mast cells. PD-L2 is also expressed on 50% to 70% of resting peritoneal B1 cells, but not on conventional B2 B cells. PD-L2 can also be induced on monocytes and macrophages by GM-CSF, IL-4, and IFN-γ. PD-L2 expression has also been observed on tumor lines.

PD-1 and its ligands have been shown to have important roles in regulating immune defenses against microbes that cause acute and chronic infections. The PD-1:PD-L pathways appear to play important roles in the outcome of infection, and the regulation of the delicate balance between effective antimicrobial immune defenses and immune-mediated tissue damage.

A number of microorganisms that cause chronic infection appear to have exploited the PD-1:PD-L pathways to evade the immune responses and establish persistent infection. Studies in the lymphocytic choriomeningitis virus (LCMV) model of chronic viral infection were the first to show a role for the PD-1:PD-L pathway during chronic infection (Barber D L et al. 2006. Nature 439:682-87). Viruses that cause chronic infections can render virus-specific T cells nonfunctional and thereby silence the antiviral T cell response (Wherry E J and Ahmed R. 2004. J. Virol. 78:5535-45). Functional dysregulation, also termed herein as “exhaustion,” of CD8 T cells is an important reason for ineffective viral control during chronic infections and is characteristic of chronic LCMV infection in mice, as well as of HIV, HBV, HCV, and HTLV infection in humans and SIV infection in primates.

In chronic viral infections in humans, several groups have shown that PD-1 expression is high on HIV-specific (Petrovas C et al. 2006. J. Exp. Med. 203:2281-92; Day C L et al. 2006. Nature 443:350-54; Trautmann L et al. 2006. Nat. Med. 12:1198-202), HBV-specific (Boettler T et al. 2006. J. Virol. 80:3532-40; Boni C et al. 2007. J. Virol. 81:4215-25), and HCV-specific T cells (Bengsch B. et al., 2010 PLoS Pathog. 6(6); Urbani S et al. 2006. J. Virol. 80:11398-403). Blocking PD-1:PD-L interactions in vitro has been shown to reverse the exhaustion of HIV-specific, HBV-specific (Boni C et al. 2007. J. Virol. 81:4215-25), HCV-specific, and SIV-specific (Velu V et al. 2007. J. Virol. 81:5819-28) CD8 and CD4 T cells and restores proliferation and cytokine production (Petrovas C et al. 2006. J. Exp. Med. 203:2281-92; Day C L et al. 2006. Nature 443:350-54; Trautmann L et al. 2006. Nat. Med. 12:1198-202; Urbani S et al. 2006. J. Virol. 80:11398-403). Recent work shows that the HCV core, a nucleocapsid protein, can upregulate PD-1 and PD-L1 expression on healthy donor T cells and that upregulation of PD-1 is mediated by interaction of the HCV core with the complement receptor ClQBP (Yao Z Q et al. 2007. Viral Immunol. 20:276-87).

The PD-1:PD-L pathway also can play a key role in the chronicity of bacterial infections. Helicobacter pylori causes chronic gastritis and gastroduodenal ulcers and is a risk factor for development of gastric cancer. During H. pylori infection, T cell responses are insufficient to clear infection, leading to persistent infection. Gastric epithelial cells express MHC class II molecules and are thought to have important APC (antigen-presenting cell) function during H. pylori infection. Anti-PD-L1 blocking antibodies enhance T cell proliferation and IL-2 production in cultures of gastric epithelial cells exposed to H. pylori and CD4 T cells, suggesting that the PD-1:PD-L1 pathway can play an important role in inhibiting T cell responses during H. pylori infection (Das S et al. 2006. J. Immunol. 176:3000-9).

Parasitic worms also have exploited the PD-1:PD-L pathways to induce macrophages with strong suppressive function. During Taenia crassiceps infection in mice, a high percentage of CD4 T cells express PD-1, and PD-L1 and PD-L2 are upregulated on activated macrophages. Blockade of PD-L1, PD-L2, or PD-1 significantly decreased suppression of in vitro T cell proliferation by macrophages from Taenia-infected mice (Terrazas Li et al. 2005. Int. J. Parasitol. 35:1349-58). Similarly, during Schistosoma mansoni infection in mice, macrophages express high levels of PD-L1 and more modest levels of PD-L2. Anti-PD-L1 completely abrogated the ability of these macrophages to suppress T cell proliferation in vitro, whereas anti-PD-L2 had no effect (Keir M E et al., 2008. Annu Rev Immunol. 26:677-704).

The PD-1:PD-L pathways have also been shown to have distinct roles in the immune response to the protozoan parasite Leishmania mexicana (Keir M E et al., 2008. Annu Rev Immunol. 26:677-704).

Tumors express antigens that can be recognized by host T cells, but immunologic clearance of tumors is rare. Part of this failure is due to immune suppression by the tumor microenvironment. Recent work has indicated that the PD-1: PD-L pathways are involved in suppression of anti-cancer/tumor immune responses. PD-1 expression is upregulated on tumor infiltrating lymphocytes, and this can contribute to tumor immunosuppression. PD-L1 expression has been shown in situ on a wide variety of solid tumors, including breast, lung, colon, ovarian, melanoma, bladder, liver, salivary, stomach, gliomas, thyroid, thymic epithelial, head, and neck. In addition, in ovarian cancer, PD-L1 expression is inversely correlated with intraepithelial, but not stromal, infiltrating CD8 T cells, suggesting that PD-L1 inhibits the intratumor migration of CD8 T cells. Also, studies relating PD-L1 expression on tumors to disease outcome show that PD-L1 expression strongly correlates with unfavorable prognosis in kidney, ovarian, bladder, breast, gastric, and pancreatic cancer but not small cell lung cancer (Keir M E et al., 2008. Annu Rev Immunol. 26:677-704).

The PD-1 pathway can also play a role in hematologic malignancies. PD-1 is highly expressed on the T cells of angioimmunoblastic lymphomas, and PD-L1 is expressed on the associated follicular dendritic cell network. In nodular lymphocyte-predominant Hodgkin lymphoma, the T cells associated with lymphocytic and/or histiocytic (L&H) cells express PD-1. PD-1 and PD-L1 are expressed on CD4 T cells in HTLV-1-mediated adult T cell leukemia and lymphoma. PD-L2 has been identified as being highly expressed in mantle cell lymphomas. PD-L1 is expressed on multiple myeloma cells but not on normal plasma cells, and T cell expansion in response to myeloma cells is enhanced in vitro by PD-L1 blockade. PD-L1 is expressed on some primary T cell lymphomas, particularly anaplastic large cell T lymphomas (Keir M E et al., 2008. Annu Rev Immunol. 26:677-704).

Measuring DD1α and/or PD-1 Expression

In one aspect, the expression of DD1α and/or PD-1 can be used as a biomarker to identify a patient diagnosed with an immune-related disease who is more likely to respond to an immunotherapy targeting DD1α and/or PD-1 (e.g., anti-DD1α and/or anti-PD-1 treatment for immune-related diseases where an upregulation of immune response is desirable, or DD1α and/or PD-1 agonist treatment for immune-related dieases where a suppression of immune response is desirable). In some embodiments, the expression of DD1α and/or PD-1 can be used as a biomarker to identify a cancer patient who is more likely to respond to a cancer immunologic and/or an immunotherapy (e.g., comprising anti-DD1α and/or anti-PD-1 treatment). Other embodiments where DD1α and/or PD-1 can act as a biomarker for patient segmentation include identifying asthma or allergy patients, or patients having an immune disease or disorder such as bacterial and/or fungal infection that are more likely to respond to immunotherapies. When DD1α and/or PD-1 expression is used in this manner, the DD1α and/or PD-1 proteins are referred to herein as “biomarker(s).”

DD1α and/or PD-1 expression can be detected by any suitable method, including e.g., detection of protein levels or detection of mRNA expression levels. DD1α and/or PD-1 polypeptides can be detected in any form that can be found in a biological sample (e.g., a tissue biopsy such as a tumor biospy) obtained from a subject, or in any form that can result from manipulation of the biological sample (e.g., as a result of sample processing). Modified forms of DD1α and/or PD-1 can include modified proteins that are a product of allelic variants, splice variants, post-translational modification (e.g., glycosylation, proteolytic cleavage (e.g., fragments of a parent protein), glycosylation, phosphorylation, lipidation, oxidation, methylation, cysteinylation, sulphonation, acetylation, and the like), oligomerization, de-oligomerization (to separate monomers from a multimeric form of the protein), denaturation, and the like.

In some embodiments, a biological sample for use in the methods and systems as disclosed herein is a peripheral biological fluid sample, for example, any one of the samples selected from: blood, plasma, serum, urine, mucus or cerebral spinal fluid obtained from the subject. A biological sample can be taken from any biological source, e.g., a body tissue, a tumor, circulating tumor cells, exosomes, or a body fluid, of a subject (e.g., blood, plasma or serum). Other usable body fluids include cerebrospinal fluid (CSF), urine and tears. Some non-limiting examples of biological samples include a blood sample, a urine sample, a semen sample, a lymphatic fluid sample, a cerebrospinal fluid sample, a plasma sample, a serum sample, a pus sample, an amniotic fluid sample, a biopsy sample, a needle aspiration biopsy sample, a swab sample, a mouthwash sample, a cancer sample, a tumor sample, a tissue sample, a cell sample, a cell lysate sample, a crude cell lysate sample, a production sample, a drug preparation sample, a biological molecule production sample, a protein preparation sample, a lipid preparation sample, a carbohydrate preparation sample, or a combination of such samples.

Biological fluid samples, particularly peripheral biological fluid samples may be tested without prior processing of the sample as allowed by some assay formats. Alternatively, many peripheral biological fluid samples will be processed prior to testing. Processing can take the form of isolation and/or elimination of cells (nucleated and non-nucleated), such as erythrocytes, leukocytes, and platelets in blood samples, and can also include the elimination of certain proteins, such as certain clotting cascade proteins from blood. In some examples, the peripheral biological fluid sample is collected in a container comprising EDTA.

In other embodiments, the biological sample is a tumor sample. In other embodiments, the biological sample is a biopsy sample from a site of suspected tumor growth (e.g., skin biopsy for melanoma diagnosis or cells from a solid tumor). A biopsy sample can include e.g., tissue biopsy, fine needle aspiration, core needle biopsy, vacuum assisted biopsy, open surgical biopsy, isolation of circulating cancer cells, among others.

In some embodiments, the biological sample can be stored, for example as frozen biological sample prior to subjecting to the detection of DD1α and/or PD-1 expression, as described herein.

Detection of DD1α and/or PD-1 expression can be performed separately or together. In some embodiments only DD1α expression is detected, while in other embodiments the expression of both proteins is determined. When both proteins are detected, such detection can be conducted in the same or different biological samples, the same or separate assays, and can be conducted in the same or different reaction mixtures. Where DD1α and/or PD-1 are assayed in the same reaction mixture in e.g., an immunoassay, detection of the proteins in the sample can be accomplished using, for example, antibodies having different, detectably distinct labels so that one can distinguish between detection of specific immunocomplexes containing DD1α and/or PD-1. For example, the primary antibodies can have different detectable labels (e.g., different optically detectable labels that provide for different excitation and/or emission wavelengths). In another example, the secondary antibody specific for each target are differently detectably labeled.

As described herein, the level of DD1α and/or PD-1 can be measured in a biological sample from a subject. The expression level can be measured using any available measurement technology that is capable of specifically determining the level of the proteins in a biological sample. The measurement may be either quantitative or qualitative, so long as the measurement is capable of indicating whether the level of DD1α and/or PD-1 is the same as, or above or below the reference threshold value for each protein measured.

The measured level of DD1α and/or PD-1 can be a primary measurement of the level of each protein measuring the quantity of the biomarker protein itself, such as by detecting the number of biomarker protein molecules in the sample) or it may be a secondary measurement of the biomarker (a measurement from which the quantity of the biomarker protein can be but is not necessarily deduced, such as a measure of enzymatic activity or a measure of nucleic acid, such as mRNA, encoding the biomarker protein). Qualitative data may also be derived or obtained from primary measurements.

Commonly, biomarker protein levels may be measured using an affinity-based measurement technology. Affinity-based measurement technology utilizes a molecule that specifically binds to the biomarker protein being measured (an “affinity reagent,” such as an antibody or aptamer), although other technologies, such as spectroscopy-based technologies (e.g., matrix-assisted laser desorption ionization-time of flight, MALDI-TOF spectroscopy) or assays measuring bioactivity (e.g., assays measuring mitogenicity of growth factors) can be used. Affinity-based technologies can include antibody-based assays (immunoassays) and assays utilizing aptamers (nucleic acid molecules which specifically bind to other molecules), such as ELONA. Additionally, assays utilizing both antibodies and aptamers are also contemplated (e.g., a sandwich format assay utilizing an antibody for capture and an aptamer for detection).

Immunoassay techniques commonly known in the art can be used in the systems and methods as disclosed herein, and include, for example, radioimmunoassay, ELISA (enzyme-linked immunosorbant assay), “sandwich” immunoassays, immunoradiometric assays, immunodiffusion assays, in situ immunoassays (using colloidal gold, enzyme or radioisotope labels, for example), Western blot analysis, immunoprecipitations, immunofluorescence assays, immunoelectrophoresis assays, fluoroimmunoassay (FiA), immunoradiometric assay (IRMA), immunoenzymometric assay (IEMA), immunoluminescence assay and immunofluorescence assay (Madersbacher S, Berger P. Antibodies and immunoassays. Methods 2000; 21:41-50).

Affinity-based assays may be in competition or direct reaction formats, utilize sandwich-type formats, and can further be heterogeneous (e.g., utilize solid supports) or homogenous (e.g., take place in a single phase) and/or utilize immunoprecipitation. Many assays involve the use of labeled affinity reagent (e.g., antibody, polypeptide, or aptamer); the labels may be, for example, enzymatic, fluorescent, chemiluminescent, radioactive, or dye molecules. Assays which amplify the signals from the probe are also known; examples of which are assays which utilize biotin and avidin, and enzyme-labeled and mediated immunoassays, such as ELISA and ELONA assays. For example, the biomarker concentrations from biological fluid samples may be measured by LUMINEX® assay or ELISA. Either of the biomarker or reagent specific for the biomarker can be attached to a surface and levels can be measured directly or indirectly.

In an “immunohistochemistry assay” a section of tissue is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or beta-galactosidase), or chemical methods (e.g., DAB/Substrate chromagen). The sample is then analyzed microscopically, most preferably by light microscopy of a sample stained with a stain that is detected in the visible spectrum, using any of a variety of such staining methods and reagents known to those skilled in the art.

Alternatively, “radioimmunoassays” can be employed. A radioimmunoassay is a technique for detecting and measuring the concentration of an antigen using a labeled (e.g., radioactively or fluorescently labeled) form of the antigen. Examples of radioactive labels for antigens include 3H, 14C, and 125I.

Other techniques can be used to detect the expression level in a biological sample and can be performed according to a practitioner's preference, and based upon the present disclosure and the type of biological sample (i.e. plasma, urine, tissue sample etc.). One such technique is Western blotting (Towbin et at., Proc. Nat. Acad. Sci. 76:4350 (1979). In one embodiment, the level of DD1α and/or PD-1 can be determined based on gel electrophoresis techniques, in particular SDS-PAGE, especially two dimensional PAGE (2D-PAGE), preferably two dimensional SDS-PAGE (2D-SDS-PAGE).

In one embodiment, the level of DD1α and/or PD-1 in a biological sample can be determined by mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are incorporated herein in their entirety by reference.

In some embodiments, antibodies, polyclonal, monoclonal and chimeric antibodies useful in the methods as disclosed herein can be purchased from a variety of commercial suppliers, or may be manufactured using well-known methods, e. g., as described in Harlow et al., Antibodies: A Laboratory Manual, 2nd Ed; Cold. Spring Harbor Laboratory Press, Cold Spring Harbor, N.Y. (1988). Commercial antibodies can be purchased, for example, from Sigma-Aldrich™, CalBiochem™, Abcam™, Santa-Cruz Biotechnologies™, novus Bio™, U.S. Biologicals™, Millipore™, LifeSpan™, Abnova™, Cell Signalling Technologies™, etc.

In some embodiments, the methodologies described herein can be combined with machines, computer systems and media to produce an automated system for determining the level of DD1α and/or PD-1 in a biological sample and analysis to produce e.g., a printable report which identifies, for example, the level of DD1α and/or PD-1 in a biological sample. Exemplary devices that can be used include but are not limited to electronic computational devices, including computers of all types. When the methods as described herein are implemented in a computer, the computer program that can be used to configure the computer to carry out the steps of the methods can be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods can also be provided over an electronic network, for example, over the internet, world-wide web, an intranet, or other network. In one embodiment, the data storage does not include signal or carrier waves. In one example, the methods described herein can be implemented in a system comprising a processor and a computer readable medium that includes program code means for causing the system to carry out the steps of the methods described herein. The processor may be any processor capable of carrying out the operations needed for implementation of the methods. The program code means can be any code that when implemented in the system can cause the system to carry out the steps of the methods described herein. Examples of program code means include but are not limited to instructions to carry out the methods described herein written in a high level computer language such as C++, Java, or Fortran; instructions to carry out the methods described herein written in a low level computer language such as assembly language; or instructions to carry out the methods described herein in a computer executable form such as compiled and linked machine language.

Determining p53 Activity and/or Expression

p53 is a tumor suppressor protein encoded by the TP53 gene, and generally exists in either native or mutated form. Methods for measuring p53 are known in the art and can be used to measure the level of p53 activity or expression in a sample from a subject as described herein. In some embodiments, the methods for measuring p53 as described in the Examples can be used to measure the level of p53 or expression in a sample from a subject as described herein.

In some embodiments, the level of p53 activity or expression in a sample can be determined by measuring protein levels. Exemplary methods to detect protein level of p53 include, but are not limited to antibody-based assays and immunoassays such as enzyme linked immunoabsorbant assay (ELISA), radioimmunoassay (RIA), Immunoradiometric assay (IRMA), Western blotting, immunocytochemistry or immunohistochemistry, and protein chips. Commercially available antibodies and/or immunoassays (such as ELISA) for detecting p53, e.g., from Abcam, and Pierce/Thermo Scientific, and Millipore, can be used in the methods described herein.

In some embodiments, the level of p53 activity or expression in a sample can be determined by measuring mRNA levels. Exemplary methods to detect mRNA level of p53 include, but are not limited to polymerase chain reaction (PCR), reverse transcription-PCR, real time PCR, northern blot, DNA arrays or microarrays, or any other art-recognized methods such as the one described in U.S. Pat. No. 6,110,671, the content of which is incorporated herein by reference.

In some embodiments, the level of p53 activity or expression in a sample can be determined or monitored via a reporter assay. For example, a p53-responsive construct (e.g., luciferase construct) encoding a reporter gene (e.g., a firefly luciferase reporter gene) can be used to transfect cells in a sample and thus to detect or measure p53 signaling activity. p53 reporter assay kits are commercially available (e.g., Cat. # CCS-004L from Qiagen).

Nucleic Acid Inhibitors or Antagonists of DD1α and/or PD-1

One of the approaches for inhibiting the expression of selected target polypeptides, such as DD1α and/or PD-1 is through the use of RNA interference agents. “RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease will be of at least 10%, 20%, 30%, 40%, 50%, 60%, 70%, 80%, 90%, 95% or 99% or more as compared to the expression of a target gene or the activity or level of the protein encoded by a target gene which has not been targeted by an RNA interfering agent.

The terms “RNA interference agent” and “RNA interference” as they are used herein are intended to encompass those forms of gene silencing mediated by double-stranded RNA, regardless of whether the RNA interfering agent comprises an siRNA, miRNA, shRNA or other double-stranded RNA molecule. “Short interfering RNA” (siRNA), also referred to herein as “small interfering RNA” is defined as an RNA agent which functions to inhibit expression of a target gene, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

Methods for designing an siRNA or other RNA interference agent for inhibiting e.g., DD1α and/or PD-1 as described herein are known to those of skill in the art.

In one embodiment, the RNA interference agent is delivered or administered to a subject in a pharmaceutically acceptable carrier. Additional carrier agents, such as liposomes, can be added to the pharmaceutically acceptable carrier. In another embodiment, the RNA interference agent is delivered by a vector encoding small hairpin RNA (shRNA) in a pharmaceutically acceptable carrier to the cells in an organ of an individual. The shRNA is converted by the cells after transcription into siRNA capable of targeting, for example, DD1α and/or PD-1.

In another embodiment, a nucleic acid sequence encoding the RNA interference agent is administered to the subject or cell (e.g., a plasmid or viral vector, e.g., a lentiviral vector. Such vectors can be used as described, for example, in Xiao-Feng Qin et al. Proc. Natl. Acad. Sci. U.S.A., 100: 183-188). In such an embodiment, the nucleic acid sequence can comprise an expression vector. In one embodiment, the vector is a regulatable vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In one embodiment, the RNA interference agents used in the methods described herein are taken up actively by cells in vivo following intravenous injection, e.g., hydrodynamic injection, without the use of a vector, illustrating efficient in vivo delivery of the RNA interfering agents. One method to deliver the siRNAs is by topical administration in an appropriate pharmaceutically acceptable carrier. Other delivery methods include delivery of the RNA interfering agents, e.g., the siRNAs or shRNAs, using a basic peptide by conjugating or mixing the RNA interfering agent with a basic peptide, e.g., a fragment of a TAT peptide, mixing with cationic lipids or formulating into particles. The RNA interference agents, e.g., the siRNAs targeting DD1α and/or PD-1 mRNA, can be delivered singly, or in combination with other RNA interference agents, e.g., siRNAs, such as, for example siRNAs directed to other cellular genes. siRNAs can also be administered in combination with other pharmaceutical agents which are used to treat or prevent cancer or infection.

Synthetic siRNA molecules, including shRNA molecules, can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structure.

Methods of delivering RNA interference agents, e.g., an siRNA, or vectors containing an RNA interference agent, to the target cells, e.g., tumor cell, T-cell or macrophage, or other desired target cells, for uptake include injection of a composition containing the RNA interference agent, e.g., an siRNA, or directly contacting the cell, e.g., a tumor cell, with a composition comprising an RNA interference agent, e.g., an siRNA. For example, the RNA interference agent can be delivered directly to the tumor or the blood vessel supplying the tumor. In another embodiment, the RNA interference agent, e.g., an siRNA can be injected directly into any blood vessel, such as vein, artery, venule or arteriole, via, e.g., hydrodynamic injection or catheterization. Administration can be by a single injection or by two or more injections. The RNA interference agent is delivered in a pharmaceutically acceptable carrier. One or more RNA interference agents can be used simultaneously. In one embodiment, a single siRNA that targets human DD1α or PD-1 is used. In another embodiment, one or more siRNAs that target human DD1α and/or PD-1 is used. In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNA interference effectively into cells. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein. Viral-mediated delivery of siRNAs to cells in vitro and in vivo is known in the art (see e.g., Manjunath et al. (2010) Discovery Med 9(48):418-30; Castanotto et al. (2009) Nature 457:426-433). Plasmid- or viral-mediated delivery mechanisms of shRNA can also be employed to deliver shRNAs to cells in vitro and in vivo as reviewed by Manjunath et al. (2010) Discovery Med 9(48):418-30; Castanotto et al. (2009) Nature 457:426-433; Aigner et al. (2008) Curr Pharm Des 14:3603-3619; Whitehead et al. (2009) Nat Rev Drug Discov 8:129-138; Laufer et al. (2010) RNA Technologies and Their Applications, Chapter title “Selected Strategies for the Delivery of siRNA In vitro and In vivo” Erdmann and Barciszewski (eds)). The RNA interference agents, e.g., the siRNAs or shRNAs, can be introduced along with components that perform one or more of the following activities: enhance uptake of the RNA interfering agents, e.g., siRNA, by the cell, e.g., tumor cells, T-cells, or other cells, inhibit annealing of single strands, stabilize single strands, or otherwise facilitate delivery to the target cell and increase inhibition of the target gene, e.g., DD1α and/or PD-1. The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene.

Other Inhibitors or Antagonists of DD1α and/or PD-1

Also contemplated herein are the use of peptides, peptidomimetics, aptamers, and small molecules that inhibit DD1α and/or PD-1, particularly those inhibitors that disrupt the interaction of two monomers of DD1α and/or the interaction of DD1α with PD-1.

As used interchangeably herein, the term “antagonist” or “inhibitor” is used in the broadest sense, and includes any molecule that partially or fully blocks, inhibits, or neutralizes a biological activity of a native polypeptide disclosed herein. Suitable antagonist molecules specifically include antagonist antibodies or antibody fragments, fragments or amino acid sequence variants of native polypeptides, peptides, antisense oligonucleotides, small organic molecules, recombinant proteins or peptides, etc. Methods for identifying antagonists of a polypeptide can comprise contacting a polypeptide with a candidate agonist or antagonist molecule and measuring a detectable change in one or more biological activities normally associated with the polypeptide.

The term “DD1α antagonist” is used interchangeably with the terms “DD1α inhibitor” and “anti-DD1α therapy” and refers to an agent that interferes with the normal functioning of DD1α, either by decreasing transcription or translation of DD1α-encoding nucleic acid, or by inhibiting or blocking DD1α polypeptide activity, or both. Examples of DD1α antagonists include, but are not limited to antisense polynucleotides, interfering RNAs, catalytic RNAs, RNA-DNA chimeras, DD1α-specific aptamers, anti-DD1α antibodies, DD1α-binding fragments of anti-DD1α antibodies, DD1α-binding small molecules, DD1α-binding peptides, and other polypeptides that specifically bind DD1α (including, but not limited to DD1α-binding fragments of one or more DD1α ligands, optionally fused to one or more additional domains), such that the interaction between the DD1α antagonist and DD1α results in a reduction or cessation of DD1α activity or expression. In some embodiments, it will be understood by one of ordinary skill in the art that in some instances, a DD1α antagonist can antagonize one DD1α activity without affecting another DD1α activity. For example, a desirable DD1α antagonist for use in certain of the methods herein is a DD1α antagonist that antagonizes DD1α activity in response to homophilic interaction with another DD1α and/or heterophilic interaction with PD-1, e.g., without affecting or minimally affecting the other DD1α interactions.

In some embodiments, a DD1α inhibitor is an agent that directly or indirectly inhibits or reduces the DD1α-mediated suppression of T cell proliferation. Accordingly, a DD1α inhibitor can target the DD1αreceptor itself or its corresponding ligand, or any of DD1α's upstream molecules. Examples of DD1α inhibitors include, among others, anti-DD1αmolecules. A DD1α inhibitor can be a protein, a peptide, a peptidomimetic, an aptamer, a nucleic acid, an antibody, a small molecule, a vaccine, or any combinations thereof.

In some embodiments, the inhibitor can be a peptide. “Polypeptide,” “peptide”, and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. In one embodiment, the peptide acts as a dominant negative for DD1α and/or PD-1, thereby disrupting the interaction of DD1α and/or PD-1 or DD1α with another monomer of DD1α.

Polypeptides derived from another polypeptide can have one or more mutations relative to the starting polypeptide, e.g., one or more amino acid residues which have been substituted with another amino acid residue or which has one or more amino acid residue insertions or deletions. A polypeptide “derived” from another polypeptide will retain therapeutically or physiologically relevant biological activity of the polypeptide from which it is derived. Relevant activity in this context includes, for example, reducing DD1α and/or PD-1expression. By “retain” in such context is meant at least 50% retention, preferably at least 60%, at least 70%, at least 80%, at least 90%, at least 95%, at least 99% or even 100% or greater retention.

A polypeptide can comprise an amino acid sequence which is not naturally occurring. Such variants necessarily have less than 100% sequence identity or similarity with a starting polypeptide molecule known to inhibit DD1α and/or PD-1 activity. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule. In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity. As with polypeptides derived from another, a variant of this kind will retain a therapeutically or physiologically relevant biological activity of the polypeptide from which it is a variant.

In one embodiment, a polypeptide useful in the methods described herein consists of, consists essentially of, or comprises an amino acid sequence, or is a fragment thereof derived from SEQ ID NO:1 or SEQ ID NO:2, provided that the polypeptide disrupts DD1α and/or PD-1 activity and/or interaction. In some embodiments, the polypeptide that disrupts DD1α can disrupt homophilic interaction of DD1α with DD1α and/or heterophilic interaction of DD1α with PD-1. In some embodiments, the polypeptide that disrupts PD-1 activity can disrupt functional interaction of PD-1 with DD1α, PD-L1 and/or PD-L2.

The polypeptides described herein can comprise conservative amino acid substitutions at one or more amino acid residues, e.g., at essential or non-essential amino acid residues but will retain a therapeutically or physiologically relevant activity of an inhibitory peptide as that term is described herein. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, in a conservative substitution variant, a nonessential amino acid residue in the polypeptide is preferably replaced with another amino acid residue from the same side chain family.

The term “variant” as used herein refers to a polypeptide or nucleic acid that is “substantially similar” to a wild-type DD1α and/or PD-1 polypeptide or nucleic acid, and as such inhibits the interaction between DD1α and/or PD-1 due to a dominant negative effect. A molecule is said to be “substantially similar” to another molecule if both molecules have substantially similar structures (i.e., they are at least 50% similar in amino acid sequence as determined by BLASTp alignment set at default parameters) and are substantially similar in at least one therapeutically or physiologically relevant function (e.g., inhibition of DD1α signaling (e.g., homophilic and/or heterophilic interaction of DD1α), and/or and/or PD-1 signaling) A variant differs from the naturally occurring polypeptide or nucleic acid by one or more amino acid or nucleic acid deletions, additions, substitutions or side-chain modifications, yet retains one or more therapeutically relevant, specific functions or desired biological activities of the naturally occurring molecule (e.g., interacts with other DD1αmonomers and/or PD-1 to sequester endogenous DD1α and/or PD-1). In particular, such peptides will also lack the ability to induce DD1α and/or PD-1 signaling.

Amino acid substitutions include alterations in which an amino acid is replaced with a different naturally-occurring or a non-conventional amino acid residue. Some substitutions can be classified as “conservative,” in which case an amino acid residue contained in a polypeptide is replaced with another naturally occurring amino acid of similar character either in relation to polarity, side chain functionality or size. Substitutions encompassed by variants as described herein can also be “non-conservative,” in which an amino acid residue which is present in a peptide is substituted with an amino acid having different properties (e.g., substituting a charged or hydrophobic amino acid with an uncharged or hydrophilic amino acid), or alternatively, in which a naturally-occurring amino acid is substituted with a non-conventional amino acid. Also encompassed within the term “variant,” when used with reference to a polynucleotide or polypeptide, are variations in primary, secondary, or tertiary structure, as compared to a reference polynucleotide or polypeptide, respectively (e.g., as compared to a wild-type polynucleotide or polypeptide). Polynucleotide changes can result in amino acid substitutions, additions, deletions, fusions and truncations in the polypeptide encoded by the reference sequence. Variants can also include insertions, deletions or substitutions of amino acids in the peptide sequence. To be therapeutically useful, such variants will retain a therapeutically or physiologically relevant activity as that term is used herein.

The term “derivative” as used herein refers to peptides which have been chemically modified, for example by ubiquitination, labeling, pegylation (derivatization with polyethylene glycol) or addition of other molecules. A molecule is also a “derivative” of another molecule when it contains additional chemical moieties not normally a part of the molecule. Such moieties can improve the molecule's solubility, absorption, biological half-life, etc. The moieties can alternatively decrease the toxicity of the molecule, or eliminate or attenuate an undesirable side effect of the molecule, etc. Moieties capable of mediating such effects are disclosed in Remington's Pharmaceutical Sciences, 18th edition, A. R. Gennaro, Ed., MackPubl., Easton, Pa. (1990). The term “functional” when used in conjunction with “derivative” or “variant” refers to a polypeptide which possesses a therapeutically or physiologically relevant biological activity that is substantially similar to a biological activity of the entity or molecule of which it is a derivative or variant. By “substantially similar” in this context is meant that at least 50% of the relevant or desired biological activity of a corresponding wild-type peptide is retained. In the instance of reducing DD1α and/or PD-1 activity, preferably the derivatives retains at least 60%, at least 70%, at least 80%, at least 90%, at least 95%, or more, including 100% or even more (i.e., the derivative or variant has improved activity relative to wild-type) of the DD1α and/or PD-1 binding activity of the wild-type, while having a reduced ability to activate the DD1α and/or PD-1 signaling pathways.

In another embodiment, the inhibitor of DD1α and/or PD-1 can be a small molecule. As used herein, the term “small molecule” refers to a chemical agent including, but not limited to peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Essentially any small molecule inhibitor of DD1α and/or PD-1 can be used in the treatment of cancer and/or infection using the methods described herein.

In some embodiments, the inhibitor of DD1α and/or PD-1 can be a bispecific or multispecific polypeptide agent as described in the section “Biospecific and Multispecific Polypeptide Agents for Targeting DD1α and/or PD-1” below.

Agonists of DD1α and/or PD-1

The term “agonist” is used in the broadest sense and includes any molecule that mimics or stimulates a biological activity of a native polypeptide disclosed herein. Suitable agonist molecules specifically include agonist antibodies or antibody fragments, fragments or amino acid sequence variants of native polypeptides, peptides, antisense oligonucleotides, small organic molecules, recombinant proteins or peptides, etc. Methods for identifying agonists of a polypeptide can comprise contacting a polypeptide with a candidate agonist or antagonist molecule and measuring a detectable change in one or more biological activities normally associated with the polypeptide.

As used herein, the term “DD1α agonist” refers to an agent that enhances or stimulates the normal functioning of DD1α, by increasing transcription or translation of DD1α-encoding nucleic acid, and/or by inhibiting or blocking activity of a molecule that inhibits DD1αexpression or DD1α activity, and/or by enhancing normal DD1α activity (including, but not limited to enhancing the stability of DD1α or enhancing binding of DD1α to another DD1α and/or PD-1). For example, the DD1α agonist can be selected from an antibody, an antigen-binding fragment, an aptamer, an interfering RNA, a small molecule, a peptide, an antisense molecule, and another binding polypeptide. In another example, the DD1α agonist can be a polynucleotide selected from an aptamer, interfering RNA, or antisense molecule that interferes with the transcription and/or translation of a DD1α-inhibitory molecule. It will be understood by one of ordinary skill in the art that in some instances, a DD1α agonist can agonize one DD1α activity without affecting another DD1α activity. For example, a desirable DD1α agonist for use in certain of the methods herein is a DD1α agonist that agonizes DD1α binding interaction with another DD1α and/or PD-1, e.g., without affecting or minimally affecting the other DD1α interactions.

In some embodiments, a DD1α agonist is an agent that directly or indirectly enhances or stimulates the DD1α-mediated suppression of T-cell proliferation. Accordingly, a DD1α agonist can target the DD1αreceptor or its corresponding ligand, or any of DD1α's upstream molecules. Examples of DD1α agonists include, among others, DD1α-mimic molecules. The DD1α agonist can be a protein, a peptide, peptidomimetic, an aptamer, a nucleic acid, an antibody, a small molecule, a vaccine, a fusion protein, or any combinations thereof.

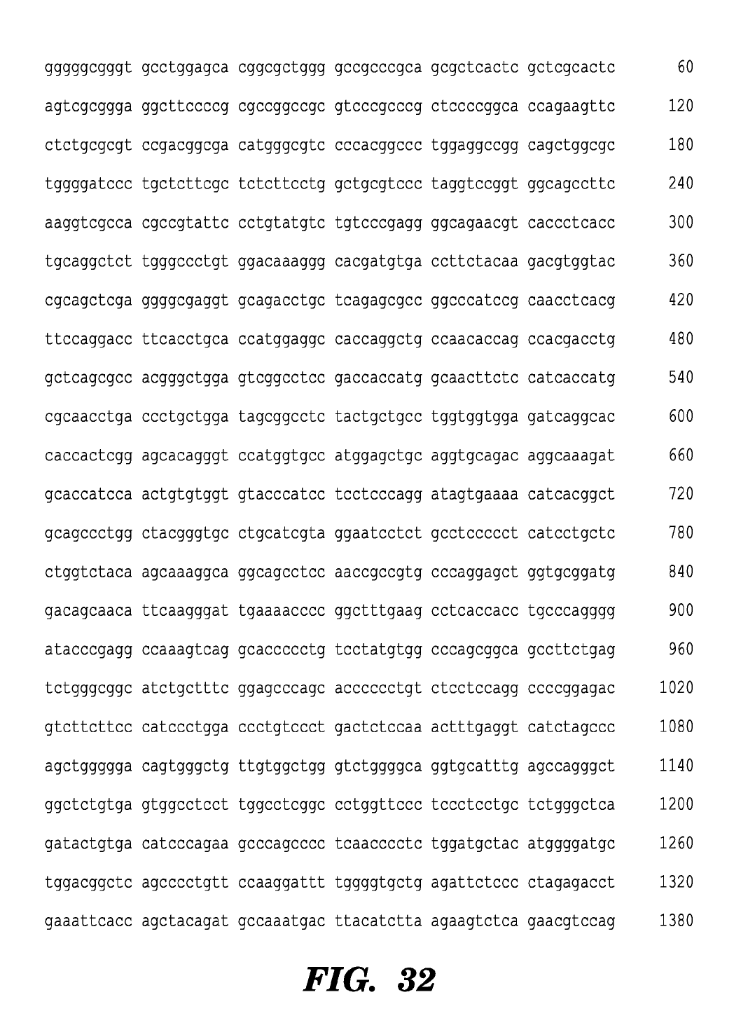

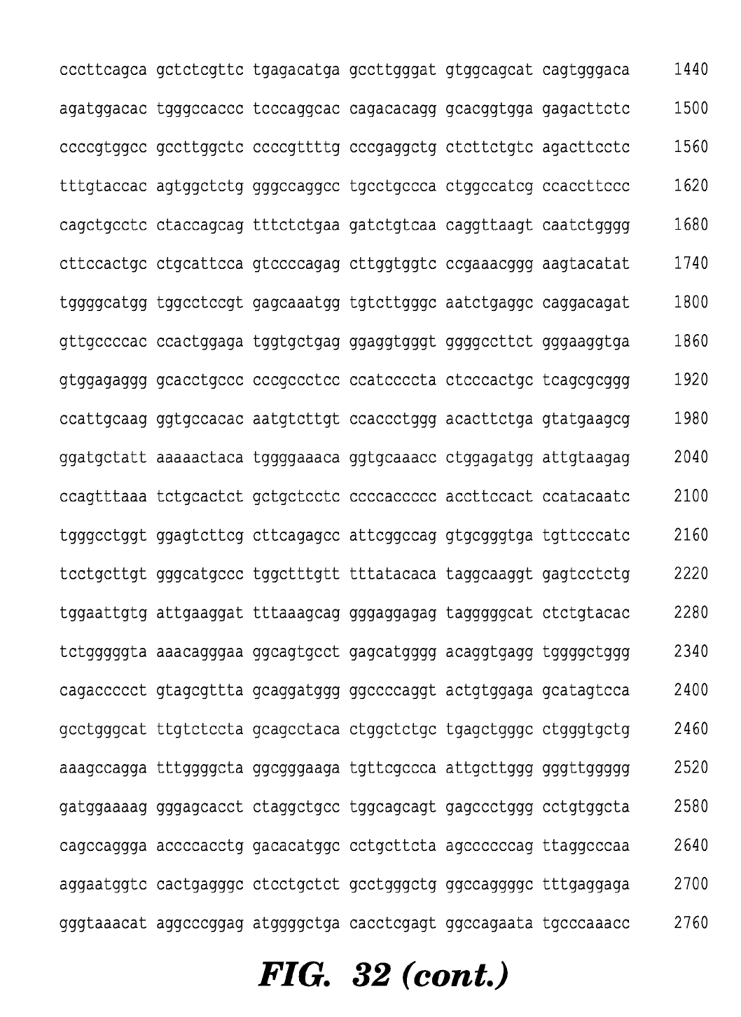

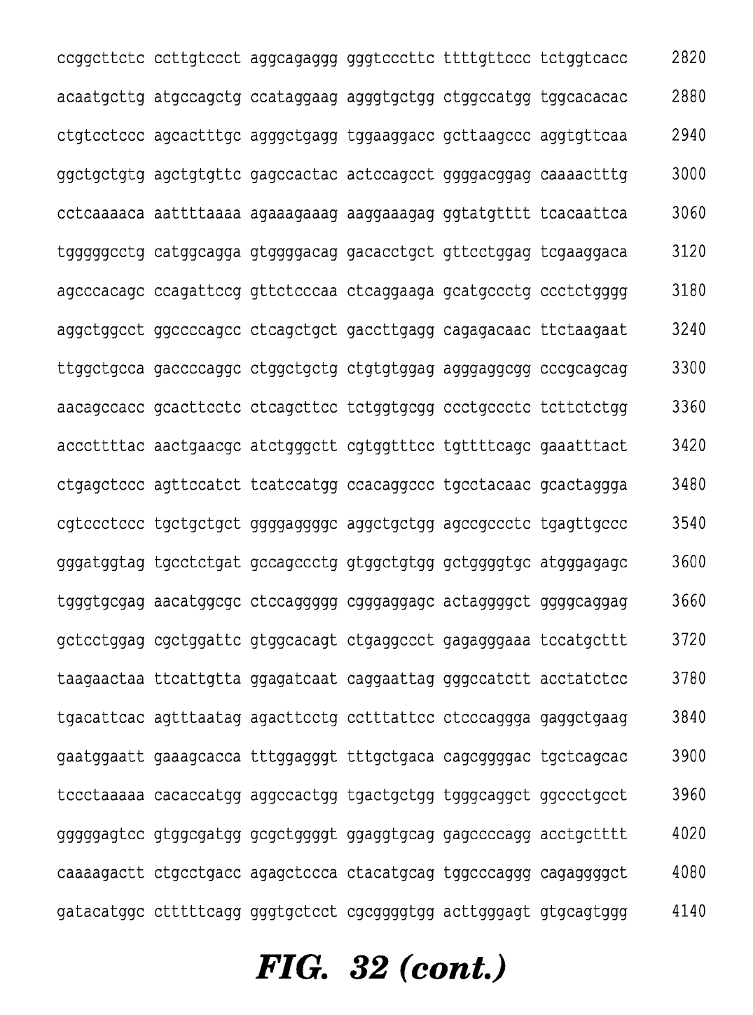

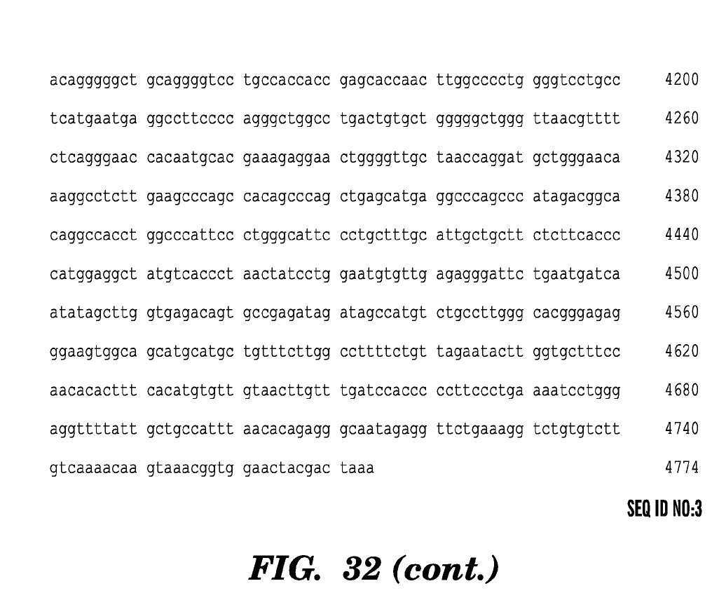

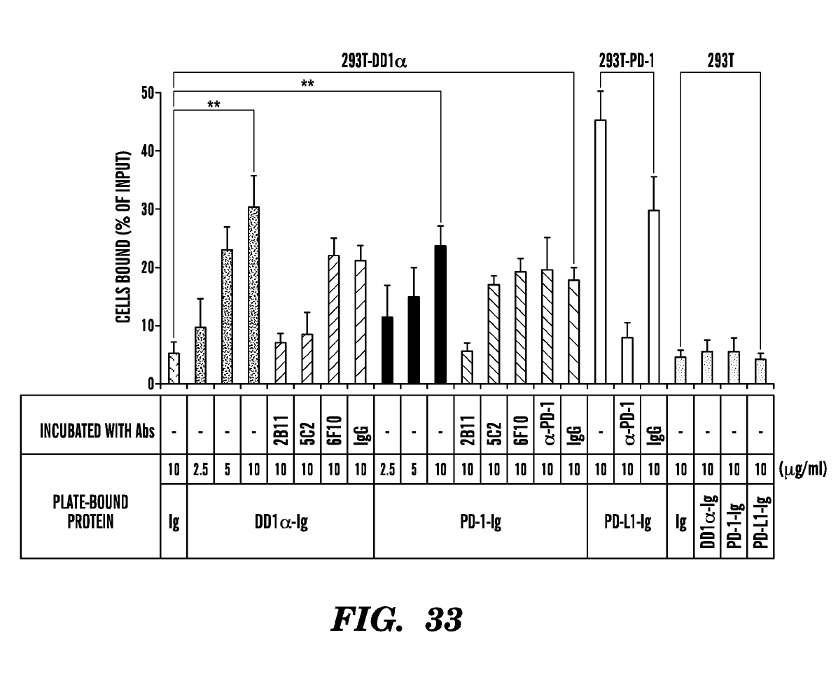

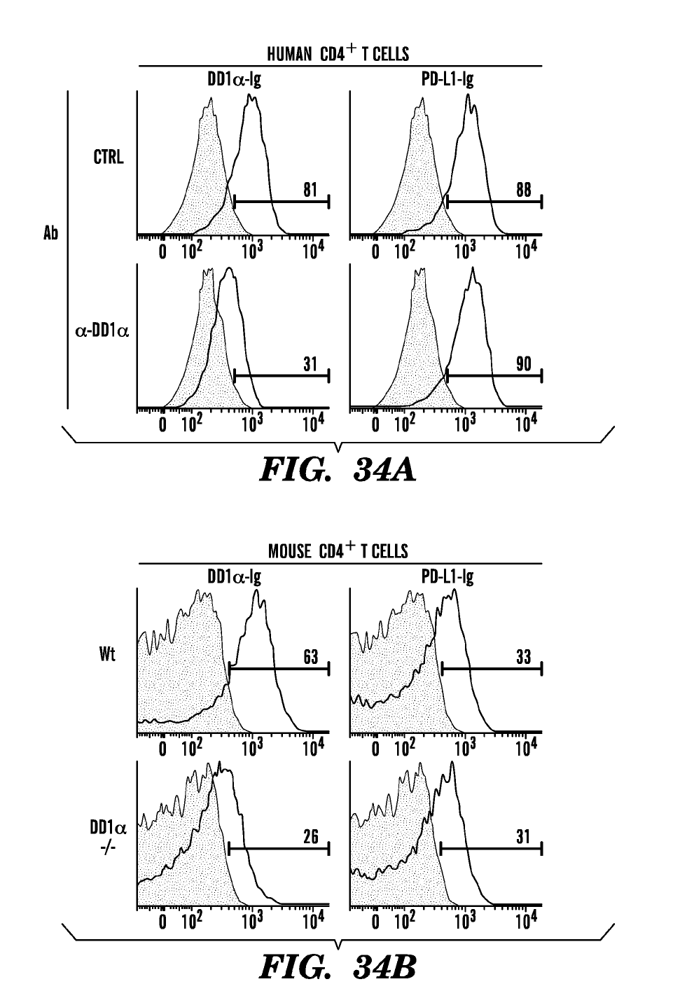

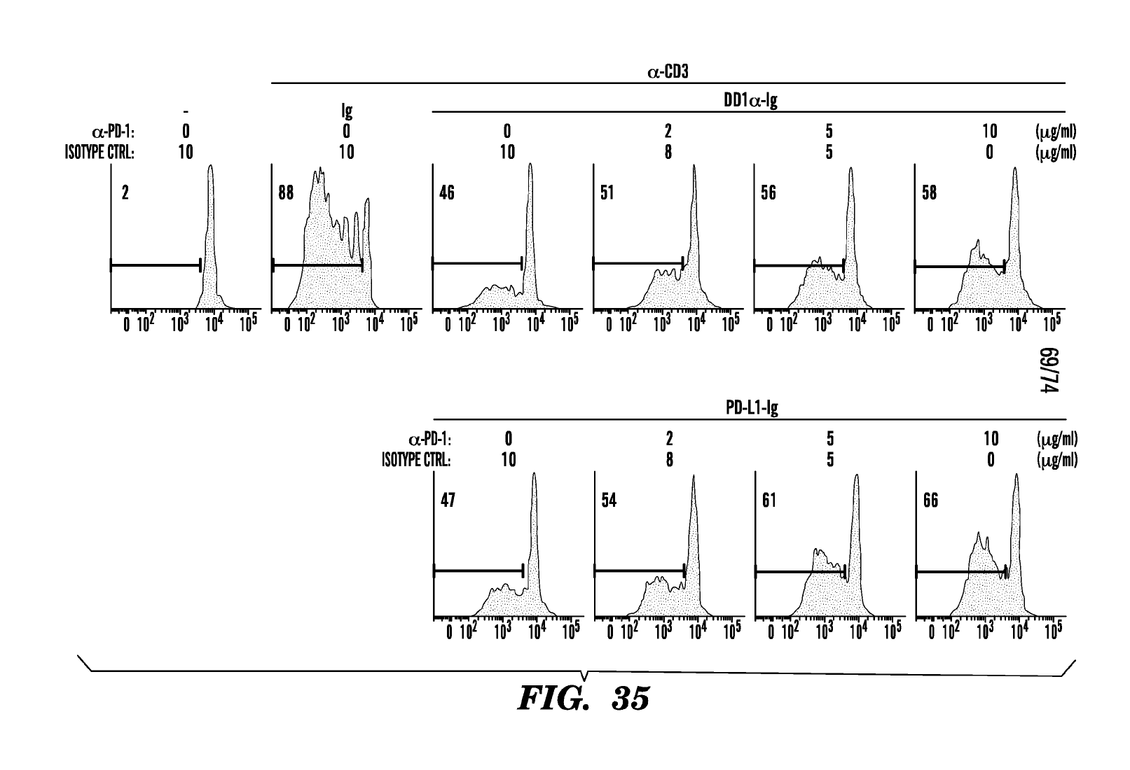

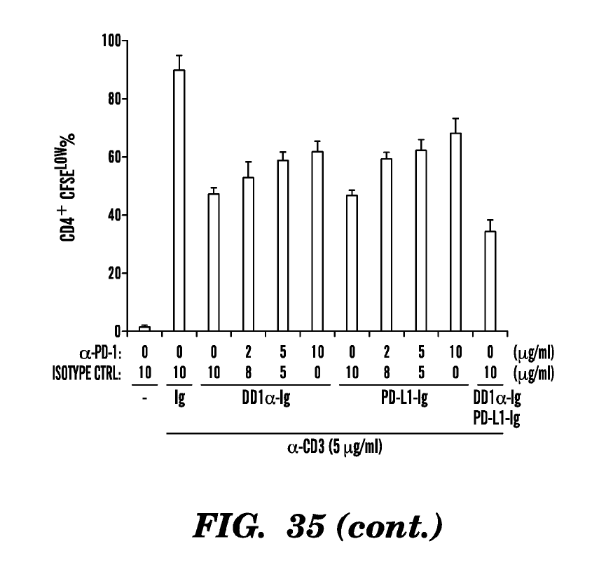

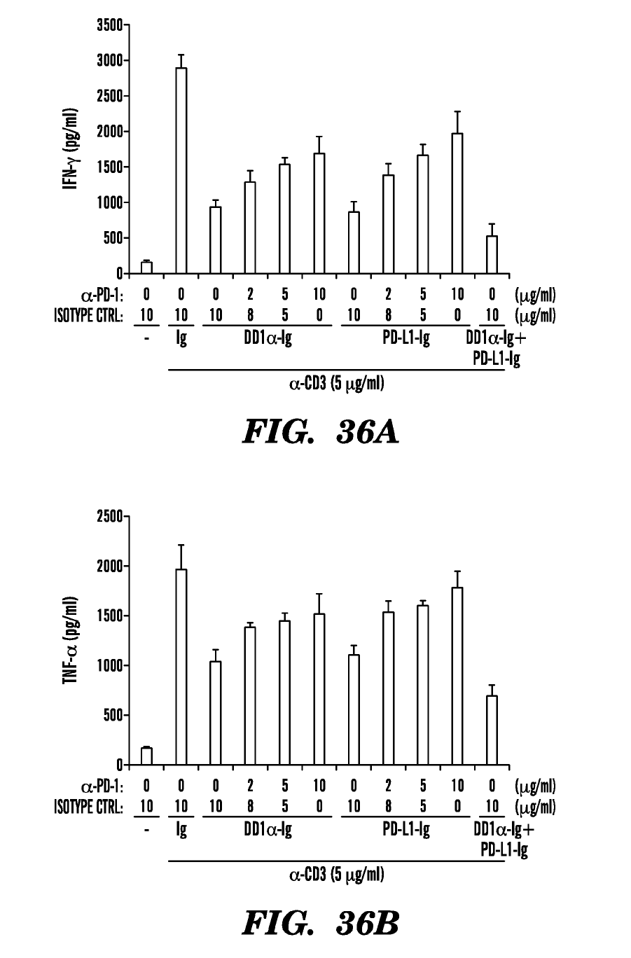

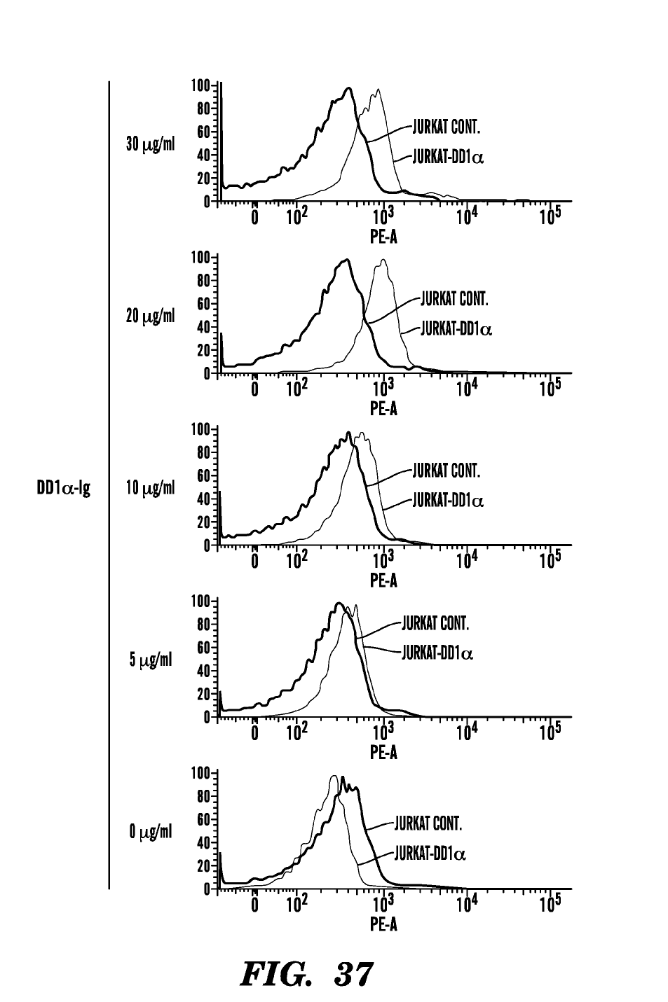

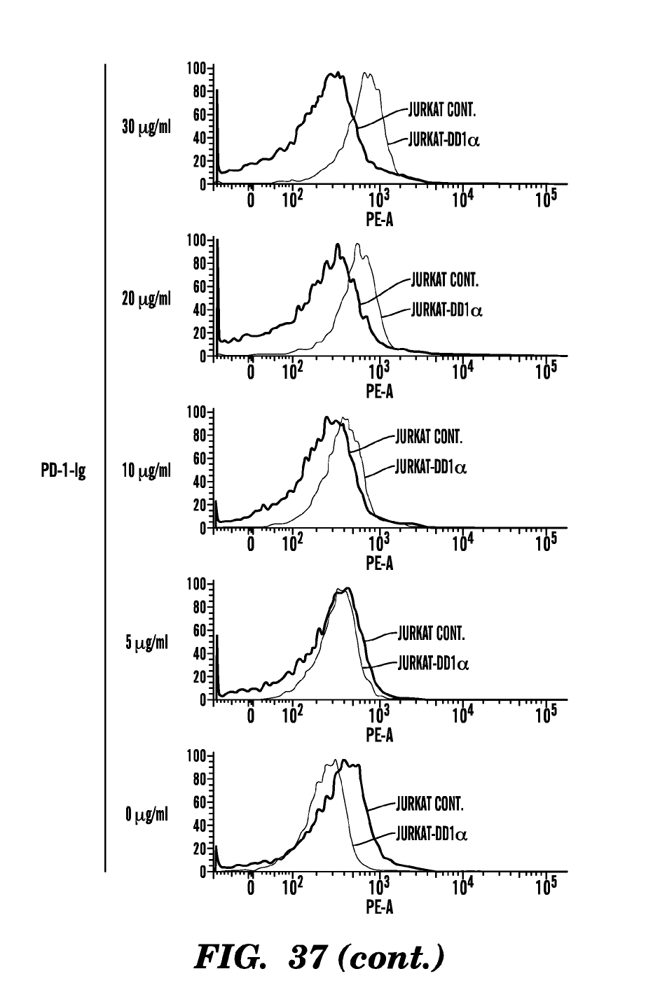

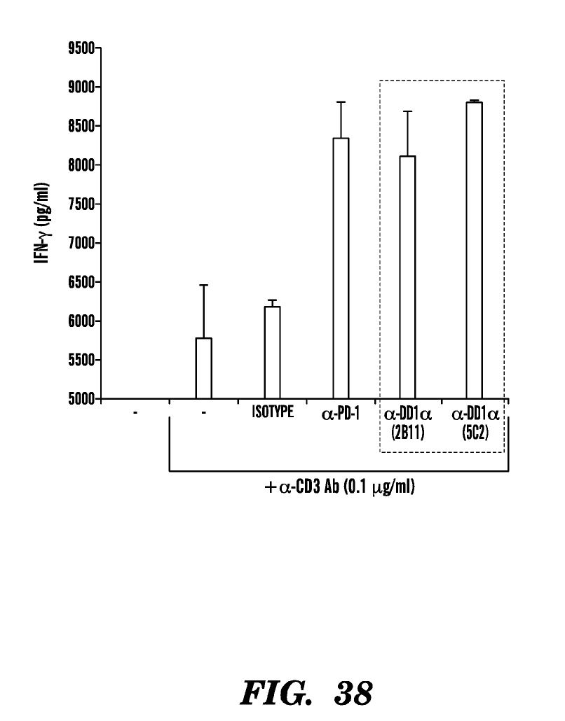

DD1α agonists can be obtained from known sources or prepared using known techniques such as recombinant or synthetic technology. The nucleic acid and protein sequences of DD1α are known in the art, e.g., accessible at world wide web from NCBI. Thus, one of skill in the art can generate DD1α agonists based on these sequences using art-recognized molecular technologies such as cloning and expression technologies. For example, a human DD1α agonist (e.g., a DD1αmolecule) can be generated based on the nucleic acid sequence of human DD1α as shown in FIG. 32 (SEQ ID NO: 3) or a functional fragment thereof (that encodes a peptide that mimics or stimulates homophilic and/or heterophilic interaction of DD1α as described herein) or based on the human peptide sequence as shown in SEQ ID NO: 1.

As used herein, the term “PD-1 agonist” refers to an agent that enhances or stimulates the normal functioning of PD-1, by increasing transcription or translation of PD-1-encoding nucleic acid, and/or by inhibiting or blocking activity of a molecule that inhibits PD-1 expression or PD-1activity, and/or by enhancing normal PD-1 activity (including, but not limited to enhancing the stability of PD-1 or enhancing binding of PD-1 to DD1α, PD-L1 and/or PD-L2). For example, the PD-1 agonist can be selected from an antibody, an antigen-binding fragment, an aptamer, an interfering RNA, a small molecule, a peptide, an antisense molecule, and another binding polypeptide. In another example, the PD-1 agonist can be a polynucleotide selected from an aptamer, interfering RNA, or antisense molecule that interferes with the transcription and/or translation of a PD-1 inhibitory molecule. It will be understood by one of ordinary skill in the art that in some instances, a PD-1 agonist can agonize one PD-1 activity without affecting another PD-1 activity. For example, a desirable PD-1 agonist for use in certain of the methods herein is a PD-1 agonist that agonizes PD-1 binding interaction with DD1α, PD-L1 and/or PD-L2, e.g., without affecting or minimally affecting any of the other PD-1 interactions.

In some embodiments, a PD-1 agonist is an agent that directly or indirectly enhances or stimulates the PD-1-mediated suppression of T-cell stimulatory response. Accordingly, a PD-1 agonist can target the PD-1 receptor or its corresponding ligand such as PD-L1 and/or PD-L2, or any of PD-1's upstream molecules. Examples of PD-1 agonists include, without limitations, PD-1 ligands such as PD-L1 and/or PD-L2. The PD-1 agonist can be a protein, a peptide, peptidomimetic, an aptamer, a nucleic acid, an antibody, a small molecule, a vaccine, a fusion protein, or any combinations thereof.

PD-1 agonists can be obtained from known sources or prepared using known techniques such as recombinant or synthetic technology. The nucleic acid and protein sequences of PD-1 and its ligands such as PD-L1 and PD-L2 of different species (e.g., but not limited to human and mouse) are known in the art, e.g., accessible at world wide web from NCBI. Thus, one of skill in the art can generate PD-1 agonists based on these sequences using art-recognized molecular technologies such as cloning and expression technologies. For example, a human PD-1 agonist (e.g., a PD-L1 molecule) can be generated based on the nucleic acid sequence of human PD-L1, e.g., listed in NCBI under Accession No. NM_001267706, or based on the corresponding protein sequence of human PD-L1, e.g., listed in NCBI under Accession No. NP_001254635.

### Reference Values

The terms “reference value,” “reference level,” “reference sample,” and “reference” are used interchangeably herein and refer to the level of p53, DD1α and/or PD-1 expression in a known sample against which another sample (e.g., one obtained from a subject lacking detectable tumor, cancer or infection) is compared. A reference value is useful for determining the amount of p53, DD1α and/or PD-1 expression or the relative increase/decrease of such expressional levels/ratios in a biological sample. A reference value serves as a reference level for comparison, such that samples can be normalized to an appropriate standard in order to infer the sensitivity of a subject to treatment with an immunotherapy agent such as an anti-DD1α and/or anti-PD-1 antibody, or a DD1α and/or PD-1 agonist.

In one embodiment, a biological standard is obtained at an earlier time point (e.g., prior to the onset of cancer or infection) from the same individual that is to be tested or treated as described herein. Alternatively, a standard can be from the same individual having been taken at a time after the onset or diagnosis of a disease or disorder (e.g., cancer, asthma, allergy, and/or infection). In such instances, the reference value can provide a measure of the efficacy of treatment. It can be useful to use as a reference for a given patient a level or ratio from a sample taken after diagnosis of a disease or disorder (e.g., cancer, asthma, allergy, and/or infection) but before the administration of any therapy to that patient.

Alternatively, a reference value can be obtained, for example, from a known biological sample from a different individual (e.g., not the individual being tested) that is e.g., substantially free of a disease or disorder (e.g., cancer, asthma, allergy, and/or infection) diagnosed in the tested individual. A known sample can also be obtained by pooling samples from a plurality of individuals to produce a reference value or range of values over an averaged population, wherein a reference value represents an average level of p53, DD1α and/or PD-1 expression and/or activity among a population of individuals (e.g., a population of individuals lacking detectable cancer or infection). Thus, the level of p53, DD1α and/or PD-1 in a reference value obtained in this manner is representative of an average level of this marker in a general population of individuals lacking detectable disease or disorder (e.g., cancer, asthma, allergy, and/or infection). An individual sample is compared to this population reference value by comparing expression of p53, DD1α and/or PD-1 from a sample relative to the population reference value. In some embodiments where an upregulation of immune response is desirable to produce a therapeutic effect (e.g., in treatment of cancer, asthma, allergy, and/or infection), a decrease in the amount of p53, DD1α and/or PD-1, compared to a respective reference defined herein, indicates or predicts a decreased sensitivity to an anti-DD1α and/or anti-PD-1 immunotherapy, while an increase in the amount of p53, DD1α and/or PD-1, compared to a respective reference defined herein, indicates or predicts that the subject will be more sensitive to an anti-DD1α and/or anti-PD-1 immunotherapy. In some embodiments where a suppression of immune response is desirable to produce a therapeutic effect (e.g., in treatment of an autoimmune disease), an increase in the amount of p53, DD1α, and/or PD-1, as compared to a respective reference defined herein, indicates or predicts a decreased sensitivity to a DD1α agonist and/or PD-1 agonist therapy, while a decrease in the amount of p53, DD1α, and/or PD-1, as compared to a respective reference defined herein, indicates or predicts that the subject will be more sensitive to a DD1α agonist and/or PD-1 agonist therapy It should be noted that there is often variability among individuals in a population, such that some individuals will have higher levels of p53, DD1α and/or PD-1 expression, while other individuals have lower levels of expression. However, one skilled in the art can make logical inferences on an individual basis regarding the detection and treatment of a disease or disorder (e.g., cancer, allergy, asthma, and/or infection) as described herein.

In one embodiment, a range of values for p53, DD1α and/or PD-1 in e.g., a tissue biopsy can be defined for a plurality of individuals with or without a detectable disease or condition (e.g., cancer, allergy, asthma, and/or infection). Provided that the number of individuals in each group is sufficient, one can define a range of p53, DD1α and/or PD-1 values for each population. These values can be used to define cut-off points for selecting a therapy or for monitoring progression of disease. Thus, one of skill in the art can determine the level of p53, DD1α and/or PD-1 and compare the value to the ranges in each particular sub-population to aid in determining the status of disease and the recommended course of treatment. Such value ranges are analogous to e.g., HDL and LDL cholesterol levels detected clinically. For example, LDL levels below 100 mg/dL are considered optimal and do not require therapeutic intervention, while LDL levels above 190 mg/dL are considered ‘very high’ and will likely require some intervention. One of skill in the art can readily define similar parameters for p53, DD1α and/or PD-1 expression in a variety of statues for various dieases or conditions (e.g., cancer, allergy, asthma, and/or infection). These value ranges can be provided to clinicians, for example, on a chart, programmed into a PDA etc.

A standard comprising a reference value or range of values can also be synthesized. A known amount of p53, DD1α and/or PD-1 (or a series of known amounts) can be prepared within the typical expression range for p53, DD1α and/or PD-1 that is observed in a general population. In one embodiment, a recombinant p53, DD1α and/or PD-1 is used as a standard for generating a reference value or set of values. This method has an advantage of being able to compare the extent of disease in one or more individuals in a mixed population. This method can also be useful for subjects who lack a prior sample to act as a reference value or for routine follow-up post-diagnosis. This type of method can also allow standardized tests to be performed among several clinics, institutions, or countries etc.

Bispecific and Multispecific Polypeptide Agents for Targeting DD1α and/or PD-1

Contemplated herein are bispecific and multispecific polypeptide agents that specifically bind to PD-1 and/or DD1α when these molecules are expressed on the surface of a cell or one or more interacting cells, such as a tumor cell, T-cell or macrophage. The polypeptide agents can comprise at least one polypeptide domain having a binding site with binding specificity for a PD-1 target, and at least one polypeptide domain having a binding site with binding specificity for a DD1α target. Alternatively, the polypeptide agents can comprise at least two polypeptide domains each having a binding site with binding specificity for DD1α(e.g., a DD1α/DD1αbispecific or multispecific polypeptide agent). As described herein, such polypeptide agents can selectively bind to double positive cells that co-express both PD-1 and DD1α, or can bind to DD1α/DD1α and PD-1/DD1αpairs on separate cells that are in close proximity to one another.

Accordingly, polypeptides that specifically bind cell-surface antigens, such as antibodies and antigen-binding fragments thereof, can be formatted into polypeptide agents as described herein to provide agents that can selectively bind to a cell or cells expressing DD1α and/or PD-1. Because these bispecific and multispecific polypeptide agents selectively bind cells that have potentially interactive DD1α and/or PD-1 monomers, the efficacy of treatment is expected to be much higher than treating with either agent alone or a combination of single specificity agents. In addition, an agent that binds only PD-1 may inhibit DD1α:PD-1 interaction, but it will not inhibit DD1α:DD1α interaction. To address homophilic interaction, an agent that binds DD1α must be included. Thus, to inhibit the entire DD1α/PD-1 axis, inhibition of both DD1α and PD-1 activities must be employed. While separate agents that target the respective proteins individually could be useful, a bispecific agent that binds both can have additional benefits in terms of kinetics and pharmacodynamics.

DD1α binding agents can thus be screened in vitro or in vivo for those that block or disrupt homophilic interaction to identify agents that, when combined with anti-PD-1 agents will have activity superior to anti-DD1α agents that block only DD1α: PD-1 interaction. It is noted that the DD1αsite involved in DD1α:DD1α homophilic interaction is not necessarily the same site of DD1α involved in DD1α: PD-1 heterophiic interaction. It is thus important to recognize that some agents, e.g., antibodies that recognize DD1α, may disrupt or block DD1α: PD-1 interaction without necessarily disrupting or blocking DD1α:DD1α homophilic interaction. That is, not every antibody or agent that specifically binds DD1α and inhibits one or the other of homophilic and heterophilic interaction will necessarily interfere with both homophilic and heterophilic intreractions. Thus, it is contemplated that a bispecific agent can include, for example, a binding specificity that interferes with DD1α homophilic interaction and a binding specificity that interferes with DD1α: PD-1 heterophilic interaction. By this approach, the entire DD1α/PD-1 axis can be targeted with a single agent. To the extent that the homophilic interaction would not necessarily be inhibited by all agents that simply bind DD1α, this approach provides for superior modulation of the whole axis.

In some embodiments of the aspects described herein, a polypeptide agent can be formatted as a bispecific polypeptide agent as described herein, and in e.g., US 2010/0081796 and US 2010/0021473, the contents of which are herein incorporated by reference in their entireties. In other embodiments of the aspects described herein, a polypeptide agent can be formatted as a multispecific polypeptide agent, for example as described in WO 03/002609, the entire teachings of which are incorporated herein by reference.

Bispecific and multispecific polypeptide agents can comprise immunoglobulin variable domains that have different binding specificities. Such bispecific and multispecific polypeptide agents can comprise combinations of heavy and light chain domains. For example, a bispecific polypeptide agent can comprise a V_{H} domain and a V_{L} domain, which can be linked together in the form of an scFv (e.g., using a suitable linker such as Gly₄Ser) that binds one target, i.e., DD1α or PD-1. A construct that includes, e.g., an scFv that binds DD1α and an scFv that binds PD-1, is said to be bispecific for DD1α and PD-1. Similarly, a construct that includes e.g., an scFv that binds DD1α and a second scFv that binds DD1α is said to be bispecific for DD1α. Similar arrangements can be applied in the context of, e.g., a bispecific F(ab′)₂ construct.

Single domain antibody constructs are also contemplated for the development of bispecific reagents. In some embodiments of the aspects described herein, the bispecific and multispecific polypeptide agents may not comprise complementary V_{H}/V_{L} pairs which form an antigen-binding site that binds to a single antigen or epitope co-operatively as found in conventional two chain antibodies. Instead, in some embodiments, the bispecific and multispecific polypeptide agents can comprise a V_{H}/V_{L} complementary pair, wherein the V domains each have different binding specificities, such that two different epitopes or antigens are specifically bound.

In addition, in some embodiments, the bispecific and multispecific polypeptide agents comprise one or more C_{H} or C_{L} domains. A hinge region domain can also be included in some embodiments. Such combinations of domains can, for example, mimic natural antibodies, such as IgG or IgM, or fragments thereof, such as Fv, scFv, Fab or F(ab′)₂ molecules. Other structures, such as a single arm of an IgG molecule comprising V_{H}, V_{L}, C_{H1} and C_{L} domains, are also encompassed within the embodiments described herein. Alternatively, in another embodiment, a plurality of bispecific polypeptide agents are combined to form a multimer. For example, two different bispecific polypeptide agents can be combined to create a tetra-specific molecule. It will be appreciated by one skilled in the art that the light and heavy variable regions of a bispecific or multispecific polypeptide agent produced according to the methods described herein can be on the same polypeptide chain, or alternatively, on different polypeptide chains. In the case where the variable regions are on different polypeptide chains, then they can be linked via a linker, generally a flexible linker (such as a polypeptide chain), a chemical linking group, or any other method known in the art.

In different embodiments of the aspects described herein, the bispecific and multispecific polypeptide agents can be formatted as bi- or multispecific antibodies or antigen-binding fragments thereof, or into bi- or multispecific non-antibody structures. Suitable formats include, for example, any suitable polypeptide structure in which an antibody variable domain, or one or more of the complementarity determining regions (CDRs) thereof, can be incorporated so as to confer binding specificity for antigen on the structure. A variety of suitable antibody formats are known in the art, such as bispecific IgG-like formats (e.g., chimeric antibodies, humanized antibodies, human antibodies, single chain antibodies, heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single variable domain (e.g., V_{H}, V_{L}, V_{HH}), a diabody (dAb), and modified versions of any of the foregoing (e.g., modified by the covalent attachment of polyalkylene glycol (e.g., polyethylene glycol, polypropylene glycol, polybutylene glycol) or other suitable polymer).

A bispecific or multispecific polypeptide agent can be formatted using a suitable linker such as (Gly₄Ser)₁₁, where n=from 1 to 8, e.g., 2, 3, 4, 5, 6 or 7. If desired, bispecific or multispecific polypeptide agents can be linked to an antibody Fc region, comprising one or both of C_{H2} and C_{H3} domains, and optionally a hinge region. For example, vectors encoding bispecific or multispecific polypeptide agents linked as a single nucleotide sequence to an Fc region can be used to prepare such polypeptides.

In some embodiments of the aspects described herein, antigen-binding fragments of antibodies can be combined and/or formatted into non-antibody multispecific polypeptide structures to form multivalent complexes, which bind target molecules having the same epitope, thereby providing superior avidity. For example, natural bacterial receptors such as SpA can been used as scaffolds for the grafting of CDRs to generate ligands which bind specifically to one or more epitopes. Details of this procedure are described in e.g., U.S. Pat. No. 5,831,012, herein incorporated by reference in its entirety. Other suitable scaffolds include those based on fibronectin and affibodies. Details of suitable procedures are described in e.g., WO 98/58965, herein incorporated by reference in its entirety. Other suitable scaffolds include lipocallin and CTLA4, as described in e.g., van den Beuken et al., J. Mol. Biol. 310:591-601 (2001), and scaffolds such as those described in e.g., WO 00/69907 (Medical Research Council), herein incorporated by reference in their entireties, which are based for example on the ring structure of bacterial GroEL or other chaperone polypeptides. In some embodiments, protein scaffolds can be combined. For example, CDRs specific for PD-1 and DD1α can be grafted onto a CTLA4 scaffold and used together with immunoglobulin V_{H} or V_{L} domains to form a bispecific or multispecific polypeptide agent. Likewise, fibronectin, lipocallin and other scaffolds can be combined in other embodiments.

In some embodiments of the aspects described herein, the bispecific or multispecific polypeptide agents can be formatted as fusion proteins that contain a first antigen-binding domain that is fused directly to a second antigen-binding domain. If desired, in some embodiments, such a format can further comprise a half-life extending moiety. For example, the bispecific or multispecific polypeptide agent can comprise a first antigen-binding domain specific for PD-1, that is fused directly to a second antigen-binding domain specific for DD1α, that is fused directly to an antigen-binding domain that binds serum albumin.

Generally, the orientation of the polypeptide domains that have a binding site with binding specificity for a target, and whether a bispecific or multispecific polypeptide agent comprises a linker, are a matter of design choice. However, some orientations, with or without linkers, can provide better binding characteristics than other orientations. All orientations are encompassed by the aspects and embodiments described herein, and bispecific or multispecific polypeptide agents that contain an orientation that provides desired binding characteristics can be easily identified by screening.

Accordingly, in one aspect, described herein is a multispecific agent comprising at least one binding site that specifically binds to a DD1αmolecule, and at least one binding site that specifically binds to a PD-1 molecule. In one embodiment of this aspect, the DD1α molecule bound by the multispecific agent has the sequence set forth in SEQ ID NO: 1, or is an allelic or splice variant of SEQ ID NO:1. In one embodiment of this aspect, the PD-1 molecule bound by the multispecific agent has the sequence set forth in SEQ ID NO: 2, or is an allelic or splice variant of SEQ ID NO: 2.

In some embodiments, the multispecific agent can further comprise a binding site that specifically binds to other immune inhibitory molecule-based inhibitory chimeric antigen receptors (iCARs) for T-cell therapy. In some embodiments, the multispecific agent can comprise a binding site that specifically binds to PD-1 and a binding site that specifically binds to CTLA-4.

It is to be understood that the bispecific or multispecific polypeptide agents described herein will generally bind to naturally occurring or synthetic analogs, variants, mutants, alleles, parts and fragments of a DD1α and/or PD-1 target; or at least to those analogs, variants, mutants, alleles, parts and fragments of a DD1α and/or PD-1 target, that contain one or more antigenic determinants or epitopes that are essentially the same as the antigenic determinant(s) or epitope(s) to which the bispecific or multispecific polypeptide agents described herein bind on the DD1α and PD-1 target. In some embodiments, the amino acid sequences and polypeptides described herein bind to some analogs, variants, mutants, alleles, parts and fragments of a DD1α and/or PD-1 target, but not to others.

In some embodiments of the aspects described herein, the binding sites of the bispecific polypeptide agents, such as the bispecific antibodies, are directed against a target's ligand interaction site. In other embodiments of the aspects described herein, the binding sites of the bispecific polypeptide agents are directed against a site on a target in the proximity of the ligand interaction site, in order to provide steric hindrance for the interaction of the target with its receptor or ligand. Preferably, the site against which the bispecific polypeptide agents described herein are directed is such that binding of the target to its receptor or ligand is modulated, and in particular, inhibited or prevented.

By binding to a PD-1 ligand interaction site, a bispecific polypeptide agent or multispecific polypeptide agent described herein can reduce or inhibit the activity or expression of PD-1. As used herein, a bispecific polypeptide agent or multispecific polypeptide agent that specifically binds to PD-1 has the ability to reduce the activity or expression of PD-1 in a cell (e.g., T cells such as CD8+ T cells) by at least 5%, at least 10%, at least 20%, at least 30%, at least 40%, at least 50%, at least 60%, at least 70%, at least 80%, at least 90%, at least 95% or up to and including 100% relative to untreated control levels.

By binding to a DD1α ligand interaction site, a bispecific polypeptide agent or multispecific polypeptide agent can reduce or inhibit the activity or expression of DD1α. As used herein, a bispecific polypeptide agent or multispecific polypeptide agent that specifically binds to DD1α has the ability to reduce the activity or expression of DD1α in a cell (e.g., T cells such as CD8+ T cells) by at least 5%, at least 10%, at least 20%, at least 30%, at least 40%, at least 50%, at least 60%, at least 70%, at least 80%, at least 90%, at least 95%, or up to and including 100% relative to untreated control levels.

Thus, in some embodiments of the aspects described herein, a binding site of a bispecific or multispecific polypeptide agent is directed against a ligand interaction site on DD1α such that the interaction of DD1α with another monomer of DD1α is modulated, and in particular inhibited or prevented. In other embodiments, a binding site of a bispecific or multispecific polypeptide agent is directed against a ligand interaction site on DD1α such that the interaction of DD1α with PD-1 is modulated, and in particular inhibited or prevented. In other embodiments, a binding site of a bispecific or multispecific polypeptide agent is directed against a ligand interaction site on PD-1, such that the interaction of PD-1 with PD-L1 and/or PD-L2 is modulated, and in particular inhibited or prevented. In other embodiments, a bispecific or multispecific polypeptide agent as described herein is directed against a ligand interaction site on DD1α such that the interaction of DD1α with a second monomer of DD1α is modulated, and in particular inhibited or prevented, while the interaction of DD1α with PD-1 is not necessarily modulated, inhibited, or prevented. In other embodiments, a bispecific or multispecific polypeptide agent as described herein is directed against a ligand interaction site on DD1α such that the interaction of DD1α with PD-1 is modulated, and in particular inhibited or prevented, while the interaction of DD1α with another DD1αmonomer is not modulated, inhibited, or prevented.

Accordingly, in some embodiments of the aspects described herein, a ligand interaction site of PD-1 comprises amino acid residues 41-136 of SEQ ID NO: 2. In some embodiments, a ligand interaction site on PD-1 comprises any of the amino acid residues selected from the group consisting of amino acids 64, 66, 68, 73, 74, 75, 76, 78, 90, 122, 124, 126, 128, 130, 131, 132, 134, and 136 of SEQ ID NO:2. In some embodiments, a ligand interaction site on PD-1 comprises any of the amino acid residues selected from the group consisting of amino acids 78, 126, and 136 of SEQ ID NO: 2.

Antibodies suitable for practicing the methods described herein are preferably monoclonal, and can include, but are not limited to human, humanized or chimeric antibodies, comprising single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain at least two antigen or target binding sites that specifically bind DD1α/DD1α or PD-1/DD1α. The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with the invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) or Marks et al., J. Mol. Biol. 222:581-597 (1991), for example.

The term “antibody fragment,” as used herein, refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and C_{H1} domains; (ii) the Fab′ fragment, which is a Fab fragment having one or more cysteine residues at the C-terminus of the C_{H1} domain; (iii) the Fd fragment having V_{H} and C_{H1} domains; (iv) the Fd′ fragment having V_{H} and C_{H1} domains and one or more cysteine residues at the C-terminus of the C_{H1} domain; (v) the Fv fragment having the V_{L} and V_{H} domains of a single arm of an antibody; (vi) the dAb fragment (Ward et al., Nature 341, 544-546 (1989)) which consists of a V_{H} domain; (vii) isolated CDR regions; (viii) F(ab′)₂ fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}-C_{H1}-V_{H}-C_{H1}) which, together with complementary light chain polypeptides, form a pair of antigen binding regions (Zapata et al. Protein Eng. 8(10):1057-1062 (1995); and U.S. Pat. No. 5,641,870).

In another aspect, bispecific antibodies having an IgG-like format are provided. Such formats have the conventional four chain structure of an IgG molecule (2 heavy chains and two light chains), in which one antigen-binding region (comprised of a V_{H} and a V_{L} domain) specifically binds PD-1 and the other antigen-binding region (also comprised of a V_{H} and a V_{L} domain) specifically binds DD1α. In some embodiments, each of the variable regions (2 V_{H} regions and 2 V^{L} regions) is replaced with a dAb or single variable domain. The dAb(s) or single variable domain(s) that are included in an IgG-like format can have the same specificity or different specificities. In some embodiments, the IgG-like format is tetravalent and can have two, three or four specificities. For example, the IgG-like format can be bispecific and comprise 3 dAbs that have the same specificity and another dAb that has a different specificity; bispecific and comprise two dAbs that have the same specificity and two dAbs that have a common but different specificity; trispecific and comprise first and second dAbs that have the same specificity, a third dAb with a different specificity and a fourth dAb with a different specificity from the first, second and third dAbs; or tetraspecific and comprise four dAbs that each have a different specificity. Antigen-binding fragments of IgG-like formats (e.g., Fab, F(ab′)₂, Fab′, Fv, scFv) can be prepared as is known to one of skill in the art, and as described herein.

### Methods for Making Bispecific Polypeptide Agents

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule is usually done by affinity chromatography steps, but the product yields are low. Similar procedures are disclosed in WO 93/08829, and in Traunecker et al, EMBO J., 10:3655-3659 (1991), herein incorporated by reference in their entireties.

According to another approach, described in WO96/27011, herein incorporated by reference in its entirety, the interface between a pair of antibody molecules can be engineered to maximize the percentage of heterodimers which are recovered from recombinant cell culture. Such an approach is particularly useful for the generation of bispecific antibodies that bind DD1α and PD-1. The interfaces can comprise at least a part of the CH₃ domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

In one aspect, the bispecific antibodies described herein include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies can be made using any convenient cross-linking methods. Suitable cross-linking agents are well known in the art, and are disclosed in U.S. Pat. No. 4,676,980, along with a number of cross-linking techniques. In one embodiment, the bispecific antibodies do not comprise a heteroconjugate.

Techniques for generating bispecific antibodies from antibody fragments have also been described in the literature. For example, bispecific antibodies can be prepared using chemical linkage. For example, Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. A bispecific antibody specific for PD-1 and DD1α produced using this method can be used in any of the compositions and methods described herein.

In some embodiments, a bispecific antibody specific for PD-1/DD1α or DD1α/DD1α can be produced using any of the methods described in U.S. Patent Application No.: 20100233173; U.S. Patent Application No.: 20100105873; U.S. Patent Application No.: 20090155275; U.S. Patent Application No.: 20080071063; and U.S. Patent Application No.: 20060121042, the contents of each of which are herein incorporated in their entireties by reference. In some embodiments, a bispecific antibody specific for PD-1/DD1α or DD1α/DD1α can be produced using any of the methods described in U.S. Patent Application No.: 20090175867 and U.S. Patent Application No.: 20110033483 the contents of which are herein incorporated in their entireties by reference.

In some embodiments, the bispecific antibodies can be made by the direct recovery of Fab′-SH fragments recombinantly expressed, e.g., in E. coli, and can be chemically coupled to form bispecific antibodies. For example, Shalaby et al., J. Exp. Med, 175: 217-225 (1992) describe the production of a fully humanized bispecific antibody F(ab′)₂ molecule. As described by Shalaby et al., each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets (see e.g., Shalaby et al, supra). Accordingly, this method can be used to generate a bispecific antibody to PD-1/DD1α or DD1α/DD1α to restore T-cell mediated anti-cancer function.

Various techniques for making and isolating bispecific antibody fragments directly from recombinant cell culture have also been described, and can be used in the generation of the bispecific antibodies that specifically bind PD-1/DD1α or DD1α/DD1α. For example, bispecific antibodies have been produced using leucine zippers (Kostelny et al., J. Immunol, 148(5):1547-1553 (1992)). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{H} and V_{L} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994). Alternatively, the antibodies can be “linear antibodies” as described in Zapata et al. Protein Eng. 8(10):1057-1062 (1995). Briefly, these antibodies comprise a pair of tandem Fd segments (V_{H}-C_{H1}-V_{H}-C_{H1}) which form a pair of antigen binding regions. Linear antibodies can be bispecific or multispecific.

Antibodies useful in the present methods can be described or specified in terms of the particular CDRs they comprise. The compositions and methods described herein encompass the use of an antibody or derivative thereof comprising a heavy or light chain variable domain, where the variable domain comprises (a) a set of three CDRs, and (b) a set of four framework regions, and in which the antibody or antibody derivative thereof specifically binds PD-1/DD1α or DD1α/DD1α.

Also provided herein are chimeric antibody derivatives of the bispecific and multispecific polypeptide agents, i.e., antibody molecules in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Chimeric antibody molecules can include, for example, one or more antigen binding domains from an antibody of a mouse, rat, or other species, with human constant regions. A variety of approaches for making chimeric antibodies have been described and can be used to make chimeric antibodies containing the immunoglobulin variable region which recognizes the selected antigens, i.e., PD-1/DD1α or DD1α/DD1α, on the surface of tumor cells, macrophages and/or T-cells. See, for example, Takeda et al., 1985, Nature 314:452; Cabilly et al., U.S. Pat. No. 4,816,567; Boss et al.; Tanaguchi et al., European Patent Publication EP171496; European Patent Publication 0173494, United Kingdom patent GB 2177096B).

The bispecific and multispecific polypeptide agents described herein can also be a humanized antibody derivative. Humanized forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

Chemical conjugation can also be used to generate the bispecific or multispecific antibodies described herein, and is based on the use of homo- and heterobifunctional reagents with E-amino groups or hinge region thiol groups. Homobifunctional reagents such as 5,5′-Dithiobis(2-nitrobenzoic acid) (DNTB) generate disulfide bonds between the two Fabs, and O-phenylenedimaleimide (O-PDM) generate thioether bonds between the two Fabs (Brenner et al., 1985, Glennie et al., 1987). Heterobifunctional reagents such as N-succinimidyl-3-(2-pyridylditio) propionate (SPDP) combine exposed amino groups of antibodies and Fab fragments, regardless of class or isotype (Van Dijk et al., 1989).

In some embodiments, the antibodies described herein, i.e., antibodies that are useful for treating e.g., cancer or infection and are specific for PD-1/DD1α or DD1α/DD1α, include derivatives that are modified, i.e., by the covalent attachment of any type of molecule to the antibody such that covalent attachment does not prevent the antibody from binding to PD-1/DD1α or DD1α/DD1α. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids.

Accordingly, the bispecific or multispecific antibodies described herein for use in the treatment of chronic immune conditions can be generated by any suitable method known in the art. Monoclonal and polyclonal antibodies against both PD-1 and DD1α are known in the art. To the extent necessary, e.g., to generate antibodies with particular characteristics or epitope specificity, the skilled artisan can generate new monoclonal or polyclonal anti-PD-1 and anti-DD1α antibodies as discussed below or as known in the art. In other embodiments, the bispecific and multispecific antibodies and antigen-binding fragments thereof described herein can utilize PD-1 binding site sequences from monoclonal antibodies against human PD-1, such as, MDX-1106 (ONO-4538), a fully human IgG4 anti-PD-1 blocking antibody (Journal of Clinical Oncology, 2008 Vol 26, No 15S); CT-011 (CureTech, LTD, previously CT-AcTibody or BAT), a humanized monoclonal IgG1 antibody (Benson D M et al., Blood. 2010 May 11), or those obtained from, clone NAT (Abcam), clone EH12.2H7 (Biolegend), clone J116 (eBioscience), clone MIH4 (eBioscience), clone J105 (eBioscience), or clone 192106 (R& D systems). Similarly, the bispecific and multispecific antibodies and antigen-binding fragments thereof described herein can utilize DD1α binding site sequences from monoclonal antibodies against human DD1α, such as those obtained from, e.g., LIFESPAN BIOSCIENCES, ATLAS ANTIBODIES, AVIVA SYSTEMS BIOLOGY, SANTA CRUZ BIOTECHNOLOGY, ABNOVA, GENETEX, NOVUS BIOLOGICS, or as manufactured using the methods described herein.

Polyclonal antibodies specific for DD1α and/or PD-1 can be produced by various procedures well known in the art. For example, DD1α and/or PD-1 polypeptides or fragments thereof can be administered to various host animals including, but not limited to rabbits, mice, rats, etc. to induce the production of sera containing polyclonal antibodies specific for the protein. Polyclonal antibodies are preferably raised in animals by multiple subcutaneous (sc) or intraperitoneal (ip) injections of the relevant antigen, e.g., a PD-1 fragment and an adjuvant. It can be useful to conjugate the antigen to a protein that is immunogenic in the species to be immunized, e.g., keyhole limpet hemocyanin, serum albumin, bovine thyroglobulin, or soy-bean trypsin inhibitor using a bifunctional or derivatizing agent, for example, maleimidobenzoyl sulfosuccinimide ester (conjugation through cysteine residues), N-hydroxy-succinimide (through lysine residues), glutaraldehyde, succinic anhydride, SOCl₂, or R¹NCNR, where R and R¹ are different alkyl groups.

Animals can be immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with 1/5 to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Various other adjuvants can be used to increase the immunological response, depending on the host species, and include but are not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Suitable adjuvants are also well known to one of skill in the art.

In another example, antibodies useful in the methods and compositions described herein can also be generated using various phage display methods known in the art, such as isolation from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

In phage display methods, functional antibody domains are displayed on the surface of phage particles which carry the nucleic acid sequences encoding them. In a particular embodiment, such phage can be utilized to display antigen binding domains expressed from a repertoire or combinatorial antibody library (e.g., human or murine). Examples of phage display methods that can be used to make the antibodies described herein include those disclosed in Brinkman et al, 1995, J. Immunol. Methods 182:41-50; Ames et al., 1995, J. Immunol. Methods 184:177-186; Kettleborough et al, 1994, Eur. J. Immunol. 24:952-958; Persic et al., 1997, Gene 187:9-18; Burton et al., 1994, Advances in Immunology, 191-280; PCT Application No. PCT/GB91/01134; PCT Publications WO 90/02809; WO 91/10737; WO 92/01047; WO 92/18619; WO 93/1 1236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, the contents of which are herein incorporated by reference in their entireties.

As used herein, a “chimeric antibody” refers to a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science, 1985, 229:1202; Oi et al, 1986, Bio-Techniques 4:214; Gillies et al., 1989, J. Immunol. Methods 125:191-202; U.S. Pat. Nos. 5,807,715; 4,816,567; and 4,816,397, the contents of which are herein incorporated by reference in their entireties.

“Humanized antibodies,” as the term is used herein, refer to antibody molecules from a non-human species, where the antibodies that bind the desired antigen, i.e., PD-1/DD1α or DD1α/DD1α, have one or more CDRs from the non-human species, and framework and constant regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., 1988, Nature 332:323. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology, 1991, 28(4/5):489-498; Studnicka et al., 1994, Protein Engineering 7(6):805-814; Roguska. et al, 1994, PNAS 91:969-973), and chain shuffling (U.S. Pat. No. 5,565,332), the contents of which are herein incorporated by reference in their entireties. Accordingly, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), the contents of which are herein incorporated by reference in their entireties, by substituting rodent CDRs or CDR sequences for the corresponding sequences of a human antibody. Accordingly, such “humanized” antibodies are chimeric antibodies (U.S. Pat. No. 4,816,567, the contents of which are herein incorporated by reference in its entirety) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework can be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen, i.e., DD1α and/or PD-1, and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding. Humanized antibodies and affinity matured variants thereof are described in, for example, U.S. Pat. No. 6,884,879 issued Feb. 26, 2005, the contents of which are herein incorporated by reference in its entirety.

Completely human antibodies are particularly desirable for the therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741, the contents of which are herein incorporated by reference in their entireties.

Human antibodies can also be produced using transgenic mice which express human immunoglobulin genes, and upon immunization are capable of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. The transgenic mice are immunized in the normal fashion with a selected antigen, e.g., all or a portion of PD-1/DD1α or DD1α. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see, Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, the contents of which are herein incorporated by reference in their entireties. In addition, companies such as Abgenix, Inc. (Freemont, Calif.) and Medarex (Princeton, N.J.) can be engaged to provide human antibodies directed against a selected antigen using technology similar to that described above. See also, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992), the contents of which are herein incorporated by reference in their entireties.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. Phage display can be performed in a variety of formats; for their review see, e.g., Johnson, Kevin S, and Chiswell, David J., Current Opinion in Structural Biology 3:564-571 (1993). A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

Human antibodies can also be generated by in vitro activated B cells (see U.S. Pat. Nos. 5,567,610 and 5,229,275, the contents of which are herein incorporated by reference in their entireties).

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

Further, the bispecific and multispecific antibodies to PD-1 or DD1αdescribed herein can, in turn, be utilized to generate anti-idiotype antibodies that “mimic” proteins described herein using techniques well known to those skilled in the art. (See, e.g. Greenspan & Bona, 1989, FASEB J. 7(5):437-444; and Nissinoff. 1991, J. Immunol. 147(8):2429-2435). Fab fragments of such anti-idiotypes can be used in therapeutic regimens to elicit an individual's own immune response against PD-1 or DD1αpresent on tumor cells, macrophages or T-cells.

In some embodiments of these aspects, amino acid sequence modification(s) of the antibodies or antibody fragments described herein are contemplated. For example, it can be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics, e.g., specifically binds to PD-1/DD1α or DD1α/DD1α. The amino acid changes also can alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include antibodies with an N-terminal methionyl residue, or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated.

Substantial modifications in the biological properties of the antibodies or antibody fragments thereof described herein are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), H is (H).

Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: H is, Lys, Arg; (5) residues that influence chain orientation: Gly, Pro; (6) aromatic: Trp, Tyr, Phe. Non-conservative substitutions will entail exchanging a member of one of these classes for another class.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

Any cysteine residue not involved in maintaining the proper conformation of the antibody also can be substituted, generally with serine, to improve the oxidative stability of the molecule and prevent aberrant crosslinking Conversely, cysteine bond(s) can be added to the antibody to improve its stability (particularly where the antibody is an antibody fragment such as an Fv fragment).

A particularly preferred type of substitutional variant involves substituting one or more hypervariable region residues of the parent antibody or antibody fragment thereof described herein (e.g., a humanized or human antibody). Generally, the resulting variant(s) selected for further development will have improved biological properties relative to the parent antibody from which they are generated. A convenient way for generating such substitutional variants involves affinity maturation using phage display, a method known to those of skill in the art.

Another type of amino acid variant of the antibodies or antibody fragments thereof described herein alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine can also be used.

Addition of glycosylation sites to the antibodies or antibody fragments thereof described herein is conveniently accomplished by altering the amino acid sequence such that it contains one or more of the above-described tripeptide sequences (for N-linked glycosylation sites). The alteration can also be made by the addition of, or substitution by, one or more serine or threonine residues to the sequence of the original antibody (for O-linked glycosylation sites).

Where the antibody or antibody fragment thereof described herein comprises an Fc region, the carbohydrate attached thereto can be altered. For example, antibodies with a mature carbohydrate structure that lacks fucose attached to an Fc region of the antibody are described in US Pat Appl No US 2003/0157108 A1, Presta, L. See also US 2004/0093621 A1 (Kyowa Hakko Kogyo Co., Ltd). Antibodies with a bisecting N-acetylglucosamine (GlcNAc) in the carbohydrate attached to an Fc region of the antibody are referenced in WO03/011878, Jean-Mairet et al. and U.S. Pat. No. 6,602,684, Umana et al. Antibodies with at least one galactose residue in the oligosaccharide attached to an Fc region of the antibody are reported in WO97/30087, Patel et al. See, also, WO98/58964 (Raju, S.) and WO99/22764 (Raju, S.) concerning antibodies with altered carbohydrate attached to the Fc region thereof.

It can be desirable to modify an antibody or antibody fragment thereof described herein with respect to effector function, e.g., so as to enhance antigen-dependent cell-mediated cytotoxicity (ADCC) and/or complement dependent cytotoxicity (CDC) of the antibody. This can be achieved by introducing one or more amino acid substitutions in an Fc region of the antibody or antibody fragment thereof. Alternatively or additionally, cysteine residue(s) can be introduced in the Fc region, thereby allowing interchain disulfide bond formation in this region. A homodimeric antibody thus generated can have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity can also be prepared using heterobifunctional cross-linkers as described in Wolff et al. Cancer Research 53:2560-2565 (1993). Alternatively, an antibody can be engineered which has dual Fc regions and can thereby have enhanced complement lysis and ADCC capabilities. See e.g., Stevenson et al. Anti-Cancer Drug Design 3:219-230 (1989), WO2000/042072, WO99/51642, U.S. Pat. No. 6,194,551B1, U.S. Pat. No. 6,242,195B1, U.S. Pat. No. 6,528,624B1 and U.S. Pat. No. 6,538,124. The antibodies can comprise an amino acid substitution at one or more of amino acid positions 270, 322, 326, 327, 329, 313, 333 and/or 334 of the Fc region thereof (Eu numbering of residues).

To increase the serum half-life of the antibody or antibody fragment thereof described herein, one can incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG.sub.1, IgG₂, IgG₃, or IgG₄) that is responsible for increasing the in vivo serum half-life of the IgG molecule.

Nucleic acid molecules encoding amino acid sequence variants of the antibody or antibody fragment thereof described herein are prepared by a variety of methods known in the art. These methods include, but are not limited to isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

Administration of Agents that Target DD1α and/or PD-1 (e.g., Bispecific or Multispecific Polypeptide Agents)

Agents that target DD1α and/or PD-1 (e.g., DD1α antagonists and/or agonists, and/or PD-1 antagonists and/or agonists, including, e g., the bispecific or multispecific polypeptide agents described herein) can be administered to a subject in need thereof by any appropriate route which results in an effective treatment in the subject. As used herein, the terms “administering,” and “introducing” are used interchangeably and refer to the placement of a bispecific or multispecific polypeptide agent into a subject by a method or route which results in at least partial localization of such agents at a desired site, such as a tumor, such that a desired effect(s) is produced.

In some embodiments, agents that target DD1α and/or PD-1 (e.g., DD1α antagonists and/or agonists, and/or PD-1 antagonists and/or agonists, including, e g., the bispecific or multispecific polypeptide agents described herein) is administered to a subject having a chronic immune condition by any mode of administration that delivers the agent systemically or to a desired surface or target, and can include, but is not limited to injection, infusion, instillation, and inhalation administration. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, intratumoral, and intrasternal injection and infusion. In preferred embodiments, the bispecific or multispecific polypeptide agents for use in the methods described herein are administered by intravenous infusion or injection.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of the bispecific or multispecific polypeptide agent other than directly into a target site, tissue, or organ, such as a tumor site, such that it enters the subject's circulatory system and, thus, is subject to metabolism and other like processes.

For the clinical use of the methods described herein, administration of the bispecific or multispecific polypeptide agents can include formulation into pharmaceutical compositions or pharmaceutical formulations for parenteral administration, e.g., intravenous; mucosal, e.g., intranasal; ocular, or other mode of administration. In some embodiments, the bispecific or multispecific polypeptide agents described herein can be administered along with any pharmaceutically acceptable carrier compound, material, or composition which results in an effective treatment in the subject. Thus, a pharmaceutical formulation for use in the methods described herein can contain a bispecific or multispecific polypeptide agent as described herein in combination with one or more pharmaceutically acceptable ingredients.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, a bispecific or multispecific polypeptide agent. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (19) pH buffered solutions; (20) polyesters, polycarbonates and/or polyanhydrides; (21) bulking agents, such as polypeptides and amino acids (22) serum components, such as serum albumin, HDL and LDL; (23) C2-C12 alcohols, such as ethanol; and (24) other non-toxic compatible substances employed in pharmaceutical formulations. Release agents, coating agents, preservatives, and antioxidants can also be present in the formulation. The terms such as “excipient”, “carrier”, “pharmaceutically acceptable carrier” or the like are used interchangeably herein.

Agents that target DD1α and/or PD-1 (e.g., DD1α antagonists and/or agonists, and/or PD-1 antagonists and/or agonists, including, e g., the bispecific or multispecific polypeptide agents described herein) can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (79) nasally. Additionally, a bispecific or multispecific polypeptide agent can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

Parenteral dosage forms of the agents that target DD1α and/or PD-1 (e.g., DD1α antagonists and/or agonists, and/or PD-1 antagonists and/or agonists, including, e.g., the bispecific or multispecific polypeptide agents described herein) can also be administered to a subject with a chronic immune condition by various routes, including, but not limited to subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to sodium chloride injection, Ringer's injection, dextrose Injection, dextrose and sodium chloride injection, and lactated Ringer's injection; water-miscible vehicles such as, but not limited to ethyl alcohol, polyethylene glycol, and propylene glycol; and non-aqueous vehicles such as, but not limited to corn oil, cottonseed oil, peanut oil, sesame oil, ethyl oleate, isopropyl myristate, and benzyl benzoate.

Agents that target DD1α and/or PD-1 (e.g., DD1α antagonists and/or agonists, and/or PD-1 antagonists and/or agonists, including, e g., the bispecific or multispecific polypeptide agents described herein) can be packaged in a pressurized aerosol container together with suitable propellants, for example, hydrocarbon propellants like propane, butane, or isobutane with conventional adjuvants. Agents that target DD1α and/or PD-1 (e.g., DD1α antagonists and/or agonists, and/or PD-1 antagonists and/or agonists, including, e g., the bispecific or multispecific polypeptide agents described herein) can also be administered in a non-pressurized form such as in a nebulizer or atomizer. Agents that target DD1α and/or PD-1 (e.g., DD1α antagonists and/or agonists, and/or PD-1 antagonists and/or agonists, including, e.g., the bispecific or multispecific polypeptide agents described herein) can also be administered directly to the airways in the form of a dry powder, for example, by use of an inhaler.

Suitable powder compositions include, by way of illustration, powdered preparations of a bispecific or multispecific polypeptide agent thoroughly intermixed with lactose, or other inert powders acceptable for intrabronchial administration. The powder compositions can be administered via an aerosol dispenser or encased in a breakable capsule which can be inserted by the subject into a device that punctures the capsule and blows the powder out in a steady stream suitable for inhalation. The compositions can include propellants, surfactants, and co-solvents and can be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S, and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The formulations of the agents that target DD1α and/or PD-1 (e.g., DD1α antagonists and/or agonists, and/or PD-1 antagonists and/or agonists, including, e g., the bispecific or multispecific polypeptide agents described herein) further encompass anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to hermetically sealed foils, plastics, unit dose containers (e.g., vials) with or without desiccants, blister packs, and strip packs.

In some embodiments of the aspects described herein, an agent that targets DD1α and/or PD-1 (e.g., DD1α antagonist and/or agonist, and/or PD-1 antagonist and/or agonist, including, e g., bispecific or multispecific polypeptide agent described herein) can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Chemg-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of a compound of formula (I) is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the bispecific or multispecific polypeptide agents described herein. Examples include, but are not limited to those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS). (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to Duolite. A568 and Duolite. AP143 (Rohm&Haas, Spring House, Pa. USA).

In some embodiments, agents that target DD1α and/or PD-1 (e.g., DD1α antagonists and/or agonists, and/or PD-1 antagonists and/or agonists, including, e g., the bispecific or multispecific polypeptide agents described herein) for use in the methods described herein can be administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred when the disorder occurs continuously in the subject, for example where the subject has continuous or chronic symptoms of a viral infection. Each pulse dose can be reduced and the total amount of a bispecific or multispecific polypeptide agent administered over the course of treatment to the patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals can be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

### Dosage and Administration

As used herein, the term “treatment” includes prophylaxis and therapy. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals as well as other veterinary subjects. Preferably, the patients or subjects are human.

In one aspect, the methods described herein provide a method for treating a tumor, cancer or infection in a subject. In one embodiment, the subject can be a mammal. In another embodiment, the mammal can be a human, although the approach is effective with respect to all mammals. The method comprises administering to the subject an effective amount of a pharmaceutical composition comprising an anti-DD1α, anti-PD-1, or other immunologic based therapy in a pharmaceutically acceptable carrier. In some embodiments, the method comprises administering to the subject an effective amount of a pharmaceutical composition comprising an inhibitor of DD1α and/or PD-1, for example, a binding protein, such as an antibody or a peptide. In other embodiments, the inhibitor of DD1α and/or PD-1 comprises a small molecule or an RNA interference molecule (e.g., siRNA, shRNA etc.).

The dosage range for the agent depends upon the potency, and includes amounts large enough to produce the desired effect, e.g., anti-tumor, anti-cancer effect. The dosage should not be so large as to cause unacceptable adverse side effects. Generally, the dosage will vary with the type of inhibitor (e.g., an antibody or fragment, small molecule, siRNA, etc.), and with the age, condition, and sex of the patient. The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

Administration of the doses recited above can be repeated for a limited period of time. In some embodiments, the doses are given once a day, or multiple times a day, for example but not limited to three times a day. In another embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

A therapeutically effective amount is an amount of an agent that is sufficient to produce a statistically significant, measurable change in at least one symptom of a cancer or infection. Such effective amounts can be gauged in clinical trials as well as animal studies for a given agent.

It is contemplated herein that the agents can be delivered intravenously (by bolus or continuous infusion), orally, by inhalation, intranasally, intraperitoneally, intramuscularly, subcutaneously, intracavity, and can be delivered by peristaltic means, if desired, or by other means known by those skilled in the art. The agent can be administered systemically, if so desired.

In one embodiment, the agent is administered to a subject for an extended period of time. Sustained contact with an antibody or peptide composition can be achieved by, for example, repeated administration of the antibody or peptide composition over a period of time, such as one week, several weeks, one month or longer. More preferably, the pharmaceutically acceptable formulation used to administer the active compound provides sustained delivery, such as “slow release” of the agent to a subject. For example, the formulation can deliver the agent or composition for at least one, two, three, or four weeks after the pharmaceutically acceptable formulation is administered to the subject. In some embodiments, a subject to be treated in accordance with the methods described herein is treated with the active composition for at least 30 days (either by repeated administration or by use of a sustained delivery system, or both).

Preferred approaches for sustained delivery include use of a polymeric capsule, a minipump to deliver the formulation, a biodegradable implant, or implanted transgenic autologous cells (as described in e.g., U.S. Pat. No. 6,214,622). Implantable infusion pump systems (such as e.g., Infusaid™; see such as Zierski, J. et al, 1988; Kanoff, R. B., 1994) and osmotic pumps (sold by Alza Corporation™) are available in the art. Another mode of administration is via an implantable, externally programmable infusion pump. Suitable infusion pump systems and reservoir systems are also described in e.g., U.S. Pat. No. 5,368,562 by Blomquist and U.S. Pat. No. 4,731,058 by Doan, developed by Pharmacia Deltec™ Inc.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, an agent can be targeted to a tissue by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. The addition of an antibody to an agent permits the agent to accumulate additively at the desired target site (e.g., tumor site). Antibody-based or non-antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

### Chronic Immune Conditions

Certain aspects of the methods described herein are based, in part, on the discovery that DD1α (or PD-1) expressed on tumor cells interacts with DD1α or PD-1 on the surface of a T-cell, thereby reducing T cell proliferation or activation and impairing immune clearance of tumor cells. Accordingly, the methods using the bispecific and multispecific polypeptide agents described herein are useful in the treatment of subjects having a chronic immune condition, such as cancer or a persistent infection, where an immune response is suppressed, insufficient, inhibited, or abrogated, due to the decreased proliferation of a population of immune cells, such as CD8+ T cells.

Immunosuppression of a host immune response plays a role in a variety of chronic immune conditions, such as in persistent infection and tumor immunosuppression. Recent evidence indicates that this immunosuppression can be mediated by immune inhibitory receptors expressed on the surface of an immune cell, and their interactions with their ligands. Accordingly, by inhibiting the activity and/or expression of such inhibitory receptors, an immune response to a persistent infection or to a cancer or tumor that is suppressed, inhibited, or unresponsive, can be enhanced or uninhibited.

In some embodiments of the methods described herein, the subject being administered the bispecific or multispecific polypeptide agent that is specific for PD-/DD1α or DD1α/DD1α has a persistent infection with a bacterium, virus, fungus, or parasite. “Persistent infections” refer to those infections that, in contrast to acute infections, are not effectively cleared by the induction of a host immune response. During such persistent infections, the infectious agent and the immune response reach equilibrium such that the infected subject remains infectious over a long period of time without necessarily expressing symptoms. Persistent infections often involve stages of both silent and productive infection without rapidly killing or even producing excessive damage of the host cells. Persistent infections include for example, latent, chronic and slow infections. Persistent infection occurs with viruses including, but not limited to human T-Cell leukemia viruses, Epstein-Barr virus, cytomegalovirus, herpesviruses, varicella-zoster virus, measles, papovaviruses, prions, hepatitis viruses, adenoviruses, parvoviruses and papillomaviruses.

In a “chronic infection,” the infectious agent can be detected in the subject at all times. However, the signs and symptoms of the disease can be present or absent for an extended period of time. Non-limiting examples of chronic infection include hepatitis B (caused by hepatitis B virus (HBV)) and hepatitis C (caused by hepatitis C virus (HCV)) adenovirus, cytomegalovirus, Epstein-Barr virus, herpes simplex virus 1, herpes simplex virus 2, human herpes virus 6, varicella-zoster virus, hepatitis B virus, hepatitis D virus, papilloma virus, parvovirus B19, polyomavirus BK, polyomavirus JC, measles virus, rubella virus, human immunodeficiency virus (HIV), human T cell leukemia virus I, and human T cell leukemia virus IL Parasitic persistent infections can arise as a result of infection by, for example, Leishmania, Toxoplasma, Trypanosoma, Plasmodium, Schistosoma, and Encephalitozoon.

In a “latent infection,” the infectious agent (such as a virus) is seemingly inactive and dormant such that the subject does not always exhibit signs or symptoms. In a latent viral infection, the virus remains in equilibrium with the host for long periods of time before symptoms again appear; however, the actual viruses cannot typically be detected until reactivation of the disease occurs. Non-limiting examples of latent infections include infections caused by herpes simplex virus (HSV)-1 (fever blisters), HSV-2 (genital herpes), and varicella zoster virus VZV (chickenpox-shingles).

In a “slow infection,” the infectious agents gradually increase in number over a very long period of time during which no significant signs or symptoms are observed. Non-limiting examples of slow infections include AIDS (caused by HIV-1 and HIV-2), lentiviruses that cause tumors in animals, and prions.

In addition, persistent infections that can be treated using the methods described herein include those infections that often arise as late complications of acute infections. For example, subacute sclerosing panencephalitis (SSPE) can occur following an acute measles infection or regressive encephalitis can occur as a result of a rubella infection.

Additional examples of infectious viruses include: Retroviridae; Picornaviridae (for example, polio viruses, hepatitis A virus; enteroviruses, human coxsackie viruses, rhinoviruses, echoviruses); Calciviridae (such as strains that cause gastroenteritis); Togaviridae (for example, equine encephalitis viruses, rubella viruses); Flaviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses); Poxyiridae (variola viruses, vaccinia viruses, pox viruses); and Iridoviridae (such as African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses). The compositions and methods described herein are contemplated for use in treating infections with these viral agents.

Examples of fungal infections include but are not limited to: aspergillosis; thrush (caused by Candida albicans); cryptococcosis (caused by Cryptococcus); and histoplasmosis. Thus, examples of infectious fungi include, but are not limited to Cryptococcus neoformans, Histoplasma capsulatum, Coccidioides immitis, Blastomyces dermatitidis, Chlamydia trachomatis, Candida albicans. The compositions and methods described herein are contemplated for use in treating infections with these fungal agents.

Examples of infectious bacteria include: Helicobacter pyloris, Borelia burgdorferi, Legionella pneumophilia, Mycobacteria sps (such as M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracia, corynebacterium diphtheriae, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, and Actinomyces israelli. The compositions and methods described herein are contemplated for use in treating infections with these bacterial agents. Other infectious organisms (such as protists) include: Plasmodium falciparum and Toxoplasma gondii. The compositions and methods described herein are contemplated for use in treating infections with these agents.

In some embodiments of the aspects described herein, the methods further comprise administering an effective amount of a viral, bacterial, fungal, or parasitic antigen in conjunction with the bispecific or multispecific polypeptide agent that specifically binds PD-1/DD1α or DD1α/DD1α. Non-limiting examples of suitable viral antigens include: influenza HA, NA, M, NP and NS antigens; HIV p24, pol, gp41 and gp120; Metapneumovirus (hMNV) F and G proteins; Hepatitis C virus (HCV) E1, E2 and core proteins; Dengue virus (DEN1-4) E1, E2 and core proteins; Human Papilloma Virus L1 protein; Epstein Barr Virus gp220/350 and EBNA-3A peptide; Cytomegalovirus (CMV) gB glycoprotein, gH glycoprotein, pp 65, IE1 (exon 4) and pp 150; Varicella Zoster virus (VZV) 1E62 peptide and glycoprotein E epitopes; Herpes Simplex Virus Glycoprotein D epitopes, among many others. The antigenic polypeptides can correspond to polypeptides of naturally occurring animal or human viral isolates, or can be engineered to incorporate one or more amino acid substitutions as compared to a natural (pathogenic or non-pathogenic) isolate.

In other embodiments of the methods described herein, the subject having a chronic immune condition being administered the bispecific or multispecific polypeptide agent that specifically binds PD-1/DD1α or DD1α/DD1α has a cancer or tumor.

Studies have shown defective or suppressed immune responses in patients diagnosed with cancer. Interference with suppressive immunoregulatory signals in the tumor microenvironment can overcome this effect and permit anti-tumor immunity effects. Accordingly, provided herein are methods to treat a subject having a cancer or tumor comprising administering an effective amount of a bispecific or multispecific polypeptide agent that is specific for PD-1/DD1α or DD1α/DD1α.

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Examples of cancer that can be treated with the methods and compositions described herein include but are not limited to carcinoma, lymphoma, blastoma, sarcoma, and leukemia. More particular examples of such cancers include, but are not limited to basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some embodiments described herein, the methods further comprise administering a tumor or cancer antigen to a subject being administered the bispecific or multispecific polypeptide agent that is specific for PD-1/DD1α or DD1α/DD1α as described herein.

A number of tumor antigens have been identified that are associated with specific cancers. As used herein, the terms “tumor antigen” and “cancer antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER-2, mucins (i.e., MUC-1), prostate-specific antigen (PSA), and prostatic acid phosphatase (PAP). In addition, viral proteins such as hepatitis B (HBV), Epstein-Barr (EBV), and human papilloma (HPV) have been shown to be important in the development of hepatocellular carcinoma, lymphoma, and cervical cancer, respectively. However, due to the immunosuppression of patients diagnosed with cancer, the immune systems of these patients often fail to respond to the tumor antigens.

In some embodiments of the methods described herein, the methods further comprise administering a chemotherapeutic agent to the subject being administered the bispecific or multispecific polypeptide agent that is specific for PD-1/DD1α or DD1α/DD1α. Non-limiting examples of chemotherapeutic agents can include alkylating agents such as thiotepa and CYTOXAN™; cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaIl (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN. doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK. polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL. paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, Ill.), and TAXOTERE doxetaxel (Rhone-Poulenc Rorer, Antony, France); chloranbucil; GEMZAR, gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-alpha, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation.

### Pharmaceutical Compositions

Provided herein are compositions that are useful for treating and preventing tumors, cancer and infections. In one embodiment, the composition is a pharmaceutical composition. The composition can comprise a therapeutically or prophylactically effective amount of a DD1α and/or PD-1 antibody (e.g., a bispecific antibody) or another immunologic therapy.

The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention. Formulations suitable for parenteral administration, such as, for example, by intraarticular (in the joints), intravenous, intramuscular, intradermal, intraperitoneal, and subcutaneous routes, and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

The compositions described herein include, but are not limited to therapeutic compositions useful for practicing the therapeutic methods described herein. Therapeutic compositions contain a physiologically tolerable carrier together with an active agent as described herein, dissolved or dispersed therein as an active ingredient. In a preferred embodiment, the therapeutic composition is not immunogenic (e.g., allergenic) when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic compositions described herein can include pharmaceutically acceptable salts of the components therein.

Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

While any suitable carrier known to those of ordinary skill in the art can be employed in the pharmaceutical compositions of this invention, the type of carrier will vary depending on the mode of administration. Compositions can be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) can also be employed as carriers for the pharmaceutical compositions of this invention. Suitable biodegradable microspheres are disclosed, for example, in U.S. Pat. Nos. 4,897,268 and 5,075,109. Such compositions can also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions as described herein can be formulated as a lyophilizate. Compounds can also be encapsulated within liposomes using well known technology. The compositions described herein can be administered as part of a sustained release formulation (i.e., a formulation such as a capsule or sponge that effects a slow release of compound following administration). Such formulations can generally be prepared using well known technology and administered by, for example, oral, rectal or subcutaneous implantation, or by implantation at the desired target site. Sustained-release formulations can contain a polypeptide, polynucleotide dispersed in a carrier matrix and/or contained within a reservoir surrounded by a rate controlling membrane. Carriers for use within such formulations are biocompatible, and can also be biodegradable; preferably the formulation provides a relatively constant level of active component release. The amount of active compound contained within a sustained release formulation depends upon the site of implantation, the rate and expected duration of release and the nature of the condition to be treated or prevented.

### Efficacy of Treatment

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with a chronic immune condition, such as, but not limited to a chronic infection or a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

For example, in some embodiments, the methods described herein comprise administering an effective amount of the bispecific or multispecific polypeptide agents described herein to a subject in order to alleviate a symptom of persistent infection. As used herein, “alleviating a symptom of a persistent infection” is ameliorating any condition or symptom associated with the persistent infection. Alternatively, alleviating a symptom of a persistent infection can involve reducing the infectious microbial (such as viral, bacterial, fungal or parasitic) load in the subject relative to such load in an untreated control. As compared with an equivalent untreated control, such reduction or degree of prevention is at least 5%, 10%, 20%, 40%, 50%, 60%, 80%, 90%, 95%, or 100% as measured by any standard technique. Desirably, the persistent infection is completely cleared as detected by any standard method known in the art, in which case the persistent infection is considered to have been treated. A patient who is being treated for a persistent infection is one who a medical practitioner has diagnosed as having such a condition. Diagnosis can be by any suitable means. Diagnosis and monitoring can involve, for example, detecting the level of microbial load in a biological sample (for example, a tissue biopsy, blood test, or urine test), detecting the level of a surrogate marker of the microbial infection in a biological sample, detecting symptoms associated with persistent infections, or detecting immune cells involved in the immune response typical of persistent infections (for example, detection of antigen specific T cells that are anergic and/or functionally impaired).

The term “effective amount” as used herein refers to the amount of a bispecific or multispecific polypeptide agent having specificity for PD-1/DD1α or DD1α/DD1α, needed to alleviate at least one or more symptom of the disease or disorder, and relates to a sufficient amount of pharmacological composition to provide the desired effect, i.e., reverse the impaired proliferation of T cells in a subject having a chronic immune condition, such as cancer or a persistent infection. The term “therapeutically effective amount” therefore refers to an amount of a bispecific or multispecific polypeptide agent using the methods as disclosed herein, that is sufficient to effect a particular effect when administered to a typical subject. An effective amount as used herein would also include an amount sufficient to delay the development of a symptom of the disease, alter the course of a symptom disease (for example but not limited to slow the progression of a symptom of the disease), or reverse a symptom of the disease. Thus, it is not possible to specify the exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the bispecific or multispecific polypeptide agent), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

Embodiments of various aspects described herein can be defined in any of the following numbered paragraphs:
1. A method of identifying a cancer patient who is more likely to respond to an anti-DD1α and/or anti-PD-1 therapy, the method comprising:
   a. measuring the level of p53 activity or expression in a sample from a cancer patient;
   b. comparing the level of p53 or expression in the sample with a p53 reference; and
   c. identifying the cancer patient to be more likely to respond to an anti-DD1α and/or anti-PD-1 therapy, when the level of p53 activity or expression is greater than the p53 reference; or identifying the cancer patient to be more likely to respond to an alternative, proinflammatory immunotherapy without an anti-DD1α or anti-PD-1 therapy, when the level of p53 activity or expression is the same as or less than the p53 reference.
2. A method of identifying a patient diagnosed to have asthma or allergy who is more likely to respond to an anti-DD1α and/or anti-PD-1 therapy, the method comprising:
   a. measuring the level of p53 activity or expression in a sample from a patient diagnosed to have asthma or allergy;
   b. comparing the level of p53 or expression in the sample with a p53 reference; and
   c. identifying the patient diagnosed to have asthma or allergy to be more likely to respond to an anti-DD1α and/or anti-PD-1 therapy, when the level of p53 activity or expression is greater than the p53 reference; or identifying the patient to be more likely to respond to an alternative, proinflammatory immunotherapy without an anti-DD1α or anti-PD-1 therapy.
3. The method of paragraph 1 or 2, further comprising administering an anti-DD1α and/or anti-PD-1 therapy to the patient when the level of p53 activity or expression is greater than the p53 reference; or administering an alternative, proinflammatory immunotherapy without an anti-DD 1α60 and/or anti-PD-1 therapy when the level of p53 activity or expression is the same as or less than the p53 reference.
4. The method of paragraph 3, further comprising increasing the dose of the anti-DD1α and/or anti-PD-1 therapy over a period of time.
5. The method of any of paragraphs 1-4, wherein the alternative, proinflammatory immunotherapy comprises an activator of a proinflammatory T cell response pathway and/or a suppressor of an anti-inflammatory T cell response pathway.
6. The method of paragraph 5, wherein the activator of the proinflammatory T cell response and/or suppressor of the anti-inflammatory T cell response pathway comprises a TIGIT inhibitor, a Fgl2 inhibitor, a TIM-3 inhibitor, an anti-galectin-9 molecule, a CTLA-4 antagonist, a Lag-3 antagonist, an agonist of an immune checkpoint activating molecule, an antagonist of an immune checkpoint inhibitory molecule, or any combination thereof
7. The method of any of paragraphs 1-6, wherein the patient has been receiving an anti-cancer therapy, anti-asthma therapy, anti-allergy therapy, and/or immunotherapy.
8. The method of any of paragraphs 1-7, wherein the p53 reference corresponds to the level of p53 activity or expression in a normal healthy subject.
9. The method of any of paragraphs 1-7, wherein the p53 reference corresponds to the level of p53 activity or expression in a normal tissue of the same type or lineage as the sample.
10. The method of any of paragraphs 1-7, wherein the p53 reference corresponds to a threshold level of p53 activity or expression.
11. The method of any of paragraphs 1-10, wherein the sample is a blood sample.
12. The method of any of paragraphs 1-11, wherein the sample is a tissue biopsy sample.
13. The method of any of paragraphs 1-12, wherein the anti-PD-1 therapy comprises a PD-1 inhibitor, a PD-L1 inhibitor, and/or a PD-L2 inhibitor.
14. The method of any of paragraphs 1-13, wherein the anti-DD1α therapy comprises an agent that inhibits homophilic interactions between DD1α molecules and/or an agent that inhibits heterophilic interactions between DD1αmolecules and PD-1 molecules.
15. The method of paragraph 13 or 14, wherein the inhibitor or agent is selected from the group consisting of a protein, a peptide, a nucleic acid, an antibody, a small molecule, a vaccine, and combinations thereof
16. A method of identifying a patient diagnosed to have an inflammatory disease or disorder who is more likely to respond to a DD1α agonist and/or PD-1 agonist therapy, the method comprising:
   a. measuring the level of p53 activity or expression in a sample from a patient diagnosed to have an inflammatory disease or disorder;
   b. comparing the level of p53 or expression in the sample with a p53 reference; and
   c. identifying the patient to be more likely to respond to a DD1α agonist and/or PD-1 agonist therapy, when the level of p53 activity or expression is lower than the p53 reference; or identifying the patient to be more likely to respond to an alternative, anti-inflammatory immunotherapy without a DD1α agonist or PD-1 agonist therapy, when the level of p53 activity or expression is the same as or greater than the p53 reference.
17. The method of paragraph 16, further comprising administering a DD1α agonist and/or PD-1 agonist therapy to the patient when the level of p53 activity or expression is lower than the p53 reference; or administering an alternative, anti-inflammatory immunotherapy without a DD1αagonist and/or PD-1 agonist therapy when the level of p53 activity or expression is the same as or greater than the p53 reference.
18. The method of paragraph 16 or 17, wherein the alternative, anti-inflammatory immunotherapy comprises a suppressor of a proinflammatory T cell response pathway and/or an activator of an anti-inflammatory T cell response pathway.
19. The method of paragraph 18, wherein the suppressor of the proinflammatory T cell response and/or activator of the anti-inflammatory T cell response pathway comprises a TIGIT agonist, a Fgl2 agonist, a TIM-3 agonist, a galectin-9 molecule, a CTLA-4 agonist, a Lag-3 agonist, an antagonist of an immune checkpoint activating molecule, an agonist of an immune checkpoint inhibitory molecule, or any combination thereof
20. The method of any of paragraphs 16-19, wherein the patient has been receiving an immunotherapy.
21. The method of any of paragraphs 16-20, wherein the p53 reference corresponds to the level of p53 activity or expression in a normal healthy subject.
22. The method of any of paragraphs 16-20, wherein the p53 reference corresponds to the level of p53 activity or expression in a normal tissue of the same type or lineage as the sample.
23. The method of any of paragraphs 16-20, wherein the p53 reference corresponds to a threshold level of p53 activity or expression.
24. The method of any of paragraphs 16-23, wherein the sample is a blood sample.
25. The method of any of paragraphs 16-23, wherein the sample is a tissue biopsy sample.
26. The method of any of paragraphs 16-25, wherein the PD-1 agonist therapy comprises a PD-1 agonist, a PD-L1 agonist, and/or a PD-L2 agonist.
27. The method of any of paragraphs 16-26, wherein the DD1α agonist therapy comprises an agent that increases homophilic interactions between DD1α molecules and/or an agent that increases heterophilic interactions between DD1αmolecules and PD-1 molecules.
28. The method of paragraph 26 or 27, wherein the agonist or agent is selected from the group consisting of a protein, a peptide, a nucleic acid, an antibody, a small molecule, a vaccine, and combinations thereof
29. The method of any of paragraphs 16-28, wherein the inflammatory disease or disorder is selected from the group consisting of infection, autoimmune diseases, acute inflammation, chronic inflammation, and combinations thereof.
30. A method of treating infection with a bacterial or fungal pathogen, the method comprising administering a treatment comprising an agent that antagonizes DD1α activity to a subject infected with said pathogen.
31. The method of paragraph 30 wherein said administering inhibits macrophage activity against host cell constituents while permitting pathogen phagocytosis by macrophages.
32. The method of paragraph 30 wherein said agent antagonizes the homophilic interaction of DD1α with DD1α.
33. The method of any of paragraphs 30-32 wherein said agent antagonizes the functional interaction of DD1α with PD-1.
34. The method of paragraph 30 or 31 wherein said agent antagonizes the homophilic interaction of DD1α with DD1α and antagonizes the functional interaction of DD1α with PD-1.
35. The method of paragraph 34 wherein said agent comprises a moiety that binds DD1α and a moiety that binds PD-1.
36. The method of paragraph 35 wherein said moieties comprise antigen-binding domains of antibodies that specifically bind DD1α and PD-1, respectively.
37. The method of paragraph 35 or 36 wherein said moiety that binds DD1α is attached to said moiety that binds PD-1 via a linker moiety.
38. The method of any of paragraphs 30-37 wherein the treatment or the agent further disrupts the functional interaction of PD-1 with PD-L1 and/or PD1 with PD-L2.
39. A method of treating cancer, the method comprising administering to a cancer patient in need thereof a treatment comprising an agent that antagonizes the homophilic interaction of DD1α with DD1α.
40. The method of paragraph 39 wherein said agent further antagonizes the functional interaction of DD1α with PD-1.
41. The method of paragraph 40 wherein said agent comprises a moiety that binds DD1α and a moiety that binds PD-1.
42. The method of paragraph 41 wherein said moiety that binds DD1α is attached to said moiety that binds PD-1 via a linker moiety.
43. The method of paragraph 41 or 42 wherein said moieties comprise antigen-binding domains of antibodies that specifically bind DD1α and PD-1, respectively.
44. The methods of any of paragraphs 39-43, wherein the treatment or the agent further disrupts binding of PD-1 with PD-L1 or PD-L2.
45. The methods of any of paragraphs 30-44, wherein the agent is a peptide.
46. The method of any of paragraphs 30-45, wherein the patient is determined to have a level of p53 activity or expression greater than a p53 reference.
47. The method of any of paragraphs 30-46, wherein the patient is determined to have a level of DD1α activity or expression greater than a DD1α reference.
48. The method of any of paragraphs 30-47, wherein the patient is determined to have a level of PD-1 activity or expression greater than a PD-1 reference.

### EXAMPLES

Multicellular organisms are challenged everyday with death and its consequences. The inefficient clearance of dying cells has been coupled to abnormal immune responses such as unresolved inflammation and autoimmune conditions. Here the inventors show that p53, a master regulator of apoptosis, controls phagocytosis-mediated clearance of dead cells through its target DD1α. Importantly, the inventors demonstrate that p53/DD1α signaling plays an indispensable role in “eat-me” signaling-mediated phagocytosis, indicating that p53 executes not only the pro-apoptotic pathway but also post-apoptotic events. Macrophages from DD1α-deficient mice were impaired in phagocytosis in vitro, and DD1α-deficient mice showed profound in vivo defects in clearing dying cells in organs. Multiple organ damage indicative for immune dysfunction in the DD1α deficient mice as well as its homology to the B7 family ligands and receptors, indicate the involvement of DD1α signaling in immune surveillance. The inventors further discovered that DD1α functions as a negative T cell checkpoint regulator in association with a key inhibitor of immune checkpoints, PD-1, indicating a novel bidirectional inhibitory interaction between PD-1 and DD1α. DD1α receptor activation is an important step in making sure no cell corpses are left behind after cell death. Furthermore, p53 induction of DD1α, as well as PD-1 and its ligand PD-L1, functions to ensure the efficient generation of precise immune responses. Therefore, p53 may also serve as a guardian for immune integrity.

Data are presented herein regarding the identification of a novel post-apoptotic target gene of p53, DD1αwhich is highly responsive to stresses, induced in apoptotic cells, and highly expressed in immune cells including macrophage, dendritic cells, monocytes, myeloid and T cells. DD1α is an IgV domain receptor, also known as Gi24/VISTA/PD-1H/Dies1 (40-43), and was recently identified as a potential negative regulator/ligand of T cell function although its role(s) in the immune system are unclear and no physiological ligands or receptors have yet been found (41). In the present study, the inventors have identified DD1α as a direct target of p53 and shown that it functions as an “eat-me” signal, engulfment ligand of apoptotic cells. Unlike other typical engulfment receptors that recognize PtdSer as a ligand, the inventors found that macrophages use the DD1α receptor to recognize and engulf apoptotic cells via the intercellular homophilic interaction of its IgV domain on dying cells. The inventors also show that loss of DD1α or p53 impairs the engulfment of apoptotic cells following apoptotic stress, leading to accumulation of dying cells within tissues and the development of a severe autoimmune phenotype in vivo. Furthermore, the inventors discovered that DD1α, which shares homology to B7 family member PD-L1, functions as a negative immune-checkpoint regulator that is critical in modulating immune response/T cell function in association with PD-1, a key co-inhibitory immunoreceptor of T cell tolerance. As DD1α inhibits T-cell function contributing to the tumor's ability to evade the immune system, inhibiting a DD1α-mediated checkpoint on the immune system may enhance the anti-tumor T-cell response.

### Example 1: p53-DD1α is an ‘Eat-Me’ Signal Pathway Mediating Apoptotic Cell Engulfment

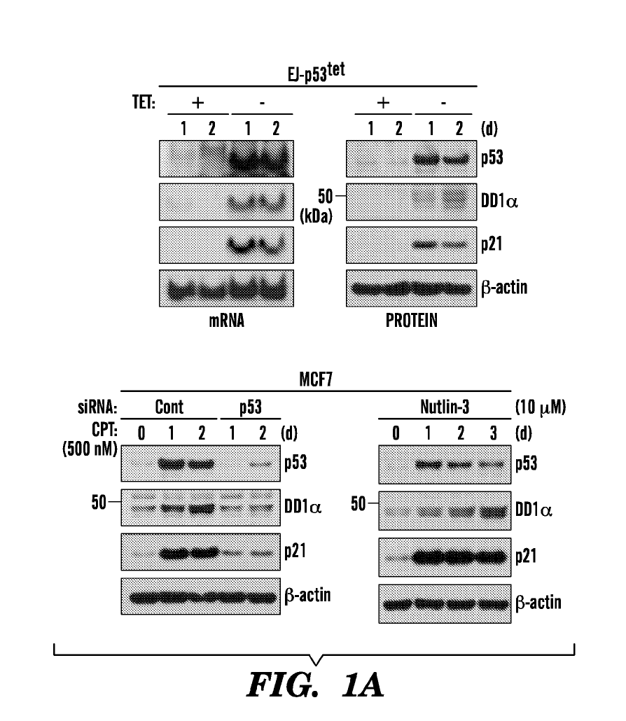

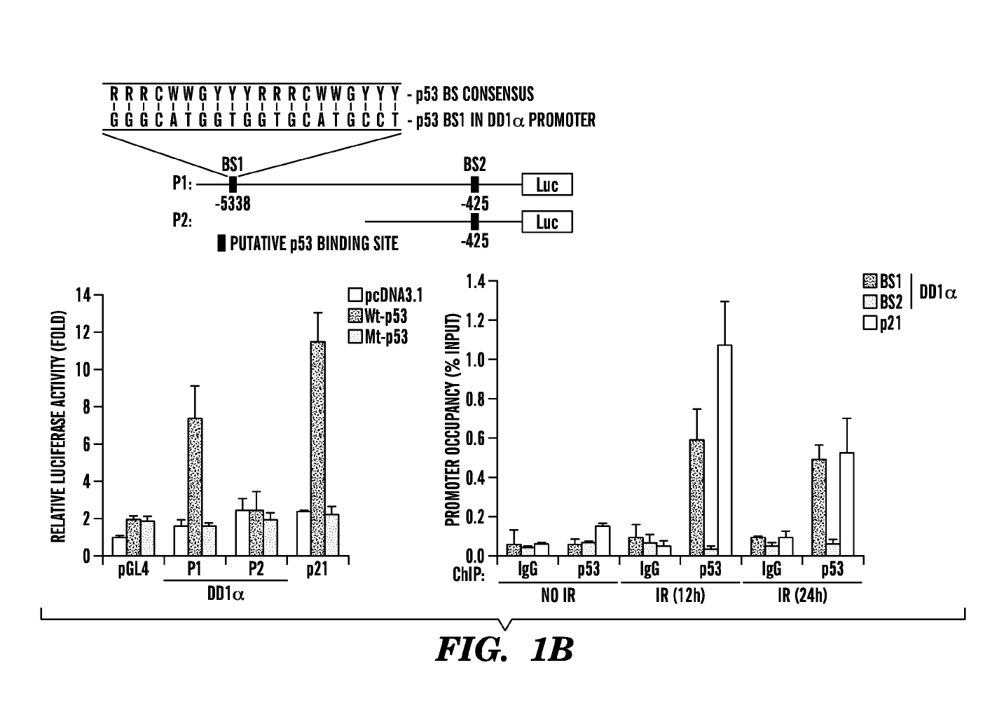

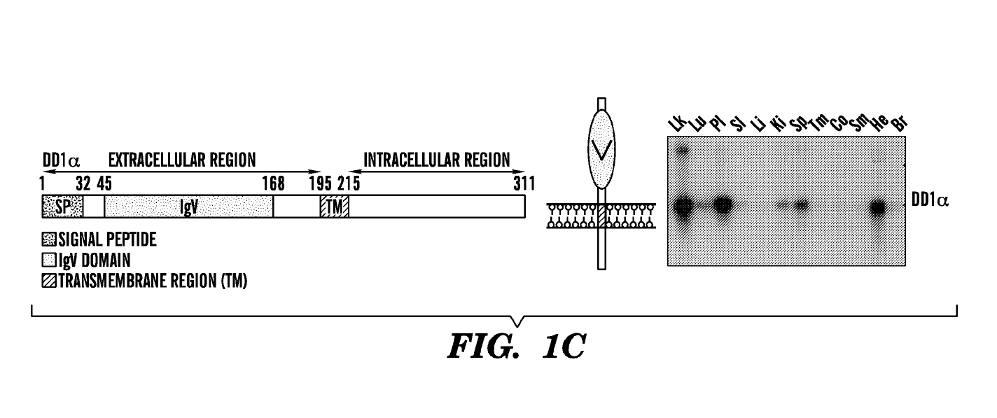

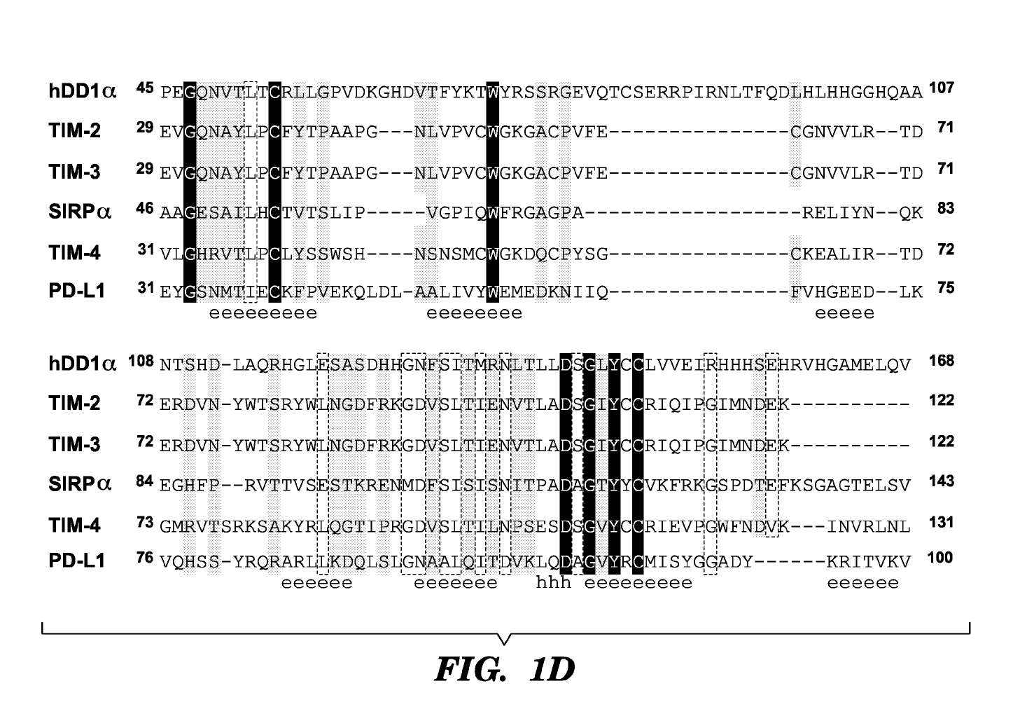

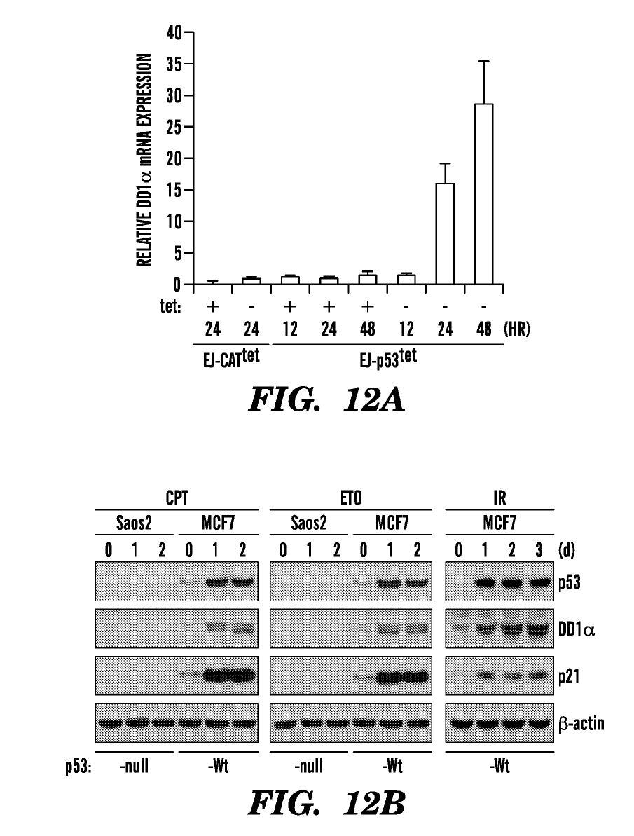

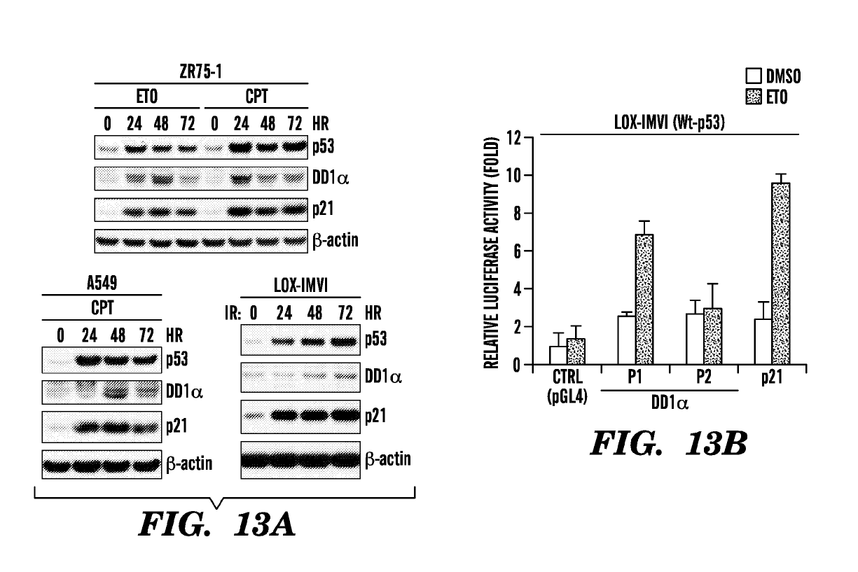

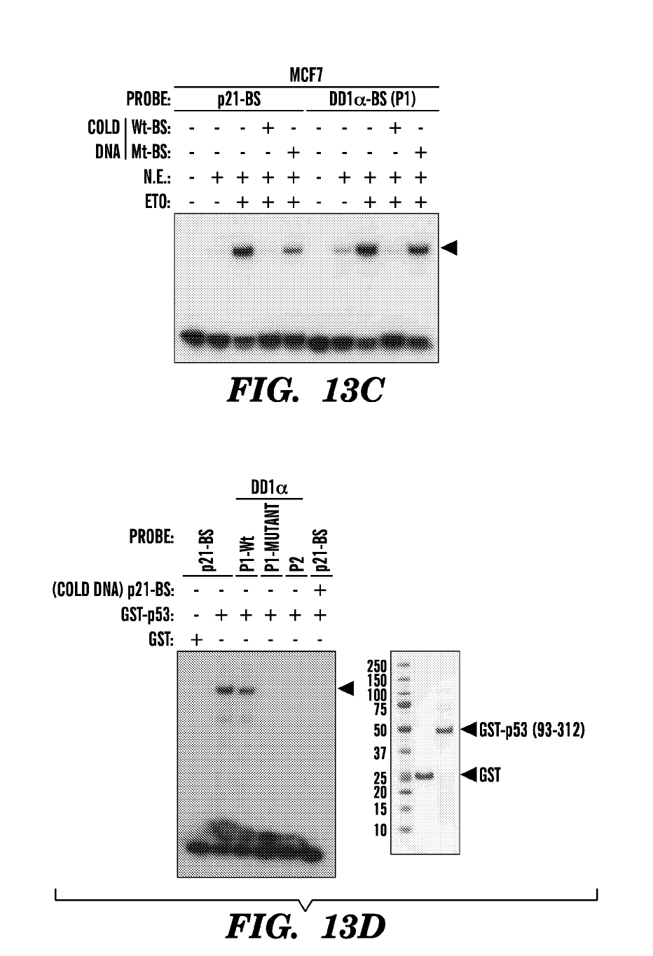

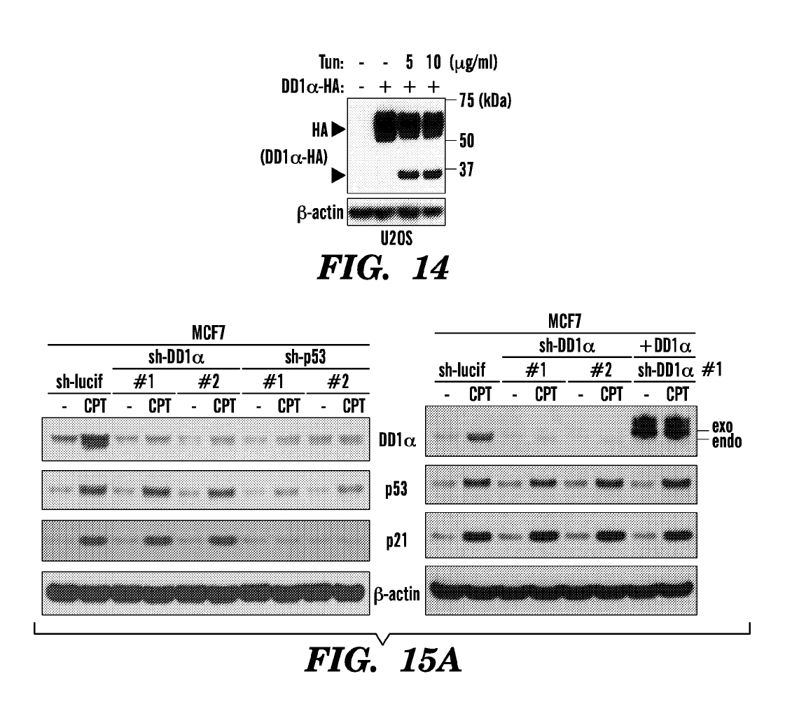

Through microarray analysis of cDNA expression following p53 induction (44), the inventors identified a transmembrane receptor, DD1α, as a direct p53 target gene (FIGS. 1A & B). mRNA and protein level of DD1α were induced in response to genotoxic stress in a p53-dependent manner (FIG. 1A, FIGS. 12 and 13). The characterization of candidate p53 DNA binding consensus sequences using reporter gene assays, ChIP-RT-PCR and electrophoretic mobility shift assay demonstrated that DD1α is a direct transcriptional target of p53 and that the consensus p53-binding site located at −5401 to −5420 of the DD1αpromoter is responsible for p53-dependent DD1αtransactivation. DD1α is a member of immunoglobulin superfamily and is also known as Gi24NISTA/PD-1H/Dies1(40-43). Analysis of its peptide sequence indicated that DD1α contains a signal peptide and a transmembrane region located in the middle (from 195 aa to 215 aa) (FIG. 1C). The extracellular region of DD1α includes the immunoglobulin variable (IgV) set (from 45 aa to 168 aa), which contains several potential N-linked glycosylation sites. DD1α protein was migrated at approximately 50 kDa by western blot analysis due to possible glycosylation (FIG. 1A, FIG. 14) since after treatment with the glycosylation inhibitor tunicamycin, it migrated at the predicted size of ˜30 kDa (FIG. S3). Northern blotting of various human tissues demonstrated high expression levels of DD1α in blood leukocytes, placenta, spleen and heart, and low levels in lung, kidney, small intestine and brain (FIG. 1C). Analysis of public database information also indicated that DD1α is expressed mainly in immune cells, including macrophages, dendritic cells, monocytes, myeloid and T cells.

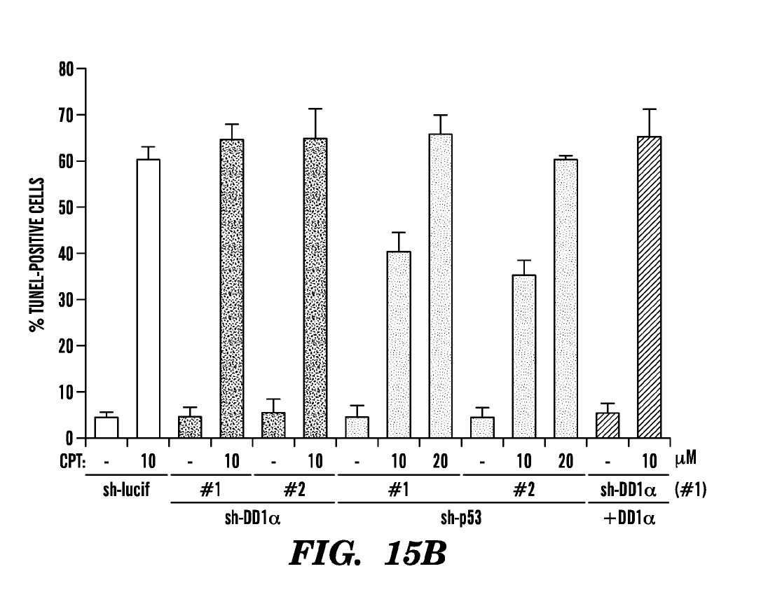

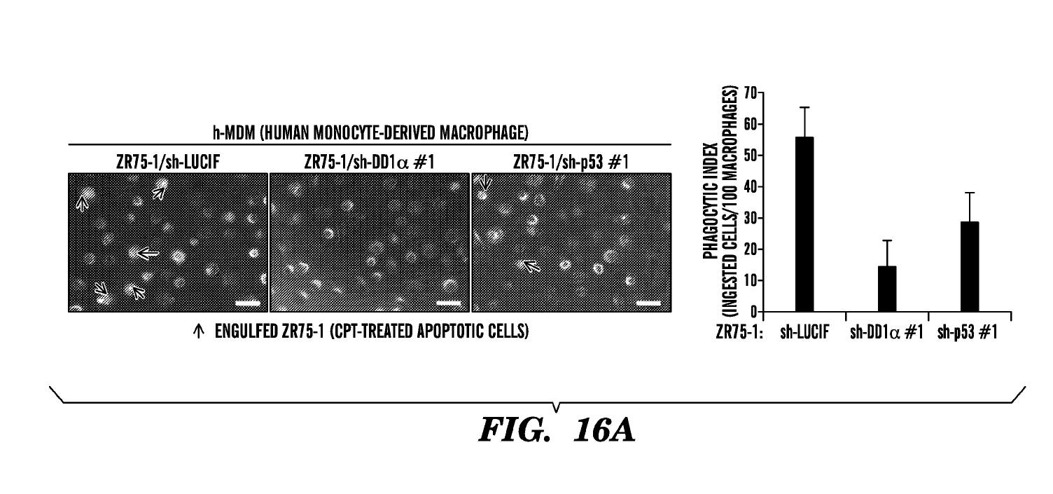

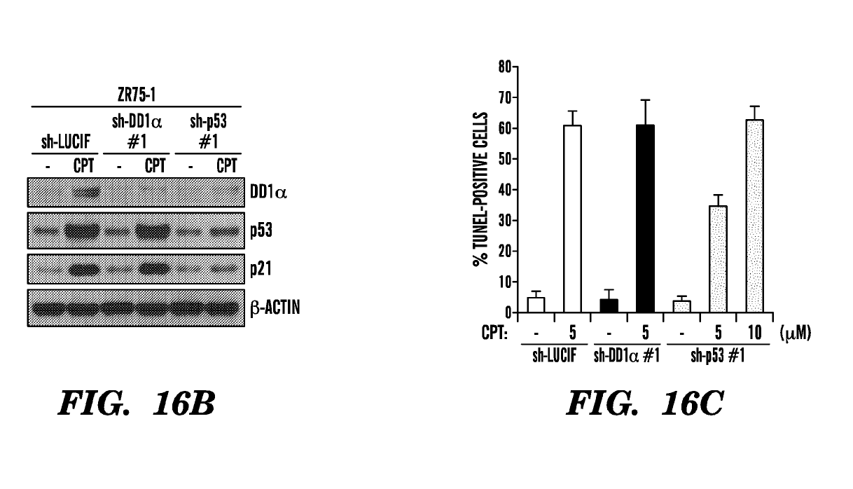

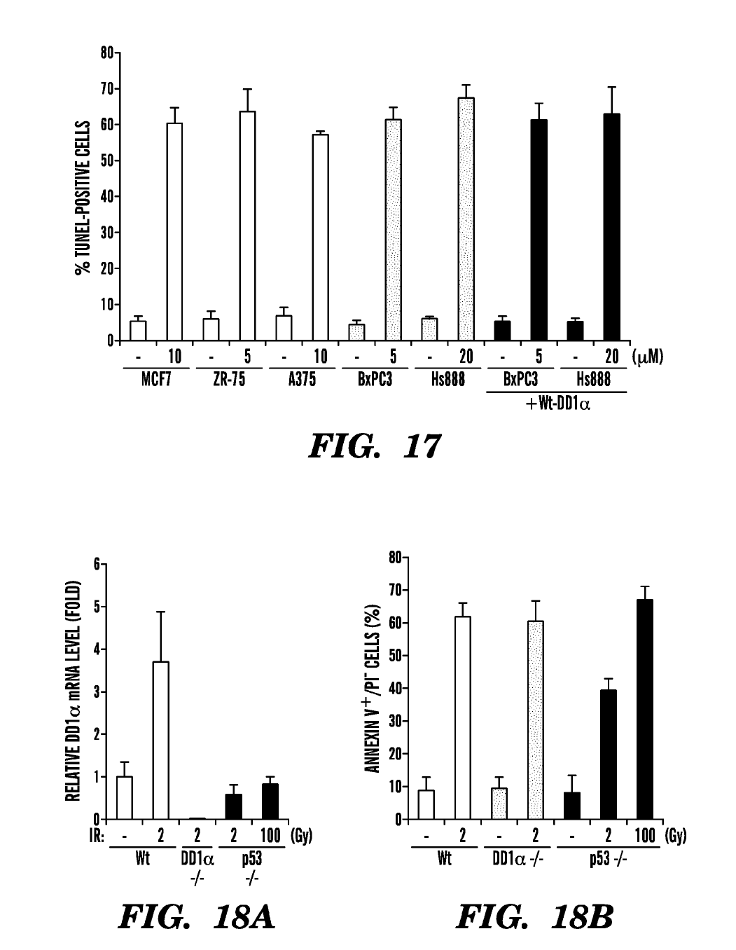

Although the inventors identified DD1α as a novel bona fide p53 target gene, the depletion of endogenous DD1α by shRNAs targeting different sequences in DD1α mRNA did not affect the DNA damage-induced apoptotic responses (FIGS. 15B, 16C, and 18B) in various cell systems carrying wt-p53, indicating that DD1α may not function as a typical p53 target gene. DD1α was found to exhibit similarity with several members of the immunoglobulin superfamily where the homology primarily localizes to the IgV domain. It has been well established that TIM family molecules as well as SIRPα play an important role in phagocytosis of apoptotic cells. It was therefore investigated whether p53-DD1α induction on apoptotic cells could link dying cells to phagocytes for promoting scavenging function. To determine whether p53-regulated DD1α on apoptotic cells plays a critical role in dead/apoptotic cell engulfment, the inventors first depleted DD1α and p53 expression in response to apoptotic stimuli camptothecin (CPT) via two different DD1α shRNAs or p53 shRNAs in MCF7 cells (FIG. 15A). Approximately 60 percent of MCF7 cells were apoptotic (TUNEL-positive) demonstrating equal potential for phagocytosis under all shRNAs-expressing conditions (FIG. 15). Using a pH-sensitive dye (pHrodo), known to emit red fluorescence only when located in the phagosome (pH-5) to distinguish engulfed cells from unengulfed cells, the phagocytotic index (ingested cells/100 macrophages) was calculated (45).

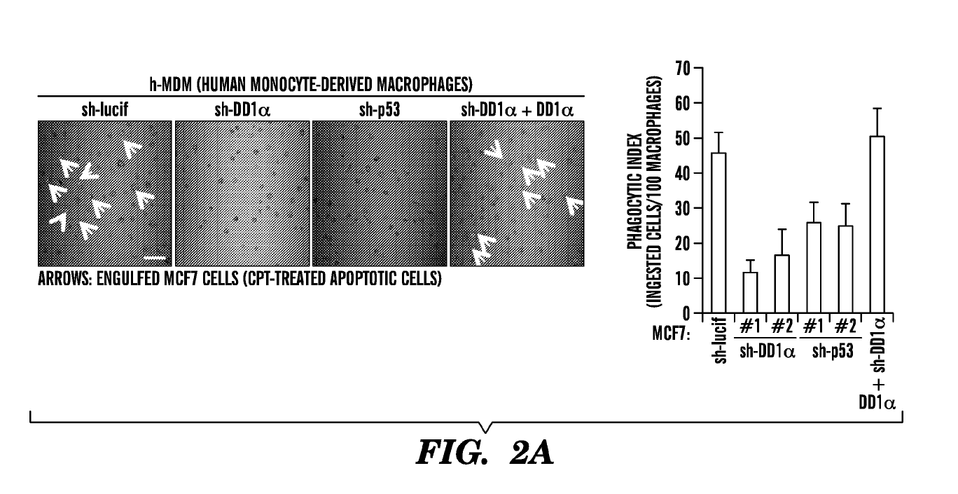

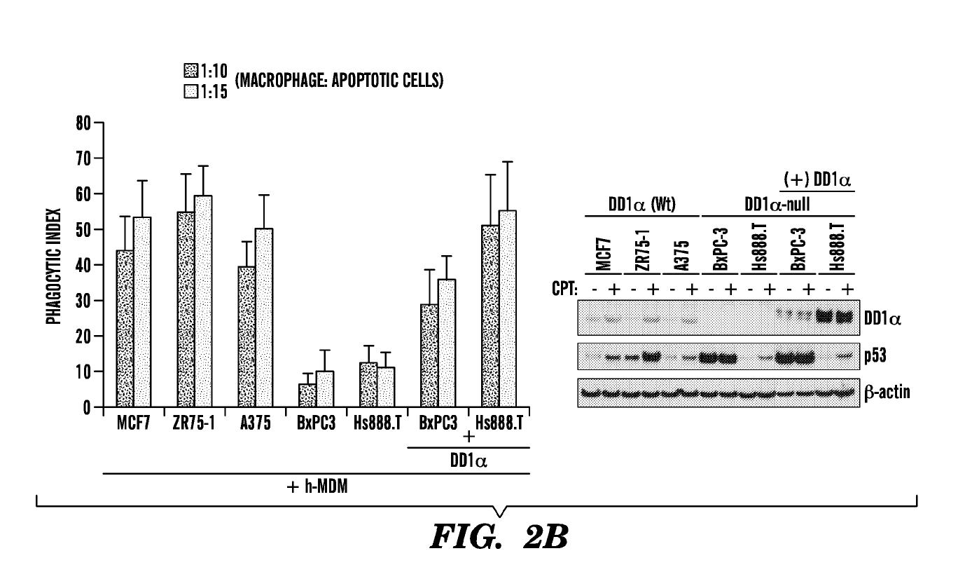

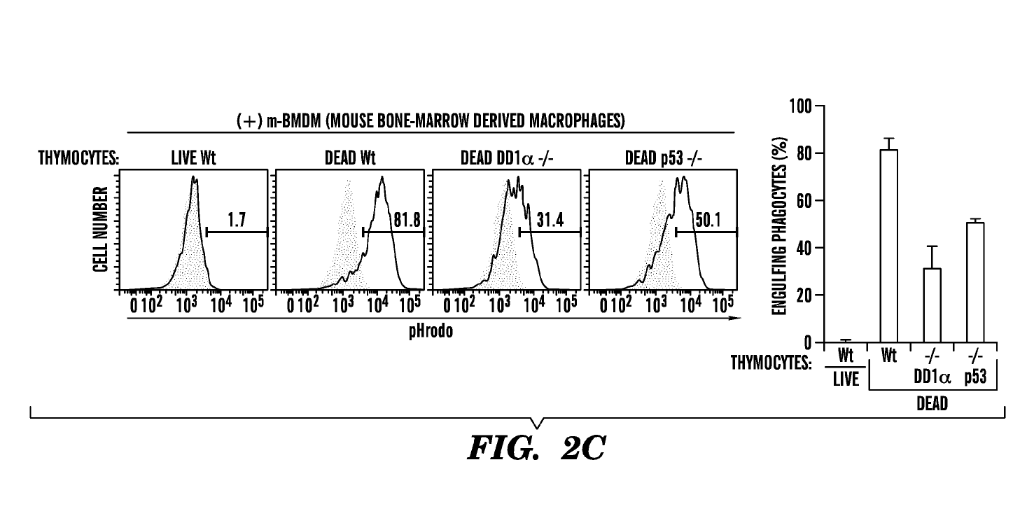

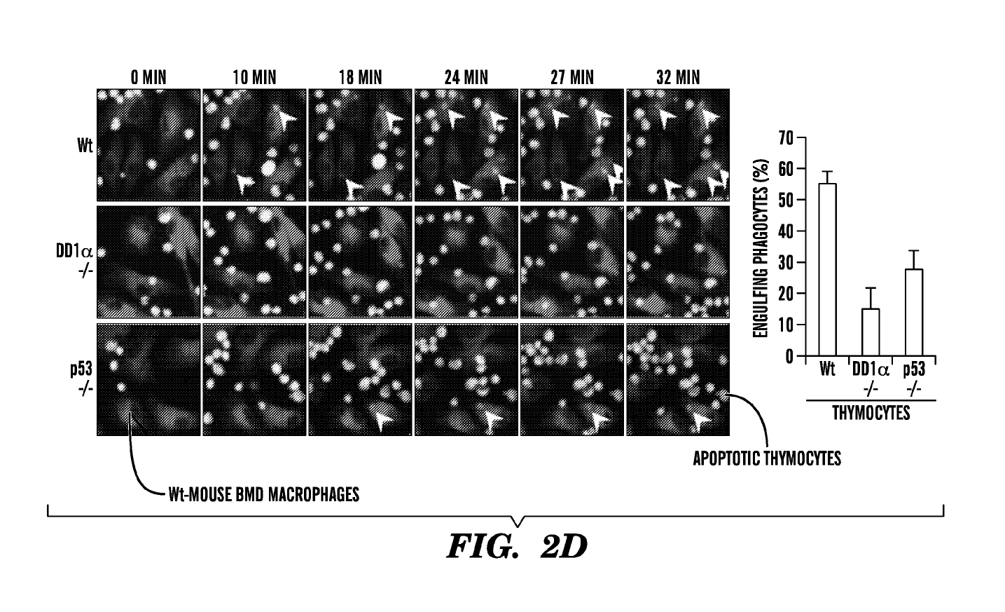

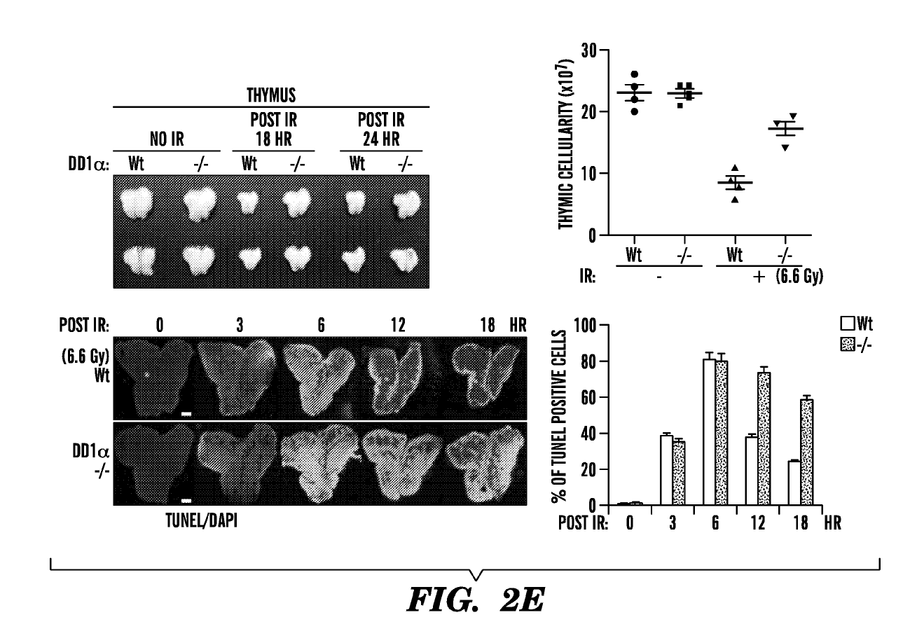

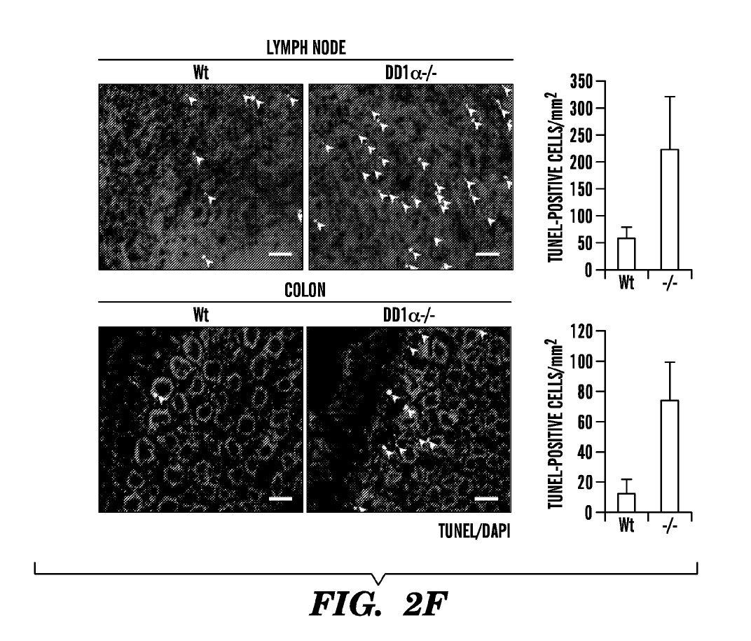

To examine the functional role of DD1α or p53 in dead cell engulfment, freshly isolated human monocyte-derived macrophages were used as phagocytes while CPT-treated apoptotic MCF7 cells with DD1α- or p53-depletion were used as prey. When control MCF7 cells transfected with luciferase shRNA were induced into apoptosis (˜60% were apoptotic cells) and incubated with macrophages, the phagocytic index (the number of engulfed dead cells per 100 macrophages) was ˜50 or higher (FIG. 2A), indicating that macrophages efficiently engulfed most of the apoptotic MCF7 cells present. However, when DD1α- or p53-depleted MCF7 cells were also induced into apoptosis (˜60% apoptotic population) and mixed with macrophages, macrophages engulfed a significantly lower number of apoptotic cells, and the phagocytic index was 10-25 for DD1α-depleted and ˜30 for p53-depleted MCF7 cells (FIG. 2A). Re-expression of DD1α in DD1α-depleted MCF7 cells restored the ability of engulfment to similar levels as control cells (FIG. 2A). ZR75-1 cells with wt-p53 were also used to carry out the phagocytosis assay, when transfected with sh-lucif, sh-DD1α or sh-p53. Consistent with the behavior of MCF7 cells, when approximately 60% apoptotic ZR75-1 cells were incubated with human macrophages, DD1α- or p53-depletion led to significantly less engulfment by macrophages as compared to control (FIG. S5). The inventors also used two human cancer cell lines (BxPC-3 and Hs888.T) that had very low DD1α expression and found that DD1α was not induced by the apoptotic agent CPT (FIG. 2B right). Apoptotic cells of both BxPC-3 (pancreatic adenocarcinoma) and Hs888.T (osteosarcoma) were less efficiently engulfed by phagocytes, compared to wild-type DD1α expressing cell lines such as MCF7, ZR75-1, and A375 (FIG. 2B left). However, ectopic expression of DD1α-HA into BxPC-3 and Hs888.T cells restored the ability of engulfment of dead cells by macrophages, indicating that DD1α expression is sufficient for apoptotic cell engulfment by phagocytes (FIG. 2B).

The inventors further examined the effects of p53- or DD1α-deficiency on the phagocytosis of apoptotic cells using genetically modified mouse cells. Thymocytes isolated from wildtype (wt), DD1α−/−, and p53−/− mice were exposed to IR to induce apoptosis and activate DD1α expression. In wt mouse thymocytes but not p53−/− thymocytes, IR induced DD1αexpression, indicating that the regulation of DD1α by p53 is also conserved in mice (FIG. S7A). The IR-induced apoptotic responses of wt and DD1α−/− thymocytes were comparable, but p53−/− thymocytes showed significantly less apoptosis in response to IR, consistent with prior reports (46). To use a similar apoptotic population of cells for the phagocytosis assay, the inventors increased the dose of IR to p53−/− thymocytes (FIG. 18B) to reach similar apoptosis level. Approximately 60˜70% of apoptotic thymocytes from wt, DD1α−/−, and p53−/− were incubated with bone marrow-derived macrophages (m-BMDMs) from Wt mice, and the phagocytic potential was assessed via two different methods: flow cytometry-based analysis and time lapse image-based analysis. Measurement of the BMDMs with increased red pHrodo signals among total BMDMs by flow cytometry revealed that apoptotic DD1α−/− or p53−/− thymocytes were less efficiently engulfed by BMDMs as compared to wt thymocytes (FIG. 2C). Time lapse imaging analyses with CFSE-labeled wt, DD1α−/− and p53−/− thymocytes and PKH26 red-labeled phagocytic BMDMs consistently showed defective phagocytosis of DD1α−/− or p53−/− thymocytes by BMDMs (FIG. 2D). Together, these data demonstrate that the p53/DD1αpathway is required for engulfment of apoptotic cells by phagocytes, which indicated that the phagocytic activity of the macrophages was impaired by the deficiency of DD1α.

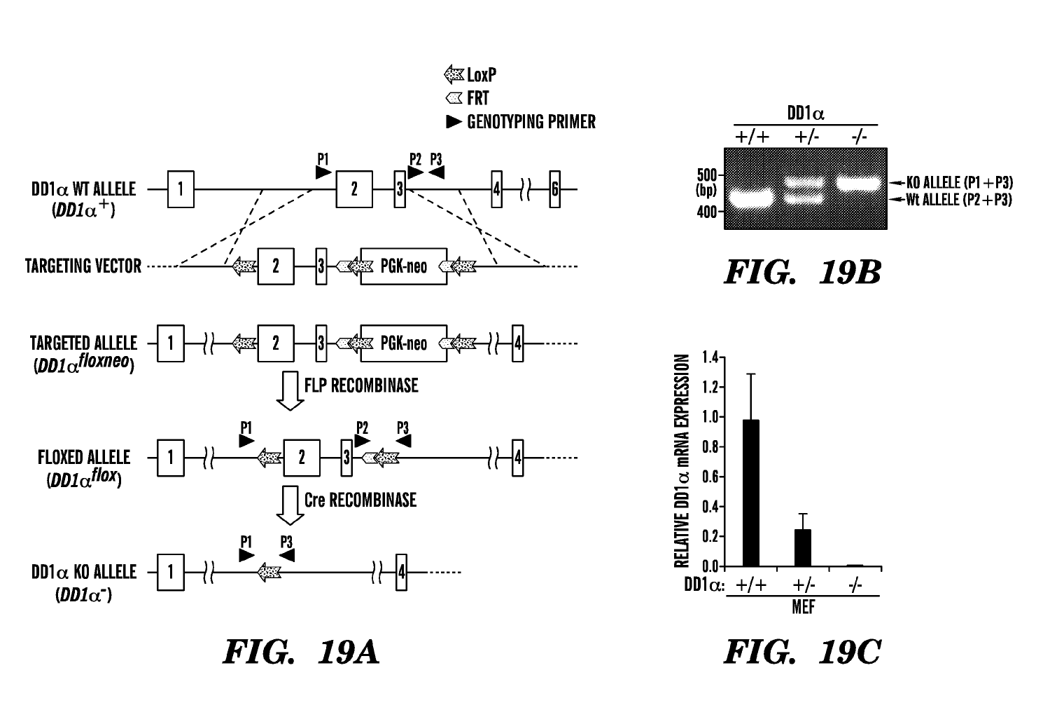

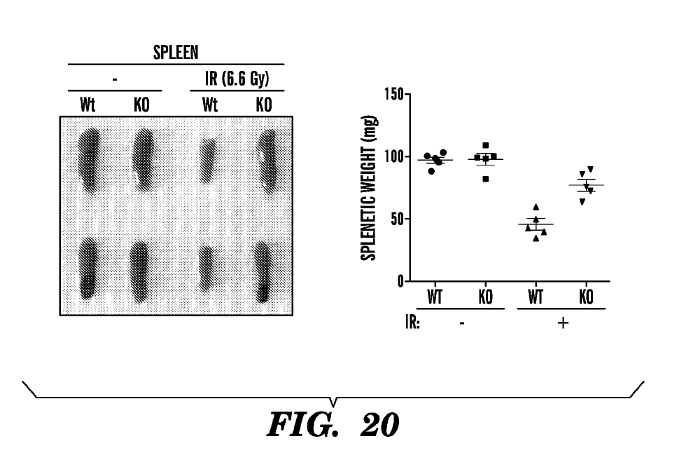

The inventors next addressed whether DD1α-deficiency causes any defects in dead cell clearance in vivo using DD1α-deficient mice. As shown in FIG. 19, DD1α−/− mice were generated using the Flp-Cre system, and DD1α deficiency was confirmed in vivo. The inventors established an in vivo model of apoptotic cell clearance via total body exposure of IR, in which a significant population of thymocytes undergoes synchronous apoptosis with subsequent rapid clearance by resident phagocytes. Whereas Wt mice showed a decrease in overall thymic size due to dead cell removal at the indicated times after IR exposure, DD1α−/− mice showed a significantly limited reduction in thymic size after IR exposure (FIG. 2E). Quantitation of cell numbers confirmed that IR-exposed DD1α−/− mice retained a significantly elevated thymic cell population as compared to those of controls (FIG. 2E). The inventors observed very similar changes in the spleen, another radiosensitive sensitive organ. IR clearly decreased spleen size and weight of Wt mice, but DD1α−/− mice showed significantly less size reduction after IR (FIG. 20). The inventors also examined the presence of apoptotic cells in whole-mount of thymus at different time points after IR exposure, using TUNEL staining Quantitation of TUNEL-positive cells was calculated as a fraction of DAPI-positive cells using an imaging analysis program. In wt and DD1α−/− mice, irradiation of thymocytes with IR induced a similar extent of apoptosis at 3 hours (approximately 40%) and 6 hours (more than 80%). After 6 hours, apoptotic cells in Wt thymus were cleared rapidly-down to 40% at 12 hours and 20% at 18 hours. However, the clearance of apoptotic cells in DD1α−/− thymus was significantly delayed, with two-fold more apoptotic cells remaining in DD1α−/− thymus as compared to Wt thymus (FIG. 2E). These data show a major role for DD1α in apoptotic corpse clearance in vitro and in vivo. Since apoptotic cells are typically cleared rapidly in vivo, they are rarely detectable histologically at any given moment. It was thus examined whether DD1α−/− mice display defects in scavenging apoptotic cells via TUNEL staining of tissue sections. In lymph nodes and colons, the inventors observed 4-5 folds increases in TUNEL-positive cells within DD1α−/− mice as compared to Wt control (FIG. 2F). From these results, it was concluded that the ability of macrophages to engulf apoptotic cells is impaired in the absence of DDa, indicating that DD1α expression can serve as an “eat me signal” for engulfment by phagocytes.

### Example 2: Intercellular Homophilic Interaction of DD1α Receptor Contributes to the Engulfment of Apoptotic Cells

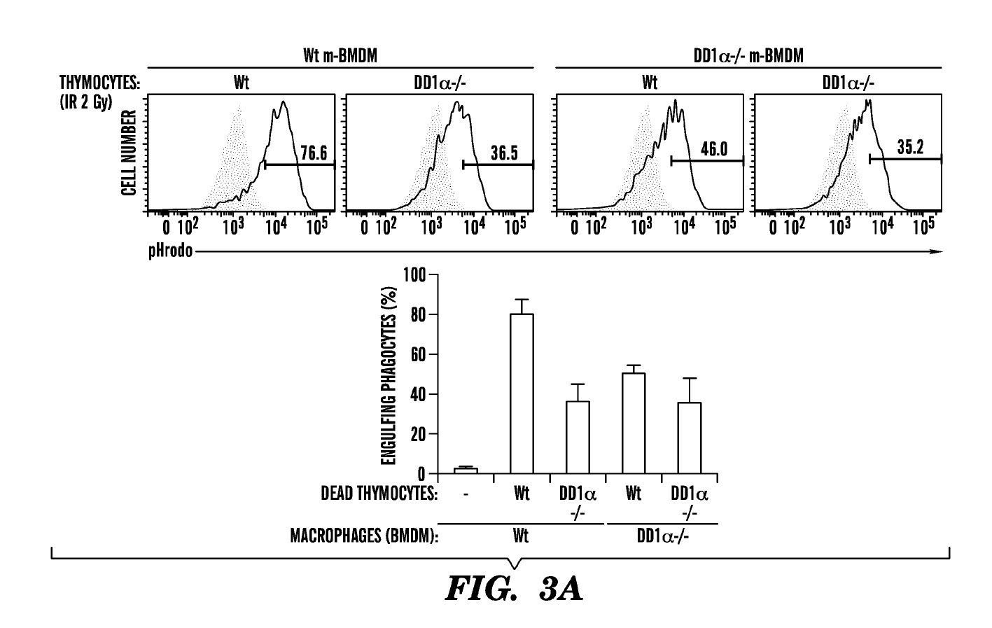

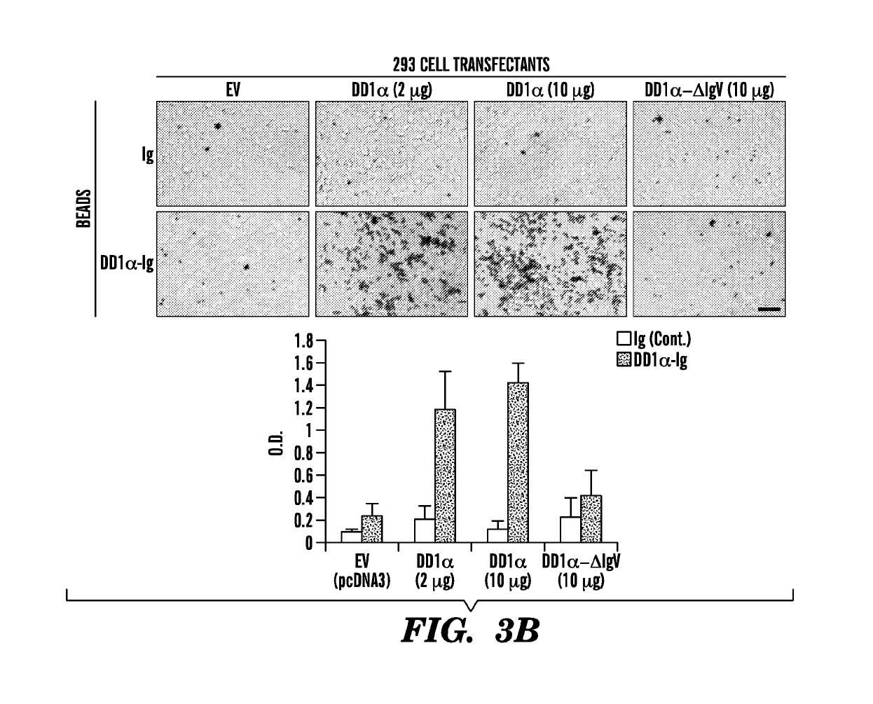

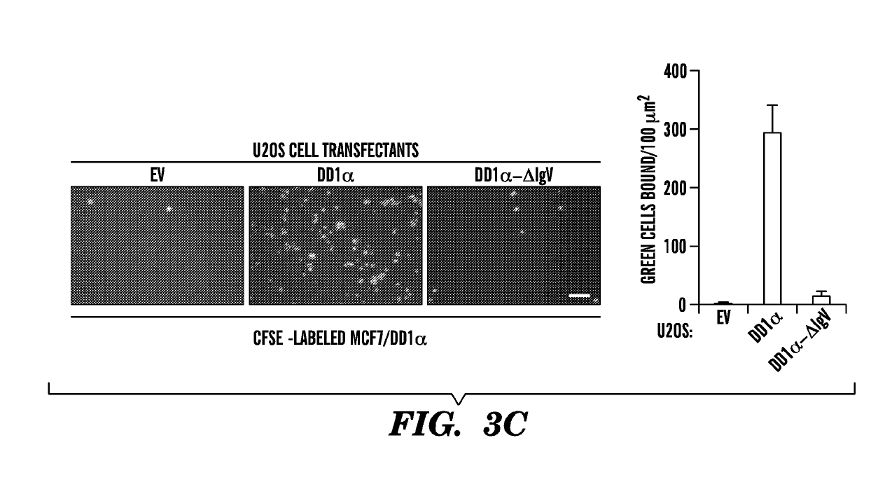

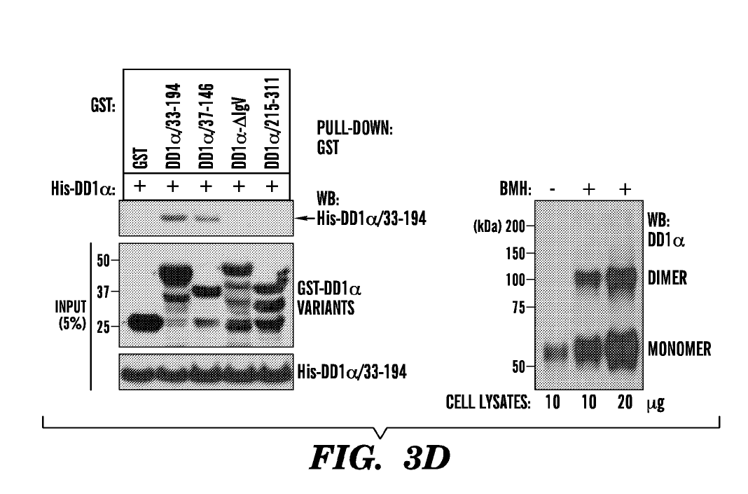

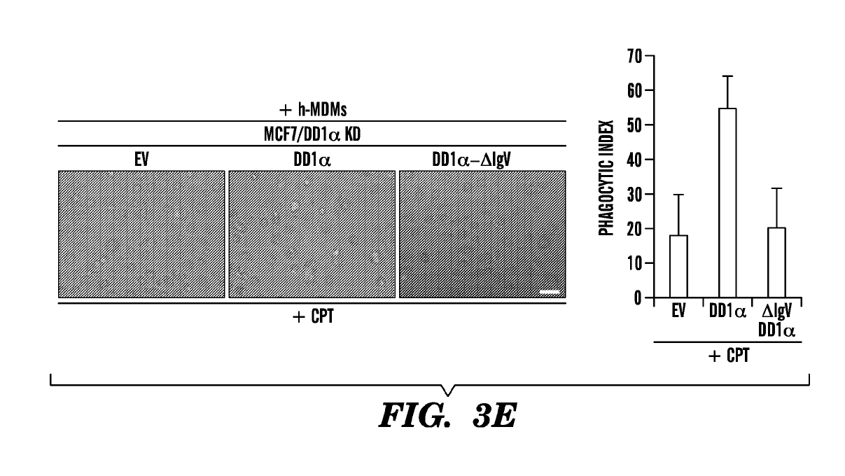

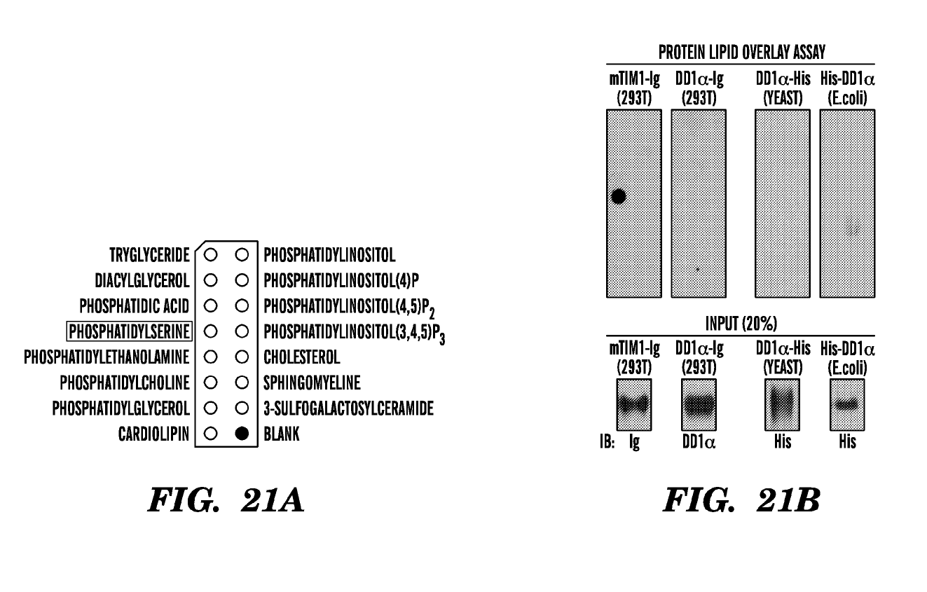

DD1α is highly expressed on blood leukocytes (FIG. 1C). Recent studies (41, 42) of a public expression data-base also indicated that DD1α is highly expressed in macrophages and dendritic cells. Because of the high expression of DD1α in professional phagocytes, it was investigated whether DD1α expression in macrophages is also necessary for the engulfment of apoptotic cells. The inventors used wt- or DD1α-null thymocytes as prey and incubated with wild-type or DD1α−/− BMDMs isolated from DD1α−/− mice for quantitation of phagocytosis analysis using flow cytometry (FIG. 3A). DD1α−/− BMDMs were significantly deficient in uptake of pHrodo-labeled apoptotic wt-thymocytes, as compared to wt BMDMs, but showed at a similar diminished engulfment level when DD1α-null apoptotic thymocytes were used as prey. These results demonstrate that DD1α participates not only on apoptotic cells but also on macrophages in engulfment of apoptotic cells. Dying/apoptotic cells expose phosphatidylserine (PtdSer) on the outer leaflet of the surface membrane of apoptotic cells, which is considered as the primary ‘eat-me signal’ recognized by PtdSer receptor of phagocytes (1, 4). Therefore, it was examined whether PtdSer is a ligand of DD1αreceptor by protein lipid overlay assay. When membranes, spotted with 15 different lipids, were probed with DD1α recombinant proteins purified from several hosts such as mammalian cells (293T), yeast or E. coli, DD1α did not bind to any lipids including PtdSer, indicating that PtdSer is not the ligand of DD1α(FIG. 21). These data indicate that both DD1α on apoptotic cells and DD1α on phagocytes are required and interactive for the engulfment of apoptotic cells. Therefore, to examine the possibility of the DD1α-DD1α homophilic interactions, the inventors studied interactions between soluble protein and cell-surface-expressed receptor molecules, using DD1α-Fc fusion soluble protein (DD1α-Ig), the extracellular IgV domain, and control Ig proteins, in 293 cells transfected with wt-DD1α or mutant-DD1α (IgV domain-deletion). DD1α-Ig proteins (but not control Ig proteins) bound to complete DD1α proteins expressed on the cell surface, but deletion of the IgV domain abolished this interaction, indicating the importance of homophilic DD1α binding via the IgV domain (FIG. 3B). To analyze intercellular DD1α-DD1α binding, cell-cell adhesion assay was employed. U2OS cells transfected with full length DD1α, mutant DD1α with IgV domain deletion or empty vector were incubated with CFSE-stained MCF7 cells with DD1α overexpression (MCF7/DD1α). After thorough washing to remove unbound cells the binding of fluorescently-labeled MCF7 cells to U2OS cells was quantified. U2OS cells with full-length DD1α overexpression bound to labeled MCF7/DD1α cells at significantly higher levels than control vector- or IgV domain deleted DD1αmutant-transfected U2OS cells, confirming the requirement of IgV domain for intercellular homophilic DD1α interactions (FIG. 3C).

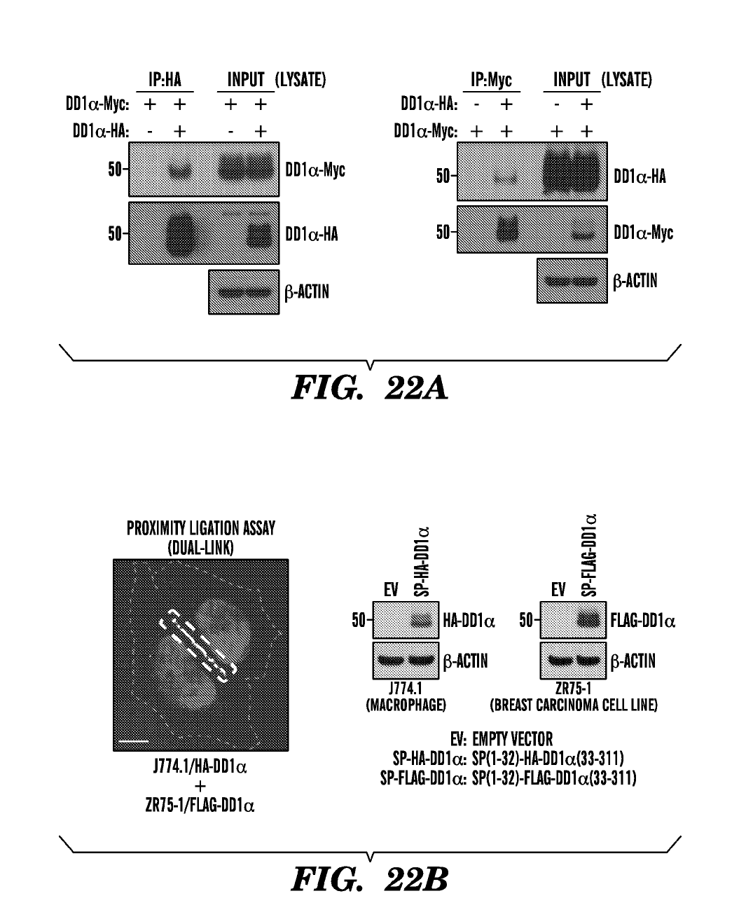

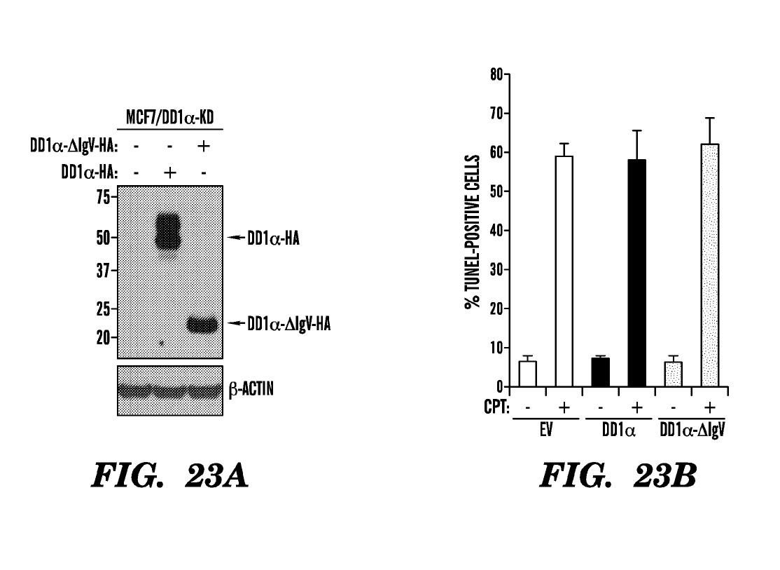

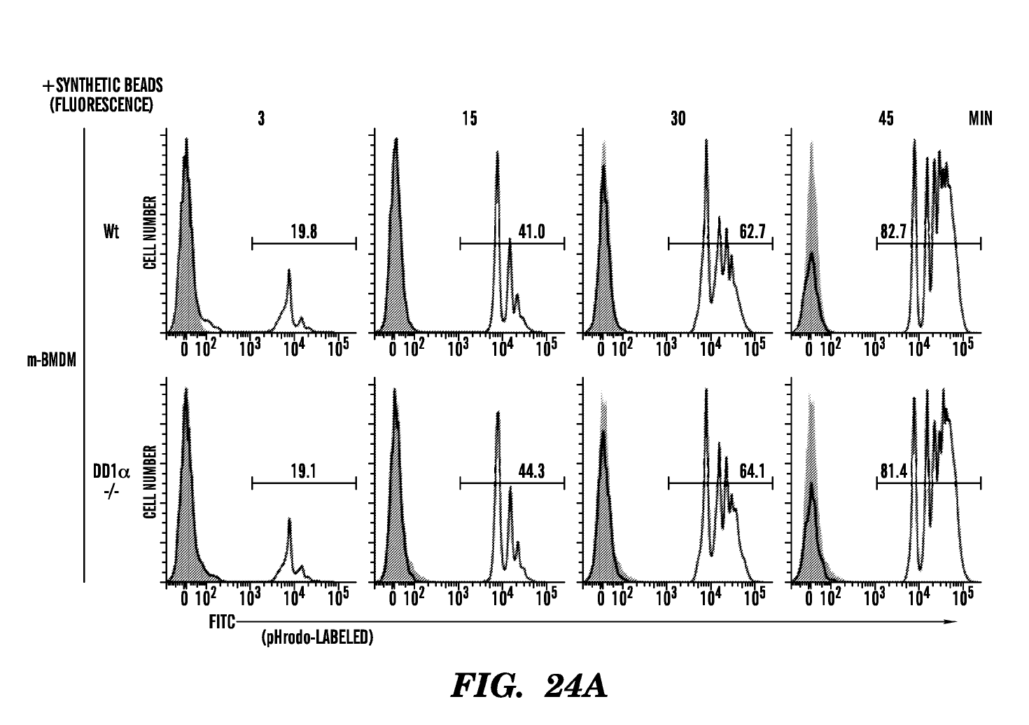

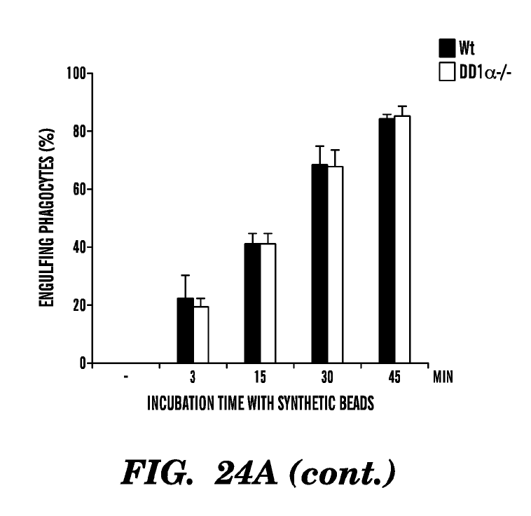

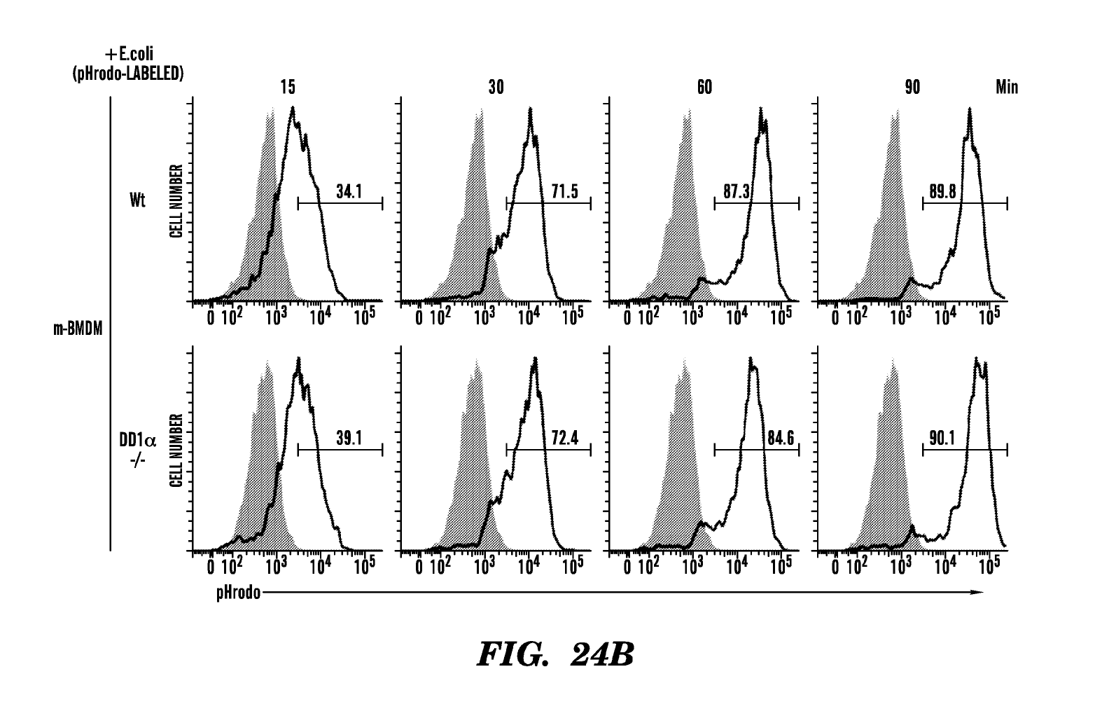

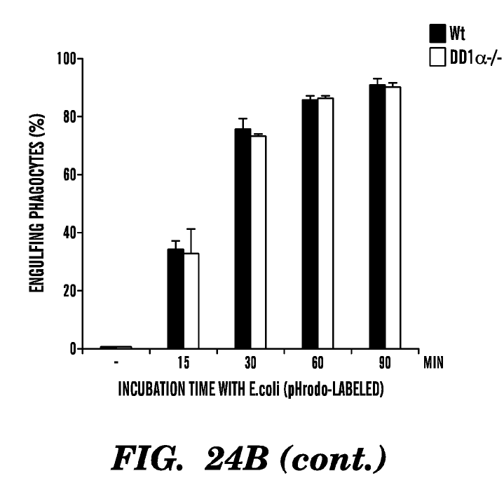

The inventors further mapped the binding region for DD1α-DD1αbinding by in vitro binding analysis using GST- and His-fused DD1α variants, and observed that the extracellular (33-194) DD1α region or the immunoglobulin domain (37-146) were able to bind to His-fused extracellular only DD1α (33-194) but not with IgV-deleted DD1α or the cytoplasmic region of DD1α, indicating that the IgV domain is essential for DD1α-DD1α homophilic interactions (FIG. 3D left panel). In addition, the formation of DD1α dimer was detected on a non-reducing gel after treatment with BMH, a crosslinker for covalent conjugation between sulfhydryl groups (FIG. 3D right). Homophilic intermolecular interaction for the DD1α receptor at intercellular junctions was further confirmed by two other experiments. First, binding between DD1α-Myc and DD1α-HA was detected in lysates from co-culturing both DD1α-Myc-transfected 293T and DD1α-HA-transfected 293T by reciprocal coimmunoprecipitation (FIG. 22A). Second, the inventors performed a proximity ligation assay using the DUOLINK™ technology to detect the association of DD1α-DD1α molecules between two different cell types J774.1 (macrophage) and ZR75-1 (breast carcinoma cell). As shown in FIG. 22B, co-localization dots were detected at intercellular junctions. It was next investigated whether the intercellular homophilic DD1α interaction functionally contributes to apoptotic cell engulfment using the homophilic binding-defective DD1α mutant, IgV-deleted DD1α (DD1α-AIgV). The full-length DD1α, mutant DD1α-AIgV or empty control vector were transfected into DD1α-endogenous-depleted MCF7 cells (FIG. 23A) followed by induction of apoptosis (FIG. 23B). Apoptotic MCF7 cells were incubated with human monocyte-derived macrophages (h-MDM) for phagocytosis analysis. Apoptotic MCF7 cells expressing the full-length DD1α were efficiently engulfed by human macrophages whereas apoptotic MCF7 cells expressing DD1α-AIgV mutant or control vector led to significantly diminished phagocytosis by macrophages (FIG. 3E). Together, these data from biochemical and cellular assays demonstrate that there is a relevant homophilic intermolecular interaction for the DD1α receptor at intercellular junctions, which contributes to the engulfment of apoptotic cells. Of note, phagocytosis analysis of wt- and DD1α−/− bone-marrow-derived macrophages (BMDMs) with synthetic beads (FIG. 24A) or bacteria (E. coli) (FIG. 24B) further revealed that DD1α−/− BMDMs had no defect in phagocytic ability (FIG. 24B), indicating that DD1α-mediated engulfment is dependent upon its homophilic intercellular interaction but not on DD1α-independent targets.

### Example 3: DD1α-Deficient Mice Develop Spontaneous Inflammation and a Systemic Autoimmune Phenotype

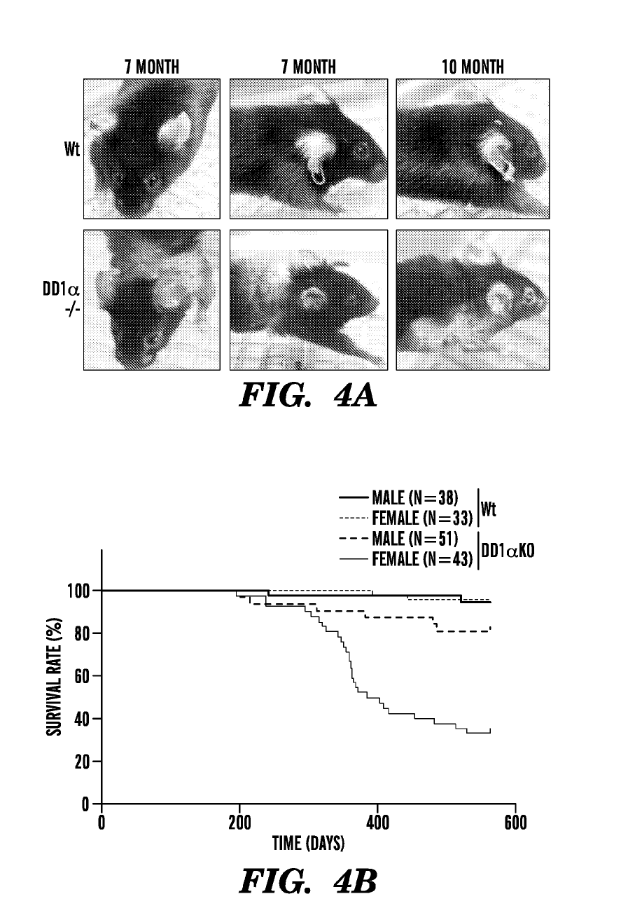

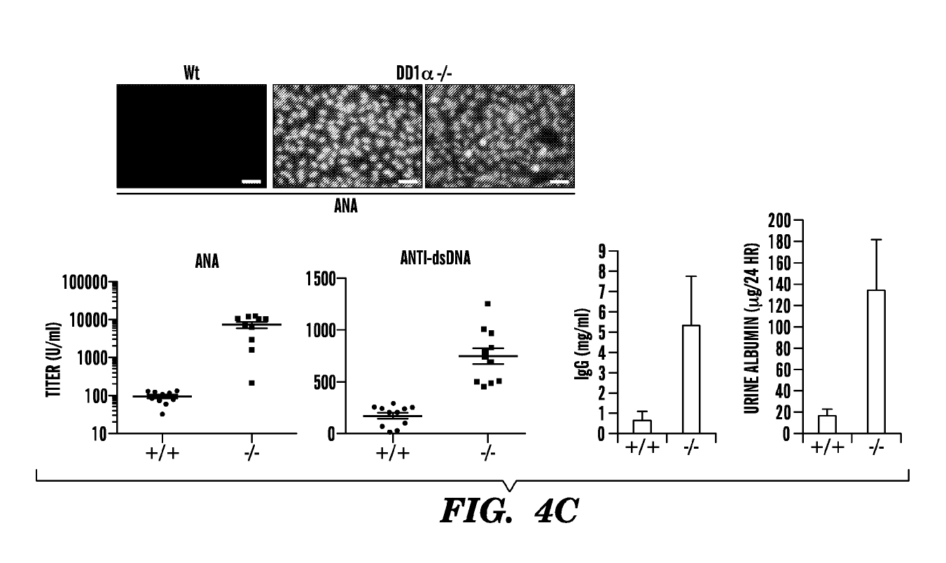

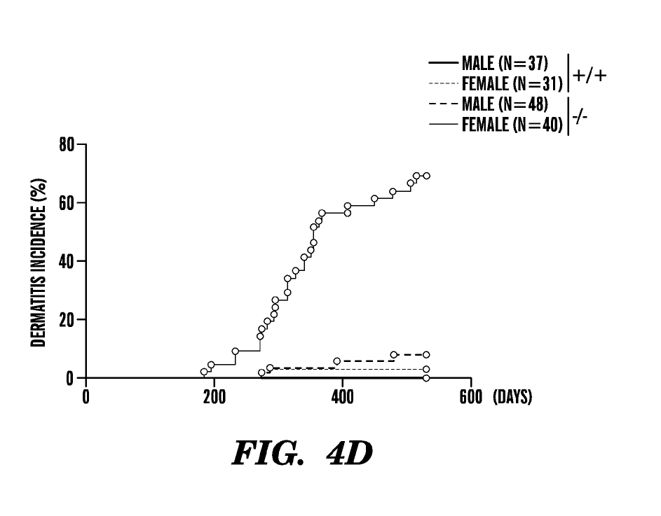

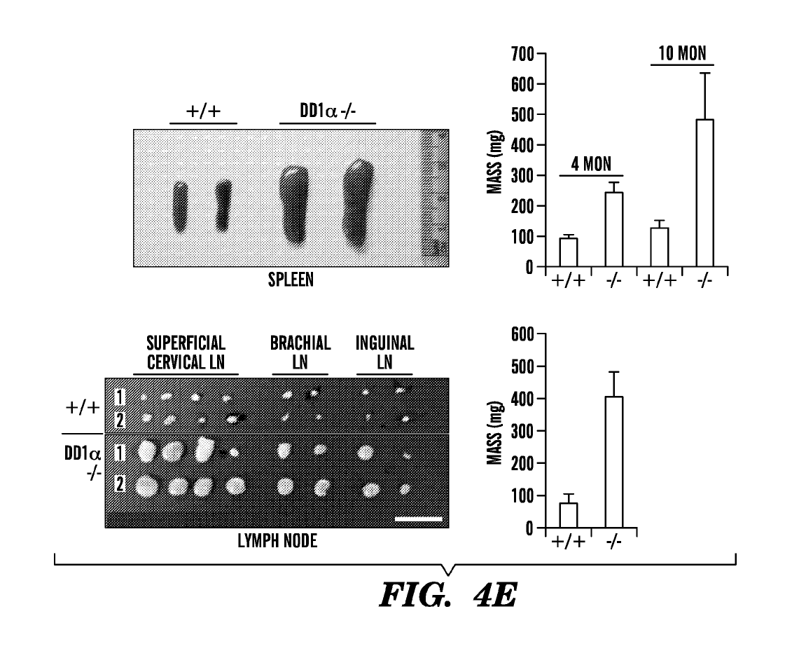

DD1α-null mice are viable, born at the expected Mendelian frequency, are and indistinguishable in appearance from Wt littermates at the early age. Strikingly, at the age of 6-7 months, the inventors observed significantly greater incidence of severe skin inflammation and early death in adult female DD1α−/− mice (FIG. 4A). A majority (˜78%) of female DD1α−/− mice had ulcerative dermatitis and 53%, 23%, and 10% of female DD1α−/− mice showed otitis, eye-related inflammation, and seizures, respectively (Table 1). As shown in FIG. 4B, female DD1α−/− mice started to die beginning at 8 months of age. By 14 months, 60% of female DD1α−/− mice had died. In contrast, ˜60% of male DD1α−/− mice showed no statistically significant difference in survival from Wt control mice (FIG. 4B). It is now established that impaired clearance of apoptotic cells and accumulated corpse of dead cells can cause susceptibility to autoimmune disease (1, 4, 47). Since DD1α-null mice showed severe skin inflammation, which is known to be one of the clinical manifestations of systemic autoimmune disease, it was examined whether DD1α−/− mice showed increased autoantibody titers, a hallmark of autoimmune disease. Starting at −6 months of age, elevated anti-nuclear antibodies were detected the sera from female DD1α−/− mice. Additionally, anti-dsDNA antibodies were also found positive in a majority of the serum from DD1α−/− mice (FIG. 4C).

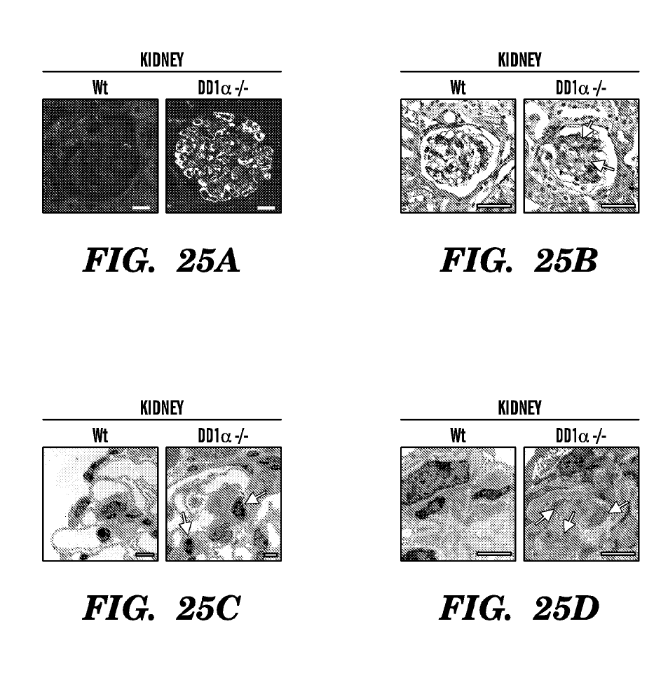

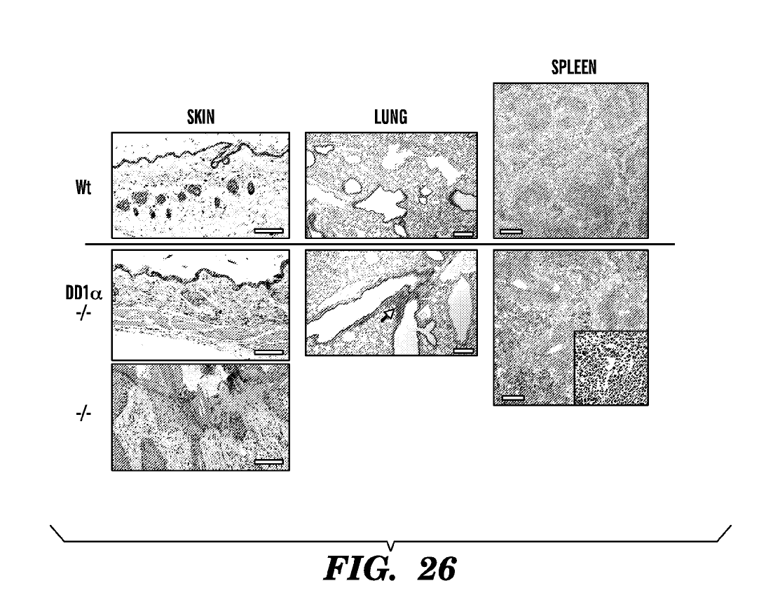

Furthermore, total IgG in sera from 7-10 month-old DD1α−/− mice were elevated to −6 fold in comparison to age-matched wt-mice (FIG. 4C). One of the most commonly targeted organs in systemic autoimmune disorders is the kidney (48). Consistent with other evidence of autoimmune activity, in renal glomeruli of DD1α−/− mice, the inventors observed immune complex deposition identified by immunofluorescence (FIG. 25A) and diffuse mesangial expansion as shown by PAS-positive material and cellular debris (FIG. 25B). Electron microscope analysis also showed an expanded mesangium with electron-dense deposits and neutrophils within capillary lumens of DD1α−/− mice (FIG. 25C). Numerous large electron-dense deposits were also observed by electron micrograph of the glomerular mesangium of DD1α−/− glomeruli (FIG. 25D). Consistent with the above kidney phenotypes, 24 hour urine collection revealed that these DD1α−/− mice produce high levels of proteinuria (FIG. 4C). Together, the DD1α−/− kidney revealed a phenotype of active glomerulonephritis with a mesangioproliferative pattern of injury and immune complex deposition. In addition, the inventors observed extensive splenomegaly and lymphadenopathy in DDa−/− mice by 10 months of age (FIG. 4E). Further histological analysis revealed extensive inflammatory infiltrates in skin and lung and significant extramedullary hematopoiesis, including both erythroid and myeloid hyperplasia, was evident, indicating a major immune dysregulated phenotype of DD1α−/− mice (FIG. 26).

### Example 4: DD1α Functions as a Checkpoint Regulator for T Cell Tolerance

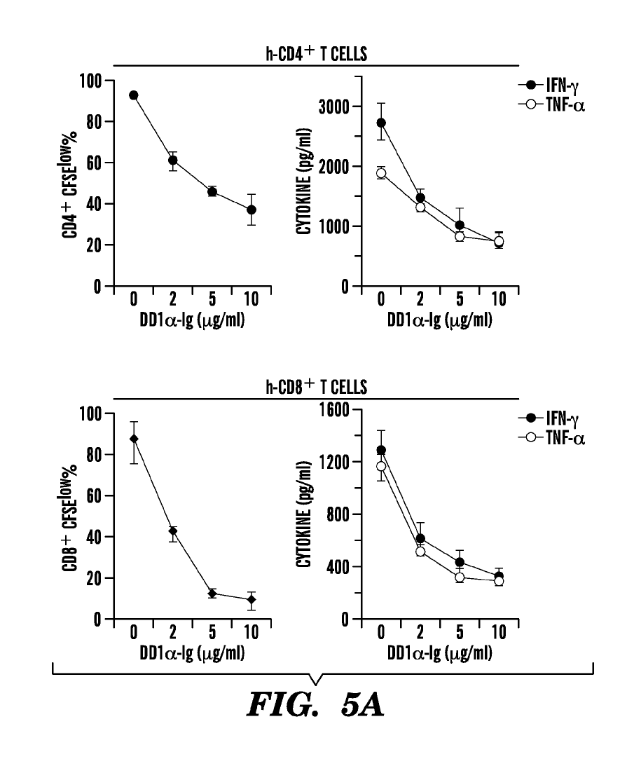

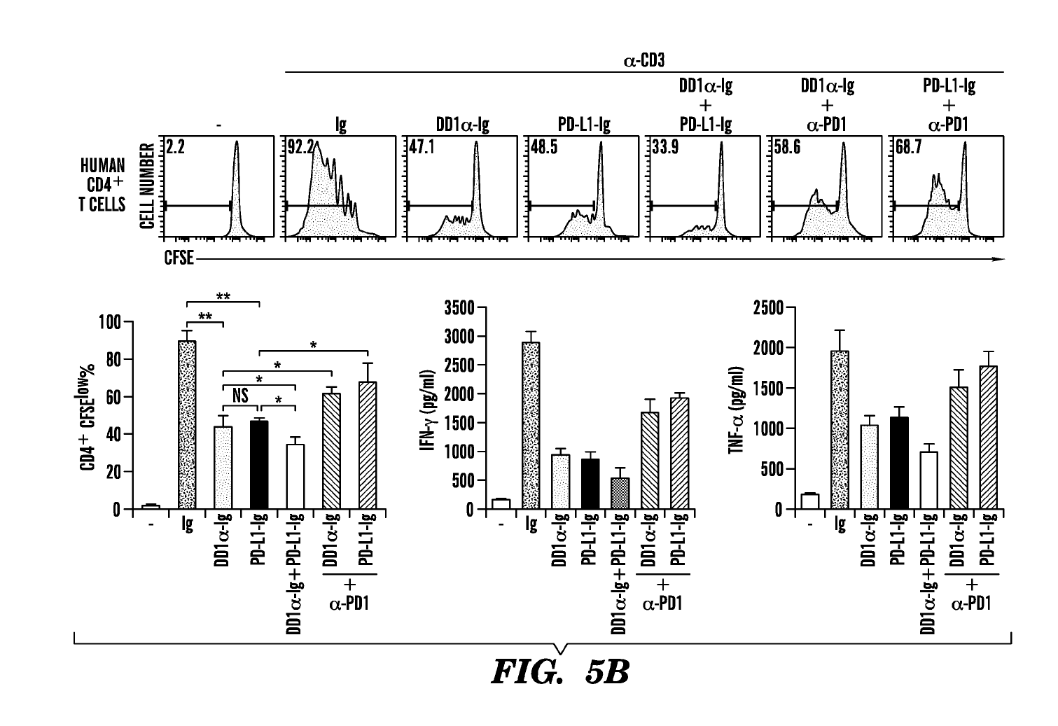

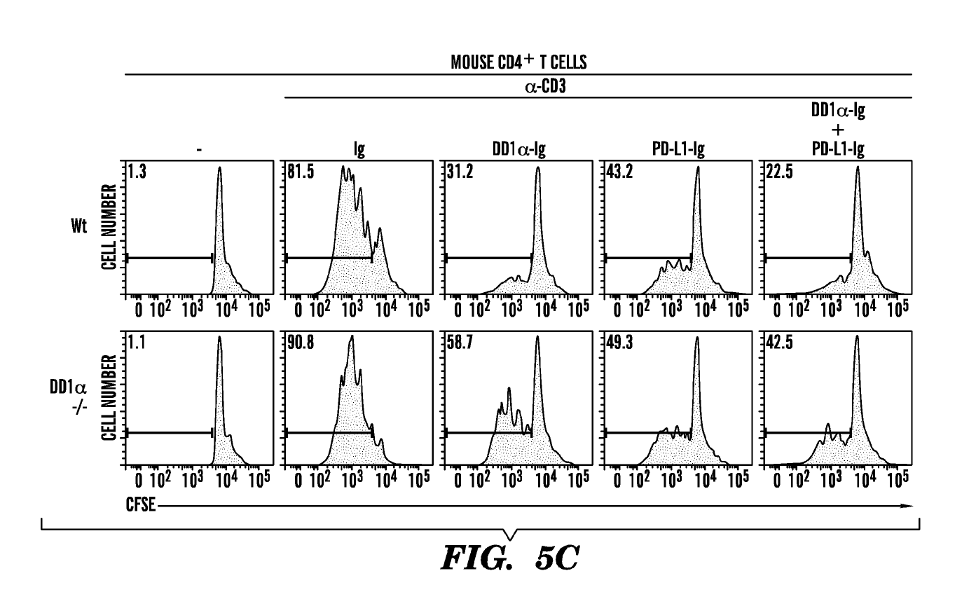

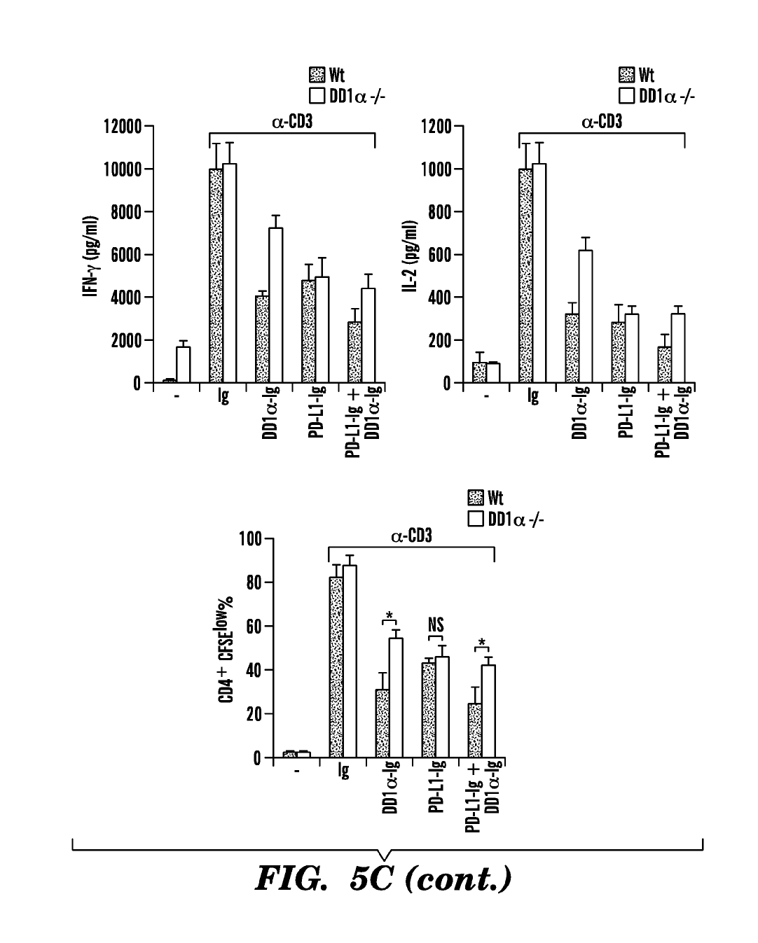

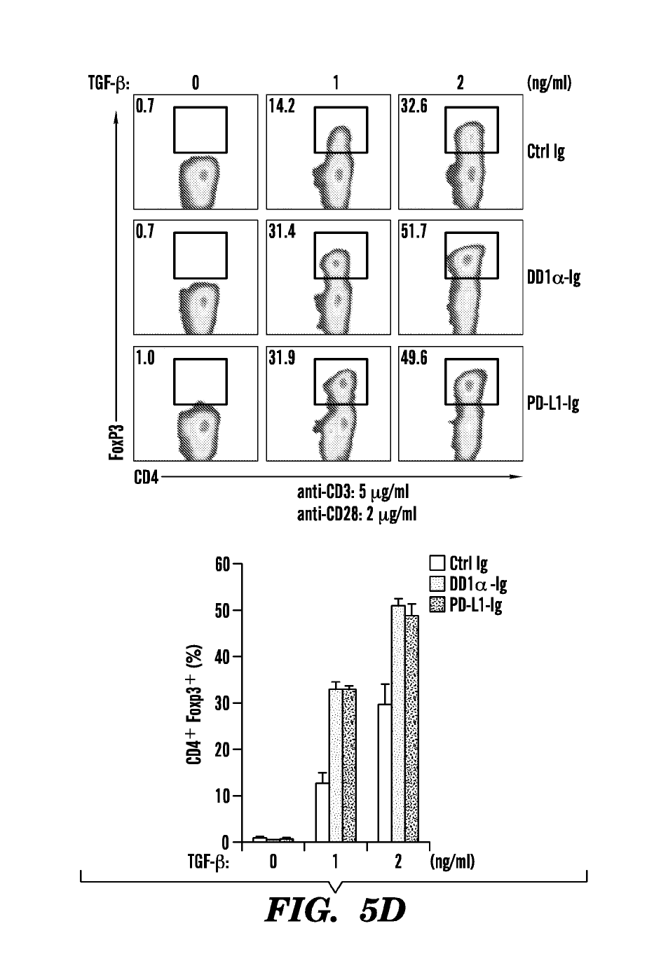

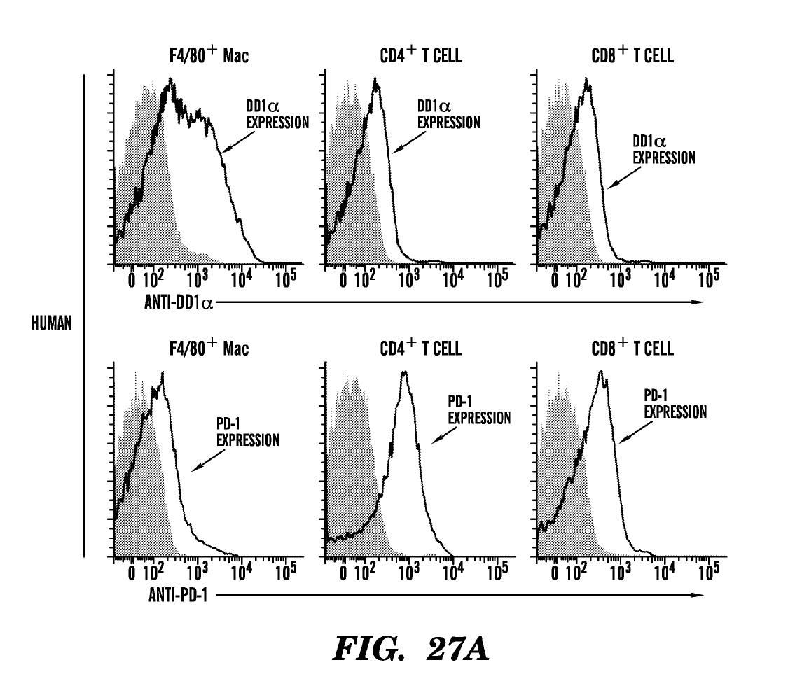

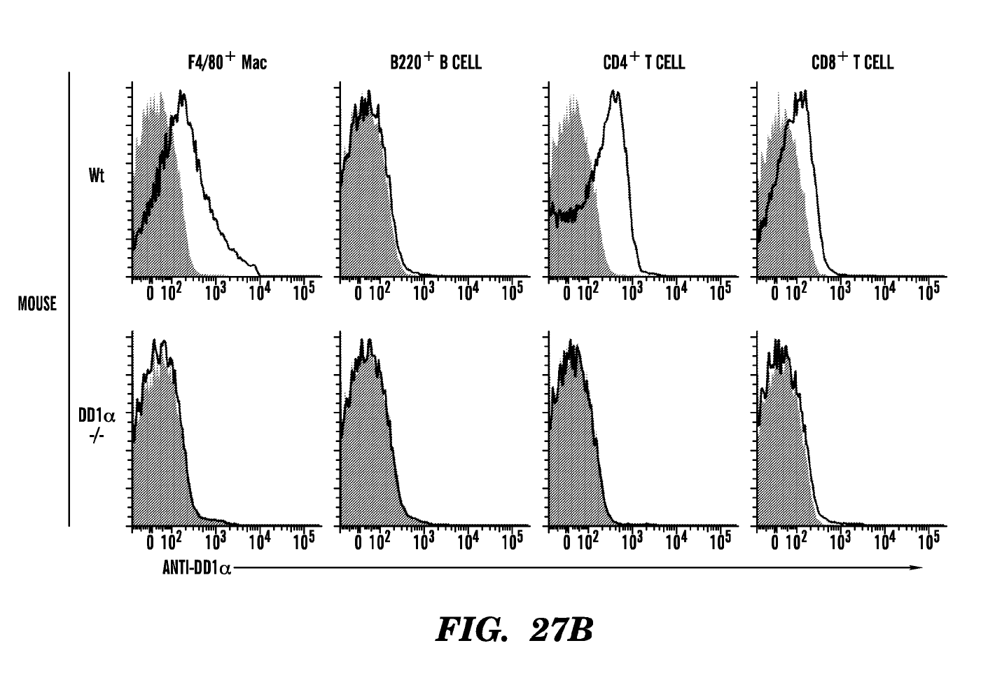

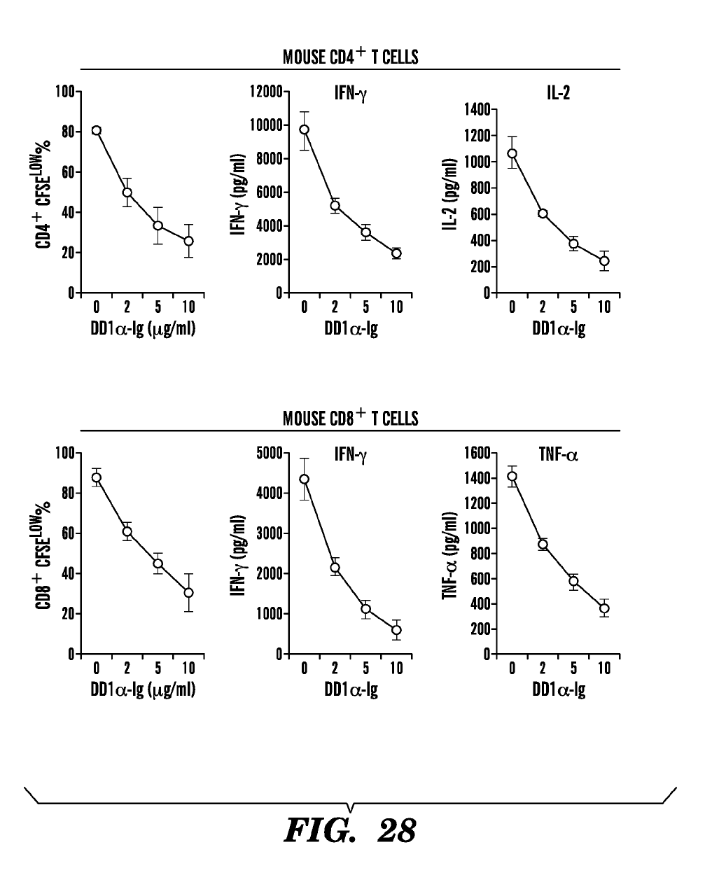

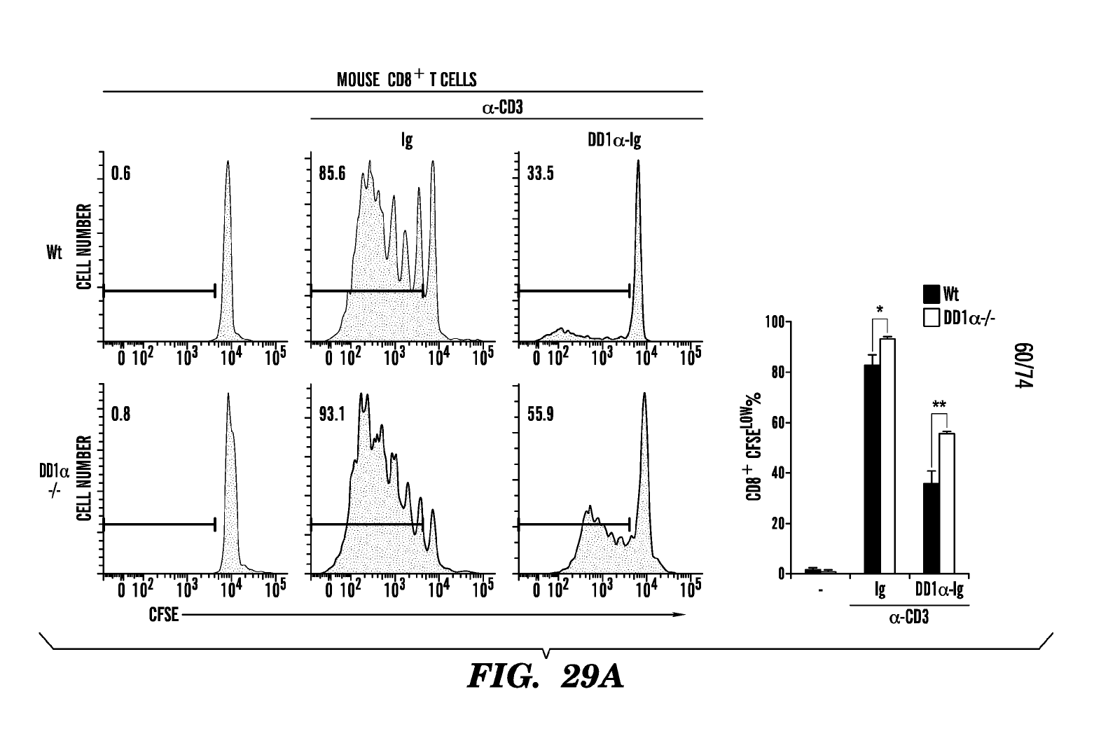

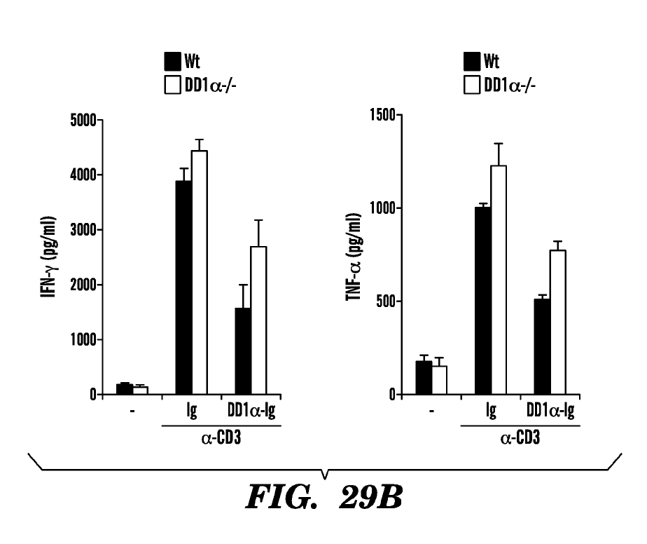

The findings described herein demonstrate an essential role for DD1α in dead cell clearance by phagocytes as an “eat-me” signal on apoptotic cells and/or an “eat-me” signal receptor on phagocytes. As shown in FIG. 27, DD1α was expressed not only on macrophages but also on CD4+ and CD8+ T cells, and barely detectable expression on B220+B cells, which suggested the potential role of DD1α as a receptor or ligand on T cells. DD1α is a member of the immunoglobulin superfamily and is also known as Gi24NISTA/PD-1H/Dies1 (40-43) with homology to the B7 family ligand PD-L1 co-inhibitory molecule in its the IgV region (FIG. 1D). Moreover, a recent report showed that VISTA expression on APCs functions as a negative regulator/ligand of T cell response in vitro and VISTA overexpression on tumor cells interferes with protective antitumor immunity in vivo mice (41). It was also demonstrated that intercellular homophilic interactions of DD1α receptor are essential for its active role in dead cell clearance (FIG. 3). The inventors built upon these findings and hypothesized that the constitutive expression of DD1α on both APCs and T cells contributes to the regulation of T cell responses. To test this hypothesis, the inventors first examined the role of DD1α in human CD4+ and CD8+ T cell activation. Purified CD4+ and CD8+ T cells from human blood were incubated with various concentrations of human DD1α-Ig protein, together with 3 μg/ml of anti-CD3 antibody for T cell stimulation. DD1α-Ig protein strongly inhibited CD4+ and CD8+ T cell proliferation stimulated by anti-CD3 in a dose-dependent manner. T cell activation-induced production of IFN-γ and TNF-α were clearly blocked by DD1α (FIG. 5A). The inventors also observed an inhibitory effect of DD1α on mouse CD4+ and CD8+ T cell activation in a dose-dependent manner (FIG. 28), as previously reported (41). Human CD4+ T cell inhibitory activity of DD1α (˜45% inhibition: from 92.2% proliferation of Ig control to 47.1% proliferation of DD1α-Ig) was shown to a similar extent as that of PD-L1-Ig (˜44% inhibition: from 92.2% proliferation of Ig control to 48.5% proliferation of PD-L1-Ig). However, the combined treatment of DD1α-Ig and PD-L1-Ig only slightly increased suppression of CD4+ T cell activation (to ˜58% inhibitory activity). As this is only 14% more than DD1α-Ig or PD-L1-Ig alone, these data indicated that the mechanism for T cell inhibition by DD1α and PD-L1 may be overlapping. The inventors next tested whether DD1α inhibits CD4+ T cell proliferation via PD-1, a well-characterized co-inhibitory receptor for PD-L1 ligand (49). After pre-incubation of human CD4+ T cells with anti-PD-1 blocking antibody, the effect of DD1α-Ig on anti-CD3-stimulated CD4+ T cell activation was assessed. DD1α-Ig-mediated CD4+ T cell inhibition was reversed up to ˜12% by the addition of anti-PD-1 antibody (from 47.1% proliferation of DD1α-Ig to 58.6% proliferation of both DD1α-Ig and anti-PD-1 Ab), indicating that DD1α functions, at least partially, as a PD-1 ligand for the inhibition of CD4+ T cell activation (FIG. 5B). Since intercellular homophilic DD1α interaction is required for its scavenger function (FIG. 3) and DD1α is expressed on the surface of CD4+ and CD8+ T cells (FIG. 27), the inventors further examined whether DD1α on CD4+ T cells is responsible for DD1α-mediated inhibition of T cell activation. CD4+ and CD8+ T cells from wt- or DD1α−/− mice were used for DD1α-Ig-mediated T cell inhibition experiments. It was found that engagement of DD1α-Ig strongly inhibited anti-CD3-induced CD4+ or CD8+ T cell proliferation (˜50% inhibition) as expected. However, DD1α−/− CD4+ or CD8+ T cells were significantly less sensitive to DD1α-Ig (˜30% inhibition) (FIG. 5C) although DD1α-deficiency did not prevent the inhibition completely, indicating that DD1α-deficiency in T cells impaired DD1α-mediated inhibitory activity against T cell stimulation. These data indicate that homophilic DD1α interactions are important for the DD1α-mediated T cell inhibitory role, and that DD1α functions as a receptor on T cells. Clearly, DD1α deficiency on CD4+ or CD8+ T cells did not show complete blocking of DD1α-Ig-mediated CD4+ or CD8+ T cell inhibition (FIG. 5C, FIG. 28). These data indicate that other co-inhibitory receptor(s) on T cells are involved in the DD1α-mediated CD4+ or CD8+ T cell inhibition. Interestingly, DD1α−/− CD8+ T cells showed relatively higher basal activation than wt CD8+ T cells when stimulated with anti-CD3 antibody, indicating that DD1α on CD8+ T cells could itself modulate anti-CD3-induced T cell activation (FIG. 29). Regulatory T (Treg) cells are essential for the maintenance of peripheral tolerance, and the regulating role for PD-L1 in iTreg cell development has been identified (50). It was next investigated whether DD1α might regulate iTreg cell conversion. With naïve CD4+CD62L+ T cells from Wt mice, the inventors assessed the effect of DD1α on the frequency of Foxp3+CD4+ T cells induced by anti-CD3 and anti-CD28 in the absence or presence of TGF-6. DD1α-Ig but not control Ig significantly enhanced iTreg cell development in the presence of TGF-6 (14.2% vs. 31.4% at 1 ng/ml TGF-6, 32.6% vs. 51.7% at 2 ng/ml), as did PD-L1-Ig (FIG. 5D). However, DD1α-Ig alone without TGF-6 did not affect iTreg cell generation. These data indicate that DD1α and TGF-β have synergistic roles in regulating Foxp3+ iTreg cell development.

### Example 5: DD1α Physically Associates with PD-1, and PD-1 Functions as a Co-Inhibitory Ligand for DD1α Receptor on T Cells

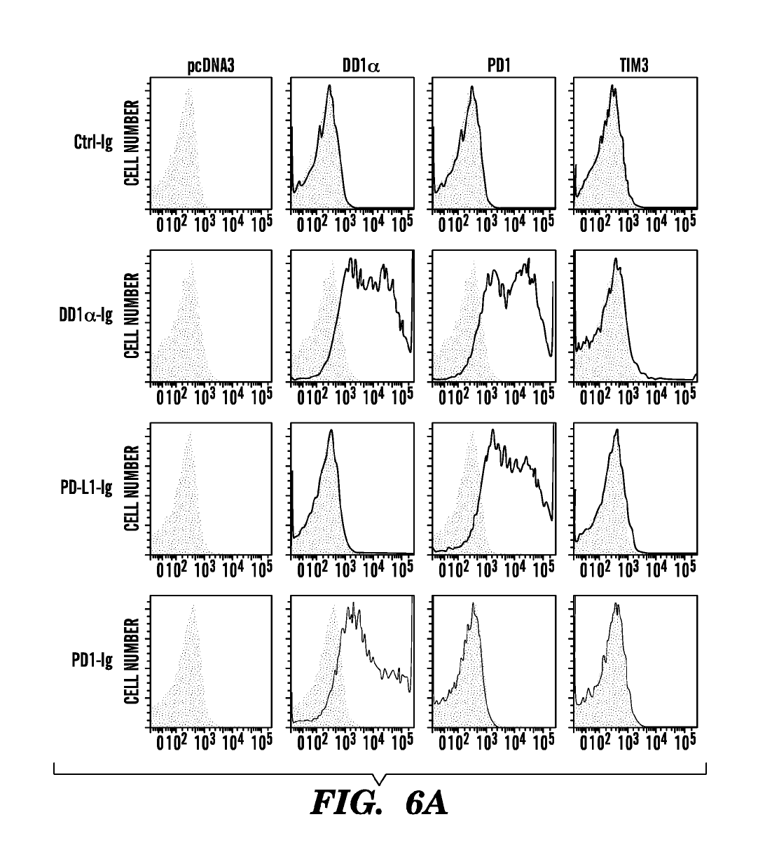

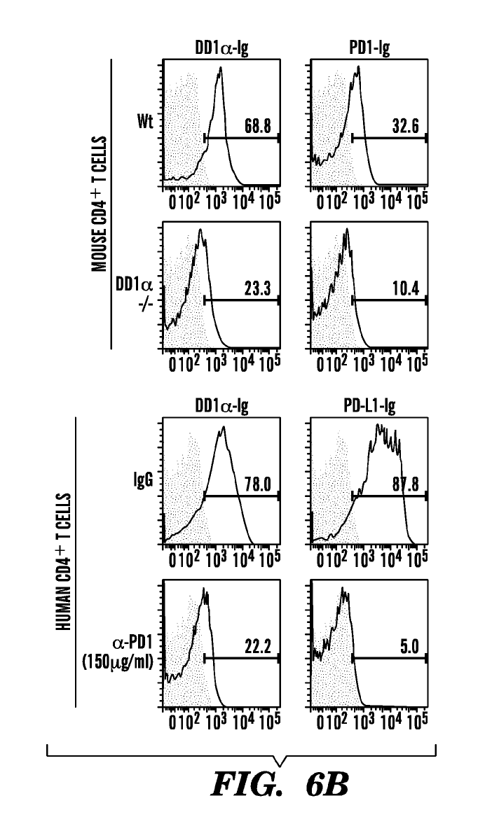

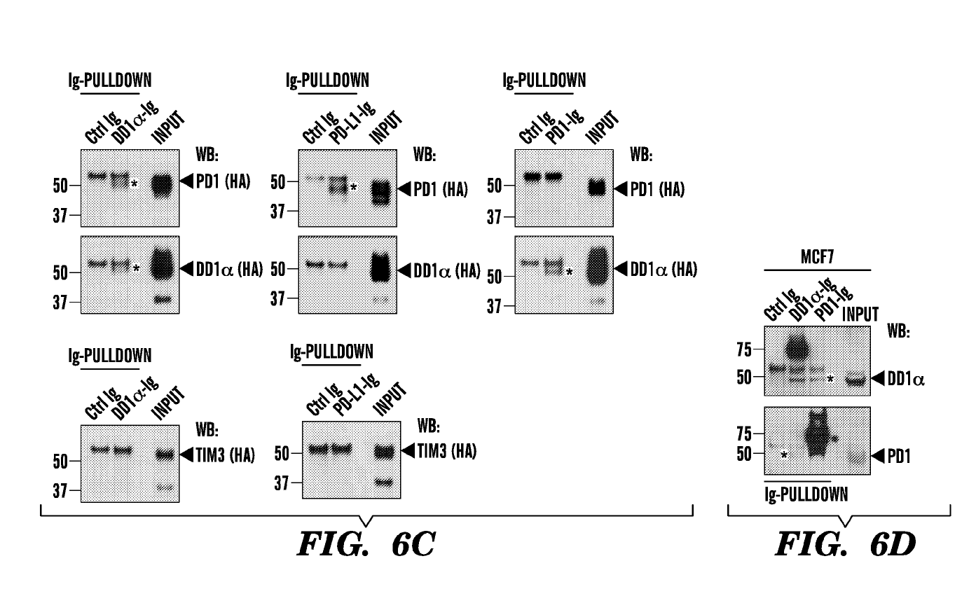

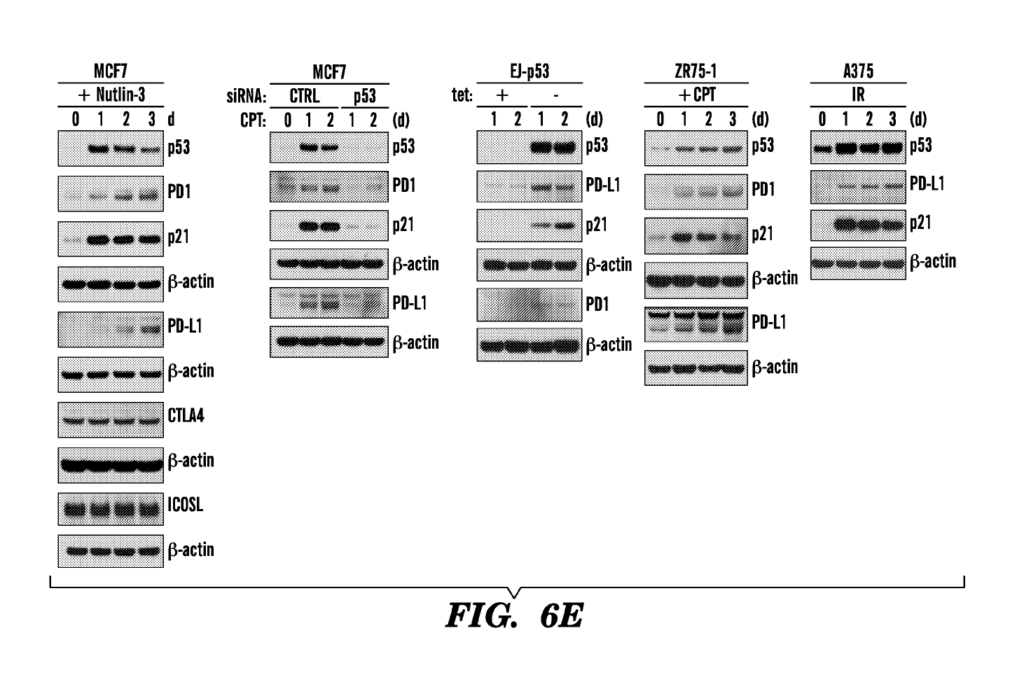

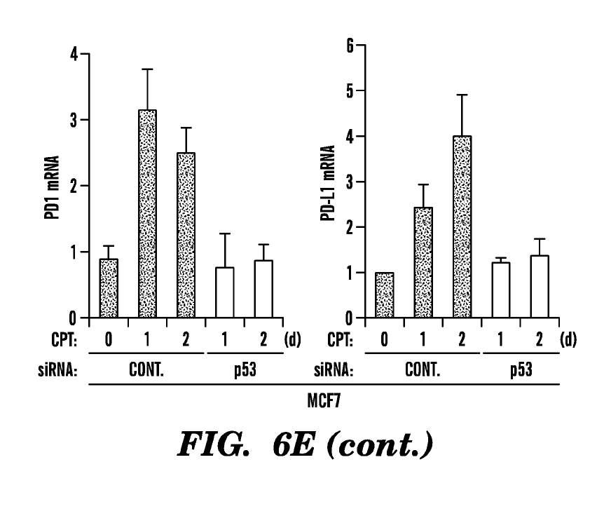

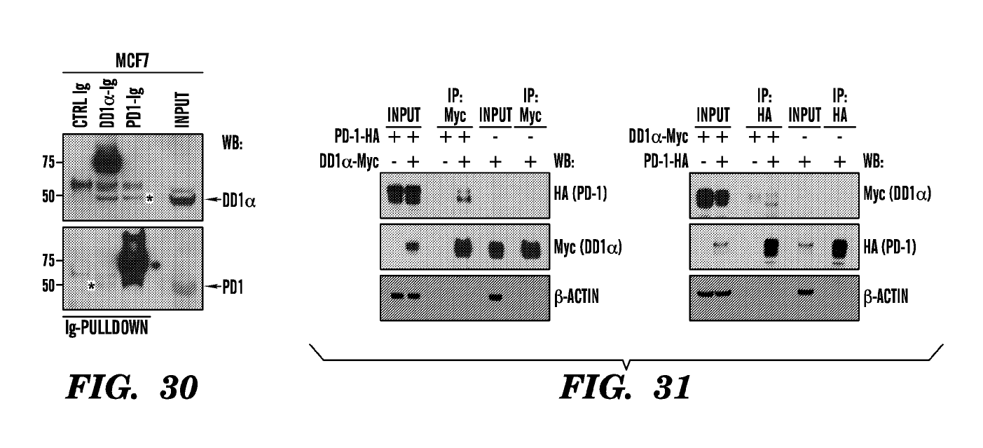

Based on the results that both PD-1 and DD1α receptors on T cells contribute to the co-inhibitory roles for DD1α(FIG. 5), it was tested whether DD1α could physically and functionally associate with PD-1 receptor. The inventors generated 293T cell transfectants expressing empty vector, DD1α, PD-1 or TIM3 on the cell surface and stained with Ig-fusion proteins: control Ig, DD1α-Ig, PD-L1-Ig, or PD-1-Ig. DD1α-Ig specifically bound to DD1α- and PD-1-transfected cells but not to PD-L1 or TIM-3 transfectants (FIG. 6A). Conversely, PD-1-Ig bound to DD1α but not to TIM-3 or PD-1 transfectants, and PD-L1-Ig bound to PD-1 but not to DD1α or TIM-3 transfectants (FIG. 6A). These data indicate that DD1α may interact with PD-1 receptor, as well as DD1α, but not with PD-L1 or TIM-3, which share homology with DD1α (FIG. 1D). To confirm the specificity of this interaction, the inventors carried out Ig fusion protein-pull down assays using lysates from PD-1, DD1α, or TIM3-transfected 293T cells. Consistent with the staining experiments (FIG. 6A), DD1α-Ig pulled down PD-1 and DD1α, and reciprocally, DD1α was also pulled down by PD-1-Ig (FIG. 6B). The inventors further validated the binding of DD1α to PD-1 receptor or DD1αreceptor on T cells by testing whether DD1α−/− mouse T cells or anti-PD-1 antibody could block DD1α-PD-1 or DD1α-DD1αbinding on CD4+ T cells. The inventors preincubated human CD4+ T cells with or without anti-PD-1 blocking Ab (150 μg/ml), and then stained them with DD1α-Ig or PD-1-Ig. This preincubation with anti-PD-1 antibody significantly diminished the binding of DD1α-Ig to human CD4+ T cells but did not completely remove the binding (FIG. 6C), while preincubation with anti-PD-1 eliminated more than 90% of the PD-L1-Ig binding to CD4+ T cells. These data indicate that DD1αfunctions through at least two receptors on T cells: PD-1 receptor and DD1αreceptor although PD-1 molecule is the only receptor for PD-L1 on T cells. In addition, the inventors further confirmed the specific interaction between DD1α and PD-1 receptor on T cells using wt- or DD1α−/− mouse CD+ T cells. The binding of DD1α-Ig or PD-1-Ig to the surface of DD1α−/− CD4+ T cells was significantly lower than its binding to wt-CD4+ T cells (FIG. 6C, lower panel). These experiments indicate that the staining seen on T cells represents a specific interaction between DD1α and PD-1, which could be blocked by prior incubation with anti-PD-1 or in DD1α-deficient T cells. The binding of DD1α to endogenous PD-1 or DD1αreceptor was also tested by Ig fusion protein-pull down assay with lysates of Nutlin-3-treated MCF7 cells with wt-p53. Endogenous DD1α receptor was detected in the resulting pull down complex with DD1α-Ig and PD-1-Ig; reciprocally, PD-1 was found in the DD1α-Ig pull down (FIG. 30).

As PD-1 specifically bound to DD1α on CD4+ T cells, it was further examined whether PD-1 has a co-inhibitory or co-stimulatory function as a ligand by association with DD1α receptor on CD4+ T cells. The effect of PD-1-Ig was examined on CD4+ T cells from wt- or DD1α−/− mice. It was found that PD-1-Ig significantly inhibited anti-CD3-induced CD4+ T cell proliferation (˜45% inhibition) with similar inhibitory activity as compared to DD1α-Ig and PD-L1-Ig. The PD-1-Ig-mediated inhibition of CD4+ T cell stimulation was diminished in DD1α−/− CD4+ T cells (reversed by ˜30%) (FIG. 6D), indicating that DD1α receptor on CD4+ T cells is required for PD-1-mediated CD4+ T cell inhibition and that PD-1 serves as a co-inhibitory ligand for DD1α receptor on T cells.

Given the apparent functional interaction between DD1α and PD-1 on T cells, the inventors sought to determine whether the co-inhibitory molecules such as PD-1 and PD-L1 might be regulated by p53 as seen for DD1α. Unexpectedly, both PD-1 and PD-L1 were induced by a p53 activator, Nutlin-3 (10 μM), in MCF7 cells, whereas the co-inhibitory receptor CTLA-4 and the co-stimulatory ligand ICOSL did not respond to p53 induction (FIG. 6E). Genotoxic stimuli also activated PD-1 and PD-L1 in a p53-dependent manner. The inventors also verified p53-mediated activation of PD-1 or PD-L1 expression in several p53 expression systems (FIG. 6E). Together, these findings indicate a novel role for tumor suppressor p53 in regulating expression of immune checkpoint regulators, including PD-1, PD-L1 and DD1α, and indicating a role of p53 in immune surveillance.

### SUMMARY

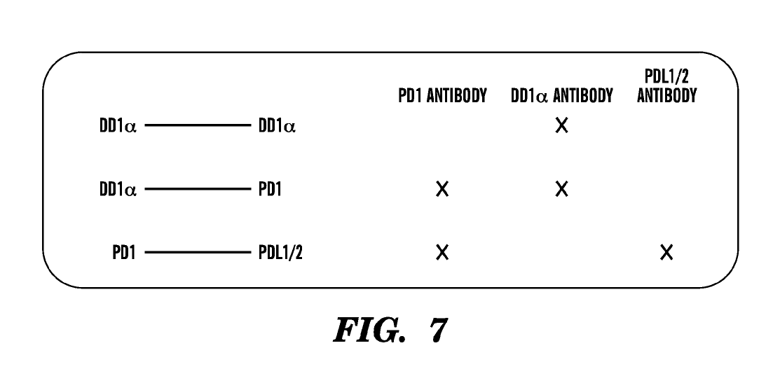

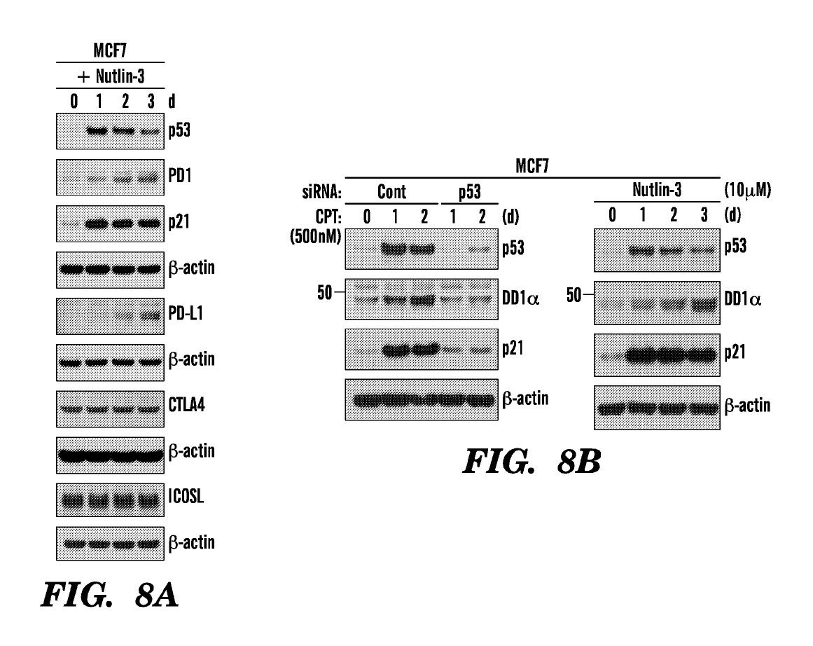

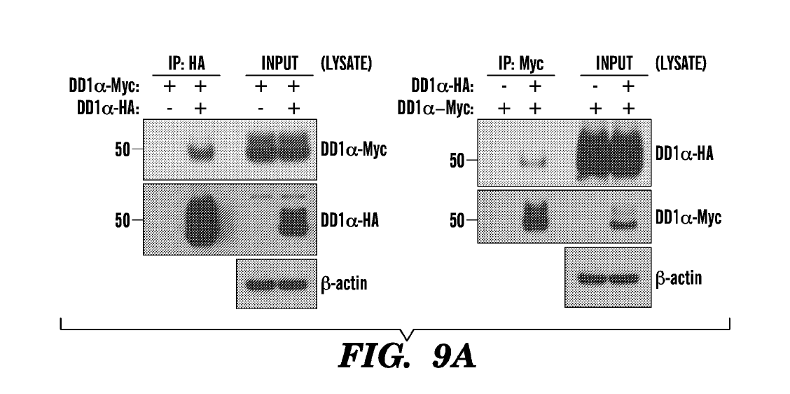

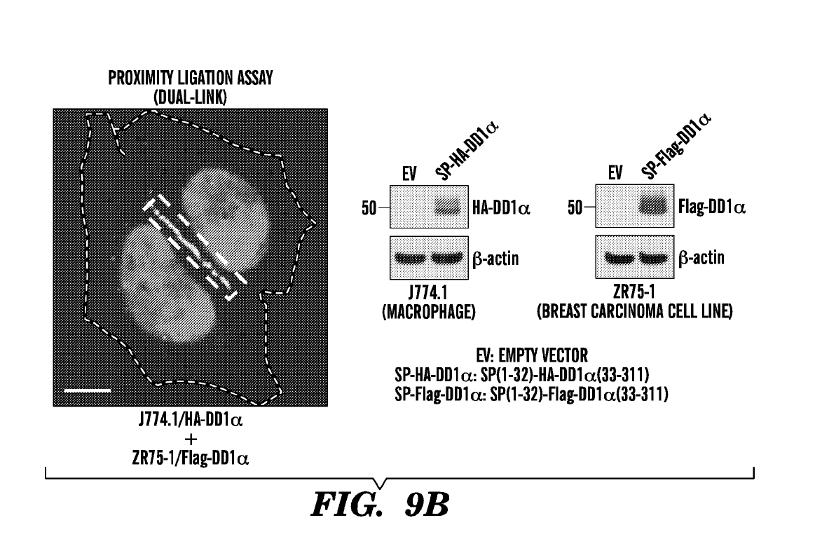

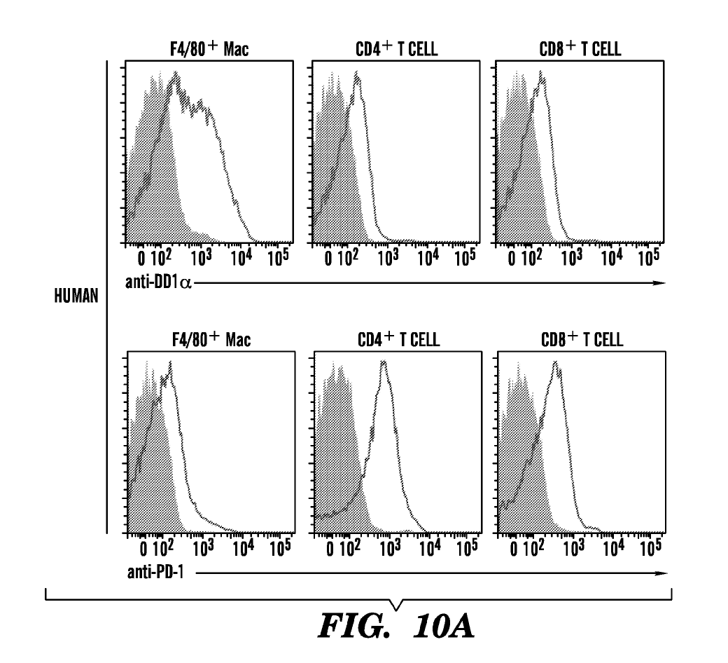

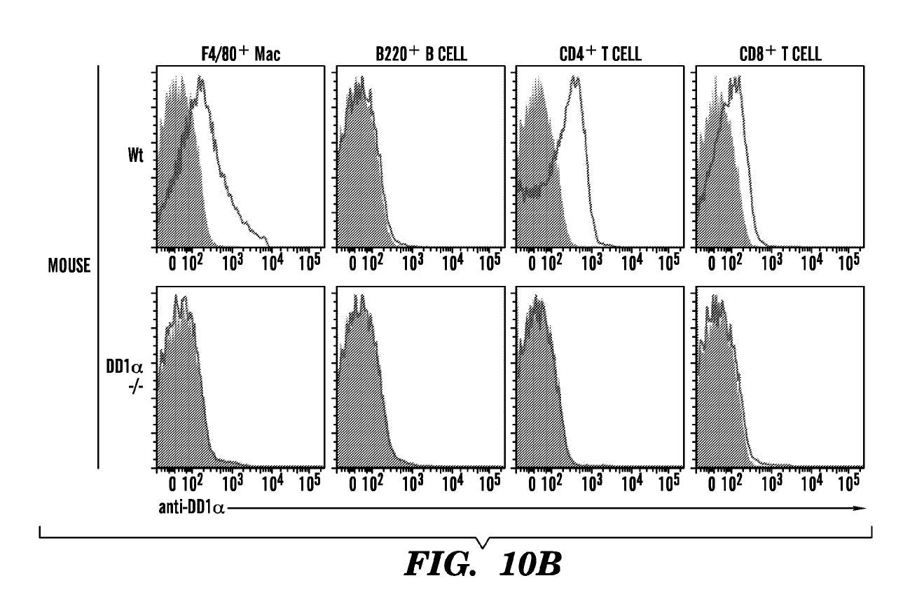

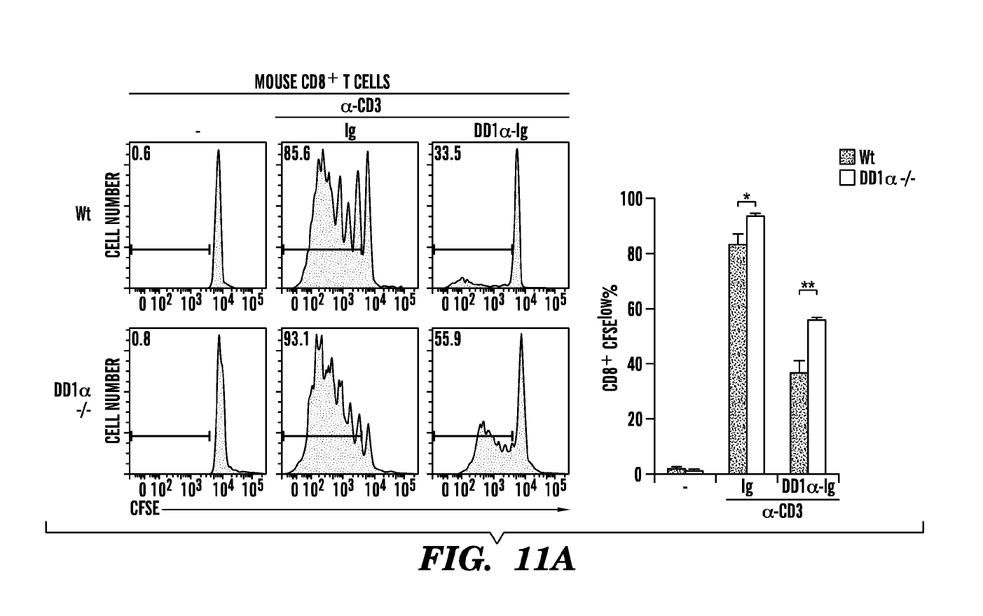

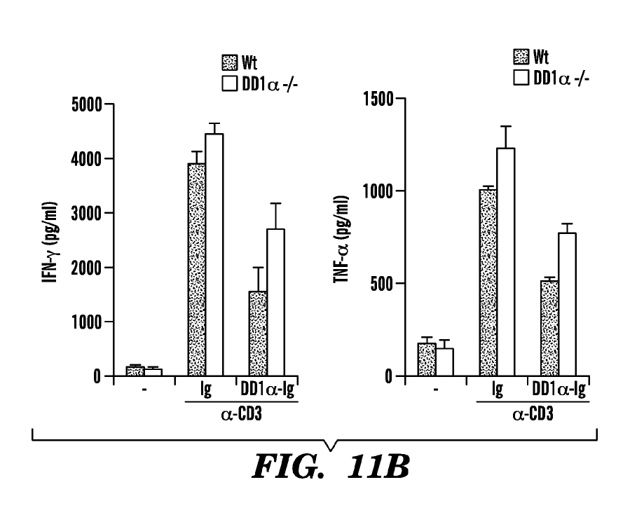

The findings described herein reveal that the tumor suppressor p53-DD1α pathway regulates a common immune program by which healthy tissues maintain homeostatic control via efficient dead cell clearance and proper immune tolerance. Although these two essential activities are seemingly interrelated, the complexity of these processes is demonstrated by the many receptors and signaling pathways involved in the recognition and removal of apoptotic cells by macrophages and stringent discrimination of self-antigens from non-self-antigens (1, 51-53). Here the inventors have presented evidence that DD1α is a receptor that engages in homophilic intermolecular interaction/dimerization at intercellular junctions of apoptotic cells and macrophages, which is critical for engulfment of apoptotic cells, unlike other typical scavenger receptors that recognize PtdSer on the surface of dead cells can be a key factor in a variety of human pathologies including autoimmune diseases, asthma, atherosclerosis, degenerative disorders (Alzheimer's disease), infections, and cancer (1, 2, 4, 6). Mice deficient in scavenger receptors develop spontaneous autoimmune disease (54-57) while deficiencies in dead cell clearance do not always elicit autoimmunity because these apoptotic cells in these deficiencies are still able to trigger a downstream immunosuppressive signal to inhibit autoimmunity (58, 59). In a normal immune system, phagocytic engulfment of apoptotic cells is accompanied by induction of a certain degree of immune tolerance in order to prevent self-antigen recognition through the activation of immune checkpoint pathways as well as the production of anti-inflammatory cytokines (51, 52, 60). The inventors show that DD1α-deficient mice showed normality in health at early age, but by 6 to 7 months after birth, severe autoimmune phenotypes emerged predominantly in female mice. These results imply that the role(s) of DD1αreceptor seems to be critical in dead cell clearance, and its loss directly affects immune tolerance resulting in the development of severe inflammation and systemic autoimmune phenotypes. Thus, there appears to be a link or direct cross-talk between DD1α-mediated phagocytic engulfment of dead cells and immune tolerance. Supporting this hypothesis, DD1α is highly expressed in immune cells including macrophages and T cells, and functions as a receptor through homophilic DD1α-DD1α binding at intercellular junctions. It was further demonstrated that DD1α plays an important role as an intercellular homophilic receptor on T cells, indicating that DD1α is a key-connecting molecule linking post-apoptotic processes to immune surveillance via a unique intercellular DD1α-DD1α interaction (FIG. 7). Recently, other groups have also identified DD1α as a B7 family member (41) and introduced DD1α/VISTA as a novel negative checkpoint protein ligand of the T cell. These studies indicated that identification of its receptor would be important for understanding mechanisms of VISTA-mediated inhibition of T cell immunity (41). We report that DD1α/VISTA functions as a receptor on T cells and undergoes homophilic DD1α interactions that are required for DD1α-mediated T cell inhibition. It was also shown that DD1α inhibits T cell activation via two receptors: PD-1, a well-characterized co-inhibitory receptor, and DD1αitself. These results indicate the possibility of a new combined paradigm with which to explain restricted generation of specific immune responses only to contexts in which they are needed (FIG. 7). The inventors identify a specific interaction between DD1α and PD-1, and demonstrate that this association is functionally immunoregulatory for T cell stimulation. More importantly, these data indicate that PD-1 can function as a ligand for DD1α receptor on T cells, which forces a new view of the key immunomodulatory interactions that govern T cell activation and tolerance.

These findings establish a novel concept for cancer-driven control of immune surveillance: p53-dependent genotoxic stress-mediated expression of DD1α and related immune checkpoint inhibitors such as PD-1 and PD-L1 molecules on cancer cells may permit their interaction with T cells through intercellular homophilic binding (DD1α/DD1α), and/or heterophilic (PD-1/DD1α or PD-L1/PD-1) binding in order to impose suppression of the immune response and escape from immune surveillance (FIG. 7).

Importantly, expression of DD1α, PD-1 and PD-L1 are significantly increased in multiple human cancers (61-64). Since both PD-1 and DD1αfunctionally and biochemically cooperated to serve as negative regulators of immune cell activation, targeting the PD-1/DD1α, DD1α/DD1α, and/or DD1α/PD-1 pathways can deliver potential therapeutic opportunities for cancer immunotherapy.

Together, these studies reveal a layer of immune regulation in which DD1αpermits efficient clearance of apoptotic cells, interacts functionally with PD-1, and acts to prevent autoimmunity. As a direct target of p53, DD1α a activation can extend the repertoire of p53 activities to “guardian of the immune integrity.”

### REFERENCES

1. S. Nagata, R. Hanayama, K. Kawane, Autoimmunity and the clearance of dead cells. Cell 140, 619 (Mar. 5, 2010).
2. M. R. Elliott, K. S. Ravichandran, Clearance of apoptotic cells: implications in health and disease. The Journal of cell biology 189, 1059 (Jun. 28, 2010).
3. Y. Fuchs, H. Steller, Programmed cell death in animal development and disease. Cell 147, 742 (Nov. 11, 2011).
4. S. Nagata, Apoptosis and autoimmune diseases. Annals of the New York Academy of Sciences 1209, 10 (October, 2010).
5. R. W. Vandivier, P. M. Henson, I. S. Douglas, Burying the dead: the impact of failed apoptotic cell removal (efferocytosis) on chronic inflammatory lung disease. Chest 129, 1673 (June, 2006).
6. I. K. Poon, C. D. Lucas, A. G. Rossi, K. S. Ravichandran, Apoptotic cell clearance: basic biology and therapeutic potential. Nature reviews. Immunology, (Jan. 31, 2014).
7. V. A. Fadok et al., Exposure of phosphatidylserine on the surface of apoptotic lymphocytes triggers specific recognition and removal by macrophages. Journal of immunology 148, 2207 (Apr. 1, 1992).
8. D. Park et al., BAT′ is an engulfment receptor for apoptotic cells upstream of the ELMO/Dock180/Rac module. Nature 450, 430 (Nov. 15, 2007).
9. N. Kobayashi et al., TIM-1 and TIM-4 glycoproteins bind phosphatidylserine and mediate uptake of apoptotic cells. Immunity 27, 927 (December, 2007).
10. S. Y. Park et al., Rapid cell corpse clearance by stabilin-2, a membrane phosphatidylserine receptor. Cell death and differentiation 15, 192 (January, 2008).
11. M. Miyanishi et al., Identification of Tim4 as a phosphatidylserine receptor. Nature 450, 435 (Nov. 15, 2007).
12. R. H. DeKruyff et al., T cell/transmembrane, Ig, and mucin-3 allelic variants differentially recognize phosphatidylserine and mediate phagocytosis of apoptotic cells. Journal of immunology 184, 1918 (Feb. 15, 2010).
13. R. Hanayama et al., Identification of a factor that links apoptotic cells to phagocytes. Nature 417, 182 (May 9, 2002).
14. H. Paidassi et al., Clq binds phosphatidylserine and likely acts as a multiligand-bridging molecule in apoptotic cell recognition. Journal of immunology 180, 2329 (Feb. 15, 2008).
15. M. P. Chao, R. Majeti, I. L. Weissman, Programmed cell removal: a new obstacle in the road to developing cancer. Nature reviews. Cancer 12, 58 (January, 2012).
16. S. Jaiswal et al., CD47 is upregulated on circulating hematopoietic stem cells and leukemia cells to avoid phagocytosis. Cell 138, 271 (Jul. 23, 2009).
17. P. A. Oldenborg et al., Role of CD47 as a marker of self on red blood cells. Science 288, 2051 (Jun. 16, 2000).
18. R. Majeti et al., CD47 is an adverse prognostic factor and therapeutic antibody target on human acute myeloid leukemia stem cells. Cell 138, 286 (Jul. 23, 2009).
19. S. B. Willingham et al., The CD47-signal regulatory protein alpha (SIRPa) interaction is a therapeutic target for human solid tumors. Proc Natl Acad Sci USA 109, 6662 (Apr. 24, 2012).
20. K. Weiskopf et al., Engineered SIRPalpha variants as immunotherapeutic adjuvants to anticancer antibodies. Science 341, 88 (Jul. 5, 2013).
21. D. P. Lane, Cancer. p53, guardian of the genome. Nature 358, 15 (Jul. 2, 1992).
22. B. Vogelstein, D. Lane, A. J. Levine, Surfing the p53 network. Nature 408, 307 (Nov. 16, 2000).
23. Y. Aylon, M. Oren, Living with p53, dying of p53. Cell 130, 597 (Aug. 24, 2007).
24. K. H. Vousden, C. Prives, Blinded by the Light: The Growing Complexity of p53. Cell 137, 413 (May 1, 2009).
25. J. P. Kruse, W. Gu, Modes of p53 regulation. Cell 137, 609 (May 15, 2009).
26. C. Munoz-Fontela et al., Transcriptional role of p53 in interferon-mediated antiviral immunity. The Journal of experimental medicine 205, 1929 (Aug. 4, 2008).
27. C. Munoz-Fontela et al., p53 serves as a host antiviral factor that enhances innate and adaptive immune responses to influenza A virus. Journal of immunology 187, 6428 (Dec. 15, 2011).
28. C. Rivas, S. A. Aaronson, C. Munoz-Fontela, Dual Role of p53 in Innate Antiviral Immunity. Viruses 2, 298 (January, 2010).
29. E. A. Komarova et al., p53 is a suppressor of inflammatory response in mice. FASEB journal: official publication of the Federation of American Societies for Experimental Biology 19, 1030 (June, 2005).
30. S. J. Zheng, S. E. Lamhamedi-Cherradi, P. Wang, L. Xu, Y. H. Chen, Tumor suppressor p53 inhibits autoimmune inflammation and macrophage function. Diabetes 54, 1423 (May, 2005).
31. J. S. Park et al., p53 controls autoimmune arthritis via STAT-mediated regulation of the Th17 cell/Treg cell balance in mice. Arthritis and rheumatism 65, 949 (April, 2013).
32. A. P. Kuan, P. L. Cohen, p53 is required for spontaneous autoantibody production in B6/lpr lupus mice. European journal of immunology 35, 1653 (May, 2005).
33. T. Mori et al., Identification of the interferon regulatory factor 5 gene (IRF-5) as a direct target for p53. Oncogene 21, 2914 (Apr. 25, 2002).
34. B. T. Hummer, X. L. Li, B. A. Hassel, Role for p53 in gene induction by double-stranded RNA. Journal of virology 75, 7774 (August, 2001).
35. M. Taura et al., p53 regulates Toll-like receptor 3 expression and function in human epithelial cell lines. Molecular and cellular biology 28, 6557 (November, 2008).
36. D. J. Jung et al., Foxp3 expression in p53-dependent DNA damage responses. The Journal of biological chemistry 285, 7995 (Mar. 12, 2010).
37. H. Kawashima et al., Tumor suppressor p53 inhibits systemic autoimmune diseases by inducing regulatory T cells. Journal of immunology 191, 3614 (Oct. 1, 2013).
38. A. Lujambio et al., Non-cell-autonomous tumor suppression by p53. Cell 153, 449 (Apr. 11, 2013).
39. N. Raj, L. D. Attardi, Tumor suppression: p53 alters immune surveillance to restrain liver cancer. Current biology: CB 23, R527 (Jun. 17, 2013).
40. M. A. Sakr et al., GI24 enhances tumor invasiveness by regulating cell surface membrane-type 1 matrix metalloproteinase. Cancer science 101, 2368 (November, 2010).
41. L. Wang et al., VISTA, a novel mouse Ig superfamily ligand that negatively regulates T cell responses. The Journal of experimental medicine 208, 577 (Mar. 14, 2011).
42. D. B. Flies, S. Wang, H. Xu, L. Chen, Cutting edge: A monoclonal antibody specific for the programmed death-1 homolog prevents graft-versus-host disease in mouse models. Journal of immunology 187, 1537 (Aug. 15, 2011).
43. L. Aloia, S. Parisi, L. Fusco, L. Pastore, T. Russo, Differentiation of embryonic stem cells 1 (Dies1) is a component of bone morphogenetic protein 4 (BMP4) signaling pathway required for proper differentiation of mouse embryonic stem cells. The Journal of biological chemistry 285, 7776 (Mar. 5, 2010).
44. L. Brown et al., CDIP, a novel pro-apoptotic gene, regulates TNFalpha-mediated apoptosis in a p53-dependent manner. Embo J 26, 3410 (Jul. 25, 2007).
45. J. Suzuki, D. P. Denning, E. Imanishi, H. R. Horvitz, S. Nagata, Xk-related protein 8 and CED-8 promote phosphatidylserine exposure in apoptotic cells. Science 341, 403 (Jul. 26, 2013).
46. S. W. Lowe, E. M. Schmitt, S. W. Smith, B. A. Osborne, T. Jacks, p53 is required for radiation-induced apoptosis in mouse thymocytes. Nature 362, 847 (Apr. 29, 1993).
47. L. E. Munoz, K. Lauber, M. Schiller, A. A. Manfredi, M. Herrmann, The role of defective clearance of apoptotic cells in systemic autoimmunity. Nature reviews. Rheumatology 6, 280 (May, 2010).
48. C. A. O'Callaghan, Renal manifestations of systemic autoimmune disease: diagnosis and therapy. Best practice & research. Clinical rheumatology 18, 411 (June, 2004).
49. G. J. Freeman et al., Engagement of the PD-1 immunoinhibitory receptor by a novel B7 family member leads to negative regulation of lymphocyte activation. The Journal of experimental medicine 192, 1027 (Oct. 2, 2000).
50. L. M. Francisco et al., PD-L1 regulates the development, maintenance, and function of induced regulatory T cells. The Journal of experimental medicine 206, 3015 (Dec. 21, 2009).
51. D. R. Green, H. M. Beere, Apoptosis. Gone but not forgotten. Nature 405, 28 (May 4, 2000).
52. T. A. Ferguson, J. Choi, D. R. Green, Armed response: how dying cells influence T-cell functions. Immunological reviews 241, 77 (May, 2011).
53. 0. Acuto, V. Di Bartolo, F. Michel, Tailoring T-cell receptor signals by proximal negative feedback mechanisms. Nature reviews. Immunology 8, 699 (September, 2008).
54. R. Rodriguez-Manzanet et al., T and B cell hyperactivity and autoimmunity associated with niche-specific defects in apoptotic body clearance in TIM-4-deficient mice. Proc Natl Acad Sci USA 107, 8706 (May 11, 2010).
55. R. Hanayama et al., Autoimmune disease and impaired uptake of apoptotic cells in MFG-E8-deficient mice. Science 304, 1147 (May 21, 2004).
56. Z. G. Ramirez-Ortiz et al., The scavenger receptor SCARF1 mediates the clearance of apoptotic cells and prevents autoimmunity. Nature immunology 14, 917 (September, 2013).
57. M. Botto et al., Homozygous Clq deficiency causes glomerulonephritis associated with multiple apoptotic bodies. Nature genetics 19, 56 (May, 1998).
58. Y. Hirose et al., Inhibition of Stabilin-2 elevates circulating hyaluronic acid levels and prevents tumor metastasis. Proc Natl Acad Sci USA 109, 4263 (Mar. 13, 2012).
59. K. Wong et al., Phosphatidylserine receptor Tim-4 is essential for the maintenance of the homeostatic state of resident peritoneal macrophages. Proc Natl Acad Sci USA 107, 8712 (May 11, 2010).
60. V. A. Fadok et al., Macrophages that have ingested apoptotic cells in vitro inhibit proinflammatory cytokine production through autocrine/paracrine mechanisms involving TGF-beta, PGE2, and PAF. The Journal of clinical investigation 101, 890 (Feb. 15, 1998).
61. H. Dong et al., Tumor-associated B7-H1 promotes T-cell apoptosis: a potential mechanism of immune evasion. Nature medicine 8, 793 (August, 2002).
62. Y. Iwai et al., Involvement of PD-L1 on tumor cells in the escape from host immune system and tumor immunotherapy by PD-L1 blockade. Proc Natl Acad Sci USA 99, 12293 (Sep. 17, 2002).
63. W. Zou, L. Chen, Inhibitory B7-family molecules in the tumor microenvironment. Nature reviews. Immunology 8, 467 (June, 2008).
64. R. Hino et al., Tumor cell expression of programmed cell death-1 ligand 1 is a prognostic factor for malignant melanoma. Cancer 116, 1757 (Apr. 1, 2010).

### Example 6: DD1α Regulates T Cell Activation Through DD1α on T Cells

The inventors have discovered that DD1αregulates T cell activation through DD1α on T cells. To characterize function of DD1α receptor on CD4+ T cell in DD1α-mediated T cell inhibition, purified CD4+ T cells from Wt and DD1α−/− mice were stimulated with 2.5 μg/ml anti-CD3 alone or together with DD1α-Ig, PD-L1-Ig or control Ig. The proliferations of CD4+ T cells were determined by percentage of CFSE-diluted CD4+ cells on day 3 and IFN-γ and levels in culture supernatants on day 2 were analyzed by ELISA. Combinational inhibitory effect of DD1α and PD-L1 on anti-CD3-induced CD4+ T cell activation was also examined. To characterize inhibitory effect of DD1α on the activation of CD8+ T cells from Wt and DD1α−/− mice, purified mouse CD8+ T cells from Wt and DD1α−/− mice were stained with 1 μM CFSE and stimulated with plate-bound 2.5 μg/ml anti-CD3 antibody together with DD1α-Ig protein or control Ig protein. The proliferations of CD8+ T cells were determined by percentage of CD8+ T cells containing diluted CFSE signal on day 3. The cytokine levels in culture supernatant were analyzed by ELISA on day 2.

### Example 7: Exemplary Methods Used in the Examples Described Herein Plasmids, Transfection and Lentivirus

For mammalian expression vector, full length human DD1α cDNA was subcloned into pcDNA3.1(−) with HA or Myc tagged at the C-terminus using standard procedure. Immunoglobulin V domain (aa 45-168)-deleted mutant DD1α-ΔIgV was generated by PCR-mediated deletion method. For Fc-tagged soluble DD1α proteins (DD1α-Ig), the extracellular region of DD1α were cloned upstream of the mouse IgG-Fc region (DD1α-Ig) in the pCMV6-AC-FC vector (OriGene). Plasmid encoding TIM1-Ig was a gift from Terry B. Strom (Transplant Institute, Beth Israel Deaconess Medical Center and Harvard Medical School, Boston, Mass.). For recombinant DD1αdeletion mutants, DD1α(33-194), DD1α(37-146), DD1α-ΔIgV (33-311, ΔIgV) and DD1α (215-311) were generated by PCR and subcloned into pGEX-6P-1 (GE health). DD1α(33-194) was amplified and subcloned into pRSET vector (Invitrogen). For mammalian expression-vector constructs encoding PD-1-HA and Tim3-HA, full length human PD-1 and Tim3 cDNA (Sino biological) were cloned into pcDNA3.1(−) tagged with HA at C-terminus. Transfections were performed using Lipofectamine 2000 (Invitrogen).

Plasmids encoding lentivirus expressing DD1α-HA were generated by cloning DD1α-HA into pLenti CMV Neo DEST. Cells were transduced with lentivirus in the presence of polybrene. Infected cells were selected in complete appropriate medium containing 10% FBS and G418 and were tested 1 week after infection.

### Antibodies and Additional Reagents

The following antibodies were used for Western blot analyses or flow cytometry analysis: p53 (DO-1, Santa Cruz), p21 (DSC60, Cell Signaling Technology), β-actin (AC-15, Sigma-Aldrich), His (Invitrogen), GST (B-14, Santa Cruz), HA (F-7, Santa Cruz), Myc (9E10, Santa Cruz), anti-mouse DD1α (MH5A, BioLegend), anti-human PD-1 (J116, eBioscience), anti-mouse PD-1 (J43, eBioscience), anti-human PD-L1 (M1H1, eBioscience), CTLA4 (C-19, Santa Cruz), ICOSL (LifeSpan BioSciences), anti-human CD14 APC (61D3, eBioscience), anti-mouse F4/80 APC (BM8, eBioscience) and anti-mouse CD45R/B220 (RA3-6B2, eBioscience). Rabbit polyclonal and mouse monoclonal anti-DD1α antibody was raised against human DD1α (aa 33-311) as an immunogen and affinity-purified. Beads immobilized with anti-HA antibody (Roche) or anti-Myc (9B11, Cell signaling) were used for immunoprecipitations. Recombinant human PD-1-Ig, mouse PD-Ig, human PD-L1-Ig, mouse PD-L1-Ig and control Ig proteins were from R&D Systems.

### Cells and Culture Conditions

EJ-p53 and EJ-CAT cells were cultured in the presence or absence of tetracycline (1 μg/ml) in Dulbecco's modified Eagle's medium (DMEM) containing 10% fetal bovine serum (FBS) (Gibco), 100 units/ml of penicillin, and 100 μg/ml of streptomycin at 37° C. (1). Human MCF7, ZR75-1, Saos-2, U205, A549, A375, HEK293T, Hs888.T cells and J774 cells were maintained in DMEM (Cellgro) supplemented with 10% FBS and antibiotics. LOX-IMVI and BxPC-3 cells were grown in RPMI (Cellgro) supplemented with 10% FBS and antibiotics. The BxPC-3 cells was provided by Dipak Panigrahy(Beth Israel Deaconess Medical Center, Boston, Mass.). For treatment of DNA damaging agents, cells were grown to −50% confluency prior to exposure to DNA damaging agent, camptothecin (CPT, 0.5-20 μM), etoposide (25 μM) or ionizing irradiation (13 Gy).

shRNA and siRNA Experiments

Plasmids (pLKO.1-puro) expressing shRNAs against human DD1α #1 (5′-GCAGAGACAACTTCTAAGAAT-3′ (SEQ ID NO: 4); TRCN0000145473, Sigma-Aldrich) and DD1α #2 (5′-GCACGATGTGACCTTCTACAA-3′ (SEQ ID NO: 5); TRCN0000140372, Sigma-Aldrich) and pLKO.1-puro Luciferase shRNA Control Plasmid DNA (Sigma-Aldrich) were purchased. p53 siRNA (Validated Stealth RNAi siRNA, oligo ID: VHS40367) and control siRNA (Stealth RNAi Negative Control Low GC) were purchased from Invitrogen. shRNA plasmids and siRNAs were introduced into cells by transfection using Lipofectamine 2000 (Invitrogen) or X-tremeGENE siRNA Transfection Reagent (Roche), repectively. MCF7 or ZR75-1 cells stably expressing shRNAs were generated by selection with puromycin (1.0 μg/ml for MCF7, 1.25 μg/ml for ZR75-1).

### RNA Analysis

RNA was prepared with TRIzol Reagent (Invitrogen) according to the manufacturer's protocol. Total RNAs of human tissues were purchased from Clontech. The RNAs were analyzed for the northern blot analysis as described previously (2). For real time quantitative PCR analysis, total RNAs were extracted with the Qiagen RNeasy kit with QlAshredder. cDNA was synthesized using the iScript cDNA Synthesis Kit (Bio-Rad). Primers were as follows: For human; DD1α: 5′-GATGTGACCTTCTACAAGACG-3′ (SEQ ID NO: 6) and 5′-GTCCTGGAACGTGAGGTTGC-3′ (SEQ ID NO: 7); PD-1: 5′-CCAGGATGGTTCTTAGACTCCC-3′ (SEQ ID NO: 8) and 5′-TTTAGCACGAAGCTCTCCGAT-3′ (SEQ ID NO: 9); PD-L1:5′-TGGCATTTGCTGAACGCATTT-3′ (SEQ ID NO: 10) and 5′-TGCAGCCAGGTCTAATTGTTTT-3′ (SEQ ID NO: 11); RPLPO: 5′-CAGATTGGCTACCCAACTGTT-3′ (SEQ ID NO: 12) and 5′-GGGAAGGTGTAATCCGTCTCC-3′ (SEQ ID NO: 13): For mouse; DD1α: 5′-GGAACCCTGCTCCTTGCTATT-3′ (SEQ ID NO: 14) and 5′-TTGTAGATGGTCACATCGTGC-3′ (SEQ ID NO: 15); RplpO: 5′-TCGGGTCCTAGACCA-3′ (SEQ ID NO: 16) and 5′-AGATTCGGGATATGCTGTTGGC-3′ (SEQ ID NO: 17). qPCR analysis was performed using an Icycler IQTM5 real time system (Bio-Rad) with SYBER Green (Roche) for detection. Expression levels of genes analysed by qPCR were normalized relative to levels of human RPLPO or mouse RplpO expression.

### Reporter Plasmid Generation and Luciferase Assay

1.7 kb of human DD1αpromoter region was amplified by PCR and digested with SacI and Xhol, and was subsequently subcloned into the luciferase expression vector (pGL4.21[luc2P/Puro], Promega). Transciption start site is marked as +1. For 6 kb promoter-report construct, the upstream of DD1α promoter from 1.7 kb to 6 kb was amplified by PCR and digested with Kpnl and SacI, and then ligated into 1.7 kb DD1α promoter-luciferase construct. All constructs were verified by DNA sequencing. For luciferase assay, the luciferase reporter plasmids were transfected into cells. Vector encoding Renilla (pRL-TK) was also cotransfected for normalization of the transfection efficiency. The luciferase activity was determined using Dual Luciferase Reporter Assay System (Promega).

### Chromatin Immunoprecipitation (ChIP)

Chromatin immunoprecipiation of ionizing radiation-exposed MCF7 cells were performed following the manufacturer's protocol (Upstate). Immunoprecipitation was carried out at 4° C. overnight with anti-p53 polyclonal antibody and mouse IgG (Santa Cruz) as a negative control. Immunoprecipitated DNA was analyzed by qPCR using following primers. DD1α-BS1: 5′-ATATAGCAAGACCCCACCTCTACA-3′ (SEQ ID NO: 18), 5′-CACAGTGCAGCAACAAATAGAAGT-3′ (SEQ ID NO: 19) DD1α-B52: 5′-CCTCAGGCTCTGAATCTACAGTTA-3′ (SEQ ID NO: 20), 5′-GGGACAGAGACTTGTACCCTACAT-5′ (SEQ ID NO: 21) p21: 5′-CTCACATCCTCCTTCTTCAG-3′ (SEQ ID NO: 22), 5′-CACACACAGAATCTGACTCCC-3′ (SEQ ID NO: 23)

The amount of immunoprecipitated DNA was normalized to inputs.

### Electrophoretic Mobility Shift Assay

After ETO treatment, nuclear extracts were collected from MCF cells using general nuclear extraction method. 5 μg of nuclear extracts or purified recombinant p53 was incubated with 32P-labeled oligonucleotides in gel shift binding buffer (5 mM Tris-HCl at pH 7.5, 20 mM NaCl, 0.5 mM MgCl₂, 0.25 mM EDTA, 0.2% NP-40, 2.5% glycerol, 1 mM DTT, 20 μg/ml BSA and 40 μg/ml poly(dI-dC). The DNA-protein complexes were resolved by electrophoresis through 5% polyacrylamide gel containing 0.5% TBE. Gels were dried and exposed to X-ray film. Unlabeled wild type oligonucleoride or unlabeled mutant oligonucleotide was added in 100-fold excess as specific or non-specific competitior. Oligonucleotides corresponding to the DNA binding consensus sequence for human p53 (3, 4) were used.

(SEQ ID NO: 24)

| | |
|---|---|
| DD1α-BS1: | 5′-GCTGGGCATGGTGGTGCATGCCTGTA-3′ |

(SEQ ID NO: 25)

| | |
|---|---|
| DD1α-BS1 Mut: | 5′-GCTTTTTGCAGAGGTGTGCAAAGGTA-3′ |

(SEQ ID NO: 26)

| | |
|---|---|
| DD1α-BS2: | 5′-GTTAAACTGGCTCCAGCTTGCCTAGC-3′ |

(SEQ ID NO: 27)

| | |
|---|---|
| p21-BS: | 5′-TGTCCGGGCATGTCCGGGCATGTCCGGG-3′ |

(SEQ ID NO: 28)

| | |
|---|---|
| p21-BS Mut: | 5′-TGTCCGGGAATTTCCGGGAATTTCCGGG-3′ |

### Preparation of Human and Mouse Macrophages

For human monocyte-derived macrophage (h-MDM), human peripheral blood mononuclear cells were prepared from normal blood obtained from Massachusetts General Hospital blood donor center (protocol number 2012P002174). Monocytes were isolated by adhering mononuclear cells to culture plates for one hour at 37° C., after which non-adherent cells were removed by washing. The remaining cells were >95% CD14 positive. Adherent cells were then incubated in DMEM/F12 medium plus 10% FBS, 1% penicillin-streptomycin for 7 days to allow terminal differentiation of monocytes to macrophages. For mouse bone marrow-derived macrophages (m-BMDM), femurs and tibias were harvested from 5 to 6-week old mice and the marrow was flushed and placed into a sterile suspension of PBS. The bone marrow suspension was cultured in DMEM/F12 medium plus 10% FBS, 1% penicillin-streptomycin-glutamine with 40 ng/ml recombinant murine macrophage colony stimulating factor (M-CSF, Peprotech). Six days later, more than 90% of the adherent cells were CD11b positive and 80% of the cells were F4/80 positive.

### Generation of Apoptotic Cells.

Apoptosis of cancer cells were induced by CPT (0.5-20 μM). To generate apoptotic thymocytes, thymocytes isolated from mice were exposed to IR (2-100 Gy) with constant cell concentration of 1.0×10⁶ thymocytes/ml. For phagocytosis assays, apoptotic cells were labeled with pHrodo or CFSE (carboxyfluorescein diacetate succinimidyl ester) (Invitrogen). Cells were used when 60-70% of cells were apoptotic, as defined by annexin V-positive and propidium iodide-negative staining or TUNEL staining by flow cytometry.

### Phagocytosis Assay

For flow cytometry analysis of phagocytosis, 2.0×105 BMDMs were plated in the 24-well plate one day before the phagocytosis assay. Next day, the cells were starved for 30 minutes with DMEM/F12 containing 2% FBS and incubated with fluorescently labeled targets such as 4.0×106 pHrodo™(Invitrogen)-stained apoptotic thymocytes, lmg/ml pHrodo™ Red E. Coli BioParticles (Invitrogen) or 2-μm carboxylate-modified latex beads (invitrogen) in 150 μl of the uptake buffer (DMEM/F15 containing 2% FBS, 0.2% penicillin-streptomycin). After incubation for the indicated time, the cells were extensively washed with cold PBS, trypsinized and resuspended in cold medium containing 1% NaN3, and analyzed by flow cytometry. Forward and side-scatter parameters were used to distinguish unengulfed targets from phagocytes. The data was analyzed using FlowJo software. Fluorescent signal-positive BMDMs were considered to be phagocytes engulfing targets. For time-lapse image analysis of phagocytosis, CFSE (Invitrogen)-labeled apoptotic thymocytes were added to BMDM with 1:5 ratio (BMDM:thymocyte). The individual BMDMs were monitored by time-lapse confocal microscopy imaging (Nikon Eclipse Ti and Zeiss LSM 510), with images being taken at 1-2 min intervals. For image-based analysis of phagocytosis of human cancer cells, human monocyte-derived macrophage (MDM) were prepared from human peripheral blood and incubated with pHrodo™-labled apoptotic cancer cells with 1:10 ratio (MDM:cancer cell). 2 hours after coincubation, wells were washed thoroughly with cold serum-free RPMI five times and examined under fluorescence microscope (Nikon Eclipse Ti or Zeiss AxioObserver.Z1) using bright field or texas red filter set. Phagocytic index was calculated using the following formula: phagocytic index=number of ingested cells/(number of macrophages/100), as descried previously (5). At least 400 macrophages were counted per well.

### Generation and Genotyping of DD1α Knockout Mice

A targeting vector for the mouse DD1α gene was engineered by InGenious Targeting Laboratory, Inc. (Stony Brook, N.Y.). In brief, a PGK-neomycin cassette flanked by loxP and FRT sites was inserted downstream of exon 3. A third loxP site was inserted upstream of exon 2. The targeted iTL BA1 (C57BL/6×129/SvEv) hybrid embryonic stem cells containing the linearized construct were microinjected into C57BL/6 blastocysts. Germline transmission was achieved by backcrossing chimeras to wild-type C57BL/6 mice. To generate the whole body DD1α knockout mice, DD1α^{floxeno} mice were first bred with β-Actin/Flp mice (The Jackson Laboratory; Cat. No. 003800) to remove the PGK-neomycin cassette and then bred with EIIa-Cre mice (The Jackson Laboratory; Cat. No. 003724). Mice with DD1α heterozygous allele (DD1α^{+/-} were backcrossed with C57BL/6J mice for at least 7 generations before being intercrossed to generate mice homozygous for the null allele (DD1α−^{/-}. Routine genotyping was achieved on genomic DNA isolated from tail snips of mice with three primers to identify wild-type and null alleles: P1, 5′-TCCTTGTGCAGGACAGAGTT-3′ (SEQ ID NO: 29); P2, 5′-CTAATGGCACAGCAGGGACT-3′ (SEQ ID NO: 30); and P3, 5′-CAACAAATCACGGTGGAGTG-3′ (SEQ ID NO: 31). All animal experiments were approved by the Subcommittee on Research Animal Care of Massachusetts General Hospital (Protocol #2005N000022).

TABLE 1
Symptoms of DD1α-/- mice. DD1α-/- mice (40 females, 31 males) and control Wt mice (48
females, 37 males) were observed over a 19 month-period. Table 1 summarizes the symptoms (ulcerative
dermatitis, otitis, seizure, and eye lesion) and incidences of symptoms.

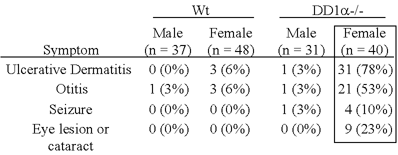

Analysis of Apoptotic Cell Clearance in vivo

Four-to-five-week-old Wt and DD1α−/− mice were exposed to 6.6 Gy of IR. At the indicated time points after exposure of IR, the mice were euthanized and thymuses and spleens were harvested. For quantification of total number of thymocytes in thymus, thymocytes were resuspended with PBS supplemented with 5% FBS. The cells were mixed with 30 μl of AccuCount Particles (Spherotech) and counted by flow cytometry. To monitor the TUNEL-positive apoptotic cells in thymus, 6 μm cryosections of whole thymuses were stained using the In situ Cell Death Red kit and DAPI according to manufacturer's instructions (Roche). Whole thymus images were obtained using automated image stitching method under fluorescence microscope (Olympus FSX100). To quantify the clearance of TUNEL-positive cells, the percentage of TUNEL-positive cells were determined by percent of the TUNEL-positive cells per DAPI-positive cells using imaging analysis program (CellSens Dimension, Olympus).

### Mouse Serologic and Urine Analysis

The levels of total IgG, ANA and ant-dsDNA in mouse serum were determined by Enzyme-linked immunosorbent assay according to the manufacturer's instructions (Bio-Rad) with mouse serum at a dilution of 1:10 to 1:100. For immunofluorescent analysis of ANA, HEp-2 slides were used. The images were analyzed by fluorescence microscope (Olympus FSX100). For mouse urine analysis, 24 h albuminuria was measured using metabolic cages. Of note, mice were fed a 5% sucrose drinking solution during the 24 h analysis in the metabolic cages. 10 μl of urine was analyzed by SDS-PAGE and Coomassie blue staining. Bovine serum albumin (0.25, 0.5, 1.0, 2.5 and 5.0 μg) served as standard. Signals were quantified using ImageJ software (NIH). Resulting values of the 5 multiplication of the ‘size of the area’ and the ‘mean gray value’ of each albumin standards were used for construction of a standard curve and its associated mathematical function. Values were translated into albumin concentrations and extrapolated to 24 h urine volume.

### Histology and Transmission Electron Microscopy

Organs were fixed by retrograde vascular perfusion with 4% paraformaldehyde in PBS, removed, and immersed in the same fixative for a maximum of 2 days until further processing for histology, immunohistochemistry or transmission electron microscopy (TEM) as previously described (6). For immunocomplex analysis, frozen sections were used. 4 μm paraffin-processed formalin-fixed tissue sections were stained with Periodic Acid Schiff (PAS) or hematoxylin/eosin (H&E). Images were taken with an Olympus BX53 microscope with DP72 camera and processed using Adobe PhotoShop software. Ultra-thin 80 nm sections of resin-embedded kidney tissue were mounted on copper grids, treated with uranyl acetate and lead citrate, and examined by a pathologist (A.W.) in a blinded fashion using a JEOL 1010 transmission electron microscope (Tokyo, Japan).

### Homophilic Interaction Assay

Protein A/G-purified DD1α-Ig proteins or Ig proteins (control) were covalently coupled to 6 μm blue carboxilated microparticles as recommended by the manufacturer's instructions (Polyscience, Inc.). 293T cells were transfected with the plasmids expressing full length DD1α, DD1α-ΔIgV or control empty vector as indicated in six-well culture plates for 24 hours. The transfected cells were incubated with the Ig proteins-coated beads in PBS containing 2% FBS at room temperature. After 30 min, unbound beads were washed with cold PBS and cell monolayers were examined under an inverted microscope and the binding was determined by measuring the optical density (O.D.) at 492 nm.

### In Situ Proximity Ligation Assay

The assay was performed with the use of a Duolink® using PLA® Technology (Sigma-Aldrich). J774.1 macrophages and ZR75-1 breast cancer cells were transfected with plasmids encoding DD1α-HA or DD1α-Myc, respectively for 24 hours. The transfected cells were trypsinized and co-cultured on slide glasses in six-well culture plates. After 24 hours, cells were fixed, permeabilized, blocked, and then incubated with rabbit anti-HA antibody and mouse anti-Myc antibody at 4° C. overnight. The cells were then subjected to an in situ proximity ligation assay with PLA probes, according to the manufacturer's protocol. All images were taken with a Nikon Eclipse Ti confocal microscope and processed using Adobe Photoshop software with minimal adjustment of brightness or contrast.

### In Vitro Binding Assay

His-fused DD1α (33-194) and GST-fused DD1α variants were purified from bacteria using Ni-NTA agarose (Life Technologies) or glutathione-sepharose beads (GE health), respectively. The His-fused DD1α (33-194) protein was indubated at 4° C. for 4 hours in a binding buffer containing 50 mM Tris, pH 7.5, 150 mM NaCl, 2 mM EDTA, 1 mM DTT, 0.1% NP-40, and 5 mg/ml BSA with GST-fused proteins immobilized on glutathione-agarose beads. The bound His-fused protein was eluted from the beads and analyzed by SDS-PAGE and western blot using anti-His antibody.

### Protein Lipid Overlay Assay

Membrane lipid strips (Echelon Bioscience) pre-spotted with 15 different biologically active lipids, at 100 pmol per spot were purchased. Nonspecific binding of membranes were blocked by incubation for 18 hours with 3% BSA in TBS-T (50 mM Tris at pH 7.4, 0.5 M NaCl and 0.1% Tween-20). After blocking, membranes were incubated for 2 hours with 2 μg/ml of soluble recombinant proteins such as DD1α-Ig purified from mammalian cell, DD1α-His purified from yeast, His-DD1α purified from bacteria and TIM-1-Ig, then were washed and incubated with indicated antibodies, followed by incubation for 1 hour with a horseradish peroxidase-labeled secondary antibody. Binding was detected by chemiluminescent detection.

### Human and Mouse T Cell Activation Assay

The cell culture plates were coated with anti-CD3 (OKT3, eBioscience for human T cells; 145-2C11, eBioscience for mouse T cells) and various concentrations of DD1α-Ig or PD-L1-Ig. The amount of Ig protein was kept constant at 10 μg/ml by the addition of control Ig protein. Human CD4+ or CD8+ T cells were purified from freshly isolated PBMCs using CD4+ T Cell Isolation Kit II, human or CD8+ T Cell Isolation Kit, human (Miltenyi Biotec). Mouse CD4+ and CD8+ T cells were purified from splenocytes freshly isolated from mice using CD4+ T Cell Isolation Kit II, mouse or CD8a+ T Cell Isolation Kit II, mouse (Miltenyi Biotec). Purity was confirmed to be over 90% by flow cytometry. The T cells were labeled with 1 μM CFSE, quenched by cold FBS and incubated in the anti-CD3 & Ig proteins-coated plates. On day 2 after stimulation, supernatant was collected and cytokines were assayed by ELISA. On day 3, cells were stained with anti-CD4-APC (OKT4, eBioscience for human CD4+ T cells; RM4-5, eBioscience for mouse CD4+ T cells) or anti-CD8-APC (SK1, eBioscience for human CD8+ T cells; 53-6.7, eBioscience for mouse CD8+ T cells) and then analyzed for CFSE dilution by flow cytometry.

iTreg Cell Development Assay

The cell culture plates were coated with anti-CD3 (145-2C11, eBioscience) together with mDD1α-Ig, mPD-L1-Ig or control Ig proteins. CD4+CD62L+naïve T cells were purified from splenocytes freshly isolated from Wt mice using CD4+CD62L+ T Cell Isolation Kit II (Miltenyi Biotec) and cultured in complete RPMI-1640 media added by anti-CD28 (37.51, eBioscience) and TGF-β (BioLegend) on the anti-CD3 & Ig proteins-coated plates. On day 3, cells were stained with anti-CD4-APC (RM4-5, eBioscience) and anti-mouse Foxp3-PE (FJK-16s, eBioscience) and analyzed for Foxp3 expression on CD4+ T cells by flow cytometry.

### Statistical Analysis.

Statistics were calculated using Prism 6.0 software (GraphPad Software). For analysis of statistical difference between experiments involving two groups, a Student's two-tailed t-test was applied. A one-way ANOVA was applied for statistical analysis of three or more groups. For survival curves, p values were obtained using a log-rank test from Kaplan-Meier survival curves. Significance was defined when P values were <0.05.

### REFERENCES FOR EXAMPLE 7

1. L. Fang, G. Li, G. Liu, S. W. Lee, S. A. Aaronson, p53 induction of heparin-binding EGF-like growth factor counteracts p53 growth suppression through activation of MAPK and PI3K/Akt signaling cascades. Embo J 20, 1931 (Apr. 17, 2001).
2. P. P. Ongusaha et al., RhoE is a pro-survival p53 target gene that inhibits ROCK I-mediated apoptosis in response to genotoxic stress. Curr Biol 16, 2466 (Dec. 19, 2006).
3. W. S. el-Deiry et al., WAF1, a potential mediator of p53 tumor suppression. Cell 75, 817 (Nov. 19, 1993).
4. T. D. Halazonetis, L. J. Davis, A. N. Kandil, Wild-type p53 adopts a ‘mutant’-like conformation when bound to DNA. Embo J 12, 1021 (March, 1993).
5. S. Jaiswal et al., CD47 is upregulated on circulating hematopoietic stem cells and leukemia cells to avoid phagocytosis. Cell 138, 271 (Jul. 23, 2009).
6. S. Hakroush et al., Effects of increased renal tubular vascular endothelial growth factor (VEGF) on fibrosis, cyst formation, and glomerular disease. Am J Pathol 175, 1883 (November, 2009).

1-48. (canceled)

49. A method of treating cancer in a subject diagnosed with cancer, the method comprising:
a. measuring the level of p53 activity or expression in a sample from a subject diagnosed with cancer;
b. comparing the level of p53 or expression in the sample with a p53 reference; and
c. when the level of p53 activity or expression is greater than the p53 reference, administering an anti-PD-1 therapy or an anti-DD1-α therapy to the subject, or, when the level of p53 activity or expression is the same as or less than the p53 reference, administering an alternative, proinflammatory immunotherapy without an anti-PD-1 therapy or an anti-DD1-α therapy.

50. The method of claim 49, wherein the alternative, proinflammatory immunotherapy comprises an activator of a proinflammatory T cell response pathway and/or a suppressor of an anti-inflammatory T cell response pathway.

51. The method of claim 50, wherein the activator of the proinflammatory T cell response and/or suppressor of the anti-inflammatory T cell response pathway comprises a TIGIT inhibitor, a Fgl2 inhibitor, a TIM-3 inhibitor, an anti-galectin-9 molecule, a CTLA-4 antagonist, a Lag-3 antagonist, an agonist of an immune checkpoint activating molecule, an antagonist of an immune checkpoint inhibitory molecule, or any combination thereof.

52. The method of claim 49, wherein the patient has been receiving an anti-cancer therapy or an immunotherapy.

53. The method of claim 49, wherein the p53 reference is the level of p53 activity or expression in a normal healthy subject.

54. The method of claim 49, wherein the p53 reference corresponds to the level of p53 activity or expression in a normal tissue of the same type or lineage as the sample.

55. The method of claim 49, wherein the sample is a blood sample or a tissue biopsy sample.

56. The method of claim 49, wherein the anti-PD-1 therapy comprises a PD-1 inhibitor, a PD-L1 inhibitor, or a PD-L2 inhibitor, and wherein the anti-DD1α therapy comprises an inhibitor of homophilic interactions between DD1αmolecules or an inhibitor of heterophilic interactions between DD1α molecules and PD-1 molecules.

57. The method of claim 56, wherein the inhibitor is selected from the group consisting of a protein, a peptide, a nucleic acid, an antibody, a small molecule, a vaccine, and combinations thereof.

58. A method treating an inflammatory disease or disorder, the method comprising:
a. measuring the level of p53 activity or expression in a sample from a patient diagnosed as having an inflammatory disease or disorder;
b. comparing the level of p53 activity or expression in the sample with a p53 reference; and
c. when the level of p53 activity or expression is lower than the p53 reference, administering a PD-1 agonist or a DD1-α agonist to the subject, or, when the level of p53 activity or expression is the same as or greater than the p53 reference, administering an alternative, anti-inflammatory immunotherapy without a DD1-α agonist or a PD-1 agonist.

59. The method of claim 58, wherein the alternative, anti-inflammatory immunotherapy comprises a suppressor of a proinflammatory T cell response pathway or an activator of an anti-inflammatory T cell response pathway, and wherein the suppressor of the proinflammatory T cell response or activator of the anti-inflammatory T cell response pathway comprises a TIGIT agonist, a Fgl2 agonist, a TIM-3 agonist, a galectin-9 molecule, a CTLA-4 agonist, a Lag-3 agonist, an antagonist of an immune checkpoint activating molecule, an agonist of an immune checkpoint inhibitory molecule, or any combination thereof.

60. The method of any of claims 16-19, wherein the patient has been receiving an immunotherapy.

61. A method of treating infection with a bacterial or fungal pathogen, the method comprising administering a treatment comprising an agent that antagonizes DD1α activity to a subject infected with said pathogen.

62. The method of claim 61, wherein said agent antagonizes the homophilic interaction of DD1α with DD1α.

63. The method of claim 61, wherein said agent antagonizes the functional interaction of DD1α with PD-1.

64. The method of claim 61, wherein said agent antagonizes the homophilic interaction of DD1α with DD1α and antagonizes the functional interaction of DD1α with PD-1.

65. The method of claim 63, wherein said agent comprises antigen-binding domains of antibodies that specifically bind DD1α and PD-1.

66. A method of treating cancer, the method comprising administering to a cancer patient in need thereof a treatment comprising an agent that antagonizes the homophilic interaction of DD1α with DD1α.

67. The method of claim 66, wherein said agent further antagonizes the functional interaction of DD1α with PD-1.

68. The method of claim 67, wherein the agent comprises antigen-binding domains of antibodies that specifically bind DD1α and PD-1.


SEQUENCE LISTING (39 sequences)

SEQ ID NO: 1 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 2 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 3 - DNA, 4774 bp, Homo sapiens
gggggcgggtgcctggagcacggcgctggggccgcccgcagcgctcactcgctcgcactcagtcgcgggaggcttccccgcgccggccgcgtcccgcccgctccccggcaccagaagttcctctgcgcgtccgacggcgacatgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcccaggatagtgaaaacatcacggctgcagccctggctacgggtgcctgcatcgtaggaatcctctgcctccccctcatcctgctcctggtctacaagcaaaggcaggcagcctccaaccgccgtgcccaggagctggtgcggatggacagcaacattcaagggattgaaaaccccggctttgaagcctcaccacctgcccaggggatacccgaggccaaagtcaggcaccccctgtcctatgtggcccagcggcagccttctgagtctgggcggcatctgctttcggagcccagcacccccctgtctcctccaggccccggagacgtcttcttcccatccctggaccctgtccctgactctccaaactttgaggtcatctagcccagctgggggacagtgggctgttgtggctgggtctggggcaggtgcatttgagccagggctggctctgtgagtggcctccttggcctcggccctggttccctccctcctgctctgggctcagatactgtgacatcccagaagcccagcccctcaacccctctggatgctacatggggatgctggacggctcagcccctgttccaaggattttggggtgctgagattctcccctagagacctgaaattcaccagctacagatgccaaatgacttacatcttaagaagtctcagaacgtccagcccttcagcagctctcgttctgagacatgagccttgggatgtggcagcatcagtgggacaagatggacactgggccaccctcccaggcaccagacacagggcacggtggagagacttctcccccgtggccgccttggctcccccgttttgcccgaggctgctcttctgtcagacttcctctttgtaccacagtggctctggggccaggcctgcctgcccactggccatcgccaccttccccagctgcctcctaccagcagtttctctgaagatctgtcaacaggttaagtcaatctggggcttccactgcctgcattccagtccccagagcttggtggtcccgaaacgggaagtacatattggggcatggtggcctccgtgagcaaatggtgtcttgggcaatctgaggccaggacagatgttgccccacccactggagatggtgctgagggaggtgggtggggccttctgggaaggtgagtggagaggggcacctgccccccgccctccccatcccctactcccactgctcagcgcgggccattgcaagggtgccacacaatgtcttgtccaccctgggacacttctgagtatgaagcgggatgctattaaaaactacatggggaaacaggtgcaaaccctggagatggattgtaagagccagtttaaatctgcactctgctgctcctcccccacccccaccttccactccatacaatctgggcctggtggagtcttcgcttcagagccattcggccaggtgcgggtgatgttcccatctcctgcttgtgggcatgccctggctttgtttttatacacataggcaaggtgagtcctctgtggaattgtgattgaaggattttaaagcaggggaggagagtagggggcatctctgtacactctgggggtaaaacagggaaggcagtgcctgagcatggggacaggtgaggtggggctgggcagaccccctgtagcgtttagcaggatgggggccccaggtactgtggagagcatagtccagcctgggcatttgtctcctagcagcctacactggctctgctgagctgggcctgggtgctgaaagccaggatttggggctaggcgggaagatgttcgcccaattgcttggggggttggggggatggaaaaggggagcacctctaggctgcctggcagcagtgagccctgggcctgtggctacagccagggaaccccacctggacacatggccctgcttctaagccccccagttaggcccaaaggaatggtccactgagggcctcctgctctgcctgggctgggccaggggctttgaggagagggtaaacataggcccggagatggggctgacacctcgagtggccagaatatgcccaaaccccggcttctcccttgtccctaggcagaggggggtcccttcttttgttccctctggtcaccacaatgcttgatgccagctgccataggaagagggtgctggctggccatggtggcacacacctgtcctcccagcactttgcagggctgaggtggaaggaccgcttaagcccaggtgttcaaggctgctgtgagctgtgttcgagccactacactccagcctggggacggagcaaaactttgcctcaaaacaaattttaaaaagaaagaaagaaggaaagagggtatgtttttcacaattcatgggggcctgcatggcaggagtggggacaggacacctgctgttcctggagtcgaaggacaagcccacagcccagattccggttctcccaactcaggaagagcatgccctgccctctggggaggctggcctggccccagccctcagctgctgaccttgaggcagagacaacttctaagaatttggctgccagaccccaggcctggctgctgctgtgtggagagggaggcggcccgcagcagaacagccaccgcacttcctcctcagcttcctctggtgcggccctgccctctcttctctggacccttttacaactgaacgcatctgggcttcgtggtttcctgttttcagcgaaatttactctgagctcccagttccatcttcatccatggccacaggccctgcctacaacgcactagggacgtccctccctgctgctgctggggaggggcaggctgctggagccgccctctgagttgcccgggatggtagtgcctctgatgccagccctggtggctgtgggctggggtgcatgggagagctgggtgcgagaacatggcgcctccagggggcgggaggagcactaggggctggggcaggaggctcctggagcgctggattcgtggcacagtctgaggccctgagagggaaatccatgcttttaagaactaattcattgttaggagatcaatcaggaattaggggccatcttacctatctcctgacattcacagtttaatagagacttcctgcctttattccctcccagggagaggctgaaggaatggaattgaaagcaccatttggagggttttgctgacacagcggggactgctcagcactccctaaaaacacaccatggaggccactggtgactgctggtgggcaggctggccctgcctgggggagtccgtggcgatgggcgctggggtggaggtgcaggagccccaggacctgcttttcaaaagacttctgcctgaccagagctcccactacatgcagtggcccagggcagaggggctgatacatggcctttttcagggggtgctcctcgcggggtggacttgggagtgtgcagtgggacagggggctgcaggggtcctgccaccaccgagcaccaacttggcccctggggtcctgcctcatgaatgaggccttccccagggctggcctgactgtgctgggggctgggttaacgttttctcagggaaccacaatgcacgaaagaggaactggggttgctaaccaggatgctgggaacaaaggcctcttgaagcccagccacagcccagctgagcatgaggcccagcccatagacggcacaggccacctggcccattccctgggcattccctgctttgcattgctgcttctcttcaccccatggaggctatgtcaccctaactatcctggaatgtgttgagagggattctgaatgatcaatatagcttggtgagacagtgccgagatagatagccatgtctgccttgggcacgggagagggaagtggcagcatgcatgctgtttcttggccttttctgttagaatacttggtgctttccaacacactttcacatgtgttgtaacttgtttgatccacccccttccctgaaaatcctgggaggttttattgctgccatttaacacagagggcaatagaggttctgaaaggtctgtgtcttgtcaaaacaagtaaacggtggaactacgactaaa

SEQ ID NO: 4 - DNA, 21 bp, Artificial Sequence
  Description of Artificial Sequence Synthetic oligonucleotide
gcagagacaacttctaagaat

SEQ ID NO: 5 - DNA, 21 bp, Artificial Sequence
  Description of Artificial Sequence Synthetic oligonucleotide
gcacgatgtgaccttctacaa

SEQ ID NO: 6 - DNA, 21 bp, Artificial Sequence
  Description of Artificial Sequence Synthetic primer
gatgtgaccttctacaagacg

SEQ ID NO: 7 - DNA, 20 bp, Artificial Sequence
  Description of Artificial Sequence Synthetic primer
gtcctggaacgtgaggttgc

SEQ ID NO: 8 - DNA, 22 bp, Artificial Sequence
  Description of Artificial Sequence Synthetic primer
ccaggatggttcttagactccc

SEQ ID NO: 9 - DNA, 21 bp, Artificial Sequence
  Description of Artificial Sequence Synthetic primer
tttagcacgaagctctccgat

SEQ ID NO: 10 - DNA, 21 bp, Artificial Sequence
  Description of Artificial Sequence Synthetic primer
tggcatttgctgaacgcattt

SEQ ID NO: 11 - DNA, 22 bp, Artificial Sequence
  Description of Artificial Sequence Synthetic primer
tgcagccaggtctaattgtttt

SEQ ID NO: 12 - DNA, 21 bp, Artificial Sequence
  Description of Artificial Sequence Synthetic primer
cagattggctacccaactgtt

SEQ ID NO: 13 - DNA, 21 bp, Artificial Sequence
  Description of Artificial Sequence Synthetic primer
gggaaggtgtaatccgtctcc

SEQ ID NO: 14 - DNA, 21 bp, Artificial Sequence
  Description of Artificial Sequence Synthetic primer
ggaaccctgctccttgctatt

SEQ ID NO: 15 - DNA, 21 bp, Artificial Sequence
  Description of Artificial Sequence Synthetic primer
ttgtagatggtcacatcgtgc

SEQ ID NO: 16 - DNA, 15 bp, Artificial Sequence
  Description of Artificial Sequence Synthetic primer
tcgggtcctagacca

SEQ ID NO: 17 - DNA, 22 bp, Artificial Sequence
  Description of Artificial Sequence Synthetic primer
agattcgggatatgctgttggc

SEQ ID NO: 18 - DNA, 24 bp, Artificial Sequence
  Description of Artificial Sequence Synthetic primer
atatagcaagaccccacctctaca

SEQ ID NO: 19 - DNA, 24 bp, Artificial Sequence
  Description of Artificial Sequence Synthetic primer
cacagtgcagcaacaaatagaagt

SEQ ID NO: 20 - DNA, 24 bp, Artificial Sequence
  Description of Artificial Sequence Synthetic primer
cctcaggctctgaatctacagtta

SEQ ID NO: 21 - DNA, 24 bp, Artificial Sequence
  Description of Artificial Sequence Synthetic primer
gggacagagacttgtaccctacat

SEQ ID NO: 22 - DNA, 20 bp, Artificial Sequence
  Description of Artificial Sequence Synthetic primer
ctcacatcctccttcttcag

SEQ ID NO: 23 - DNA, 21 bp, Artificial Sequence
  Description of Artificial Sequence Synthetic primer
cacacacagaatctgactccc

SEQ ID NO: 24 - DNA, 26 bp, Artificial Sequence
  Description of Artificial Sequence Synthetic primer
gctgggcatggtggtgcatgcctgta

SEQ ID NO: 25 - DNA, 26 bp, Artificial Sequence
  Description of Artificial Sequence Synthetic primer
gctttttgcagaggtgtgcaaaggta

SEQ ID NO: 26 - DNA, 26 bp, Artificial Sequence
  Description of Artificial Sequence Synthetic primer
gttaaactggctccagcttgcctagc

SEQ ID NO: 27 - DNA, 28 bp, Artificial Sequence
  Description of Artificial Sequence Synthetic primer
tgtccgggcatgtccgggcatgtccggg

SEQ ID NO: 28 - DNA, 28 bp, Artificial Sequence
  Description of Artificial Sequence Synthetic primer
tgtccgggaatttccgggaatttccggg

SEQ ID NO: 29 - DNA, 20 bp, Artificial Sequence
  Description of Artificial Sequence Synthetic primer
tccttgtgcaggacagagtt

SEQ ID NO: 30 - DNA, 20 bp, Artificial Sequence
  Description of Artificial Sequence Synthetic primer
ctaatggcacagcagggact

SEQ ID NO: 31 - DNA, 20 bp, Artificial Sequence
  Description of Artificial Sequence Synthetic primer
caacaaatcacggtggagtg

SEQ ID NO: 32 - DNA, 20 bp, Artificial Sequence
  Description of Artificial Sequence Synthetic oligonucleotide
rrrcwwgyyyrrrcwwgyyy

SEQ ID NO: 33 - DNA, 20 bp, Artificial Sequence
  Description of Artificial Sequence Synthetic oligonucleotide
gggcatggtggtgcatgcct

SEQ ID NO: 34 - protein, 124 aa, Homo sapiens
PEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQV

SEQ ID NO: 35 - protein, 94 aa, Homo sapiens
EVGQNAYLPCFYTPAAPGNLVPVCWGKGACPVFECGNVVLRTDERDVNYWTSRYWLNGDFRKGDVSLTIENVTLADSGIYCCRIQIPGIMNDEK

SEQ ID NO: 36 - protein, 94 aa, Homo sapiens; identical to SEQ ID NO: 35

SEQ ID NO: 37 - protein, 98 aa, Homo sapiens
AAGESAILHCTVTSLIPVGPIQWFRGAGPARELIYNQKEGHFPRVTTVSESTKRENMDFSISISNITPADAGTYYCVKFRKGSPDTEFKSGAGTELSV

SEQ ID NO: 38 - protein, 101 aa, Homo sapiens
VLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNL

SEQ ID NO: 39 - protein, 100 aa, Homo sapiens
EYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKV